(19) United States
(12) Patent Application Publication
(10) Pub. No.: US 2025/0109398 A1
(43) Pub. Date: Apr. 3, 2025

(54) COMPOSITIONS FOR TREATING NEUROLOGICAL DISEASE

(71) Applicant: ASKLEPIOS BIOPHARMACEUTICAL, INC., Research Triangle Park, NC (US)

(72) Inventors: Anna TRETIAKOVA, Research Triangle Park, NC (US); Lester SUAREZ, Research Triangle Park, NC (US); Anne BRAAE, Research Triangle Park, NC (US); Michael L. ROBERTS, Research Triangle Park, NC (US); Caroline PEDDLE, Research Triangle Park, NC (US); Ileana GUERRINI, Research Triangle Park, NC (US); Juan Manuel IGLESIAS, Research Triangle Park, NC (US); Jorge Omar YANEZ-CUNA, Research Triangle Park, NC (US); Philippe MOULLIER, Research Triangle Park, NC (US)

(73) Assignee: ASKLEPIOS BIOPHARMACEUTICAL, INC., Research Triangle Park, NC (US)

(21) Appl. No.: 18/833,081

(22) PCT Filed: Jan. 27, 2023
(86) PCT No.: PCT/US2023/011746

§ 371 Date: Jul. 25, 2024

Related U.S. Application Data
(60) Provisional application No. 63/321,335, filed on Mar. 18, 2022.
(60) Provisional application No. 63/303,673, filed on Jan. 27, 2022.

(51) Int. Cl. C12N 15/113; A61K 38/44; A61K 48/00; A61P 25/28; C12N 15/86

(52) U.S. Cl. CPC C12N 15/113; A61K 38/44; A61K 48/0066; A61P 25/28; C12N 15/86; C12N 2310/141; C12N 2750/14143; C12N 2750/14171; C12N 2830/008

(57) ABSTRACT

Aspects of the disclosure relate to compositions and methods useful for treating neurological diseases and disorders. In some embodiments, the disclosure provides a method for treating a neurological disease or disorder comprising administration of both a viral vector comprising interfering nucleic acids (e.g., artificial miRNAs) and a viral vector comprising a CYP46A1 protein. In some embodiments, the disclosure provides a method for treating Huntington's disease comprising administration of both a viral vector comprising interfering nucleic acids (e.g., artificial miRNAs) targeting the huntingtin gene (HTT) and a viral vector comprising a CYP46A1 protein. In some embodiments, the viral vector comprises a modified viral capsid, such as for preferentially targeting cells in the CNS or PNS.

### CROSS-REFERENCE TO RELATED APPLICATIONS

This application claims benefit under 35 U.S.C. § 119(e) of U.S. Provisional Application No. 63/303,673 filed Jan. 27, 2022 and U.S. Provisional Application No. 63/321,335 filed Mar. 18, 2022, the contents of each of which are incorporated herein by reference in their entireties.

### TECHNICAL FIELD

The technology described herein relates to methods for treating neurological diseases or disorders, e.g., Huntington's disease.

### BACKGROUND

Huntington's disease (HD) is a devastating inherited neurodegenerative disease caused by an expansion of the CAG repeat region in exon 1 of the huntingtin gene. While the Huntingtin protein (HTT) is expressed throughout the body, the polyglutamine expanded protein is especially toxic to medium spiny neurons in the striatum and their cortical connections. Patients struggle with emotional symptoms including depression and anxiety and with characteristic movement disturbances and chorea. There is currently no cure for Huntington's disease; therapeutic options are limited to ameliorating disease symptoms.

### SUMMARY

One aspect provided herein describes a method for treating a neurological disease or disorder in a subject in need thereof, the method comprising administering to a subject having or at risk of developing the neurological disease or disorder a therapeutically effective amount of at least one of: (a) a nucleic acid encoding at least one miRNA; and (b) a nucleic acid encoding a CYP46A1 protein. For example, a nucleic acid encoding at least one miRNA is administered alone, or is administered in combination with a nucleic acid encoding a CYP46A1 protein. Alternatively, a nucleic acid encoding a CYP46A1 protein is administered alone, or is administered in combination with a nucleic acid encoding at least one miRNA. The nucleic acid can be wild type or a codon-optimized variant.

Another aspect described herein provides a composition or combination comprising at least one of (a) an isolated nucleic acid encoding a transgene encoding one or more miRNAs; and (b) an isolated nucleic acid encoding a CYP46A1 protein. In one aspect, described herein is a composition or combination comprising: (a) a recombinant viral vector comprising an isolated nucleic acid comprising (i) a first region comprising a first adeno-associated virus (AAV) inverted terminal repeat (ITR), or a variant thereof, and (ii) a second region comprising a transgene encoding one or more miRNAs; and (b) a recombinant viral vector comprising an isolated nucleic acid encoding the CYP46A1 protein.

Another aspect described herein provides a composition or combination comprising (a) an isolated nucleic acid encoding a transgene encoding an isolated nucleic acid encoding a CYP46A1 protein. In some aspects, the composition or combination further comprises (b) an isolated nucleic acid encoding a transgene encoding one or more miRNAs. In other aspects, the composition or combination does not comprise (b) an isolated nucleic acid encoding a transgene encoding one or more miRNAs. In one aspect, described herein is a composition or combination comprising: (a) a recombinant viral vector comprising an isolated nucleic acid comprising (i) a first region comprising a first adeno-associated virus (AAV) inverted terminal repeat (ITR), or a variant thereof, and (ii) a second region comprising a transgene encoding a CYP46A1 protein. In some aspects, the composition or combination further comprises: (b) a recombinant viral vector comprising an isolated nucleic acid encoding one or more miRNAs. In other aspects, the composition or combination does not comprise (b) a recombinant viral vector comprising an isolated nucleic acid encoding one or more miRNAs.

Another aspect described herein provides a composition or combination comprising (a) an isolated nucleic acid encoding a transgene encoding an isolated nucleic acid encoding one or more miRNAs. In some aspects, the composition or combination further comprises (b) an isolated nucleic acid encoding a transgene encoding a CYP46A1 protein. In other aspects, the composition or combination does not comprise (b) an isolated nucleic acid encoding a transgene encoding a CYP46A1 protein. In one aspect, described herein is a composition or combination comprising: (a) a recombinant viral vector comprising an isolated nucleic acid comprising (i) a first region comprising a first adeno-associated virus (AAV) inverted terminal repeat (ITR), or a variant thereof, and (ii) a second region comprising a transgene encoding one or more miRNAs. In some aspects, the composition or combination further comprises: (b) a recombinant viral vector comprising an isolated nucleic acid encoding a CYP46A1 protein. In other aspects, the composition or combination does not comprise (b) a recombinant viral vector comprising an isolated nucleic acid encoding a CYP46A1 protein.

Another aspect described herein provides a method for treating a neurological disease or disorder in a subject in need thereof, the method comprising administering to a subject having or at risk of developing the neurological disease or disorder a therapeutically effective amount of at least one of (a) an isolated nucleic acid encoding a transgene encoding one or more miRNAs; and (b) an isolated nucleic acid encoding a CYP46A1 protein. In one aspect, described herein is a method for treating a neurological disease or disorder in a subject in need thereof, the method comprising administering to a subject having or at risk of developing the neurological disease or disorder a therapeutically effective amount of at least one of (a) a recombinant viral vector comprising an isolated nucleic acid comprising (i) a first region comprising a first adeno-associated virus (AAV) inverted terminal repeat (ITR), or a variant thereof, and (ii) a second region comprising a transgene encoding one or more miRNAs; and (b) a recombinant viral vector comprising an isolated nucleic acid encoding the CYP46A1 protein.

Another aspect described herein provides a method for treating a neurological disease or disorder in a subject in need thereof, the method comprising administering to a subject having or at risk of developing the neurological disease or disorder a therapeutically effective amount of (a) an isolated nucleic acid encoding a transgene encoding a CYP46A1 protein. In some aspects, the method further comprises administering to the subject a therapeutically effective amount of (b) an isolated nucleic acid encoding a transgene encoding one or more miRNAs. In other aspects, the method does not comprise administering to the subject (b) an isolated nucleic acid encoding a transgene encoding one or more miRNAs. In one aspect, described herein is a method for treating a neurological disease or disorder in a subject in need thereof, the method comprising administering to a subject having or at risk of developing the neurological disease or disorder a therapeutically effective amount of (a) a recombinant viral vector comprising an isolated nucleic acid comprising (i) a first region comprising a first adeno-associated virus (AAV) inverted terminal repeat (ITR), or a variant thereof, and (ii) a second region comprising a transgene encoding a CYP46A1 protein. In some aspects, the method further comprises administering to the subject a therapeutically effective amount of (b) a recombinant viral vector comprising an isolated nucleic acid encoding one or more miRNAs. In other aspects, the method does not comprise administering to the subject a therapeutically effective amount of (b) a recombinant viral vector comprising an isolated nucleic acid encoding one or more miRNAs.

Another aspect described herein provides a method for treating a neurological disease or disorder in a subject in need thereof, the method comprising administering to a subject having or at risk of developing the neurological disease or disorder a therapeutically effective amount of (a) an isolated nucleic acid encoding a transgene encoding one or more miRNAs. In some aspects, the method further comprises administering to the subject a therapeutically effective amount of (b) an isolated nucleic acid encoding a transgene encoding a CYP46A1 protein. In other aspects, the method does not comprise administering to the subject (b) an isolated nucleic acid encoding a transgene encoding a CYP46A1 protein. In one aspect, described herein is a method for treating a neurological disease or disorder in a subject in need thereof, the method comprising administering to a subject having or at risk of developing the neurological disease or disorder a therapeutically effective amount of (a) a recombinant viral vector comprising an isolated nucleic acid comprising (i) a first region comprising a first adeno-associated virus (AAV) inverted terminal repeat (ITR), or a variant thereof, and (ii) a second region comprising a transgene encoding one or more miRNAs. In some aspects, the method further comprises administering to the subject a therapeutically effective amount of (b) a recombinant viral vector comprising an isolated nucleic acid encoding a CYP46A1 protein. In other aspects, the method does not comprise administering to the subject a therapeutically effective amount of (b) a recombinant viral vector comprising an isolated nucleic acid encoding a CYP46A1 protein.

In some embodiments, the neurological disease or disorder is Alzheimer's disease, Parkinson's disease, Huntington's disease, Canavan disease, Leigh's disease, spinal cerebral ataxia, polyglutamine repeat spinocerebellar ataxia, Krabbe's disease, Batten's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, neuropathic pain, trauma due to spinal cord or head injury, ophthalmic diseases and disorders, Tay-Sachs disease, Lesch-Nyhan disease, epilepsy, cerebral infarcts, depression, bipolar affective disorder, persistent affective disorder, secondary mood disorder, schizophrenia, drug dependency, neuroses, psychosis, dementia, paranoia, attention deficit disorder, psychosexual disorders, sleeping disorders, pain disorders, eating or weight disorders. In some embodiments, the neurological disease or disorder is a central nervous system (CNS) disease or disorder. In some embodiments, the CNS disease or disorder is selected from Huntington's disease, Alzheimer's disease, Polyglutamine repeat spinocerebellar ataxias, Amyotrophic lateral sclerosis and Parkinson's disease.

In some embodiments, the CNS disease or disorder is Alzheimer's disease and the at least one miRNA comprises a seed sequence complementary to Amyloid Precursor Protein (APP), Presenilin 1, Presenilin 2, ABCA7, SORL1, and disease-associated alleles thereof.

In some embodiments, the CNS disease or disorder is Parkinson's disease and the at least one miRNA comprises a seed sequence complementary to SNCA, LRRK2/PARK8, PRKN, PINK1, DJ1/PARK7, VPS35, EIF4G1, DNAJC13, CHCHD2, UCHL1, GBA1, and disease-associated alleles thereof.

In some embodiments, the CNS disease is Huntington's disease and at least one miRNA comprises a seed sequence complementary to SEQ ID NO: 4, or wherein at least one miRNA comprises the sequence of any one of SEQ ID NOs: 6-17, 40-44, 50-66, 158-185, or 217-260 flanked by a miRNA backbone sequence. In some embodiments, the CNS disease is Huntington's disease and at least one miRNA comprises the sequence of any one of SEQ ID NOs: 6-17, 40-44, 50-66, 158-185, or 217-260. In some embodiments, at least one of the miRNAs hybridizes with and inhibits expression of human huntingtin. In some embodiments, the subject comprises a huntingtin gene having more than 36 CAG repeats, more than 40 repeats, or more than 100 repeats. In some embodiments, the subject is less than 20 years of age.

In some embodiments, the recombinant viral vector is selected from the group consisting of: an AAV vector, an adenovirus vector, a lentivirus vector, a retrovirus vector, a herpesvirus vector, an alphavirus vector, a poxvirus vector, a baculovirus vector, and a chimeric virus vector.

In some embodiments, the recombinant viral vector comprising (a) is the same as the recombinant viral vector comprising (b). In some embodiments, the isolated nucleic acid of (a) and (b) are comprised in separate recombinant viral vectors. In some embodiments, the isolated nucleic acid of (a) and (b) are comprised in the same recombinant viral vector.

In some embodiments, (a) and (b) are administered at substantially the same time. In some embodiments, (a) and (b) are administered at different time points. In some embodiments, the different time points are spaced by at least 1 min, at least 1 hour, at least 1 day, at least 1 week, at least 1 month, at least 1 year, or more. In some embodiments, (a) is administered prior to the administration of (b). In some embodiments, (b) is administered prior to the administration of (a). In some embodiments, the administration of (a), (b), or (a) and (b) is repeated at least once.

In some embodiments, the transgene comprises two miRNAs in tandem that are flanked by introns. In some embodiments, the flanking introns are identical. In some embodiments, the flanking introns are from the same species. In some embodiments, the flanking introns are hCG introns.

In some embodiments, the transgene comprises a promoter. In some embodiments, the promoter is a synapsin (Syn1) promoter, or a promoter of Tables 10-13.

In some embodiments, the one or more miRNAs are located in an untranslated portion of the transgene. In some embodiments, the untranslated portion is an intron. In some embodiments, the untranslated portion is between the last codon of the nucleic acid sequence encoding a protein and a poly-A tail sequence, or between the last nucleotide base of a promoter sequence and a poly-A tail sequence.

In some embodiments, the nucleic acid or viral vector further comprises a third region comprising a second adeno-associated virus (AAV) inverted terminal repeat (ITR), or a variant thereof.

In some embodiments, the ITR variant lacks a functional terminal resolution site (TRS), optionally wherein the ITR variant is a ATRS ITR.

In some embodiments, the administration results in delivery of the viral vector or isolated nucleic acid to the central nervous system (CNS) of the subject. In some embodiments, the administration is via injection, optionally intravenous injection or intrastriatal injection.

In some embodiments, the viral vector is AAV1, AAV2, AAV3b, AAV4, AAV5, AAV6, AAV7, AAV8, AAV9, AAV10, AAVrh10, AAV11, or, AAV12, or a chimera thereof. In some embodiments, the viral vector comprises a capsid protein from AAV serotype AAV1, AAV2, AAV3b, AAV4, AAV5, AAV6, AAV7, AAV8, AAV9, AAV10, AAVrh10, AAV11, or, AAV12, or a chimera thereof. In some embodiments, the capsid protein is an AAV9 capsid protein. In some embodiments, the viral vector is a self-complementary AAV (scAAV). In some embodiments, the viral vector is formulated for delivery to the central nervous system (CNS).

In some embodiments of any of the aspects, the viral vector comprises a modified viral capsid.

In some embodiments of any of the aspects, the viral vector comprises a modification to a viral capsid.

In some embodiments of any of the aspects, the modification is a chemical, non-chemical or amino acid modification of the viral capsid.

In some embodiments of any of the aspects, at least one of the capsid modifications preferentially targets cells in the CNS or PNS.

In some embodiments of any of the aspects, the chemical modification comprises a chemically-modified tyrosine residue modified to comprise a covalently-linked mono- or polysaccharide moiety.

In some embodiments of any of the aspects, the chemically-modified tyrosine residue comprises a mono-saccharide selected from galactose, mannose, N-acetylgalactosamine, bridge GalNac, and mannose-6-phosphate.

In some embodiments of any of the aspects, the chemical modification comprises a ligand covalently linked to a primary amino group of a capsid polypeptide via a —CSNH— bond.

In some embodiments of any of the aspects, the ligand comprises an arylene or heteroarylene radical covalently bound to the ligand.

In some embodiments of any of the aspects, the modified viral capsid is a chimeric capsid or a haploid capsid.

In some embodiments of any of the aspects, the modified viral capsid is a haploid capsid.

In some embodiments of any of the aspects, the modified viral capsid is a chimeric or haploid capsid further comprising a modification.

In some embodiments of any of the aspects, the modified viral capsid is an AAV serotype AAV1, AAV2, AAV3b, AAV4, AAV5, AAV6, AAV7, AAV8, AAV9, AAV10, AAVrh10, AAV11, AAV12, or a mutant modified from, a chimera, a mosaic, or a rational haploid thereof.

In some embodiments of any of the aspects, the modification changes the antigenic profile of the modified viral capsid as compared to the unmodified viral capsid.

In some embodiments of any of the aspects, the modified viral capsid can be used for repeat administration.

### BRIEF DESCRIPTION OF THE DRAWINGS

FIG. 1 is a schematic showing an HD plasmid map of pJAL130-CYP46A1, 7314 bp, see e.g., SEQ ID NO: 111 and Table 16, which shows the ITR to ITR sequence of the codon-optimized CYP46 sequence (see e.g., SEQ ID NO: 110) from the plasmid.

FIG. 2 shows the intracranial biodistribution in sagittal sections of the transgene GFP under the control of CNS-1 (see e.g., SEQ ID NO: 112), CNS-2 (see e.g., SEQ ID NO: 113), CNS-3 (see e.g., SEQ ID NO: 114), CNS-4 (see e.g., SEQ ID NO: 115), CNS-5 (see e.g., SEQ ID NO: 122), CNS-6 (see e.g., SEQ ID NO: 123), CNS-7 (see e.g., SEQ ID NO: 124) and CNS-8 (see e.g., SEQ ID NO: 125) and the control promoter hSyn1 (see e.g., SEQ ID NO: 152) delivered by intracerebroventricular (ICV) and intravenous (IV) injection. Scale bar is 1 mm.

FIG. 3A-3B show images of coronal bran sections. FIG. 3A shows the intracranial biodistribution in coronal sections of the transgene GFP under the control of CNS-1 (see e.g., SEQ ID NO: 112), CNS-2 (see e.g., SEQ ID NO: 113), CNS-3 (see e.g., SEQ ID NO: 114) and CNS-4 (see e.g., SEQ ID NO: 115) delivered by ICV. Scale bar is 1 mm. FIG. 3B shows the intracranial biodistribution in coronal sections of the transgene GFP under the control of CNS-5 (see e.g., SEQ ID NO: 122), CNS-6 (see e.g., SEQ ID NO: 123), CNS-7 (see e.g., SEQ ID NO: 124) and CNS-8 (see e.g., SEQ ID NO: 125) and the control promoter hSyn1 (see e.g., SEQ ID NO: 152) delivered by ICV. Scale bar is 1 mm.

FIG. 4 shows percentage GFP immunoreactivity in different brain regions following ICV or IV delivery of GFP driven by CNS 1-8 (see e.g., SEQ ID NOs: 112-115, 122-125) or Synapsin-1 (see e.g., SEQ ID NO: 152). The data was obtained by quantitative measurement of 10 non-overlapping RGB images of GFP staining intensity by thresholding analysis in cortex, hippocampus, striatum, midbrain and cerebellum (mean±SEM). Images were taken at ×40 magnification through discrete brain regions keeping constant settings. The foreground immunostaining was defined by averaging of the highest and lowest signals. Data is represented as the mean percentage area of immunoreactivity per field for each region of interest (n=3). With ICV delivery, expression is highest in cortex and hippocampal brain regions. CNS 1-8 (see e.g., SEQ ID NO: 112-115, 122-125) show higher expression in the hippocampus than hSyn1 control. CNS-1 (see e.g., SEQ ID NO: 112) shows higher expression in hippocampus, midbrain and cerebellum compared to hSyn1 with ICV delivery.

FIG. 5A-5B show the tissue expression pattern for the faf1 and pitx3 genes from which the CRE/proximal promoter from CNS-5, CNS-5_v2, CNS-2, CNS-3 and CNS-4 were designed. FIG. 5A shows the expression pattern of the faf1 gene in mouse PNS neurons from single cell transcriptomic data (Zeisel et al., 2018). Dark grey denotes high expression, white denotes no expression and light grey denotes low expression. faf1 is expressed in many PNS neurons. FIG. 5B shows the expression pattern of the pitx3 gene in PNS neurons from single cell transcriptomic data (Zeisel et al., 2018). Dark grey denotes high expression, white denotes no expression and light grey denotes low expression. pixt3 is expressed in sympathetic PNS neurons. faf1 is expressed in many PNS neurons so a synthetic promoter comprising CRE or proximal promoter designed from the faf1 gene such as CNS-5 and CNS-5 v2 is expected to have strong expression in the PNS. pitx3 is expressed in sympathetic PNS neurons so a synthetic promoter comprising CRE designed from the pitx3 gene such as CNS-2, CNS-3 or CNS-4 is expected to have expression in PNS sympathetic neurons. Similar analysis for lmx1b and pitx2 revealed no expression in PNS above the cut off score for the analysis (trinization score of less than 0.95; data not shown) so CNS-1, CNS-6, CNS-6_v2, CNS-7, CNS-7_v2, CNS-8 and CNS-8_v2 are not expected to be active in PNS neurons.

FIG. 6A shows the expression pattern of the HTT gene in a sagittal section from an adult mouse brain (taken from the Allen Mouse brain atlas; mouse.brain-map.org). HTT (huntingtin) is highly expressed in throughout the brain.

FIG. 6B shows the expression pattern of the CYP46A1 gene in a coronal section from an adult mouse brain (taken from the Allen Mouse brain atlas; available on the world wide web at mouse.brain-map.org). CYP46A1 is widely expressed in the brain.

FIG. 7A shows the median GFP expression of synthetic NS-specific promoters SP0013, SP0014, SP0030, SP0031, SP0032, SP0019, SP0020, SP0021, SP0022, SP0011, SP0034, SP0035, SP0036 and control promoters Synapsin-1 relative to control promoter CAG in neuroblastoma-derived SH-SY5Y cells. NTC denotes non-transfected cells. The data is collected from three biological replicates, each of which is the average of two technical replicates. Error bars are standard error.

FIG. 7B shows the transfection efficiency in neuroblastoma-derived SH-SY5Y cells when transfected with synthetic NS-specific promoters SP0013, SP0014, SP0030, SP0031, SP0032, SP0019, SP0020, SP0021, SP0022, SP0011, SP0034, SP0035, SP0036 or control promoters Synapsin-1 and CAG, operably linked to GFP. NTC denotes non-transfected cells. The data is collected from three biological replicates, each of which is the average of two technical replicates. Error bars are standard error. GFP positive % denotes the % of all cells which were GFP positive.

FIG. 8 shows levels of HTT transcript following nucleofection of patient-derived fibroblasts with plasmids containing miRNAs targeting exon 1 of the HTT gene. N=1.

FIG. 9 is a schematic showing an HD plasmid map of dbDNA-CYP46A1, 4,573 bp, see e.g., SEQ ID NO: 194, which shows the ITR to ITR sequence comprising the codon-optimized CYP46 sequence (see e.g., SEQ ID NO: 212), CAG promoter, and bGH poly(a) signal (see, e.g., Table 24) from the plasmid. This is plasmid is packed in rAAVrh.10 for administration into HD patients (see, e.g., Example 6).

### DETAILED DESCRIPTION

Aspects of the invention relate to administration of both an interfering RNA (e.g., miRNAs, such as artificial miRNAs) that when delivered to a subject are effective for reducing the expression of a pathogenic gene in the subject, and a nucleic acid encoding a CYP46A1 protein. Accordingly, methods and compositions described by the disclosure are useful, in some embodiments, for the treatment of neurological diseases or disorders.

### Treatment Methods

Methods for delivering a nucleic acid and/or a transgene (e.g., an inhibitory RNA, such as a miRNA or a nucleic acid encoding CYP46A1) to a subject are provided by the disclosure. The methods typically involve administering to a subject an effective amount of at least one of a nucleic acid encoding at least one interfering RNA/inhibitory nucleic acid capable of reducing expression of a target gene, e.g., a pathogenic gene associated with a neurological disease or disorder (e.g., huntingtin (htt) protein) and a nucleic acid encoding CYP46A1. For example, a nucleic acid encoding at least one miRNA is administered alone, or is administered in combination with a nucleic acid encoding a CYP46A1 protein. Alternatively, a nucleic acid encoding a CYP46A1 protein is administered alone, or is administered in combination with a nucleic acid encoding at least one miRNA. In some embodiments, one or both of the nucleic acids are provided in a viral vector and/or in a viral particle, e.g., a rAAV.

Accordingly, one aspect described herein provides a method for treating a neurological disease or disorder in a subject in need thereof, the method comprising administering to a subject having or at risk of developing the neurological disease or disorder a therapeutically effective amount of (a) an isolated nucleic acid encoding a transgene encoding a CYP46A1 protein. In some aspects, the method further comprises administering to the subject a therapeutically effective amount of (b) an isolated nucleic acid encoding a transgene encoding one or more miRNAs. In other aspects, the method does not comprise administering to the subject (b) an isolated nucleic acid encoding a transgene encoding one or more miRNAs. In one aspect, described herein is a method for treating a neurological disease or disorder in a subject in need thereof, the method comprising administering to a subject having or at risk of developing the neurological disease or disorder a therapeutically effective amount of (a) a recombinant viral vector comprising an isolated nucleic acid comprising (i) a first region comprising a first adeno-associated virus (AAV) inverted terminal repeat (ITR), or a variant thereof, and (ii) a second region comprising a transgene encoding a CYP46A1 protein. In some aspects, the method further comprises administering to the subject a therapeutically effective amount of (b) a recombinant viral vector comprising an isolated nucleic acid encoding one or more miRNAs. In other aspects, the method does not comprise administering to the subject a therapeutically effective amount of (b) a recombinant viral vector comprising an isolated nucleic acid encoding one or more miRNAs.

Another aspect described herein provides a method for treating a neurological disease or disorder in a subject in need thereof, the method comprising administering to a subject having or at risk of developing the neurological disease or disorder a therapeutically effective amount of (a) an isolated nucleic acid encoding a transgene encoding one or more miRNAs. In some aspects, the method further comprises administering to the subject a therapeutically effective amount of (b) an isolated nucleic acid encoding a transgene encoding a CYP46A1 protein. In other aspects, the method does not comprise administering to the subject (b) an isolated nucleic acid encoding a transgene encoding a CYP46A1 protein. In one aspect, described herein is a method for treating a neurological disease or disorder in a subject in need thereof, the method comprising administering to a subject having or at risk of developing the neurological disease or disorder a therapeutically effective amount of (a) a recombinant viral vector comprising an isolated nucleic acid comprising (i) a first region comprising a first adeno-associated virus (AAV) inverted terminal repeat (ITR), or a variant thereof, and (ii) a second region comprising a transgene encoding one or more miRNAs. In some aspects, the method further comprises administering to the subject a therapeutically effective amount of (b) a recombinant viral vector comprising an isolated nucleic acid encoding a CYP46A1 protein. In other aspects, the method does not comprise administering to the subject a therapeutically effective amount of (b) a recombinant viral vector comprising an isolated nucleic acid encoding a CYP46A1 protein.

As used herein, “neurological disease or disorder” can refer to any disease, disorder, or condition affecting or associated with the nervous system, i.e. those that affect the central nervous system (brain and spinal cord), the peripheral nervous system (PNS; e.g., peripheral nerves and cranial nerves), and the autonomic nervous system (parts of which are located in both central and peripheral nervous systems). More than 600 neurological diseases have been identified in humans. By way of non-limiting examples, the neurological disease or disorder can be Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Subcortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavemous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia-Multi-Infarct, Dementia—Semantic, Dementia-Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Alzheimer's disease, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kliiver-Bucy Syndrome, Korsakoff s Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus—Neurological Sequelae, Lyme Disease—Neurological Complications, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myoclonic Encephalopathy of Infants, Myoclonus, Myopathy, Myopathy-Congenital, Myopathy-Thyrotoxic, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy-Hereditary, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain-Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, polyglutamine repeat spinocerebellar ataxias, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, progressive muscular atrophy, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, spinal cerebral ataxia, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Von Economo's Disease, Von Hippel-Lindau Disease (VHL), Von Recklinghausen's Disease, Wallenberg's Syndrome, Werdnig-Hoffinan Disease, Wernicke-Korsakoff Syndrome, West Syndrome, Whiplash, Whipple's Disease, Williams Syndrome, Wilson Disease, Wolman's Disease, X-Linked Spinal, Bulbar Muscular Atrophy, neuropathic pain, trauma due to spinal cord or head injury, ophthalmic diseases and disorders, cerebral infarcts, depression, persistent affective disorder, secondary mood disorder, schizophrenia, drug dependency, neuroses, psychosis, dementia, paranoia, and psychosexual disorder.

In some embodiments, the CNS disease is selected from the list consisting of: dopamine transporter deficiency syndrome, an attention deficit/hyperactivity disorder (ADHD), bipolar disorder, epilepsy, multiple sclerosis, tauopathies, Alzheimer's disease, Huntington's disease, Parkinson's disease, Krabbe's disease, adrenoleukodystrophy, motor neuron disease, cerebral palsy, Batten disease, Gaucher disease, Tay Sachs disease, Rett syndrome, Sandhoff disease, Charcot-Marie-Tooth disease, Angelman syndrome, Canavan disease, Late infantile neuronal ceroid lipofuscinosis, Mucopolysaccharidosis IIIA, Mucopolysaccharidosis IIIB, Metachromatic leukodystrophy, heritable lysosomal storage diseases such as Niemann-Pick disease type C1, and/or neuronal ceroid lipofuscinoses such as Batten disease, progressive supranuclear palsy, corticobasal syndrome, and brain cancer (including astrocytomas and glioblastomas).

As used herein, “Huntington's disease”, or “HD”, refers to a neurodegenerative disease characterized by progressively worsening movement, cognitive and behavioral changes caused by a tri-nucleotide repeat expansion (e.g., CAG, which is translated into a poly-Glutamine, or PolyQ, tract) in the HTT gene that results in production of pathogenic mutant huntingtin protein (HTT, or mHTT).

As used herein, “HTT” or “huntingtin” refers to the gene which encodes the huntingtin protein. Normal huntingtin proteins function in nerve cells, and the normal HTT gene usually has from about 7 to about 35 CAG repeats at the 5′ end. The HTT gene is often mutated in patients with Huntington Disease, or at risk of developing Huntington Disease. In some embodiments, mutant huntingtin protein accelerates the rate of neuronal cell death in certain regions of the brain. Generally, the severity of HD is correlated to the size of the tri-nucleotide repeat expansion in a subject. For example, a subject having a CAG repeat region comprising between 36 and 39 repeats (SEQ ID NO: 157) is characterized as having “reduced penetrance” HD, whereas a subject having greater than 40 repeats is characterized as having “full penetrance” HD. Thus, in some embodiments, a subject having or at risk of having HD has a HTT gene comprising between about 36 and about 39 CAG repeats (e.g., 36, 37, 38 or 39 repeats). In some embodiments, a subject having or at risk of having HD has a HTT gene comprising 40 or more (e.g., 40, 45, 50, 60, 70, 80, 90, 100, 200, or more) CAG repeats (SEQ ID NO: 156). In some embodiments, a subject having a HTT gene comprising more than 100 CAG repeats develops HD earlier than a subject having fewer than 100 CAG repeats. In some embodiments, a subject having a HTT gene comprising more than 100 CAG repeats may develop HD symptoms before the age of about 20 years, and is referred to as having juvenile HD (also referred to as akinetic-rigid HD, or Westphal variant HD). The number of CAG repeats in a HTT gene allele of a subject can be determined by any suitable modality known in the art. For example, nucleic acids (e.g., DNA) can be isolated from a biological sample (e.g., blood) of a subject and the number of CAG repeats of a HTT allele can be determined by a hybridization-based method, such as PCR or nucleic acid sequencing (e.g., Illumina sequencing, Sanger sequencing, SMRT sequencing, etc.). The sequences of the HTT genes are known in a number of species, e.g., human HTT (NCBI Gene ID: 3064) mRNA sequences (NCBI Ref Seq: NM_002111.8, SEQ ID NO: 4) and protein sequences (NCBI Ref Seq: NP_0021012.4, SEQ ID NO: 5). Accordingly, in some embodiments relating to the treatment of Huntington's disease the one or more inhibitory nucleic acids (e.g., miRNAs) can hybridize to and/or reduce expression of HTT.

As used herein, “Alzheimer's disease”, or “AD”, refers to a neurodegenerative disease characterized by progressively worsening memory, disorientation, mood swings, as well as increasing difficulty with language, motivation and self-care. A number of genes can contribute to or increase the risk of AD, including Amyloid Precursor Protein (APP; NCBI Gene ID: 351), Presenilin 1 (PSEN1; NCBI Gene ID 5663), Presenilin 2 (PSEN2; NCBI Gene ID 5664), ATP binding cassette subfamily A member 7 (ABCA7; NCBI Gene ID 10347), and sortilin related receptor 1 (SORL1; NCBI Gene ID 6653). The sequences of such AD-associated genes are known in a number of species, e.g., human mRNAs and protein sequences are available in the NCBI database using the provided Gene ID numbers. These AD-associated genes and others, as well as AD-associated alleles thereof (e.g. mutations, SNPs, etc.) are known in the art and described further in, e.g., Sims et al. Nature Neuroscience 2020 23:311-22; Bellenguez et al. Current Opinion in Neurobiology 2020 61:40-48; Tabuas-Pereira et al. 2020 Neurogenetics and Psychiatric Genetics 8:1-16; and Porter et al. Chapter 15 of “Neurodegeneration and Alzheimer's Disease” 2019; each of which is incorporated by reference herein in its entirety. Accordingly, in some embodiments relating to the treatment of Alzheimer's disease, the one or more inhibitory nucleic acids (e.g., miRNAs) can hybridize to and/or reduce expression of APP, PSEN1, PSEN2, ABCA7, and/or SORL1.

“Familial Alzheimer's disease”, or “FAD,” refers to a subset of Alzheimer's disease caused by a single genetic mutation passed via autosomal dominant inheritance pattern. Three genes are known to be associated with, or causing, FAD: Presenilin 1 (PS1) on chromosome 14; Presenilin 2 (PS2) on chromosome 1; and Amyloid precursor protein (APP) on chromosome 21. Accordingly, in one embodiment, the subject has a mutation in at least one of PS1, PS2, and APP.

As used herein, “Parkinson's disease”, or “PD”, refers to a neurodegenerative disease characterized by progressively worsening shaking and stiffness and increasing problems with balance, walking, and coordination. A number of genes can contribute to or increase the risk of PD, including synuclein alpha (SNCA; NCBI Gene ID: 6622), leucine rich repeat kinase 2 (LRRK2/PARK8; NCBI Gene ID 120892), glucosylceramidase beta (GBA1; NCBI Gene ID 2629), parkin RBR E3 ubiquitin (PRKN; NCBI Gene ID 5071), PTEN induced kinase 1 (PINK1; NCBI Gene ID 65018), Parkinsonism associated deglycase (DJ1/PARK7; NCBI Gene ID 11315), VPS35 retromer complex component (VPS35; NCBI Gene ID 55737), eukaryotic translation initiation factor 4 gamma 1 (EIF4G1; NCBI Gene ID 1981), DnaJ heat shock protein family member C13 (DNAJC13; NCBI Gene ID 23317), coiled-coil-helix-coiled-coil-helix domain containing 2 (CHCHD2; NCBI Gene ID 51142), and/or ubiquitin C-terminal hydrolase L1 (UCHL1; NCBI Gene ID 7345). The sequences of such PD-associated genes are known in a number of species, e.g., human mRNAs and protein sequences are available in the NCBI database using the provided Gene ID numbers. These PD-associated genes and others, as well as PD-associated alleles thereof (e.g. mutations, SNPs, etc.) are known in the art and described further in, e.g., D'Souza et al. Acta Neuropsychiatrica 2020 32:10-22; Sardi et al. Parkinsonism & Related Disorders 2019 59:32-38; Hardy et al. Current Opinion in Genetics & Development 2009 19:254-65; Ferreria et al. Neurologica 2017 135:273-84; Jain et al. Clinical Science 2005 109:355-64; Fagan et al. European Journal of Neurology 2017 24:561-e20; Campelo et al. Parkinson's Disease 2017 4318416; and Porter et al. Chapter 15 of “Neurodegeneration and Alzheimer's Disease” 2019; each of which is incorporated by reference herein in its entirety. Accordingly, in some embodiments relating to the treatment of Parkinson's disease the one or more inhibitory nucleic acids (e.g., miRNAs) can hybridize to and/or reduce expression of SNCA, LRRK2/PARK8, PRKN, PINK1, DJ1/PARK7, VPS35, EIF4G1, DNAJC13, CHCHD2, UCHL1, and/or GBA1.

In one embodiment, the neurological disease or disorder is Huntington's disease.

When the disease or disorder is Huntington's disease, the method can further include assessing at least one primary outcome measure, at least one secondary outcome measure, or a combination thereof at a time subsequent to administering the nucleic acid (wild type or codon-optimized variant) encoding CYP46A1. The time subsequent to administering can be, for example, 2 weeks, 4 weeks, 6 weeks, 8 weeks, 10 weeks, 15 weeks, 20 weeks, 25 weeks, 30 weeks, 35 weeks, 40 weeks, 45 weeks, 50 weeks, 52 weeks, 55 weeks, 60 weeks, or longer. Non-limiting examples of primary outcome measures include incidences of dose-limiting toxicities (DLTs), treatment-emergent adverse events (TEAEs), serious adverse events (SAEs), and combinations thereof. Non-limiting examples of secondary outcome measures include anatomical and volumetric measures of brain regions impacted by Huntington's Disease assessed by MRI, a composite Unified Huntington Disease Rating Scale (cUHDRS) score, a mutant Huntingtin protein (mHTT) concentration in blood and/or cerebrospinal fluid (CSF), a level of neurofilament light chain (NfL) in blood and/or CSF, a 24OH cholesterol concentration in blood and/or CSF, a magnetic resonance spectroscopy (MRS) metabolic profile, a positron emission tomography (PET) fluoro-deoxyglucose (FDG) striatal profile, and combinations thereof.

In some embodiments, the anatomical and volumetric measures of brain regions impacted by Huntington's Disease are determined. The magnitude and variability of change from baseline, i.e., the measurement of the brain regions at the time of rAAV administration of the nucleic acid encoding CYP46A1, determined by a slope of atrophy progression on striatal structures, ventricle dilation, and cortical atrophy can be maintained (i.e., stabilized) or improved (e.g., decreased) by greater than or equal to about 0.5 vol. %, greater than or equal to about 1 vol. %, greater than or equal to about 2 vol. %, greater than or equal to about 3 vol. %, greater than or equal to about 4 vol. %, greater than or equal to about 5 vol. %, greater than or equal to about 6 vol. %, greater than or equal to about 7 vol. %, greater than or equal to about 8 vol. %, greater than or equal to about 9 vol. %, greater than or equal to about 10 vol. %, greater than or equal to about 15 vol. %, greater than or equal to about 20 vol. %, greater than or equal to about 25 vol. %, or higher, such as a change of about 0.5 vol. %, about 1 vol. %, about 2 vol. %, about 3 vol. %, about 4 vol. %, about 5 vol. %, about 6 vol. %, about 7 vol. %, about 8 vol. %, about 9 vol. %, about 10 vol. %, about 11 vol. %, about 12 vol. %, about 13 vol. %, about 14 vol. %, about 15 vol. %, about 16 vol. %, about 17 vol. %, about 18 vol. %, about 19 vol. %, about 20 vol. %, about 25 vol. %, or higher.

In some embodiments, the cUHDRS score is determined and compared to a baseline cUHDRS score, i.e., a cUHDRS score at the time of rAAV administration of the nucleic acid encoding CYP46A1. Relative to the baseline cUHDRS score, the cUHDRS score can be maintained (i.e., stabilized) or improved by greater than or equal to about 0.5%, greater than or equal to about 1%, greater than or equal to about 2%, greater than or equal to about 3%, greater than or equal to about 4%, greater than or equal to about 5%, greater than or equal to about 6%, greater than or equal to about 7%, greater than or equal to about 8%, greater than or equal to about 9%, greater than or equal to about 10%, greater than or equal to about 15%, greater than or equal to about 20%, greater than or equal to about 25%, or higher, such as an improvement of about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, or more.

In some embodiments, the mHTT concentration in blood and/or CSF is determined and compared to a corresponding baseline mHTT concentration, i.e., a mHTT concentration in the blood and/or CSF at the time of rAAV administration of the nucleic acid encoding CYP46A1. Relative to the baseline mHTT concentration, the mHTT concentration can be maintained (i.e., stabilized) or improved (i.e., decreased) by greater than or equal to about 0.5%, greater than or equal to about 1%, greater than or equal to about 2%, greater than or equal to about 3%, greater than or equal to about 4%, greater than or equal to about 5%, greater than or equal to about 6%, greater than or equal to about 7%, greater than or equal to about 8%, greater than or equal to about 9%, greater than or equal to about 10%, greater than or equal to about 15%, greater than or equal to about 20%, greater than or equal to about 25%, or higher, such as an improvement of about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, or more.

In some embodiments, the level of NfL in blood and/or CSF is determined and compared to a corresponding baseline NfL level, i.e., a NfL level in the blood and/or CSF at the time of rAAV administration of the nucleic acid encoding CYP46A1. Relative to the baseline NfL level concentration, the level of NfL can be maintained (i.e., stabilized) or improved (i.e., decreased) by greater than or equal to about 0.5%, greater than or equal to about 1%, greater than or equal to about 2%, greater than or equal to about 3%, greater than or equal to about 4%, greater than or equal to about 5%, greater than or equal to about 6%, greater than or equal to about 7%, greater than or equal to about 8%, greater than or equal to about 9%, greater than or equal to about 10%, greater than or equal to about 15%, greater than or equal to about 20%, greater than or equal to about 25%, or higher, such as an improvement of about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, or more.

In some embodiments, the 24OH cholesterol concentration in blood and/or CSF is determined and compared to a corresponding baseline 24OH cholesterol concentration, i.e., a 24OH cholesterol concentration in the blood and/or CSF at the time of rAAV administration of the nucleic acid encoding CYP46A1. Relative to the baseline 24OH cholesterol concentration, the 24OH cholesterol concentration can be maintained (i.e., stabilized) or improved (i.e., increased) by greater than or equal to about 0.5%, greater than or equal to about 1%, greater than or equal to about 2%, greater than or equal to about 3%, greater than or equal to about 4%, greater than or equal to about 5%, greater than or equal to about 6%, greater than or equal to about 7%, greater than or equal to about 8%, greater than or equal to about 9%, greater than or equal to about 10%, greater than or equal to about 15%, greater than or equal to about 20%, greater than or equal to about 25%, or higher, such as an improvement of about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, or more.

In some embodiments, the MRS metabolic profile is determined and compared to a corresponding baseline MRS metabolic profile, i.e., a MRS metabolic profile at the time of rAAV administration of the nucleic acid encoding CYP46A1. Relative to the baseline MRS metabolic profile, the MRS metabolic profile can be maintained (i.e., stabilized) or improved by greater than or equal to about 0.5%, greater than or equal to about 1%, greater than or equal to about 2%, greater than or equal to about 3%, greater than or equal to about 4%, greater than or equal to about 5%, greater than or equal to about 6%, greater than or equal to about 7%, greater than or equal to about 8%, greater than or equal to about 9%, greater than or equal to about 10%, greater than or equal to about 15%, greater than or equal to about 20%, greater than or equal to about 25%, or higher, such as an improvement of about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, or more.

In some embodiments, the PET FDG profile is determined and compared to a corresponding baseline PET FDG profile, i.e., a PET FDG profile at the time of rAAV administration of the nucleic acid encoding CYP46A1. Relative to the baseline PET FDG profile, the PET FDG profile can be maintained (i.e., stabilized) or improved by greater than or equal to about 0.5%, greater than or equal to about 1%, greater than or equal to about 2%, greater than or equal to about 3%, greater than or equal to about 4%, greater than or equal to about 5%, greater than or equal to about 6%, greater than or equal to about 7%, greater than or equal to about 8%, greater than or equal to about 9%, greater than or equal to about 10%, greater than or equal to about 15%, greater than or equal to about 20%, greater than or equal to about 25%, or higher, such as an improvement of about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, or more.

In one embodiment, the neurological disease or disorder is Alzheimer's disease.

In one embodiment, the neurological disease or disorder is Familial Alzheimer's disease.

In one embodiment, the neurological disease or disorder is Parkinson's disease.

In one embodiment, the neurological disease or disorder is Multiple Systems Atrophy.

An “effective amount” of a substance is an amount sufficient to produce a desired effect. In some embodiments, an effective amount of an isolated nucleic acid is an amount sufficient to transfect (or infect in the context of rAAV mediated delivery) a sufficient number of target cells of a target tissue of a subject. In some embodiments, a target tissue is central nervous system (CNS) tissue (e.g., brain tissue, spinal cord tissue, cerebrospinal fluid (CSF), etc.). In some embodiments, an effective amount of an isolated nucleic acid (e.g., which may be delivered via an rAAV) may be an amount sufficient to have a therapeutic benefit in a subject, e.g., to reduce the expression of a pathogenic gene or protein (e.g., HTT), to extend the lifespan of a subject, to improve in the subject one or more symptoms of disease (e.g., a symptom of Huntington's disease), etc. The effective amount will depend on a variety of factors such as, for example, the species, age, weight, health of the subject, and the tissue to be targeted, and may thus vary among subject and tissue as described elsewhere in the disclosure.

### Inhibitory RNAs

In some aspects, the disclosure provides inhibitory nucleic acids, e.g., miRNA, that specifically binds to (e.g., hybridizes with) at least two (e.g., 2, 3, 4, 5, 6, 7, 8, 9, 10, 11, 12, 13, 14, 15, 16, 17, 18, 19, 20, 21, 22, 23, 24, 25, or more) continuous bases of a target, e.g., human huntingtin mRNA (e.g., SEQ ID NO: 4). In some embodiments, the disclosure provides inhibitory nucleic acids, e.g., miRNA, that specifically binds to (e.g., hybridizes with) at least two (e.g., 2, 3, 4, 5, 6, 7, 8, 9, 10, 11, 12, 13, 14, 15, 16, 17, 18, 19, 20, 21, 22, 23, 24, 25, or more) continuous bases of exon 1 of human huntingtin mRNA (e.g., SEQ ID NO: 3). As used herein “continuous bases” refers to two or more nucleotide bases that are covalently bound (e.g., by one or more phosphodiester bond, etc.) to each other (e.g. as part of a nucleic acid molecule). In some embodiments, the at least one miRNA is about 50%, about 60% about 70% about 80% about 90%, about 95%, about 99% or about 100% identical to the two or more (e.g., 2, 3, 4, 5, 6, 7, 8, 9, 10, 11, 12, 13, 14, 15, 16, 17, 18, 19, 20, 21, 22, 23, 24, 25, or more) continuous nucleotide bases of the target, e.g., SEQ ID NOs 3 or 4. In some embodiments, the inhibitory RNA is a miRNA which is comprises or is encoded by the sequence set forth in any one of SEQ ID NOs: 6-17, 40-44, 50-66, 158-185, or 217-260.

In one aspect described herein are inhibitory RNAs that can be used for the treatment of a neurological disease or disorder. In some embodiments of any of the aspects, the nucleic acid sequence of the inhibitory RNA comprises one of SEQ ID NO: 6-17, 40-44, 50-66, 158-185, or 217-260 or a sequence that is at least 95% (e.g., at least 95%, at least 96%, at least 97%, at least 98%, or at least 99%) identical to the sequence of at least one of SEQ ID NO: 6-17, 40-44, 50-66, 158-185, or 217-260 that maintains the same functions as SEQ ID NO: 3 or 4 (e.g., HTT inhibition).

In some embodiments, the vector described herein comprises at least one miRNA, each miRNA comprising a sequence set forth in any one of SEQ ID NOs: 6-17, 40-44, 50-66, 158-185, or 217-260. In some embodiments, the vector described herein comprises at least one miRNA, each miRNA comprising a sequence set forth in any one of SEQ ID NOs: 6-17, 40-44, 50-66, 158-185, or 217-260 flanked by a miRNA backbone sequence.

In some embodiments, the vector described herein comprises at least one miRNA, each miRNA comprising a seed sequence complementary to one of SEQ ID NO: 3, 4, 18-39, or 46-49. In some embodiments, the vector described herein comprises at least one miRNA, each miRNA comprising a seed sequence complementary to one of SEQ ID NO: 3, 4, 18-39, or 46-49 flanked by a miRNA backbone sequence. In some embodiments, the vector described herein comprises at least one miRNA, each miRNA comprising a seed sequence substantially complementary to one of SEQ ID NO: 3, 4, 18-39, or 46-49. In some embodiments, the vector described herein comprises at least one miRNA, each miRNA comprising a seed sequence substantially complementary to one of SEQ ID NO: 3, 4, 18-39, or 46-49 flanked by a miRNA backbone sequence.

TABLE 1
first RNA sequences substantially complementary
to SEQ ID NO: 4

| miRNA sequence | SEQ ID NO: |
|---|---|
| 5′-AAGGACUUGAGGGACUCGA-3′ | 6 |
| 5′-AAGGACUUGAGGGACUCGAA-3′ | 7 |
| 5′-AAGGACUUGAGGGACUCGAAG-3′ | 8 |
| 5′-AAGGACUUGAGGGACUCGAAGG-3′ | 9 |
| 5′-AAGGACUUGAGGGACUCGAAGGC-3′ | 10 |

TABLE 2
second RNA sequences substantially complementary
to one or more first RNA sequences provided in
Table 1

| miRNA sequence | SEQ ID NO: |
|---|---|
| 5′-UCGAGUCCCUCAAGUCCUU-3′ | 11 |
| 5′-UUCGAGUCCCUCAAGUCCUU-3′ | 12 |
| 5′-CUUCGAGUCCCUCAAGUCCUU-3′ | 13 |
| 5′-CCUUCGAGUCCCUCAAGUCCUU-3′ | 14 |
| 5′-GCCUUCGAGUCCCUCAAGUCCUU-3′ | 15 |
| 5′-CUUCGAGUCUCAAGUCCUU-3′ | 16 |
| 5′-ACGAGUCCCUCAAGUCCUC-3′ | 17 |

TABLE 3
Target Sequences in Exon 1 of human HTT gene,
targeted by the miRNAs provided by Tables 1 and 2

| Target Sequence | SEQ ID NO: |
|---|---|
| aaggacuuga gggacucgaa | 18 |
| tccaagatgg acggccgctc a | 19 |
| ccaagatgga cggccgctca g | 20 |
| agatggacgg ccgctcaggt t | 21 |
| atggacggcc gctcaggttc t | 22 |
| gacggccgct caggttctgc t | 23 |
| cggccgctca ggttctgctt t | 24 |
| gtgctgagcg gcgccgcgag t | 25 |
| cgccgcgagt cggcccgagg c | 26 |
| accgccatgg cgaccctgga a | 27 |
| ccgccatggc gaccctggaa a | 28 |
| gaaggccttc gagtccctca a | 29 |
| cttcgagtcc ctcaagtcct t | 30 |
| ccgccgccgc ctcctcagct t | 31 |
| gccgcctcct cagcttcctc a | 32 |
| tcagccgccg ccgcaggcac a | 33 |
| gccgcaggca cagccgctgc t | 34 |
| ggcacagccg ctgctgcctc a | 35 |
| gccgctgctg cctcagccgc a | 36 |
| cggcccggct gtggctgagg a | 37 |
| ctgtggctga ggagccgctg c | 38 |
| tgtggctgag gagccgctgc a | 39 |

In some embodiments, an miRNA comprises SEQ ID NOs: 6 and 11, SEQ ID NOs: 7 and 12; SEQ ID NOs: 8 and 11; SEQ ID NOs: 8 and 16; SEQ ID NOs: 8 and 17; SEQ ID NOs: 9 and 14; or SEQ ID NOs: 10 and 15.

In some embodiments, the vector comprises a pre-miRNA having the sequence of SEQ ID NO: 40 or 41. These pre-miRNAs include scaffolds comprising SEQ ID NO: 8. Alternative first RNA sequences disclosed herein can be substituted for SEQ ID NO: 8 in either of SEQ ID NOs: 40 and 41.

In some embodiments, the vector comprises a pri-miRNA having the sequence of SEQ ID NO: 42 or 43. The pri-miRNA of SEQ ID NO: 42 includes scaffolds comprising SEQ ID NO: 8 and 16. Alternative RNA sequences disclosed herein can be substituted for SEQ ID NO: 8 and 16 in SEQ ID NO: 42. The pri-miRNA of SEQ ID NOs: 43 and 44 include scaffolds comprising SEQ ID NO: 8 and 17. Alternative RNA sequences disclosed herein can be substituted for SEQ ID NO: 8 and 17 in either of SEQ ID NOs: 43 and 44.

TABLE 4
pre- and pri-miRNAs comprising miRNAs provided in Tables 1 and 2

| | | SEQ |
|---|---|---|
| Name | Sequence | ID NO: |
| Pre- | 5′-CUUGGGAAUGGCAAGGAAGGACUUGAGGGACUCGAAGACGA | 40 |
| miR451a | GUCCCUCAAGUCCUCUCUUGCUAUACCCAGA-3′ | |
| Pre- | 5′-UGCUGAAGGACUUGAGGGACUCGAAGGUUUUGGCCACUGACU | 41 |
| miR155 | GACCUUCGAGUCUCAAGUCCUUCAGGA-3′ | |
| | gcuaagcacu ucguggccgu cgaucguuua aagggaggua gugagucgac | 42 |
| | caguggaucc uggaggcuug cugaaggcug uaugcugaag gacuugaggg | |
| | acucgaaggu uuuggccacu gacugaccuu cgagucucaa guccuucagg | |
| | acacaaggcc uguuacuagc acucacaugg aacaaauggc ccagaucugg | |
| | ccgcacucga gauaucuaga cccagcuuuc uuguacaaag ugguugaucu | |
| | agagggcccg cgguucgcug au | |
| | gcuccugggc aacgugcugg uuauugugcu gucucaucau uuuggcaaag | 43 |
| | aauuaagggc gaauucgagc ucgguaccuc gcgaaugcau cuagauaucg | |
| | gcgcuaugcu uccugugccc ccaguggggc ccuggcuggg auuucaucau | |
| | auacuguaag uuugcgauga gacacuacag uauagaugau guacuagucc | |
| | gggcaccccc agcucuggag ccugacaagg aggacaggag agaugcugca | |
| | agcccaagaa gcucucugcu cagccuguca caaccuacug acugccaggg | |
| | cacuugggaa uggcaaggaa ggacuugagg gacucgaaga cgagucccuc | |
| | aaguccucuc uugcuauacc cagaaaacgu gccaggaaga gaacucagga | |
| | cccugaagca gacuacugga agggagacuc cagcucaaac aaggcagggg | |
| | ugggggcgug ggauuggggg uaggggaggg aauagauaca uuuucucuuu | |
| | ccuguuguaa agaaauaaag auaagccagg cacaguggcu cacgccugua | |
| | aucccaccac uuucagaggc caaggcgcug gauccagauc ucgagcggcc | |
| | gcccg | |
| | agucucgugc agauggacag caccgcugag caauggaagc ggguaggccu | 44 |
| | uuggggcagc ggccaauagc agcuuugcuc cuucgcuuuc ugggcucaga | |
| | ggcugggaag gggugggucc ggggcggggc ucaggggcgg gcucaggggc | |
| | ggggcgggcg cccgaagguc cuccggaggc ccggcauucu gcacgcuuca | |
| | aaagcgcacg ucugccgcgc uguucuccuc uuccucaucu ccgggccuuu | |
| | cgacccggau cccccgggcu gcaggaauuc gagcucggua ccucgcgaau | |
| | gcaucuagau aucggcgcua ugcuuccugu gcccccagug gggcccuggc | |
| | ugggauuuca ucauauacug uaaguuugcg augagacacu acaguauaga | |
| | ugauguacua guccgggcac ccccagcucu ggagccugac aaggaggaca | |
| | ggagagaugc ugcaagccca agaagcucuc ugcucagccu gucacaaccu | |
| | acugacugcc agggcacuug ggaauggcaa ggaaggacuu gagggacucg | |
| | aagacgaguc ccucaagucc ucucuugcua uacccagaaa acgugccagg | |
| | aagagaacuc aggacccuga agcagacuac uggaagggag acuccagcuc | |
| | aaacaaggca gggguggggg cgugggauug gggguagggg agggaauaga | |
| | uacauuuucu cuuuccuguu guaaagaaau aaagauaagc caggcacagu | |
| | ggcucacgcc uguaauccca ccacuuucag aggccaaggc gcuggaucca | |
| | gaucucgagc ggccgcccg | |

In some embodiments, the inhibitory nucleic acid can comprise one or more of SEQ ID NOs: 1-102 and/or 103-249 of International Patent Publication WO2017/201258. In some embodiments, the inhibitory nucleic acid can comprise one or more of the duplex combinations selected from SEQ ID NOs: 1-249 of International Patent Publication WO2017/201258 which are provided in Tables 3-5 of International Patent Publication WO2017/201258. In some embodiments, the vector can comprise one or more of the pri-miRNAs which are provided in Table 9 or the pri-mRNAs which are provided in Table 10 of International Patent Publication WO2017/201258. The contents of International Patent Publication WO2017/201258 are incorporated by reference herein in their entirety.

In some embodiments, the inhibitory nucleic acid can comprise one or more of SEQ ID NOs: 914-1013 and/or 1014-1160 of International Patent Publication WO2018/204803. In some embodiments, the inhibitory nucleic acid can comprise one or more of the duplex combinations selected from SEQ ID NOs: 914-1160 of International Patent Publication WO2018/204803 which are provided in Tables 4-6 of International Patent Publication WO2018/204803. The contents of International Patent Publication WO2018/204803 are incorporated by reference herein in their entirety.

In some embodiments, the inhibitory nucleic acid can comprise one or more of SEQ ID NOs: 916-1015 and/or 1016-1162, of International Patent Publication WO2018/204797. In some embodiments, the inhibitory nucleic acid can comprise one or more of SEQ ID NOs: 916-1015, 1016-1162, 1164-1332, and/or 1333-1501 of International Patent Publication WO2018/204797. In some embodiments, the inhibitory nucleic acid can comprise one or more of the duplex combinations selected from SEQ ID NOs: 916-1162 of International Patent Publication WO2018/204797 which are provided in Tables 4-6 of International Patent Publication WO2018/204797. In some embodiments, the inhibitory nucleic acid can comprise one or more of the duplex combinations selected from SEQ ID NOs: 1164-1501 of International Patent Publication WO2018/204797 which are provided in Table 9 of International Patent Publication WO2018/204797. The contents of International Patent Publication WO2018/204797 are incorporated by reference herein in their entirety.

In some embodiments, the inhibitory nucleic acid can target, e.g., comprise a sequence complementary or substantially complementary to, a heterozygous SNP within a gene encoding a gain-of-function mutant huntingtin protein. In some embodiments, the SNP has an allelic frequency of at least 10% in a sample population. In some embodiments, the SNP present at a genomic site selected from the group consisting of RS362331, RS4690077, RS363125, RS363075, RS362268, RS362267, RS362307, RS362306, RS362305, RS362304, RS362303, and RS7685686. Such SNPs are described in more detail in, e.g., U.S. Pat. No. 9,343,943 which is incorporated by reference herein in its entirety. In some embodiments, the target sequence is one of SEQ ID NOs: 45-49. In some embodiments, the inhibitory nucleic acid sequence comprises one or more of SEQ ID NOs: 50-61. In some embodiments, the inhibitory nucleic acid sequence comprises at least SEQ ID NOs: 50 and 51, e.g., in a duplex. In some embodiments, the inhibitory nucleic acid sequence comprises at least SEQ ID NOs: 52 and 53, e.g., in a duplex. In some embodiments, the inhibitory nucleic acid sequence comprises at least SEQ ID NOs: 54 and 55, e.g., in a duplex. In some embodiments, the inhibitory nucleic acid sequence comprises at least SEQ ID NOs: 56 and 57, e.g., in a duplex. In some embodiments, the inhibitory nucleic acid sequence comprises at least SEQ ID NOs: 58 and 59, e.g., in a duplex. In some embodiments, the inhibitory nucleic acid sequence comprises at least SEQ ID NOs: 60 and 61, e.g., in a duplex.

TABLE 5
Target sequences

| Target Sequence | SEQ ID NO: |
|---|---|
| ccacgccugc ucccucaucc acugugugca cuucauccug | 45 |
| ccacgccugc ucccucaucu acugugugca cuucauccug | 46 |
| uaagagaugg ggacaguaau ucaacgcuag aagaaca | 47 |
| uaagagaugg ggacaguacu ucaacgcuag aagaaca | 48 |
| cagatgcc atggcctgtgct gggccag | 49 |

TABLE 6
sense and antisense (or first and second RNA sequences) that target
SNPs in human HTT gene

| | SEQ ID | | SEQ ID |
|---|---|---|---|
| Sense sequence | NO: | Antisense sequence | NO: |
| ucccucaucc acugugugaa c | 50 | gcacacagug gaugagggag c | 51 |
| ucccucaucu acugugugaa c | 52 | cgagggagua gaugacacac g | 53 |
| gggacaguaa uucaacgcgu c | 54 | agcguugaau uacugucccc a | 55 |
| gggacaguac uucaacgcgu c | 56 | accccuguca ugaaguugcg a | 57 |
| ugccauggcc ugugcugguc c | 58 | cccagcacag gccauggca c | 59 |
| ugccauggca ugugcugguc c | 60 | cccagcacau gccuaggcau c | 61 |

In one embodiment, the inhibitory nucleic acid, e.g., miRNA, can hybridize specifically to exon 1 of the HTT gene. Exemplary miRNAs that hybridize to exon 1 of the HTT gene are described in Table 17, i.e., SEQ ID NOs 158-160 and 164-166.

In one embodiment, the inhibitory nucleic acid, e.g., miRNA, can hybridize specifically to the exon1/exon2 boundary of the HTT gene. Exemplary miRNA that hybridize to exon1/exon2 boundary of the HTT gene are described in Table 17, i.e., SEQ ID NOs 161-163 and 167-169 (i.e., as indicated by the asterisks).

In one embodiment, the nucleic acid sequence of the inhibitory RNA comprises at least one of SEQ ID NO: 158-169 or a sequence that is at least 95% (e.g., at least 95%, at least 96%, at least 97%, at least 98%, or at least 99%) identical to the sequence of at least one of SEQ ID NO: 158-169 that maintains the same function of HTT inhibition.

In some embodiments, the inhibitory nucleic acid sequence comprises at least SEQ ID NOs: 158 and 164, e.g., in a duplex. In some embodiments, the inhibitory nucleic acid sequence comprises at least SEQ ID NOs: 159 and 165, e.g., in a duplex. In some embodiments, the inhibitory nucleic acid sequence comprises at least SEQ ID NOs: 160 and 166, e.g., in a duplex. In some embodiments, the inhibitory nucleic acid sequence comprises at least SEQ ID NOs: 161 and 167, e.g., in a duplex. In some embodiments, the inhibitory nucleic acid sequence comprises at least SEQ ID NOs: 162 and 168, e.g., in a duplex. In some embodiments, the inhibitory nucleic acid sequence comprises at least SEQ ID NOs: 163 and 169, e.g., in a duplex.

TABLE 17
miRNA Targeting HTT Exon 1

| | | SEQ ID | | SEQ ID |
|---|---|---|---|---|
| | Sense | NO: | SiRNA | NO: |
| He1-1 | CGCUCAGGUUCUGCUUUUA | 158 | UAAAAGCAGAACCUGAGCGGC | 164 |
| He1-4 | GCCCAGAGCCCCAUUCAUU | 159 | AAUGAAUGGGGCUCUGGGCCG | 165 |
| He1-6 | GGGACGGGUCCAAGAUGGA | 160 | UCCAUCUUGGACCCGUCCCGG | 166 |
| He1-8* | GCUGCACCGACCAAAGAAA | 161 | UUUCUUUGGUCGGUGCAGCGG | 167 |
| He1-9* | GAGCCGCUGCACCGACCAA | 162 | UUGGUCGGUGCAGCGGCUCCU | 168 |
| He1-10* | GCACCGACCAAAGAAAGAA | 163 | UUCUUUCUUUGGUCGGUGCAG | 169 |

In one embodiment, the inhibitory nucleic acid, e.g., miRNA, can hybridize specifically to an intron 1 of the HTT gene. Exemplary miRNA that hybridize to intron 1 of the HTT gene are described in Table 18, i.e., SEQ ID NOs 170-174 and 178-182.

In one embodiment, the inhibitory nucleic acid, e.g., miRNA, can hybridize specifically to an exon1/intron 1 boundary of the HTT gene. Exemplary miRNA that hybridize to exon1/intron 1 boundary of the HTT gene are described in Table 18, i.e., SEQ ID NOs 175-177 and 183-185. (i.e., as indicated by the asterisks).

In one embodiment, the nucleic acid sequence of the inhibitory RNA comprises one of SEQ ID NO: 170-185 or a sequence that is at least 95% (e.g., at least 95%, at least 96%, at least 97%, at least 98%, or at least 99%) identical to the sequence of at least one of SEQ ID NO: 170-185 that maintains the same function of HTT inhibition.

In some embodiments, the inhibitory nucleic acid sequence comprises at least SEQ ID NOs: 170 and 178, e.g., in a duplex. In some embodiments, the inhibitory nucleic acid sequence comprises at least SEQ ID NOs: 171 and 179, e.g., in a duplex. In some embodiments, the inhibitory nucleic acid sequence comprises at least SEQ ID NOs: 172 and 180, e.g., in a duplex. In some embodiments, the inhibitory nucleic acid sequence comprises at least SEQ ID NOs: 173 and 181, e.g., in a duplex. In some embodiments, the inhibitory nucleic acid sequence comprises at least SEQ ID NOs: 174 and 182, e.g., in a duplex. In some embodiments, the inhibitory nucleic acid sequence comprises at least SEQ ID NOs: 175 and 183, e.g., in a duplex. In some embodiments, the inhibitory nucleic acid sequence comprises at least SEQ ID NOs: 176 and 184, e.g., in a duplex. In some embodiments, the inhibitory nucleic acid sequence comprises at least SEQ ID NOs: 177 and 185, e.g., in a duplex.

In one embodiment, an isolated nucleic acid of the disclosure comprises a sequence encoding miRNA comprising a sequence set forth in any one of SEQ ID NOs: 158-185.

In some embodiments of any of the aspects, the nucleic acid sequence of the inhibitory RNA comprises one of SEQ ID NO: 158-185 or a sequence that is at least 95% (e.g., at least 95%, at least 96%, at least 97%, at least 98%, or at least 99%) identical to the sequence of at least one of SEQ ID NO: 158-185 that maintains the same functions as SEQ ID NO: 158-185 (e.g., HTT inhibition).

In one embodiment, a preferred miRNA molecule or equivalent or mimic or isomiR thereof comprises at least 6 of the 7 nucleotides present in the seed sequence identified in or as SEQ ID NO: 158-185 and more preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

Described herein is a recombinant AAV comprising a capsid protein and a nucleic acid encoding a transgene, wherein the transgene comprises a nucleic acid sequence encoding one or more miRNAs, and wherein each miRNA comprises a sequence set forth in any one of SEQ ID NOs: 158-185.

Described herein is a method for treating a CNS disease, e.g., Huntington's disease, the method comprising administering to a subject a recombinant AAV comprising a capsid protein and a nucleic acid encoding a transgene, wherein the transgene comprises a nucleic acid sequence encoding one or more miRNAs, and wherein each miRNA comprises a sequence set forth in any one of SEQ ID NOs: 158-185.

Described herein is a method for treating a CNS disease, e.g., Huntington's disease, the method comprising administering to a subject a recombinant AAV comprising a capsid protein and a nucleic acid encoding a transgene, wherein the transgene comprises a nucleic acid sequence encoding one or more miRNAs, and wherein each miRNA comprises a sequence set forth in any one of SEQ ID NOs: 158-185; and a CYP461 protein or a nucleic acid sequence that encodes a CYP46A1 protein.

Described herein is a method for treating a CNS disease, e.g., Huntington's disease, the method comprising administering to a subject a recombinant AAV comprising a capsid protein and a nucleic acid encoding a transgene, wherein the transgene comprises a nucleic acid sequence encoding one or more miRNAs, and wherein each miRNA comprises a sequence set forth in any one of SEQ ID NOs: 158-185; and an immune modulator.

Described herein is a method for treating a CNS disease, e.g., Huntington's disease, the method comprising administering to a subject a recombinant AAV comprising a capsid protein and a nucleic acid encoding a transgene, wherein the transgene comprises a nucleic acid sequence encoding one or more miRNAs, and wherein each miRNA comprises a sequence set forth in any one of SEQ ID NOs: 158-185; a CYP461 protein or a nucleic acid sequence that encodes a CYP46A1 protein; and an immune modulator.

Described herein is a recombinant AAV having a genome that comprises, consists essentially of, or consists of the sequence of SEQ ID NO: 194. In various embodiments, any of the elements described in the sequence of SEQ ID NO: 194 can be replaced by any of the elements described herein.

For example, in one embodiment, the enhancer element described in the sequence of SEQ ID NO: 194 can be replaced by any of the enhancer elements described herein. In one embodiment, the promoter described in the sequence of SEQ ID NO: 194 can be replaced by any of the promoters described herein. In one embodiment, the CYP46A1 transgene described in the sequence of SEQ ID NO: 194 can be replaced by any of the CYP46A1 transgenes described herein, e.g., a different codon-optimized CYP46A1 transgene. In one embodiment, the poly(A) signal sequence described in the sequence of SEQ ID NO: 194 can be replaced by any of the poly(A) signal sequences described herein. In one embodiment, the intron element described in the sequence of SEQ ID NO: 194 can be replaced by any of the intron elements described herein. In one embodiment, the protelomerase recognition sequence described in the sequence of SEQ ID NO: 194 can be replaced by any of the protelomerase recognition sequences described herein. In one embodiment, any of the linker or stuffer sequences described in the sequence of SEQ ID NO: 194 can be replaced by any of the linker or stuffer sequences described herein. In one embodiment, the ITR described in the sequence of SEQ ID NO: 194 can be replaced by any of the ITRs described herein.

In one embodiment, the sequence of SEQ ID NO: 194 is modified to include any of the miRNAs described herein.

Further described herein is a composition comprising a recombinant AAV having a genome that comprises, consists essentially of, or consists of the sequence of SEQ ID NO: 194.

Described herein is a method for treating a CNS disease or disorder, the method comprising administering to a subject a recombinant AAV having a genome that comprises, consists essentially of, or consists of the sequence of SEQ ID NO: 194. In one embodiment, the subject is further administered an immune modulator.

Described herein is a method for treating Huntington's disease, the method comprising administering to a subject a recombinant AAV a recombinant AAV having a genome that comprises, consists essentially of, or consists of the sequence of SEQ ID NO: 194.

Another aspect herein is a method for treating a CNS disease or disorder, such as Huntington's disease, the method comprising administering to a subject a recombinant AAV having a genome that comprises, consists essentially of, or consists of the sequence of SEQ ID NO: 194 that has been amended as described herein above, e.g., the CYP46A1 transgene sequence described in SEQ ID NO: 194 has been replaced by another CYP46A1 transgene sequence described herein. In one embodiment, the subject is further administered an immune modulator.

In one embodiment, the subject is further administered an immune modulator.

In some embodiments, the vector comprising at least one inhibitory nucleic acid sequence described herein, e.g., a miRNA, comprises at least one copy of the at least one inhibitory nucleic acid sequence. For example, the vector can comprise at least 1, at least 2, at least 3, at least 3, at least 4, at least 5 or more of a given miRNA.

TABLE 18
miRNA Targeting HTT Intron 1

| | | SEQ | | SEQ |
|---|---|---|---|---|
| | | ID | | ID |
| | Sense | NO: | SiRNA | NO: |
| HI-1 | GGAUGGCGGUAACCCUGCA | 170 | UGCAGGGUUACCGCCAUCCCC | 178 |
| HI-5 | GGUCAGAUUGUAAGUAUUU | 171 | AAAUACUUACAAUCUGACCAU | 179 |
| HI-6 | GCAAACCCUUGUUCUCAUU | 172 | AAUGAGAACAAGGGUUUGCAA | 180 |
| HI-8 | GGACAUAGACAGUAAGCAA | 173 | UUGCUUACUGUCUAUGUCCUU | 181 |
| HI-9 | GGAGAGCAGUGUUAGCUUA | 174 | UAAGCUAACACUGCUCUCCGA | 182 |
| HI-10* | GCUGCACCGACCGUGAGUU | 175 | AACUCACGGUCGGUGCAGCGG | 183 |
| HI-11* | GCACCGACCGUGAGUUUGG | 176 | CCAAACUCACGGUCGGUGCAG | 184 |
| HI-12* | GCCGCUGCACCGACCGUGA | 177 | UCACGGUCGGUGCAGCGGCUC | 185 |

In some embodiments, an inhibitory nucleic acid, e.g., miRNA, can hybridize specifically to, or target a polymorphism, mutation, or SNP in one of the genes disclosed herein. Methods of selecting inhibitory nucleic acid sequences that target polymorphisms, e.g., SNPs, in a HTT gene are known in the art. For example, such methods are disclosed in U.S. Pat. Nos. 8,679,750 and 7,947,658, each of which is incorporated by reference herein in its entirety. In some embodiments, the inhibitory nucleic acid can comprise a sequence, e.g., one or more of SEQ ID NOs: 1-342 of U.S. Pat. No. 8,679,750 or 7,947,658.

In some embodiments, the inhibitory nucleic acid can comprise one or more of SEQ ID NOs: 62-66.

TABLE 7
In some embodiments, the capitalized letters
comprise 2′-O-(2-methoxy)ethyl modifications.

| | SEQ ID |
|---|---|
| | NO |
| 5′-CTCAGtaacattgacACCAC-3′ | 62 |
| 5′-CTCGActaaagcaggATTTC-3 | 63 |
| 5′-CCTTCcctgaaggttCCTCC-3′ | 64 |
| 5′-GCAGGgttaccgccaTCCCC-3′ | 65 |
| 5′-CGAGAcagtcgcttcCACTT -3′ | 66 |

Further suitable sequences are known in the art, e.g., in U.S. Pat. No. 7,951,934, Miniarikova et al. Molecular Therapy—Nucleic Acids 2015 5:e297; and Kordasiweicz et al. Neuron 2012 74:1031-1044; each of which is incorporated by reference herein in its entirety.

In some embodiments of any of the aspects, the inhibitory RNA (e.g., miRNA) binds and/or targets the 5′ untranslated region (UTR) of the target. In some embodiments of any of the aspects, the inhibitory RNA (e.g., miRNA) binds and/or targets one or more exons of the target. In some embodiments of any of the aspects, the inhibitory RNA (e.g., miRNA) binds and/or targets the 5′ UTR, exon 1, CAG repeats, the CAG 5′-jumper, or a CAG 3′jumper of HTT.

In some embodiments, the inhibitory RNA and/or vector does not comprise a sequence of any of SEQ ID NOs: 67-73. In some embodiments, the inhibitory RNA and/or vector does not comprise a sequence having more than 80%, more than 85%, more than 90%, more than 95%, or more than 98% sequence identity with any of any of SEQ ID NOs: 67-73.

In some embodiments, the inhibitory RNA and/or vector does not comprise a sequence of any of SEQ ID NOs: 67-73 or 135-151. In some embodiments, the inhibitory RNA and/or vector does not comprise a sequence having more than 80%, more than 85%, more than 90%, more than 95%, or more than 98% sequence identity with any of any of SEQ ID NOs: 67-73 or 135-151.

In some embodiments, the inhibitory RNA and/or vector does comprise a sequence of any of SEQ ID NOs: 67-73. In some embodiments, the inhibitory RNA and/or vector does comprise a sequence having more than 80%, more than 85%, more than 90%, more than 95%, or more than 98% sequence identity with any of any of SEQ ID NOs: 67-73.

In some embodiments, the inhibitory RNA and/or vector does comprise a sequence of any of SEQ ID NOs: 67-73 or 135-151. In some embodiments, the inhibitory RNA and/or vector does comprise a sequence having more than 80%, more than 85%, more than 90%, more than 95%, or more than 98% sequence identity with any of any of SEQ ID NOs: 67-73 or 135-151. See e.g., International Patent Application WO 2021/127455, the contents of which are incorporated herein by reference in their entireties.

TABLE 8

| | SEQ ID NO: |
|---|---|
| CGAGGCCGGGGCGGGGCACA | 67 |
| CGGGGCGGGGCCGTGGAGGG | 68 |
| ACTGTGCCACTATGTTTTCA | 69 |
| GCCTTCATCAGCTTTTCCAG | 70 |
| GCTGCTGCTGCTGCTGCTGC | 71 |
| TGCTGGAAGGACTTGAGGGA | 72 |
| TGTTGCTGCTGCTGCTGCTG | 73 |
| TGCTGCTGCTGCTGCTGCTG | 135 |
| GGCGGCGGCGGCGGCGGCGG | 136 |
| GAGGGGTGGGGAGGCTGGGG | 137 |
| TCCTTGACCTGCTGCTGCAG | 138 |
| CCTTCCACTGGCCATGATGC | 139 |
| ACTGTGCCACTATGTTTTCA | 140 |
| TGAGGTATCAGATTGTCTAG | 141 |
| AAAttAATCTCTTACCTGAT | 142 |
| CCCAGGGCTAGCAAGGAACA | 143 |
| AATTCAGTAGCTTCCCTTAA | 144 |
| CTGGGCCCGCAGCGGAAGGG | 145 |
| TTATTGCTGTCTACTATCCG | 146 |
| TCAGTCCTTCCCAAAGCTCT | 147 |
| TAATCTCTTTACTGATATAA | 148 |
| TCAGCAGTGTTATTTCTTAC | 149 |
| AAACCGttACCAttACtGAGtt | 150 |
| AAAtCGCtGAtttGtGtAGtC | 151 |

Suitable sequences for use in inhibitory nucleic acids (e.g., miRNAs) that target AD and/or PD associated targets are known in the art, e.g., see International Patent Publication WO2011/133890, WO2012/036433, WO2013/007874; U.S. Patent Publications US2016/0264965; U.S. Pat. Nos. 7,829,694, 8,415,319, 10,125,363, 10,011,835 The contents of the foregoing references are incorporated by reference herein in their entirety.

In some embodiments of any of the aspects, the agent that treats a neurological disease or disorder is or comprises an inhibitory nucleic acid. In some embodiments of any of the aspects, inhibitors of the expression of a given gene can be an inhibitory nucleic acid. As used herein, “inhibitory nucleic acid” refers to a nucleic acid molecule which can inhibit the expression of a target, e.g., double-stranded RNAs (dsRNAs), inhibitory RNAs (iRNAs), and the like.

Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA (or modified nucleic acids as described below herein) and which mediates the targeted cleavage of an RNA transcript via an RNA-induced silencing complex (RISC) pathway. In some embodiments of any of the aspects, an iRNA as described herein effects inhibition of the expression and/or activity of a target. In some embodiments of any of the aspects, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA. In some embodiments of any of the aspects, administering an inhibitor (e.g. an iRNA) to a subject can result in a decrease in the target mRNA level in the subject by at least about 5%, about 10%, about 20%, about 30%, about 40%, about 50%, about 60%, about 70%, about 80%, about 90%, about 95%, about 99%, up to and including 100% of the target mRNA level found in the subject without the presence of the iRNA.

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target, e.g., it can span one or more intron boundaries. The other strand (the sense strand) includes a region that is complementary to the antisense strand, such that the two strands hybridize and form a duplex structure when combined under suitable conditions. Generally, the duplex structure is between 15 and 30 base pairs in length inclusive, more generally between 18 and 25 base pairs in length inclusive, yet more generally between 19 and 24 base pairs in length inclusive, and most generally between 19 and 21 base pairs in length, inclusive. Similarly, the region of complementarity to the target sequence is between 15 and 30 base pairs in length inclusive, more generally between 18 and 25 base pairs in length inclusive, yet more generally between 19 and 24 base pairs in length inclusive, and most generally between 19 and 21 base pairs in length nucleotides in length, inclusive. In some embodiments of any of the aspects, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length. Exemplary embodiments of types of inhibitory nucleic acids can include, e.g., siRNA, shRNA, miRNA, and/or amiRNA, which are well known in the art.

In some embodiments of any of the aspects, the inhibitory is a miRNA. MicroRNAs (miRNAs) are small RNAs of 17-25 nucleotides, which function as regulators of gene expression in eukaryotes. A “microRNA” or “miRNA” is a small non-coding RNA molecule capable of mediating transcriptional or post-translational gene silencing. Typically, miRNA is transcribed as a hairpin or stem-loop (e.g., having a self-complementarity, single-stranded backbone) duplex structure, referred to as a primary miRNA (pri-miRNA), which is enzymatically processed (e.g., by Drosha, DGCR8, Pasha, etc.) into a pre-miRNA. The duplex structure comprises a) a first RNA sequence a region of complementarity that is substantially complementary, and generally fully complementary, to a target sequence and b) second RNA sequence region that is complementary to the first RNA sequence strand, such that the two sequences hybridize and form a duplex structure when combined under suitable conditions. The target sequence can be derived from the sequence of an mRNA formed during the expression of the target, e.g., it can span one or more intron boundaries. Generally, the duplex structure is between 15 and 30 base pairs in length inclusive, more generally between 18 and 25 base pairs in length inclusive, yet more generally between 19 and 24 base pairs in length inclusive, and most generally between 19 and 21 base pairs in length, inclusive.

miRNAs are initially expressed in the nucleus as part of long primary transcripts called primary miRNAs (pri-miRNAs). The length of a pri-miRNA can vary. In some embodiments, a pri-miRNA ranges from about 100 to about 5000 base pairs (e.g., about 100, about 200, about 500, about 1000, about 1200, about 1500, about 1800, or about 2000 base pairs) in length. In some embodiments, a pri-miRNA is greater than 200 base pairs in length (e.g., 2500, 5000, 7000, 9000, or more base pairs in length.

Inside the nucleus, pri-miRNAs are partially digested by the enzyme Drosha, to form 65-120 nucleotide-long hairpin precursor miRNAs (pre-miRNAs) that are exported to the cytoplasm for further processing by Dicer into shorter, mature miRNAs, which are the active molecules. In animals, these short RNAs comprise a 5′ proximal “seed” region (nucleotides 2 to 8) which appears to be the primary determinant of the pairing specificity of the miRNA to the 3′ untranslated region (3′-UTR) of a target mRNA. Pre-miRNA, which is also characterized by a hairpin or stem-loop duplex structure, can also vary in length. In some embodiments, pre-miRNA ranges in size from about 40 base pairs in length to about 500 base pairs in length. In some embodiments, pre-miRNA ranges in size from about 50 to 100 base pairs in length. In some embodiments, pre-miRNA ranges in size from about 50 to about 90 base pairs in length (e.g., about 50, about 52, about 54, about 56, about 58, about 60, about 62, about 64, about 66, about 68, about 70, about 72, about 74, about 76, about 78, about 80, about 82, about 84, about 86, about 88, or about 90 base pairs in length).

Generally, pre-miRNA is exported into the cytoplasm, and enzymatically processed by Dicer to first produce an imperfect miRNA/miRNA* duplex and then a single-stranded mature miRNA molecule, which is subsequently loaded into the RNA-induced silencing complex (RISC). Typically, a mature miRNA molecule ranges in size from about 19 to about 30 base pairs in length. In some embodiments, a mature miRNA molecule is about 19, about 20, about 21, about 22, about 23, about 24, about 25, about 26, about 27, about 28, about 29, or 30 base pairs in length. In some embodiments, an isolated nucleic acid of the disclosure comprises a sequence encoding a pri-miRNA, a pre-miRNA, or a mature miRNA comprising a sequence set forth in any one of SEQ ID NOs: 6-17, 40-44, 50-66, 158-185, or 217-260.

In the context of the invention, a miRNA molecule or an equivalent or a mimic or an isomiR thereof may be a synthetic or natural or recombinant or mature or part of a mature miRNA or a human miRNA or derived from a human miRNA as further defined in the part dedicated to the general definitions. A human miRNA molecule is a miRNA molecule which is found in a human cell, tissue, organ or body fluids (i.e. endogenous human miRNA molecule). A human miRNA molecule may also be a human miRNA molecule derived from an endogenous human miRNA molecule by substitution, deletion and/or addition of a nucleotide. A miRNA molecule or an equivalent or a mimic thereof may be a single stranded or double stranded RNA molecule. Preferably a miRNA molecule or an equivalent, or a mimic thereof is from 6 to 30 nucleotides in length, preferably 12 to 30 nucleotides in length, preferably 15 to 28 nucleotides in length, more preferably said molecule has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

In a preferred embodiment, a miRNA molecule or equivalent or mimic or isomiR thereof comprises at least 6 of the 7 nucleotides present in the seed sequence of said miRNA molecule or equivalent or mimic or isomiR thereof. Preferably in this embodiment, a miRNA molecule or an equivalent or a mimic or isomiR thereof is from 6 to 30 nucleotides in length and more preferably comprises at least 6 of the 7 nucleotides present in the seed sequence of said miRNA molecule or equivalent thereof. Even more preferably a miRNA molecule or an equivalent or a mimic or isomiR thereof is from 15 to 28 nucleotides in length and more preferably comprises at least 6 of the 7 nucleotides present in the seed sequence, even more preferably a miRNA molecule has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

Accordingly, a preferred miRNA molecule or equivalent or mimic or isomiR thereof comprises at least 6 of the 7 nucleotides present in the seed sequence identified in or as SEQ ID NO: 6-17, 40-44, 50-66, 158-185, or 217-260 and more preferably has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

Delivery vehicles for miRNA include but are not limited to the following: liposomes, polymeric nanoparticles, viral systems, conjugation of lipids or receptor-binding molecules, exosomes, and bacteriophage; see e.g., Baumann and Winkler, miRNA-based therapies: Strategies and delivery platforms for oligonucleotide and non-oligonucleotide agents, Future Med Chem. 2014, 6(17): 1967-1984; U.S. Pat. Nos. 8,900,627; 9,421,173; 9,555,060; WO 2019/177550; the contents of each of which are incorporated herein by reference in their entireties.

A microRNA sequence comprises a “seed” region, i.e., a sequence in the region of positions 2-8 of the mature microRNA, which sequence has perfect Watson-Crick complementarity to the miRNA target sequence of the nucleic acid. In one embodiment, the viral genome may be engineered to include, alter or remove at least one miRNA binding site, sequence or seed region.

The term substantial complementarity means that is not required to have the first and second RNA sequence to be fully complementary, or to have the first RNA sequence and a reference or target sequence (e.g., SEQ ID NO: 3 or 4) to be fully complementary. In one embodiment, the substantial complementarity between a RNA sequence and the target consists of having no mismatches, one mismatched nucleotide, or two mismatched nucleotides. It is understood that one mismatched nucleotide means that over the entire length of the RNA sequence that base pairs with the target one nucleotide does not base pair with the target. Having no mismatches means that all nucleotides base pair with the target, and having 2 mismatches means two nucleotides do not base pair with the target.

The miRNAs and/or the transgene comprising one or more miRNAs can be provided in or comprise a scaffold sequence. As used herein, “scaffold” refers to portions of the miRNA-encoding sequence that are external to the mature duplex structure. For example, the scaffold can comprise loops and/or stem regions. Accordingly, scaffolds are useful in producing, encoding, and/or expressing the miRNAs described herein. Scaffolds used in the compositions and methods described herein can be sequences of, obtained from, and/or derived from endogenous and/or naturally-occurring miRNA scaffolds, e.g., human miRNAs. In some embodiments, the scaffold sequence used in the compositions and methods described herein can be sequences of, obtained from, and/or derived from endogenous and/or naturally-occurring miRNA scaffolds of miRNAs that are overexpressed in one or more NS and/or CNS diseases.

In one embodiment, the scaffold sequence used in the compositions and methods described herein can be sequences of, obtained from, and/or derived from endogenous and/or naturally-occurring miRNA scaffolds of miRNAs that are overexpressed in Huntington's disease. Exemplary scaffolds overexpressed in Huntington's disease are described herein in Table 24.

In one embodiment, the scaffold sequence used in the compositions and methods described herein can be sequences of, obtained from, and/or derived from endogenous and/or naturally-occurring miRNA scaffolds of miRNAs that are overexpressed in Huntington's disease.

In one embodiment, the miRNA used in the vector described herein is selected from Table 24.

TABLE 24
Overexpressed miRNAs in Huntington′s disease

| | | SEQ |
|---|---|---|
| miRNA | | ID |
| name-loci | Primary sequence (5′→3′) | NO: |
| miH12-451 | >miH12-451 | 217 |
| | CUUGGGAAUGGCAAGGAAGGACUUGAGGGACUCGAAGACGAGUCCCUCAAGUCCUCUCUUGCUAUACCCAGA | |
| Hsa-mir-9-1 | >hsa-mir-9-1 MI0000466 | 218 |
| | CGGGGUUGGUUGUUAUCUUUGGUUAUCUAGCUGUAUGAGUGGUGUGGAGUCUUCAUAAAGCUAGAUAACCGA | |
| | AAGUAAAAAUAACCCCA | |
| hsa-mir-10b | >hsa-mir-10b MI0000267 | 219 |
| | CCAGAGGUUGUAACGUUGUCUAUAUAUACCCUGUAGAACCGAAUUUGUGUGGUAUCCGUAUAGUCACAGAUU | |
| | CGAUUCUAGGGGAAUAUAUGGUCGAUGCAAAAACUUCA | |
| hsa-mir-16-1 | >hsa-mir-16-1 MI0000070 | 220 |
| | GUCAGCAGUGCCUUAGCAGCACGUAAAUAUUGGCGUUAAGAUUCUAAAAUUAUCUCCAGUAUUAACUGUGCU | |
| | GCUGAAGUAAGGUUGAC | |
| hsa-mir-16-2 | >hsa-mir-16-2 MI0000115 | 221 |
| | GUUCCACUCUAGCAGCACGUAAAUAUUGGCGUAGUGAAAUAUAUAUUAAACACCAAUAUUACUGUGCUGCUU | |
| | UAGUGUGAC | |
| hsa-mir-21 | >hsa-mir-21 MI0000077 | 222 |
| | UGUCGGGUAGCUUAUCAGACUGAUGUUGACUGUUGAAUCUCAUGGCAACACCAGUCGAUGGGCUGUCUGACA | |
| hsa-mir-27b | >hsa-mir-27b MI0000440 | 223 |
| | ACCUCUCUAACAAGGUGCAGAGCUUAGCUGAUUGGUGAACAGUGAUUGGUUUCCGCUUUGUUCACAGUGGCU | |
| | AAGUUCUGCACCUGAAGAGAAGGUG | |
| hsa-mir-29a | >hsa-mir-29a MI0000087 | 224 |
| | AUGACUGAUUUCUUUUGGUGUUCAGAGUCAAUAUAAUUUUCUAGCACCAUCUGAAAUCGGUUAU | |
| hsa-mir-30a | >hsa-mir-30a MI0000088 | 225 |
| | GCGACUGUAAACAUCCUCGACUGGAAGCUGUGAAGCCACAGAUGGGCUUUCAGUCGGAUGUUUGCAGCUGC | |
| hsa-mir-92a-1 | >hsa-mir-92a-1 MI0000093 | 226 |
| | CUUUCUACACAGGUUGGGAUCGGUUGCAAUGCUGUGUUUCUGUAUGGUAUUGCACUUGUCCCGGCCUGUUGA | |
| | GUUUGG | |
| hsa-mir-92a-2 | >hsa-mir-92a-2 MI0000094 | 227 |
| | UCAUCCCUGGGUGGGGAUUUGUUGCAUCAUCCCUGGGUGGGGAUUUGUUGCAUUACUUGUGUUCUAUAUAAA | |
| | GUAUUGCACUUGUCCCGGCCUGUGGAAGA | |
| hsa-mir-100 | >hsa-mir-100 MI0000102 | 228 |
| | CCUGUUGCCACAAACCCGUAGAUCCGAACUUGUGGUAUUAGUCCGCACAAGCUUGUAUCUAUAGGUAUGUGU | |
| | CUGUUAGG | |
| hsa-mir-122 | >hsa-mir-122 MI0000442 | 229 |
| | CCUUAGCAGAGCUGUGGAGUGUGACAAUGGUGUUUGUGUCUAAACUAUCAAACGCCAUUAUCACACUAAAUA | |
| | GCUACUGCUAGGC | |
| Hsa-miR-124-1 | >hsa-mir-124-1 MI0000443 | 230 |
| | AGGCCUCUCUCUCCGUGUUCACAGCGGACCUUGAUUUAAAUGUCCAUACAAUUAAGGCACGCGGUGAAUGCC | |
| | AAGAAUGGGGCUG | |
| hsa-mir-124-2 | >hsa-mir-124-2 MI0000444 | 231 |
| | AUCAAGAUUAGAGGCUCUGCUCUCCGUGUUCACAGCGGACCUUGAUUUAAUGUCAUACAAUUAAGGCACGCG | |
| | GUGAAUGCCAAGAGCGGAGCCUACGGCUGCACUUGAA | |
| hsa-mir-124-3 | >hsa-mir-124-3 MI0000445 | 232 |
| | UGAGGGCCCCUCUGCGUGUUCACAGCGGACCUUGAUUUAAUGUCUAUACAAUUAAGGCACGCGGUGAAUGCC | |
| | AAGAGAGGCGCCUCC | |
| hsa-mir-128-1 | >hsa-mir-128-1 MI0000447 | 233 |
| | UGAGCUGUUGGAUUCGGGGCCGUAGCACUGUCUGAGAGGUUUACAUUUCUCACAGUGAACCGGUCUCUUUUU | |
| | CAGCUGCUUC | |
| hsa-mir-135b | >hsa-mir-135b MI0000810 | 234 |
| | CACUCUGCUGUGGCCUAUGGCUUUUCAUUCCUAUGUGAUUGCUGUCCCAAACUCAUGUAGGGCUAAAAGCCA | |
| | UGGGCUACAGUGAGGGGCGAGCUCC | |
| hsa-mir-140 | >hsa-mir-140 MI0000456 | 235 |
| | UGUGUCUCUCUCUGUGUCCUGCCAGUGGUUUUACCCUAUGGUAGGUUACGUCAUGCUGUUCUACCACAGGGU | |
| | AGAACCACGGACAGGAUACCGGGGCACC | |
| hsa-mir-151b | >hsa-mir-151b MI0003772 | 236 |
| | ACCUCUGAUGUGUCAGUCUCUCUUCAGGGCUCCCGAGACACAGAAACAGACACCUGCCCUCGAGGAGCUCAC | |
| | AGUCUAGACAAACAAACCCAGGGU | |
| hsa-mir-155 | >hsa-mir-155 MI0000681 | 237 |
| | CUGUUAAUGCUAAUCGUGAUAGGGGUUUUUGCCUCCAACUGACUCCUACAUAUUAGCAUUAACAG | |
| hsa-mir-184 | >hsa-mir-184 MI0000481 | 238 |
| | CCAGUCACGUCCCCUUAUCACUUUUCCAGCCCAGCUUUGUGACUGUAAGUGUUGGACGGAGAACUGAUAAGG | |
| | GUAGGUGAUUGA | |
| hsa-mir-196a- | >hsa-mir-196a-1 MI0000238 | 239 |
| 1 | GUGAAUUAGGUAGUUUCAUGUUGUUGGGCCUGGGUUUCUGAACACAACAACAUUAAACCACCCGAUUCAC | |
| hsa-mir-196a- | >hsa-mir-196a-2 MI0000279 | 240 |
| 2 | UGCUCGCUCAGCUGAUCUGUGGCUUAGGUAGUUUCAUGUUGUUGGGAUUGAGUUUUGAACUCGGCAACAAGA | |
| | AACUGCCUGAGUUACAUCAGUCGGUUUUCGUCGAG GGC | |
| hsa-mir-196b | >hsa-mir-196b MI0001150 | 241 |
| | ACUGGUCGGUGAUUUAGGUAGUUUCCUGUUGUUGGGAUCCACCUUUCUCUCGACAGCACGACACUGCCUUCA | |
| | UUACUUCAG UUG | |
| hsa-mir-200a | >hsa-mir-200a MI0000737 | 242 |
| | CCGGGCCCCUGUGAGCAUCUUACCGGACAGUGCUGGAUUUCCCAGCUUGACUCUAACACUGUCUGGUAACGA | |
| | UGUUCAAAGG UGACCCGC | |
| hsa-mir-200c | >hsa-mir-200c MI0000650 | 243 |
| | CCCUCGUCUUACCCAGCAGUGUUUGGGUGCGGUUGGGAGUCUCUAAUACUGCCG GGUAAUGAUGGAGG | |
| hsa-mir-219a- | >hsa-mir-219a-2 MI0000740 | 244 |
| 2 | ACUCAGGGGCUUCGCCACUGAUUGUCCAAACGCAAUUCUUGUACGAGUCUGCGGCCAACCGAGAAUUGUGGC | |
| | UGGACAUCU GUGGCUGAGCUCCGGG | |
| hsa-mir-320a | >hsa-mir-320a MI0000542 | 245 |
| | CUCCCCUCCGCCUUCUCUUCCCGGUUCUUCCCGGAGUCGGGAAAAGCUGGGUUGA GAGGGCGAAAAAGGA | |
| | UG | |
| hsa-mir-330 | >hsa-mir-330 MI0000803 | 246 |
| | CUUUGGCGAUCACUGCCUCUCUGGGCCUGUGUCUUAGGCUCUGCAAGAUCAACCGAGCAAAGCACACGGCCU | |
| | GCAGAGAGGCAGCGCUCUGCCC | |
| hsa-mir-375 | >hsa-mir-375 MI0000783 | 247 |
| | CCCCGCGACGAGCCCCUCGCACAAACCGGACCUGAGCGUUUUGUUCGUUCGGCUC GCGUGAGGC | |
| hsa-mir-451a | >hsa-mir-451a MI0001729 | 248 |
| | CUUGGGAAUGGCAAGGAAACCGUUACCAUUACUGAGUUUAGUAAUGGUAAUGGUUCUCUUGCUAUACCCAGA | |
| hsa-mir-449c | >hsa-mir-449c MI0003823 | 249 |
| | GCUGGGAUGUGUCAGGUAGGCAGUGUAUUGCUAGCGGCUGUUAAUGAUUUUAACAGUUGCUAGUUGCACUCC | |
| | UCUCUGU UGCAUUCAGAAGC | |
| hsa-mir-484 | >hsa-mir-484 MI0002468 | 250 |
| | AGCCUCGUCAGGCUCAGUCCCCUCCCGAUAAACCCCUAAAUAGGGACUUUCCCGGGGGGUGACCCUGGCUUU | |
| | UUUGGCG | |
| hsa-mir-499a | >hsa-mir-499a MI0003183 | 251 |
| | GCCCUGUCCCCUGUGCCUUGGGCGGGCGGCUGUUAAGACUUGCAGUGAUGUUUAACUCCUCUCCACGUGAAC | |
| | AUCACAGCAAGUCUGUGCUGCUUCCCGUCCCUACGCUGCCUGGGCAGGGU | |
| hsa-mir-499 | >hsa-mir-499b MI0017396 | 252 |
| | GGAAGCAGCACAGACUUGCUGUGAUGUUCACGUGGAGAGGAGUUAAACAUCACU GCAAGUCUUAACAGCCG | |
| | CC | |
| hsa-mir-520f | >hsa-mir-520f MI0003146 | 253 |
| | UCUCAGGCUGUGACCCUCUAAAGGGAAGCGCUUUCUGUGGUCAGAAAGAAAAGCAAGUGCUUCCUUUUAGAG | |
| | GGUUACCGUUUGGGA | |
| hsa-miR-549a | >hsa-mir-549a MI0003679 | 254 |
| | AGACAUGCAACUCAAGAAUAUAUUGAGAGCUCAUCCAUAGUUGUCACUGUCUCAAAUCAGUGACAACUAUGG | |
| | AUGAGCUCU UAAUAUAUCCCAGGC | |
| hsa-mir-615 | >hsa-mir-615 MI0003628 | 255 |
| | CUCGGGAGGGGCGGGAGGGGGGUCCCCGGUGCUCGGAUCUCGAGGGUGCUUAUUGUUCGGUCCGAGCCUGGG | |
| | UCUCCCUC UUCCCCCCAACCCCCC | |
| hsa-mir-888 | >hsa-mir-888 MI0005537 | 256 |
| | GGCAGUGCUCUACUCAAAAAGCUGUCAGUCACUUAGAUUACAUGUGACUGACACCUCUUUGGGUGAAGGAAG | |
| | GCUCA | |
| hsa-mir-3928 | >hsa-mir-3928 MI0016438 | 257 |
| | GCUGAAGCUCUAAGGUUCCGCCUGCGGGCAGGAAGCGGAGGAACCUUGGAGCUUCGGC | |
| hsa-mir-4317 | >hsa-mir-4317 MI0015850 | 258 |
| | AAAAGGCGAGACAUUGCCAGGGAGUUUAUUUUGUAGCUCUCUUGAUAAAAUGU UUUAGCAAACAC | |
| hsa-mir-4488 | >hsa-mir-4488 MI0016849 | 259 |
| | GGUAGGGGGCGGGCUCCGGCGCUGGGACCCCACUAGGGUGGCGCCUUGGCCCCGCCCCGCCC | |
| hsa-mir-8082 | >hsa-mir-8082 MI0025918 | 260 |
| | GCCUGUGUGAUGAUGGAGCUGGGAAUACUCUGGGGAGAGAGUCCUCUUUUCAGCUGUAUUUUGCUUCCUUCC | |
| | CACACAGAC | |

### Nucleic Acids

In some aspects, the disclosure provides isolated nucleic acids that are useful for reducing (e.g., inhibiting) expression of a pathogenic gene (e.g., HTT) or which encode CYP46A1. A “nucleic acid” sequence refers to a DNA or RNA sequence. In some embodiments, proteins and nucleic acids of the disclosure are isolated. As used herein, the term “isolated” means artificially produced. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

The skilled artisan will also realize that conservative amino acid substitutions may be made to provide functionally equivalent variants, or homologs of the capsid proteins. In some aspects the disclosure embraces sequence alterations that result in conservative amino acid substitutions. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the proteins and polypeptides disclosed herein.

The isolated nucleic acids of the invention may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise, as disclosed elsewhere herein, one or more regions that encode one or more inhibitory RNAs (e.g., miRNAs) comprising a nucleic acid that targets an endogenous mRNA of a subject. The transgene may also comprise a region encoding, for example, a protein and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid (e.g., the rAAV vector) comprises at least one ITR having a serotype selected from AAV1, AAV2, AAV5, AAV6, AAV6.2, AAV7, AAV8, AAV9, AAV10, AAVrh10, AAV11, and variants thereof. In some embodiments, the isolated nucleic acid comprises a region (e.g., a first region) encoding an AAV2 ITR.

In some embodiments, the isolated nucleic acid further comprises a region (e.g., a second region, a third region, a fourth region, etc.) comprising a second AAV ITR. In some embodiments, the second AAV ITR has a serotype selected from AAV1, AAV2, AAV5, AAV6, AAV6.2, AAV7, AAV8, AAV9, AAV10, AAVrh10, AAV11, and variants thereof. In some embodiments, the second ITR is a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ATRS ITR). Without wishing to be bound by any particular theory, a rAAV vector comprising an ITR lacking a functional TRS produces a self-complementary rAAV vector, for example as described by McCarthy (2008) Molecular Therapy 16(10): 1648-1656.

In addition to the major elements identified above for the recombinant AAV vector, the vector also includes conventional control elements which are operably linked with elements of the transgene in a manner that permits its transcription, translation and/or expression in a cell transfected with the vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency {i.e., Kozak consensus sequence); sequences that enhance protein stability; and when desired, sequences that enhance secretion of the encoded product. A number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly, two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., miRNA).

In some aspects, the disclosure provides an isolated nucleic acid comprising a transgene, wherein the transgene comprises a nucleic acid sequence encoding one or more microRNAs (e.g., miRNAs).

It should be appreciated that an isolated nucleic acid or vector (e.g., rAAV vector), in some embodiments comprises a nucleic acid sequence encoding more than one (e.g., a plurality, such as 2, 3, 4, 5, 10, or more) miRNAs. In some embodiments, each of the more than one miRNAs targets (e.g., hybridizes or binds specifically to) the same target gene (e.g., an isolated nucleic acid encoding three unique miRNAs, where each miRNA targets the HTT gene). In some embodiments, each of the more than one miRNAs targets (e.g., hybridizes or binds specifically to) a different target gene.

In some aspects, the disclosure provides isolated nucleic acids and vectors (e.g., rAAV vectors) that encode one or more artificial miRNAs. As used herein “artificial miRNA” or “amiRNA” refers to an endogenous pri-miRNA or pre-miRNA (e.g., a miRNA backbone, which is a precursor miRNA capable of producing a functional mature miRNA), in which the miRNA and miRNA* (e.g., passenger strand of the miRNA duplex) sequences have been replaced with corresponding amiRNA/amiRNA* sequences that direct highly efficient RNA silencing of the targeted gene, for example as described by Eamens et al. (2014), Methods Mol. Biol. 1062:211-224. For example, in some embodiments an artificial miRNA comprises a miR-155 pri-miRNA backbone into which a sequence encoding a mature HTT-specific miRNA (e.g., any one of SEQ ID NOs: 6-17, 40-44, 50-66, 158-185, or 217-260) has been inserted in place of the endogenous miR-155 mature miRNA-encoding sequence. In some embodiments, miRNA (e.g., an artificial miRNA) as described by the disclosure comprises a miR-155 backbone sequence, a miR-30 backbone sequence, a mir-64 backbone sequence, or a miR-122 backbone sequence.

A region comprising a transgene (e.g., a second region, third region, fourth region, etc.) may be positioned at any suitable location of the isolated nucleic acid. The region may be positioned in any untranslated portion of the nucleic acid, including, for example, an intron, a 5′ or 3′ untranslated region, etc.

In some cases, it may be desirable to position the region (e.g., the second region, third region, fourth region, etc.) upstream of the first codon of a nucleic acid sequence encoding a protein (e.g., a protein coding sequence). For example, the region may be positioned between the first codon of a protein coding sequence) and 2000 nucleotides upstream of the first codon. The region may be positioned between the first codon of a protein coding sequence and 1000 nucleotides upstream of the first codon. The region may be positioned between the first codon of a protein coding sequence and 500 nucleotides upstream of the first codon. The region may be positioned between the first codon of a protein coding sequence and 250 nucleotides upstream of the first codon. The region may be positioned between the first codon of a protein coding sequence and 150 nucleotides upstream of the first codon. In some cases (e.g., when a transgene lacks a protein coding sequence), it may be desirable to position the region (e.g., the second region, third region, fourth region, etc.) upstream of the poly-A tail of a transgene. For example, the region may be positioned between the first base of the poly-A tail and 2000 nucleotides upstream of the first base. The region may be positioned between the first base of the poly-A tail and 1000 nucleotides upstream of the first base. The region may be positioned between the first base of the poly-A tail and 500 nucleotides upstream of the first base. The region may be positioned between the first base of the poly-A tail and 250 nucleotides upstream of the first base. The region may be positioned between the first base of the poly-A tail and 150 nucleotides upstream of the first base. The region may be positioned between the first base of the poly-A tail and 100 nucleotides upstream of the first base. The region may be positioned between the first base of the poly-A tail and 50 nucleotides upstream of the first base. The region may be positioned between the first base of the poly-A tail and 20 nucleotides upstream of the first base. In some embodiments, the region is positioned between the last nucleotide base of a promoter sequence and the first nucleotide base of a poly-A tail sequence.

In some cases, the region may be positioned downstream of the last base of the poly-A tail of a transgene. The region may be between the last base of the poly-A tail and a position 2000 nucleotides downstream of the last base. The region may be between the last base of the poly-A tail and a position 1000 nucleotides downstream of the last base. The region may be between the last base of the poly-A tail and a position 500 nucleotides downstream of the last base. The region may be between the last base of the poly-A tail and a position 250 nucleotides downstream of the last base. The region may be between the last base of the poly-A tail and a position 150 nucleotides downstream of the last base.

It should be appreciated that in cases where a transgene encodes more than one miRNA, each miRNA may be positioned in any suitable location within the transgene. For example, a nucleic acid encoding a first miRNA may be positioned in an intron of the transgene and a nucleic acid sequence encoding a second miRNA may be positioned in another untranslated region (e.g., between the last codon of a protein coding sequence and the first base of the poly-A tail of the transgene).

### Promoters

In some embodiments, the transgene further comprises a nucleic acid sequence encoding one or more expression control sequences (e.g., a promoter, etc.). Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene.

For nucleic acids encoding proteins, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, P et al., Human Gene Therapy, 2000; 11: 1921-1931.; and Klump, H et al., Gene Therapy, 2001; 8: 811-817).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EF1 a promoter [Invitrogen]. In some embodiments, a promoter is an enhanced chicken β-actin promoter. In some embodiments, a promoter is a U6 promoter. In some embodiments, a promoter is a human synapsin (hSYN) promoter. In some embodiments, a promoter is a GFA2 promoter.

In one embodiment, the promoter is a CAG promoter. In one embodiment, the CAG promoter has a sequence of SEQ ID NO: 193. In some embodiments, the CAG promoter comprises or consists of a sequence which is at least 60%, 65%, 70%, 75%, 80%, 85%, 90%, 91%, 92%, 93%, 94%, 95%, 96%, 97%, 98% or 99% identical to any one of SEQ ID NOs: 193. CAG promoters are capable of driving expression, e.g., of a transgene it is operatively linked to, in the target neurons and astrocytes.

(SEQ ID NO: 193)
gactagttattaatagtaatcaattacggggtcattagttcatagcccatatatggagttccgcgttacataact
tacggtaaatggcccgcctggctgaccgcccaacgacccccgcccattgacgtcaataatgacgtatgttcccat
agtaacgccaatagggactttccattgacgtcaatgggtggagtatttacggtaaactgcccacttggcagtaca
tcaagtgtatcatatgccaagtacgccccctattgacgtcaatgacggtaaatggcccgcctggcattatgccca
gtacatgaccttatgggactttcctacttggcagtacatctacgtattagtcatcgctattaccatggtcgaggt
gagccccacgttctgcttcactctccccatctcccccccctccccacccccaattttgtatttatttatttttta
attattttgtgcagcgatgggggcgggggggggggggggcgcgcgccaggcggggcggggcggggcgaggggcgg
ggcggggcgaggcggagaggtgcggcggcagccaatcagagcggcgcgctccgaaagtttccttttatggcgagg
cggcggcggcggcggccctataaaaagcgaagcgcgcggcgggcgggagtcgctgcgcgctgccttcgccccgtg
ccccgctccgccgccgcctcgcgccgcccgccccggctctgactgaccgcgttactcccacaggtgagcgggcgg
gacggcccttctcctccgggctgtaattagcgcttggtttaatgacggcttgtttcttttctgtggctgcgtgaa
agccttgaggggctccgggagggccctttgtgcggggggagcggctcggggggtgcgtgcgtgtgtgtgtgcgtg
gggagcgccgcgtgcggctccgcgctgcccggcggctgtgagcgctgcgggcgcggcgcggggctttgtgcgctc
cgcagtgtgcgcgaggggagcgcggccgggggcggtgccccgcggtgcggggggggctgcgaggggaacaaaggc
tgcgtgcggggtgtgtgcgtgggggggtgagcagggggtgtgggcgcgtcggtcgggctgcaaccccccctgcac
ccccctccccgagttgctgagcacggcccggcttcgggtgcggggctccgtacggggcgtggcgcggggctcgcc
gtgccgggcggggggtggcggcaggtgggggtgccgggcggggcggggccgcctcgggccggggagggctcgggg
gaggggcgcggcggcccccggagcgccggcggctgtcgaggcgcggcgagccgcagccattgccttttatggtaa
tcgtgcgagagggcgcagggacttcctttgtcccaaatctgtgcggagccgaaatctgggaggcgccgccgcacc
ccctctagcgggcgcggggcgaagcggtgcggcgccggcaggaaggaaatgggcggggagggccttcgtgcgtcg
ccgcgccgccgtccccttctccctctccagcctcggggctgtccgcggggggacggctgccttcgggggggacgg
ggcagggcggggttcggcttctggcgtgtgaccggcggctctagag

Underlined text (e.g., 453-819 bp of SEQ ID NO: 193) indicate CMV enhancer.

Bolded, italicized text (e.g., 821-1094 bp of SEQ ID NO: 193) indicate ACTB proximal promoter.

Double underlined text (e.g., 1191-2065 bp of SEQ ID NO: 193) indicate ACTB intron.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionein (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268: 1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a a-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3: 1002-9 (1996); alpha-fetoprotein (AFP) promoter, Arbuthnot et al., Hum. Gene Ther., 7: 1503-14 (1996)), bone osteocalcin promoter (Stein et al., Mol. Biol. Rep., 24: 185-96 (1997)); bone sialoprotein promoter (Chen et al., J. Bone Miner. Res., 11:654-64 (1996)), CD2 promoter (Hansal et al., J. Immunol., 161: 1063-8 (1998); immunoglobulin heavy chain promoter; T cell receptor a-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan. NS-specific promoters contemplated for use in the present methods and compositions also include those described in Patent Application GB2013940.8 filed Sep. 4, 2020 and GB2005732.9 filed Apr. 20, 2020, which are incorporated by reference herein in their entireties. In some embodiments, the NS-specific promoter is a promoter of Table 10, or a promoter having at least 80%, at least 85%, at least 90%, at least 95%, at least 98% identity to a promoter of Table 10. In some embodiments, the NS-specific promoter is a promoter of Table 10, or a promoter having at least 80%, at least 85%, at least 90%, at least 95%, at least 98% identity to a promoter of Table 10 and retaining the NS-specific promoter activity of the promoter of Table 10.

CNS-specific promoters contemplated for use in the present methods and compositions also include those described in International Patent Application PCT/GB2021/050939 filed Apr. 19, 2021, the contents of which are incorporated by reference herein in their entireties. In some embodiments, the CNS-specific promoter is a promoter of Tables 11-13, or a promoter having at least 80%, at least 85%, at least 90%, at least 95%, at least 98% identity to a promoter of Tables 11-13. In some embodiments, the CNS-specific promoter is a promoter of Tables 11-13, or a promoter having at least 80%, at least 85%, at least 90%, at least 95%, at least 98% identity to a promoter of Tables 11-13 and retaining the CNS-specific promoter activity of the promoter of Tables 11-13.

In some embodiments, the nucleic acid comprises one or more CREs. In some embodiments, the nucleic acid comprises one or more NS-specific CREs or CNS-specific CREs. In some embodiments, the nucleic acid comprises one or more CREs of Tables 13-15, or a CRE having at least 80%, at least 85%, at least 90%, at least 95%, at least 98% identity to a CRE of Tables 13-15. In some embodiments, the CRE is a CRE of Tables 13-15, or a CRE having at least 80, at least 85 h, at least 90%, at least 95%, at least 98% identity to a CRE of Tables 13-15 and retaining the activity of the CRE of Tables 13-15.

In some embodiments, the CRE can comprise one or more CREs known in the art. For example, in one embodiment, the one or more CREs may be selected from SEQ ID NOs: 19-24, 27, 28, 37, 38 in Patent Application GB2013940.8 filed Sep. 4, 2020. For example, in one embodiment, the one or more CREs may be selected from: SEQ ID NOs: 1-8 from WO 2019/199867A1, SEQ ID NOs: 1-7 from WO 2020/076614A1 and SEQ ID NOs: 25-51, 177-178, 188 from WO 2020/097121. The foregoing references are incorporated by reference herein in their entireties.

TABLE 10
NS-specific promoters

| NAME | SEQUENCE | Length |
|---|---|---|
| SP0013 | AACATATCCTGGTGTGGAGTAGGGGACGCTGCTCTGACAGAGGCTCGGGGGCCTGA | 795 |
| (SEQ ID | GCTGGCTCTGTGAGCTGGGGAGGAGGCAGACAGCCAGGCCTTGTCTGCAAGCAGAC | |
| NO: 74) | CTGGCAGCATTGGGCTGGCCGCCCCCCAGGGCCTCCTCTTCATGCCCAGTGAATGA | |
| | CTCACCTTGGCACAGACACAATGTTCGGGGTGGGCACAGTGCCTGCTTCCCGCCGC | |
| | ACCCCAGCCCCCCTCAAATGCCTTCCGAGAAGCCCATTGAGCAGGGGGCTTGCATT | |
| | GCACCCCAGCCTGACAGCCTGGCATCTTGGGATAAAAGCAGCACAGCCCCCTAGGG | |
| | GCTGCCCTTGCTGTGTGGCGCCACCGGCGGTGGAGAACAAGGCTCTATTCAGCCTG | |
| | TGCCCAGGAAAGGGGATCAGGGGATGCCCAGGCATGGACAGTGGGTGGCAGGGGGG | |
| | GAGAGGAGGGCTGTCTGCTTCCCAGAAGTCCAAGGACACAAATGGGTGAGGGGAGG | |
| | GCTAGGCCTGCGCACCCACCCACCGACCCCTCACCCACCGACCCGTCACCCACCGA | |
| | CCAAGGGGCACCCTGGCCTAGAGGGGATGCTGAGCGGGACCCGCCTCCTGCCTCTG | |
| | GCAGTCCCAGATGGGACTTGGACCCCGCAGTTGCTCTCTCGGACCCTAAGTTTCTA | |
| | CCCCTGGATCTAAGGCGGAGCTGGGTTTGCGGATCCCACGGTTCCCGGCGGGGCGG | |
| | GGCCCGGTCGCCCCTCCCCCTCCCCGCCCTCCTGCGCCGGGAGCAGTGCATTGTGG | |
| | GAAACTCCCGA | |
| SP0014 | AACATATCCTGGTGTGGAGTAGGGGACGCTGCTCTGACAGAGGCTCGGGGGCCTGA | 810 |
| (SEQ ID | GCTGGCTCTGTGAGCTGGGGAGGAGGCAGACAGCCAGGCCTTGTCTGCAAGCAGAC | |
| NO: 75) | CTGGCAGCATTGGGCTGGCCGCCCCCCAGGGCCTCCTCTTCATGCCCAGTGAATGA | |
| | CTCACCTTGGCACAGACACAATGTTCGGGGTGGGCACAGTGCCTGCTTCCCGCCGC | |
| | ACCCCAGCCCCCCTCAAATGCCTTCCGAGAAGCCCATTGAGCAGGGGGCTTGCATT | |
| | GCACCCCAGCCTGACAGCCTGGCATCTTGGGATAAAAGCAGCACAGCCCCCTAGGG | |
| | GCTGCCCTTGCTGTGTGGCGCCACCGGCGGTGGAGAACAAGGCTCTATTCAGCCTG | |
| | TGCCCAGGAAAGGGGATCAGGGGATGCCCAGGCATGGACAGTGGGTGGCAGGGGGG | |
| | GAGAGGAGGGCTGTCTGCTTCCCAGAAGTCCAAGGACACAAATGGGTGAGGGGATG | |
| | CGGCGAGGCGCGTGCGCACTGCCAGCTTCAGCACCGCGGACAGTGCCTTCGCCCCC | |
| | GCCTGGCGGCGCGCGCCACCGCCGCCTCAGCACTGAAGGCGCGCTGACGTCACTCG | |
| | CCGGTCCCCCGCAAACTCCCCTTCCCGGCCACCTTGGTCGCGTCCGCGCCGCCGCC | |
| | GGCCCAGCCGGACCGCACCACGCGAGGCGCGAGATAGGGGGGCACGGGCGCGACCA | |
| | TCTGCGCTGCGGCGCCGGCGACTCAGCGCTGCCTCAGTCTGCGGTGGGCAGCGGAG | |
| | GAGTCGTGTCGTGCCTGAGAGCGCAG | |
| SP0026 | AACATATCCTGGTGTGGAGTAGGGGACGCTGCTCTGACAGAGGCTCGGGGGCCTGA | 916 |
| (SEQ ID | GCTGGCTCTGTGAGCTGGGGAGGAGGCAGACAGCCAGGCCTTGTCTGCAAGCAGAC | |
| NO: 76) | CTGGCAGCATTGGGCTGGCCGCCCCCCAGGGCCTCCTCTTCATGCCCAGTGAATGA | |
| | CTCACCTTGGCACAGACACAATGTTCGGGGTGGGCACAGTGCCTGCTTCCCGCCGC | |
| | ACCCCAGCCCCCCTCAAATGCCTTCCGAGAAGCCCATTGAGCAGGGGGCTTGCATT | |
| | GCACCCCAGCCTGACAGCCTGGCATCTTGGGATAAAAGCAGCACAGCCCCCTAGGG | |
| | GCTGCCCTTGCTGTGTGGCGCCACCGGCGGTGGAGAACAAGGCTCTATTCAGCCTG | |
| | TGCCCAGGAAAGGGGATCAGGGGATGCCCAGGCATGGACAGTGGGTGGCAGGGGGG | |
| | GAGAGGAGGGCTGTCTGCTTCCCAGAAGTCCAAGGACACAAATGGGTGAGGGGAAG | |
| | CGCGCAGAGTCTGCATGCGTGAGGAAGCTCCTGGGCGCGTCACAGCCGCGCTATTC | |
| | TCAGCGTCTCTCCTTTTATGGCTCCGGAAGTGAGCTGGGGTTGCTGGCAGCCTGGC | |
| | TGGCACTGGGCTAGGCCTGCGCACCCACCCACCGACCCCTCACCCACCGACCCGTC | |
| | ACCCACCGACCAAGGGGCACCCTGGCCTAGAGGGGATGCTGAGCGGGACCCGCCTC | |
| | CTGCCTCTGGCAGTCCCAGATGGGACTTGGACCCCGCAGTTGCTCTCTCGGACCCT | |
| | AAGTTTCTACCCCTGGATCTAAGGCGGAGCTGGGTTTGCGGATCCCACGGTTCCCG | |
| | GCGGGGCGGGGCCCGGTCGCCCCTCCCCCTCCCCGCCCTCCTGCGCCGGGAGCAGT | |
| | GCATTGTGGGAAACTCCCGA | |
| SP0027 | AACATATCCTGGTGTGGAGTAGGGGACGCTGCTCTGACAGAGGCTCGGGGGCCTGA | 931 |
| (SEQ ID | GCTGGCTCTGTGAGCTGGGGAGGAGGCAGACAGCCAGGCCTTGTCTGCAAGCAGAC | |
| NO: 77) | CTGGCAGCATTGGGCTGGCCGCCCCCCAGGGCCTCCTCTTCATGCCCAGTGAATGA | |
| | CTCACCTTGGCACAGACACAATGTTCGGGGTGGGCACAGTGCCTGCTTCCCGCCGC | |
| | ACCCCAGCCCCCCTCAAATGCCTTCCGAGAAGCCCATTGAGCAGGGGGCTTGCATT | |
| | GCACCCCAGCCTGACAGCCTGGCATCTTGGGATAAAAGCAGCACAGCCCCCTAGGG | |
| | GCTGCCCTTGCTGTGTGGCGCCACCGGCGGTGGAGAACAAGGCTCTATTCAGCCTG | |
| | TGCCCAGGAAAGGGGATCAGGGGATGCCCAGGCATGGACAGTGGGTGGCAGGGGGG | |
| | GAGAGGAGGGCTGTCTGCTTCCCAGAAGTCCAAGGACACAAATGGGTGAGGGGAAG | |
| | CGCGCAGAGTCTGCATGCGTGAGGAAGCTCCTGGGCGCGTCACAGCCGCGCTATTC | |
| | TCAGCGTCTCTCCTTTTATGGCTCCGGAAGTGAGCTGGGGTTGCTGGCAGCCTGGC | |
| | TGGCACTTGCGGCGAGGCGCGTGCGCACTGCCAGCTTCAGCACCGCGGACAGTGCC | |
| | TTCGCCCCCGCCTGGCGGCGCGCGCCACCGCCGCCTCAGCACTGAAGGCGCGCTGA | |
| | CGTCACTCGCCGGTCCCCCGCAAACTCCCCTTCCCGGCCACCTTGGTCGCGTCCGC | |
| | GCCGCCGCCGGCCCAGCCGGACCGCACCACGCGAGGCGCGAGATAGGGGGGCACGG | |
| | GCGCGACCATCTGCGCTGCGGCGCCGGCGACTCAGCGCTGCCTCAGTCTGCGGTGG | |
| | GCAGCGGAGGAGTCGTGTCGTGCCTGAGAGCGCAG | |
| SP0030 | AACATATCCTGGTGTGGAGTAGGGGACGCTGCTCTGACAGAGGCTCGGGGGCCTGA | 617 |
| (SEQ ID | GCTGGCTCTGTGAGCTGGGGAGGAGGCAGACAGCCAGGCCTTGTCTGCAAGCAGAC | |
| NO: 78) | CTGGCAGCATTGGGCTGGCCGCCCCCCAGGGCCTCCTCTTCATGCCCAGTGAATGA | |
| | CTCACCTTGGCACAGACACAATGTTCGGGGTGGGCACAGTGCCTGCTTCCCGCCGC | |
| | ACCCCAGCCCCCCTCAAATGCCTTCCGAGAAGCCCATTGAGCAGGGGGCTTGCATT | |
| | GCACCCCAGCCTGACAGCCTGGCATCTTGGGATAAAAGCAGCACGGGCTAGGCCTG | |
| | CGCACCCACCCACCGACCCCTCACCCACCGACCCGTCACCCACCGACCAAGGGGCA | |
| | CCCTGGCCTAGAGGGGATGCTGAGCGGGACCCGCCTCCTGCCTCTGGCAGTCCCAG | |
| | ATGGGACTTGGACCCCGCAGTTGCTCTCTCGGACCCTAAGTTTCTACCCCTGGATC | |
| | TAAGGCGGAGCTGGGTTTGCGGATCCCACGGTTCCCGGCGGGGCGGGGCCCGGTCG | |
| | CCCCTCCCCCTCCCCGCCCTCCTGCGCCGGGAGCAGTGCATTGTGGGAAACTCCCG | |
| | A | |
| SP0031 | AACATATCCTGGTGTGGAGTAGGGGACGCTGCTCTGACAGAGGCTCGGGGGCCTGA | 632 |
| (SEQ ID | GCTGGCTCTGTGAGCTGGGGAGGAGGCAGACAGCCAGGCCTTGTCTGCAAGCAGAC | |
| NO: 79) | CTGGCAGCATTGGGCTGGCCGCCCCCCAGGGCCTCCTCTTCATGCCCAGTGAATGA | |
| | CTCACCTTGGCACAGACACAATGTTCGGGGTGGGCACAGTGCCTGCTTCCCGCCGC | |
| | ACCCCAGCCCCCCTCAAATGCCTTCCGAGAAGCCCATTGAGCAGGGGGCTTGCATT | |
| | GCACCCCAGCCTGACAGCCTGGCATCTTGGGATAAAAGCAGCACTGCGGCGAGGCG | |
| | CGTGCGCACTGCCAGCTTCAGCACCGCGGACAGTGCCTTCGCCCCCGCCTGGCGGC | |
| | GCGCGCCACCGCCGCCTCAGCACTGAAGGCGCGCTGACGTCACTCGCCGGTCCCCC | |
| | GCAAACTCCCCTTCCCGGCCACCTTGGTCGCGTCCGCGCCGCCGCCGGCCCAGCCG | |
| | GACCGCACCACGCGAGGCGCGAGATAGGGGGGCACGGGCGCGACCATCTGCGCTGC | |
| | GGCGCCGGCGACTCAGCGCTGCCTCAGTCTGCGGTGGGCAGCGGAGGAGTCGTGTC | |
| | GTGCCTGAGAGCGCAG | |
| SP0032 | TTCAGAGGTCGGATCTGAATCCAGCTCCAAGGCCCCAGCACCCAAGCCCTGACCCC | 701 |
| (SEQ ID | ATGGCTGCCTGCTGGCTGGGAGTGGCATTCTTTAGAACAGGAAAGGCAGTGAGTTC | |
| NO: 80) | TCATTGCATCAATACTTGCATTTGCTACAACAGAAGCTTTTTGTCTCTTCCTCACA | |
| | TTCCTTTAGAACACAAGCCTCCTTTTCTGCCTGTGGCCATCGGTCACTGGAGTCAG | |
| | CCTCGTGAGTGGCTTGGTGGCGGATGGCACCGTCCTTTGTGGGAGAAAACAATGTT | |
| | GCTGCCCAGGCCTTTCTGGAATGACCCCTTCCCACCCCCTGACCAGCCCCAGCAAG | |
| | GCCCGGGGCTGGCTGCCTAGTTGATTGGAGCCAAGAGTTTGCTGAATGGATGAAGG | |
| | GAGAAGGGACATCCAAGGGCTAGGCCTGCGCACCCACCCACCGACCCCTCACCCAC | |
| | CGACCCGTCACCCACCGACCAAGGGGCACCCTGGCCTAGAGGGGATGCTGAGCGGG | |
| | ACCCGCCTCCTGCCTCTGGCAGTCCCAGATGGGACTTGGACCCCGCAGTTGCTCTC | |
| | TCGGACCCTAAGTTTCTACCCCTGGATCTAAGGCGGAGCTGGGTTTGCGGATCCCA | |
| | CGGTTCCCGGCGGGGCGGGGCCCGGTCGCCCCTCCCCCTCCCCGCCCTCCTGCGCC | |
| | GGGAGCAGTGCATTGTGGGAAACTCCCGA | |
| SP0033 | TTCAGAGGTCGGATCTGAATCCAGCTCCAAGGCCCCAGCACCCAAGCCCTGACCCC | 716 |
| (SEQ ID | ATGGCTGCCTGCTGGCTGGGAGTGGCATTCTTTAGAACAGGAAAGGCAGTGAGTTC | |
| NO: 81) | TCATTGCATCAATACTTGCATTTGCTACAACAGAAGCTTTTTGTCTCTTCCTCACA | |
| | TTCCTTTAGAACACAAGCCTCCTTTTCTGCCTGTGGCCATCGGTCACTGGAGTCAG | |
| | CCTCGTGAGTGGCTTGGTGGCGGATGGCACCGTCCTTTGTGGGAGAAAACAATGTT | |
| | GCTGCCCAGGCCTTTCTGGAATGACCCCTTCCCACCCCCTGACCAGCCCCAGCAAG | |
| | GCCCGGGGCTGGCTGCCTAGTTGATTGGAGCCAAGAGTTTGCTGAATGGATGAAGG | |
| | GAGAAGGGACATCCAATGCGGCGAGGCGCGTGCGCACTGCCAGCTTCAGCACCGCG | |
| | GACAGTGCCTTCGCCCCCGCCTGGCGGCGCGCGCCACCGCCGCCTCAGCACTGAAG | |
| | GCGCGCTGACGTCACTCGCCGGTCCCCCGCAAACTCCCCTTCCCGGCCACCTTGGT | |
| | CGCGTCCGCGCCGCCGCCGGCCCAGCCGGACCGCACCACGCGAGGCGCGAGATAGG | |
| | GGGGCACGGGCGCGACCATCTGCGCTGCGGCGCCGGCGACTCAGCGCTGCCTCAGT | |
| | CTGCGGTGGGCAGCGGAGGAGTCGTGTCGTGCCTGAGAGCGCAG | |
| SP0019 | TCAGGGGTGCAGCTTTTTTTCTGTCTTTTACTCAGCCTGAGAAAGGTTGTCGTTTG | 792 |
| (SEQ ID | ACAAGGTTTGTTCAGAGGTCGGATCTGAATCCAGCTCCAAGGCCCCAGCACCCAAG | |
| NO: 82) | CCCTGACCCCATGGCTGCCTGCTGGCTGGGAGTGGCATTCTTTAGAACAGGAAAGG | |
| | CAGTGAGTTCTCATTGCATCAATACTTGCATTTGCTACAACAGAAGCTTTTTGTCT | |
| | CTTCCTCACATTCCTTTAGAACACAAGCCTCCTTTTCTGCCTGTGGCCATCGGTCA | |
| | CTGGAGTCAGCCTCGTGAGTGGCTTGGTGGCGGATGGCACCGTCCTTTGTGGGAGA | |
| | AAACAATGTTGCTGCCCAGGCCTTTCTGGAATGACCCCTTCCCACCCCCTGACCAG | |
| | CCCCAGCAAGGCCCGGGGCTGGCTGCCTAGTTGATTGGAGCCAAGAGTTTGCTGAA | |
| | TGGATGAAGGGAGAAGGGACATCCAAGTGACTGAAGAAGATCTTAACAGAAGGGCT | |
| | AGGCCTGCGCACCCACCCACCGACCCCTCACCCACCGACCCGTCACCCACCGACCA | |
| | AGGGGCACCCTGGCCTAGAGGGGATGCTGAGCGGGACCCGCCTCCTGCCTCTGGCA | |
| | GTCCCAGATGGGACTTGGACCCCGCAGTTGCTCTCTCGGACCCTAAGTTTCTACCC | |
| | CTGGATCTAAGGCGGAGCTGGGTTTGCGGATCCCACGGTTCCCGGCGGGGCGGGGC | |
| | CCGGTCGCCCCTCCCCCTCCCCGCCCTCCTGCGCCGGGAGCAGTGCATTGTGGGAA | |
| | ACTCCCGA | |
| SP0020 | CTTCCTCTTATATTTCACCAAGACTCAGTTCCTGAGCAAGAAACCACAGGCACAGC | 686 |
| (SEQ ID | AAGTGCCATGAAAAGCGGCTTTGTGTGGGGTGGGCTCTTCACACTCCAATCTCCAC | |
| NO: 83) | TTCCTTCTCAAGGCCTCAAAAAAAGTTGAAAAATGAAAACAAAAGCCCTGCTGTGT | |
| | TGAGCTGGGCTCTGGCGTTGCCATGGACCCAGGGCAAACAGCGGTGCTCCTGCTCT | |
| | GCCCCCGGCTCAGCTCATGCTGGGCCTGCACTTCTGGAAGGGAGCATGGACTTTGG | |
| | AATGACTGGTTAGAACCCAAATGAATTAATGGAATTTGACATAGTTCAAAAATAAT | |
| | AAAATGTGATACCCATGAAATGCTGATATTCTGCCTTAATTTGCCAGATTGGGGGC | |
| | CGGGCTAGGCCTGCGCACCCACCCACCGACCCCTCACCCACCGACCCGTCACCCAC | |
| | CGACCAAGGGGCACCCTGGCCTAGAGGGGATGCTGAGCGGGACCCGCCTCCTGCCT | |
| | CTGGCAGTCCCAGATGGGACTTGGACCCCGCAGTTGCTCTCTCGGACCCTAAGTTT | |
| | CTACCCCTGGATCTAAGGCGGAGCTGGGTTTGCGGATCCCACGGTTCCCGGGGGG | |
| | CGGGGCCCGGTCGCCCCTCCCCCTCCCCGCCCTCCTGCGCCGGGAGCAGTGCATTG | |
| | TGGGAAACTCCCGA | |
| SP0021 | TCAGGGGTGCAGCTTTTTTTCTGTCTTTTACTCAGCCTGAGAAAGGTTGTCGTTTG | 807 |
| (SEQ ID | ACAAGGTTTGTTCAGAGGTCGGATCTGAATCCAGCTCCAAGGCCCCAGCACCCAAG | |
| NO: 84) | CCCTGACCCCATGGCTGCCTGCTGGCTGGGAGTGGCATTCTTTAGAACAGGAAAGG | |
| | CAGTGAGTTCTCATTGCATCAATACTTGCATTTGCTACAACAGAAGCTTTTTGTCT | |
| | CTTCCTCACATTCCTTTAGAACACAAGCCTCCTTTTCTGCCTGTGGCCATCGGTCA | |
| | CTGGAGTCAGCCTCGTGAGTGGCTTGGTGGCGGATGGCACCGTCCTTTGTGGGAGA | |
| | AAACAATGTTGCTGCCCAGGCCTTTCTGGAATGACCCCTTCCCACCCCCTGACCAG | |
| | CCCCAGCAAGGCCCGGGGCTGGCTGCCTAGTTGATTGGAGCCAAGAGTTTGCTGAA | |
| | TGGATGAAGGGAGAAGGGACATCCAAGTGACTGAAGAAGATCTTAACAGAATGCGG | |
| | CGAGGCGCGTGCGCACTGCCAGCTTCAGCACCGCGGACAGTGCCTTCGCCCCCGCC | |
| | TGGCGGCGCGCGCCACCGCCGCCTCAGCACTGAAGGCGCGCTGACGTCACTCGCCG | |
| | GTCCCCCGCAAACTCCCCTTCCCGGCCACCTTGGTCGCGTCCGCGCCGCCGCCGGC | |
| | CCAGCCGGACCGCACCACGCGAGGCGCGAGATAGGGGGGCACGGGCGCGACCATCT | |
| | GCGCTGCGGCGCCGGCGACTCAGCGCTGCCTCAGTCTGCGGTGGGCAGCGGAGGAG | |
| | TCGTGTCGTGCCTGAGAGCGCAG | |
| SP0022 | CTTCCTCTTATATTTCACCAAGACTCAGTTCCTGAGCAAGAAACCACAGGCACAGC | 701 |
| (SEQ ID | AAGTGCCATGAAAAGCGGCTTTGTGTGGGGTGGGCTCTTCACACTCCAATCTCCAC | |
| NO: 85) | TTCCTTCTCAAGGCCTCAAAAAAAGTTGAAAAATGAAAACAAAAGCCCTGCTGTGT | |
| | TGAGCTGGGCTCTGGCGTTGCCATGGACCCAGGGCAAACAGCGGTGCTCCTGCTCT | |
| | GCCCCCGGCTCAGCTCATGCTGGGCCTGCACTTCTGGAAGGGAGCATGGACTTTGG | |
| | AATGACTGGTTAGAACCCAAATGAATTAATGGAATTTGACATAGTTCAAAAATAAT | |
| | AAAATGTGATACCCATGAAATGCTGATATTCTGCCTTAATTTGCCAGATTGGGGGC | |
| | CTGCGGCGAGGCGCGTGCGCACTGCCAGCTTCAGCACCGCGGACAGTGCCTTCGCC | |
| | CCCGCCTGGCGGCGCGCGCCACCGCCGCCTCAGCACTGAAGGCGCGCTGACGTCAC | |
| | TCGCCGGTCCCCCGCAAACTCCCCTTCCCGGCCACCTTGGTCGCGTCCGCGCCGCC | |
| | GCCGGCCCAGCCGGACCGCACCACGCGAGGCGCGAGATAGGGGGGCACGGGCGCGA | |
| | CCATCTGCGCTGCGGCGCCGGCGACTCAGCGCTGCCTCAGTCTGCGGTGGGCAGCG | |
| | GAGGAGTCGTGTCGTGCCTGAGAGCGCAG | |
| SP0028 | TCAGGGGTGCAGCTTTTTTTCTGTCTTTTACTCAGCCTGAGAAAGGTTGTCGTTTG | 913 |
| (SEQ ID | ACAAGGTTTGTTCAGAGGTCGGATCTGAATCCAGCTCCAAGGCCCCAGCACCCAAG | |
| NO: 86) | CCCTGACCCCATGGCTGCCTGCTGGCTGGGAGTGGCATTCTTTAGAACAGGAAAGG | |
| | CAGTGAGTTCTCATTGCATCAATACTTGCATTTGCTACAACAGAAGCTTTTTGTCT | |
| | CTTCCTCACATTCCTTTAGAACACAAGCCTCCTTTTCTGCCTGTGGCCATCGGTCA | |
| | CTGGAGTCAGCCTCGTGAGTGGCTTGGTGGCGGATGGCACCGTCCTTTGTGGGAGA | |
| | AAACAATGTTGCTGCCCAGGCCTTTCTGGAATGACCCCTTCCCACCCCCTGACCAG | |
| | CCCCAGCAAGGCCCGGGGCTGGCTGCCTAGTTGATTGGAGCCAAGAGTTTGCTGAA | |
| | TGGATGAAGGGAGAAGGGACATCCAAGTGACTGAAGAAGATCTTAACAGAAAGCGC | |
| | GCAGAGTCTGCATGCGTGAGGAAGCTCCTGGGCGCGTCACAGCCGCGCTATTCTCA | |
| | GCGTCTCTCCTTTTATGGCTCCGGAAGTGAGCTGGGGTTGCTGGCAGCCTGGCTGG | |
| | CACTGGGCTAGGCCTGCGCACCCACCCACCGACCCCTCACCCACCGACCCGTCACC | |
| | CACCGACCAAGGGGCACCCTGGCCTAGAGGGGATGCTGAGCGGGACCCGCCTCCTG | |
| | CCTCTGGCAGTCCCAGATGGGACTTGGACCCCGCAGTTGCTCTCTCGGACCCTAAG | |
| | TTTCTACCCCTGGATCTAAGGCGGAGCTGGGTTTGCGGATCCCACGGTTCCCGGCG | |
| | GGGCGGGGCCCGGTCGCCCCTCCCCCTCCCCGCCCTCCTGCGCCGGGAGCAGTGCA | |
| | TTGTGGGAAACTCCCGA | |
| SP0029 | TCAGGGGTGCAGCTTTTTTTCTGTCTTTTACTCAGCCTGAGAAAGGTTGTCGTTTG | 928 |
| (SEQ ID | ACAAGGTTTGTTCAGAGGTCGGATCTGAATCCAGCTCCAAGGCCCCAGCACCCAAG | |
| NO: 87) | CCCTGACCCCATGGCTGCCTGCTGGCTGGGAGTGGCATTCTTTAGAACAGGAAAGG | |
| | CAGTGAGTTCTCATTGCATCAATACTTGCATTTGCTACAACAGAAGCTTTTTGTCT | |
| | CTTCCTCACATTCCTTTAGAACACAAGCCTCCTTTTCTGCCTGTGGCCATCGGTCA | |
| | CTGGAGTCAGCCTCGTGAGTGGCTTGGTGGCGGATGGCACCGTCCTTTGTGGGAGA | |
| | AAACAATGTTGCTGCCCAGGCCTTTCTGGAATGACCCCTTCCCACCCCCTGACCAG | |
| | CCCCAGCAAGGCCCGGGGCTGGCTGCCTAGTTGATTGGAGCCAAGAGTTTGCTGAA | |
| | TGGATGAAGGGAGAAGGGACATCCAAGTGACTGAAGAAGATCTTAACAGAAAGCGC | |
| | GCAGAGTCTGCATGCGTGAGGAAGCTCCTGGGCGCGTCACAGCCGCGCTATTCTCA | |
| | GCGTCTCTCCTTTTATGGCTCCGGAAGTGAGCTGGGGTTGCTGGCAGCCTGGCTGG | |
| | CACTTGCGGCGAGGCGCGTGCGCACTGCCAGCTTCAGCACCGCGGACAGTGCCTTC | |
| | GCCCCCGCCTGGCGGCGCGCGCCACCGCCGCCTCAGCACTGAAGGCGCGCTGACGT | |
| | CACTCGCCGGTCCCCCGCAAACTCCCCTTCCCGGCCACCTTGGTCGCGTCCGCGCC | |
| | GCCGCCGGCCCAGCCGGACCGCACCACGCGAGGCGCGAGATAGGGGGGCACGGGCG | |
| | CGACCATCTGCGCTGCGGCGCCGGCGACTCAGCGCTGCCTCAGTCTGCGGTGGGCA | |
| | GCGGAGGAGTCGTGTCGTGCCTGAGAGCGCAG | |
| SP0011 | CTAGCCCACAGGAAATGTCTGTCTATATCCAGGCAAGTACCTTGCTCATTGGACCA | 529 |
| (SEQ ID | ACCCGAAACTGTTCAGGGAAGATCAGGGAAATCAACTCAGTTACAAATGGGATAAT | |
| NO: 88) | CATGCCCAGTAAAAACTACCTGTGGTGAATAAAGAGTTAACCCCTGTTCCATCTTA | |
| | GGTCACTATGCAGAGTACCAATGAGTACAAGAGATGGTGCCAAAGAGGGTGGCCCC | |
| | TCCCTAGCTGGGAACAGTCAACCCTTAGGAACTAGACTGTCAACACATCAGCCAGC | |
| | CAGAGACAAGGGAAACCGTGGCAACCAAGTGTTGCTGGCACATTGTGAGGTGGTGA | |
| | TGGGAACTGCAGAGGCCCTGCACAGCATGCTAATGAGCCCAGGCAAACATGAGCTC | |
| | TCCCCATAGCTGGGCTGCGGCCCAACCCCACCCCCTCAGGCTATGCCAGGGGGTGT | |
| | TGCCAGGGGCACCCGGGCATCGCCAGTCTAGCCCACTCCTTCATAAAGCCCTCGCA | |
| | TCCCAGGAGCGAGCAGAGCCAGAGC | |
| SP0034 | TCCAAAGAAAAGCCAGATAAGTAGCTGATTATTGCATAGAGCTGACAGTATCACAG | 646 |
| (SEQ ID | GAAGATCAGTAGTAGCAGCTCAAGTACAAAAAGGTTAATTAGCAATACTTAATAAG | |
| NO: 89) | AAAAACTACCTCTGGCAGGTGAAGAGTTAATCCCTGGTCAATTTTAAGCTACTCTG | |
| | CTGAGAGTACTAATAAGTGTAGGGGTTGGAGCCAATGAGGGTGACCCCTTCCTTGA | |
| | TGGGAACAGTCATCCCTTAGGAACTGCCCTGGAAAGCATCAGCCAGCCAGAAAACA | |
| | GGGAAAGAGGCTGAGAAACCGTGGTAACCAAGTTTTGCTGGCACTTTGTAAAATGG | |
| | TAACTGCAACTGCCGAGGCTGTGCAGAGAATGCTAATAAGCCTAGGACAACCTGTA | |
| | AAGAGTGGACCTAGAAAATGTCCACCCGCTAGAGAGAGGGAGCGAGCATGTGCGAT | |
| | GAGCAATAGCTGTGGACCTTACAGTTGCTGCTAACTGCCCTGGTGTGTGTGAGGGA | |
| | GAGAGAGGGAGGGAGGGAGAGAGAGCGCGCTAGCGCGAGAGAGCGAGTGAGCAAGC | |
| | GAGCAGAAAAGAGGTGGAGAGGGGGGGAATAAGAAAGAGAGAGAAGGAAAGGAGAG | |
| | AAGGCAGGAAGAAGGCAAGGGACGAGACAA | |
| SP0035 | CTAGCCCACAGGAAATGTCTGTCTATATCCAGGCAAGTACCTTGCTCATTGGACCA | 614 |
| (SEQ ID | ACCCGAAACTGTTCAGGGAAGATCAGGGAAATCAACTCAGTTACAAATGGGATAAT | |
| NO: 90) | CATGCCCAGTAAAAACTACCTGTGGTGAATAAAGAGTTAACCCCTGTTCCATCTTA | |
| | GGTCACTATGCAGAGTACCAATGAGTACAAGAGATGGTGCCAAAGAGGGTGGCCCC | |
| | TCCCTAGCTGGGAACAGTCAACCCTTAGGAACTAGACTGTCAACACATCAGCCAGC | |
| | CAGAGACAAGGGAAACCGTGGCAACCAAGTGTTGCTGGCACATTGTGAGGTGGTGA | |
| | TGGGAACTGCAGAGGCCCTGCACAGCATGCTAATGAGCCCAGGCAAACATCGCTAG | |
| | AGAGAGGGAGCGAGCATGTGCGATGAGCAATAGCTGTGGACCTTACAGTTGCTGCT | |
| | AACTGCCCTGGTGTGTGTGAGGGAGAGAGAGGGAGGGAGGGAGAGAGAGCGCGCTA | |
| | GCGCGAGAGAGCGAGTGAGCAAGCGAGCAGAAAAGAGGTGGAGAGGGGGGGAATAA | |
| | GAAAGAGAGAGAAGGAAAGGAGAGAAGGCAGGAAGAAGGCAAGGGACGAGACAA | |
| CNS-1 | CTGGGCAGAGAGGGGGCATCGGGGGCATGGCTAGGGGCCAGCACTGTGCTTCCTGG | 696 |
| (SEQ ID | GCGCCTCACCTCCTCCCTGACTCCTGGAGACTCCCAGCCCCTGTCTGGGAGATGAG | |
| NO: 91) | CATTTAGGAATCTGCTTGTGCAGGGGTGGTGGGAGGGGCCGGGGTGGAGGGCGCAT | |
| | CCCCACGGGGAGATTGGATGGAAATGGCCTGCCAGTGTGTGTGTGAGTGTGCGCCT | |
| | GTGGCAGCAGCAGAGTAAACAGCCGCTGCCCTGTCCTCTCTGCGGCCGTGGCCAGG | |
| | TACACAGGCCTGTTTGGACAGCTGCCTTGTCTGTCCGTCTGTTTGGGAGATGCTGG | |
| | CTGATAGATGGGGATGGGCGGACTGTTAACCCCTCGTTGCCTGCACTGCTATGTGC | |
| | TTCCTGCCTCATCCATGGGGTAGAAGGTAGCCAGAAGGTGGTCCTGGCTGTGCCCC | |
| | CAGCTCCTCTCTAGGGGGGAAACCTCTAGTTCTGAGTCAGGGACAGAGTGAGGAGG | |
| | GCTCCAGGGCATCAAGAGCTTGCTCCTCCCCGCACCAGGGAGCCAAGGACAGAGGA | |
| | GAAGGGGGTCTTCCCCAGTGGTGACTAGGGGCAGAATATGTCTCTGAGTGAGTGTC | |
| | TGGAGCCCTCCTCACCCCAACACCATGGGGCTGGGCATAAAAGTCAGGGCAGAGCC | |
| | ATCTATTGCTTACATTTGCTTCTG | |
| CNS-2 | GGTGTGTGGAAGGGTGAGAGGCACACACACAGACACTGAAAGAATCCTAGGCCTGG | 709 |
| (SEQ ID | TAGGCACTTAACAAATGTCTGTTACAGACCAGAATTTTATTGCTGTTAGAGACCCA | |
| NO: 92) | AGCCCCTCATAGGAACAGTGAGAAACAGGTGCAGAAAGGCGGAGTAACTTTATCTA | |
| | AAGTCATAGGCTCCCTGAATAGCAGAGCTGACACCTACAAGGAAGCGTTGGAGACC | |
| | AGATCTACCAGCTAGCCTCCCTGAGACCACGAGGTGGCGCCGCAGCACCGGCTGTG | |
| | GCCGATGCCAGCCAGGTAGCCGGTTTCCCACGTCCCCCGCACGCACGCACCTCTTT | |
| | GCTGCAGGAATCCCGGGCTGCCCCGACCTGGAGTAGGGGGGGTGGTGAGTGGGACT | |
| | GAGTCCCTAGAAGCCTGGACCCTCACTTCGTTCCTGTACATCCAGCTCGCCTGTAG | |
| | ACAGTGGGGGAGGATGAAGGGAAGAGGACTCAAGCGCAACTTTGAATCATCACGCC | |
| | TTCGACAGTCCGCGCACGTTTATTTCATTTATCTTTGAAAACGAGGGAGGGGAAGC | |
| | CTGGAGAAGGCGGGATGGGCCAAGGGTGAGTTGGCCCCCGGGGAGCTGGTCCCTGT | |
| | TCCTGGCTTTAGTCCCAGGGGCGCGGTCTGTGTGTAGGGCGGGCTGGGCATAAAAG | |
| | TCAGGGCAGAGCCATCTATTGCTTACATTTGCTTCTG | |
| CNS-3 | TGCTACCAGAGCCGGGAGAGCTGCTCGGAGACGCCTCCGGGGTGCGGGCTGGACAT | |
| (SEQ ID | GAGCAGCGGCTGCCGGTCCTGGGACTAGGCCCCGCCATTTTGGATCCGCTGACAGG | |
| NO: 93) | TCAGCGAAGTCTCTTCCTAGAGTTCCGGTGTCGTGAAGGCCGCCCTGACATCGCAA | |
| | TAGGGAATTAGTGGGAAGGGCCCTTAAATTGGGCGAGCCAAGGTGGGGGGAGGATT | |
| | GGAACAGAGACAAAAGGGAGGAGAGACGGACAGCGACAAGTGGAGAAAATCGGCGA | |
| | AACTTGAGTGGCAGAGAAGTCTGAGCGCTGAGACCCGGCGGCCCCGTGCGCCTTCC | |
| | CACCTGGCGCCGATCCACTTTCCTCGGGGTAGCGGCCCAACCCACTTCGCTGCCAG | |
| | CCGATCCCTTTTACCCGTGGCTACCGGGACCACTCTACTCTCGCCCACTTGGCTCT | |
| | GCCTAAGCGTCCTAGCCGGAGCGCGGTCTCTGCCACGTGGGGAGGGGCGCGGCCGA | |
| | GTTGCTGAAGAGCGCTTCTGATTGGCCAGAGGGCGGGGTTCTTGGCGTCTCGCCGG | |
| | CCAGACCCCTCCCTCAAAGGCGGGGCCTGGAGATCCACAGCTGGAAAGGGCGGAGC | |
| | CCCAGCAGGGCAGCTGGAAAGGGGCGGGGCCTGACGCGCGCGGCTCGCCGCGGCGG | |
| | GCTGGGGGCGCCCTGGTCTGCCATAAAGTGAATGGGCGCCGGCTGGGGGTGGCAGT | |
| | ACGCGGTGAGGCTCACTCCCTCCGAGAGTCCAGGAGCGCC | |
| CNS-4 | AAGGAGAATGGTAAACAGCAGGAGCGAAGCGGCTGAGGAGAAAGAAGAGGAAAGAA | |
| (SEQ ID | AGGCGAGACGTGGGAGGATTGGAACAGAGACAAAAGGGAGGAGAGACGGACAGCGA | |
| NO: 94) | CAAGTGGAGAAAATCGGCGAAACTTGAGTGGCAGAGAAGTCTGAGCGCTGAGACCC | |
| | GGCGGCCCCGTGCGCCTTCCCACCTGGCGCCGATCCACTTTCCTCGGGGTAGCGGC | |
| | CCAACCCACTTCGCTGCCAGCCGATCCCTTTTACCCGTGGCTACCGGGACCACTCT | |
| | ACTCTCGCCCACTTGGCTCTGCCTAAGCGTCCTAGCCGGAGCGCGGTCTCTGCCAC | |
| | GTGGGGAGGGGCGCGGCCGAGTTGCTGAAGAGCGCTTCTGATTGGCCAGAGGGCGG | |
| | GGTTCTTGGCGTCTCGCCGGCCAGACCCCTCCCTCAAAGGCGGGGCCTGGAGATCC | |
| | ACAGCTGGAAAGGGCGGAGCCCCAGCAGGGCAGCTGGAAAGGGGCGGGGCCTGACG | |
| | CGCGCGGCTCGCCGCGGCGGGCTGGGGGCGCCCTGGTCTGCCATAAAGTGAATGGG | |
| | CGCCGGCTGGGGGTGGCAGTACGCGGTGAGGCTCACTCCCTCCGAGAGTCCAGGAG | |
| | CGCCCGAGCGGAGAGGCGGCCCGGGAGCAGGGGGGCGGCCCCCACTCCGGCCGGGT | |
| | GCCCGGCCCCTGGCCCCTGCCTGCCCTCTAGATCGCCGCCGCAGCCGCCGCTACTG | |
| | GGAGTCTG | |
| CNS-5 | ttgtAATGGGAATAAGGGCAGGACtcctgggtataagtagctcagctgatcccacc | 1744 |
| (SEQ ID | ctgcttctatgtgttaattcatttattcattcattcaacaagcatttgttgaaatg | |
| NO: 95) | ctctttgtgtcaggctcagcaggaagcagtggcaataaaatggtgaacaagaaaga | |
| | ctcggggtttcttcatctatgttgatgtctgcagagaacagtatcagccttctagg | |
| | aagtttgtaatcagatacattgttagagagatacttatctagtaaattcctactca | |
| | tcctataaggctcaaaacaaatgcctctatgaaaccttccgtgattccctcaggca | |
| | gagttaagagcttcctttcctgggcctctatctccttccattagtattataactgt | |
| | ttaccagtttcccctctagactaaatttctcaaaagagagaatgaggtctctttca | |
| | gtcttctttgcatctttaaactagcctgggtcccagcctgtttgatgaaagaaaca | |
| | agaacactgatacaagccacagccccttggcaaaaaagatacccaatagcaatggc | |
| | aatgtaaaatcagttttagtaaatgaatcaagaattctgatgctttagggaaagta | |
| | atgtgaacctggcaccattaacaaattcagaactcttcttcttaggagctctctaa | |
| | ctgaacagacagagggatgtcaacccctaattcagcttgatcgtatctcagcaact | |
| | acatttaatgagacagtgggaaaaagagagctgtccacttttaaatcagcatattt | |
| | ctaactaaacaatggcaatggctaaatctttaaaatgcctatttctctcaagaaca | |
| | ctgcaatggaacatttagactttgggaaagagattagtgatttacattgctatctc | |
| | actgatttaatttaaatgctcttccaaaccaaacacacatgtgccgaagaggctac | |
| | taagaaacccaacatgcagagttctctataagtgcagccgacagtgttgactgaaa | |
| | ctaaacttggaaatccagggcactaatgcacaatatcaagcaataaaacggcatct | |
| | ctttggcaatatttaatttaaaaaagaagaaagagacaggcgaagatcaggcactg | |
| | tctgttttggaggatcaaccattctgcatttcaaagcattggtccctgcaatatcc | |
| | aggttactgtgctagaatctcgactattatatcgcagttgtgagagggagggcaaa | |
| | gatgtgtttactcagtgattaggcccttagaataagcctctagctcctagagagac | |
| | agctcaccacttattcatttgggccaattcacaaagcctaggaagattaaacatcc | |
| | atgctgagaagacaagcgaatgcagacggtgaaaaagaaataaaaattctttaaaa | |
| | actctgagatgacttcattatttttccacaaggaaactttaggaaagtgtttagtt | |
| | agagaaaaacccacattgacctctctctaaacccttaatctttcctttgtggtggc | |
| | actgctttgtggtaagcgactggctcgcctcgcccctcttttcactggaagctgag | |
| | agaaaaaagactctggagaaacagttttcgttccagggacacaaacccctgacact | |
| | gttaaacatgagatgccaggaaaacacacttaaaaaaaaaattcccactttaagct | |
| | ttagactgaatgtgagaaaggagatgataaaaagagtatcacaaGAGAATCTTCAG | |
| | GCTGTGGG | |
| CNS-6 | TTTGGCACTGTGAGCAGTTTACttgacaaattctgtcaaatatttgctttctgaaa | 1104 |
| (SEQ ID | tctcgagaattggttgaatataattgtacttaatgtttgcaaaataaataaatatg | |
| NO: 96) | ggactaaggacgttctatcattaatttgtcagaaaagagagttgtcatttctgaaa | |
| | atttaatgtcattgaagctctatttccaatagcaaaggagcactattgctaataga | |
| | cttcagagcttgaaataaataaatctttggaatcctgttgcatctcttggggtgtg | |
| | acatttgacagtcttttatagcacagaacgaaacaagtttgtgagctggaattcaa | |
| | ttgtggcgtattgattccttgcatcagtcattattccctgctgattgacaggtgaa | |
| | aattggttacgttaagtatttcatatgttatattggctgacatttgcttgcctgct | |
| | cttgtgtcaatattgttgtaaagatctccagctttatgagatagcaatagacactg | |
| | actgtggcttttgtgtgatgttccagtgtttttcctgacataatttaagacatatt | |
| | aaaaaccagcagcatcttccctcttgagaagcttaatgccaatattattgtcttcc | |
| | aggggaagatcatgtatgctcataatcgggtgctaatttccaccagtacgctcatg | |
| | tttaggcattaggcactataactgtaaaattgagccttcttgattgattcatgtca | |
| | agcctcatctcggctcctgcaggggaagtcatccggctgaccctttttacactaaa | |
| | agaagagatttgtgttcctttctttcacctggaaccatcaaattgactgaataatc | |
| | tgtaatacattagtgctgacatttgttagggagaattaaacaagacacagtaatca | |
| | ttccccagaataaaaattgtgtttgatttccagcagagttctattaaagggaggac | |
| | agaatctgtctcttccaaggtggaaaatcgtgaatattccctgcattaatgaacca | |
| | agttaacactttaattgcttatagaaccgagttctccaatgacagcattaaaagat | |
| | agggaggctctgatttaTGGTCAACACAGATTTGTAACCC | |
| CNS-7 | TCAACATGGATAACCAAAGTTCTtaaaactacgctttcaatgaacacatatccttt | 1941 |
| (SEQ ID | gagcaagactaataatgaggaatgggagccagctcctgtgatatttatgcaactac | |
| NO: 97) | taaattctcactgaagtcaatgggagtttgcttacgtaagggctgcaaactttagc | |
| | ctccagagattaaaggggaaaaaaatccttaaactctttcaacattaatattgcct | |
| | gtaaggaatccagccatgacctaagccatggagctttctgaacctagcaagtagaa | |
| | gggtaaacagtaaacaccagttattttaagcacaatctaatcagagttcaatgaga | |
| | agcaatattatatttgatctctaaggtattaatacttgtatatcactattagacat | |
| | ctttatgtagtccattatccaaacaatggcttaagtctgtggtatttaataaatca | |
| | agtttccatggccgtgagactgagtgggagtggggatgaagccttttttcttcatt | |
| | tttttttcctcaggtgcaattctgtgttaatataagagaagtgtggccttccttct | |
| | catagcactaaaagtgagataatccctgtgtaagaaatcagtaagtacggtctgct | |
| | taatctagtcccagtgtgaaactgttgacatttgttcttttttctatcattatgtg | |
| | actgggcctgttttgtgctggattaggcacaaatctcctatgcagcacatttggca | |
| | tgttactagtagtttaacttcattaataatgtatgaagaaaatgtaatccatgaca | |
| | aggaagcaaagaaaagtatttttttttttttttgcttctcccaaatcctttggaat | |
| | gagtaattattcaacattttatgtttgatgttatattttacaattcaacttccata | |
| | gtgatatttaaaaaagaaactttggcaaatgcttgcaaaaaacacaccttttacaa | |
| | ttttaaatgtgatttactgatggccagaacttgttaaacatagtaggaaattaaat | |
| | atttattcatcttatttcattttcagggccgtaaacgctccttctgagtcattccc | |
| | aataacaagaatttctaccagtaaagctattaacaggcatcaaaataggggagtgc | |
| | taaattaagatgagattgtaaaagcaaataagaacatacgcagactcgcataggag | |
| | tgcaaatgatcgtttctgattgaaatgtttatagctaaatgagtttggctgaatta | |
| | aacacaaatgttccaaaagataagccgtagctggtgcttcttttttctgtttttta | |
| | agctgctttacagacgaaaatggaactatatttggaacaatgctttctgtttttcc | |
| | atactattgatatttgtggaaagtcacaaaatggcctaaggaagctaagctcgccc | |
| | caagcagtggtcacttacaagtacttttgtactctgtactcctgtcacatttgggc | |
| | gatcagagcaacagctggggagactttttcaacaaagatgagtgtcagataatcct | |
| | gatgagattccacatccaacatcttttgtaattatgtcacattcagctgtaatgga | |
| | ataattcaagctgaaagaacaagctttgatcctttcttaaacctttccctgtggac | |
| | tggctatctaaaagatttaaagatatttctgttacaagatctagtgtttcctcaga | |
| | gaagtcatgcttctgaagcatcgtgatctacaagaacaatatcaagtttgccaaac | |
| | acatttctgaaagcatcgtgttttggggggaggggttgtatttaatgaagatatca | |
| | ataatatgctatgcttcaattttcatctaggtgatcaagattcattttcttgttct | |
| | gtcatccaaataggcagacagaaaagtgattgaaatacattaTGGAGATGTGTCAT | |
| | TGCACA | |
| CNS-8 | GCTGGTGCTTCTTTTTTCTGTTTTTTAAGCTGCTTTACAGACGAAAATGGAACTAT | 540 |
| (SEQ ID | ATTTGGAACAATGCTTTCTGTTTTTCCATACTATTGATATTTGTGGAAAGTCACAA | |
| NO: 98) | AATGGCCTAAGGAAGCTAAGCTCGCCCCAAGCAGTGGTCACTTACAAGTACTTTTG | |
| | TACTCTGTACTCCTGTCACATTTGGGCGATCAGAGCAACAGCTGGGGAGACTTTTT | |
| | CAACAAAGATGAGTGTCAGATAATCCTGATGAGATTCCACATCCAACATCTTTTGT | |
| | AATTATGTCACATTCAGCTGTAATGGAATAATTCAAGCTGAAAGAACAAGCTTTGA | |
| | TCCTTTCTTAAACCTTTCCCTGTGGACTGGCTATCTAAAAGATTTAAAGATATTTC | |
| | TGTTACAAGATCTAGTGTTTCCTCAGAGAAGTCATGCTTCTGAAGCATCGTGATCT | |
| | ACAAGAACAATATCAAGTTTGCCAAACACATTTCTGAAAGCATCGTGTTTTGGGGG | |
| | GAGGGGTTGTATTTAATGAAGATATCAATAATATGC | |

TABLE 11
CNS-specific promoters

| NAME | SEQUENCE | Length |
|---|---|---|
| CNS-1 | CTGGGCAGAGAGGGGGCATCGGGGGCATGGCTAGGGGCCAGCACTGTGCTTCCTGG | 696 |
| (SEQ ID | GCGCCTCACCTCCTCCCTGACTCCTGGAGACTCCCAGCCCCTGTCTGGGAGATGAG | |
| NO: | CATTTAGGAATCTGCTTGTGCAGGGGTGGTGGGAGGGGCCGGGGTGGAGGGCGCAT | |
| 112) | CCCCACGGGGAGATTGGATGGAAATGGCCTGCCAGTGTGTGTGTGAGTGTGCGCCT | |
| | GTGGCAGCAGCAGAGTAAACAGCCGCTGCCCTGTCCTCTCTGCGGCCGTGGCCAGG | |
| | TACACAGGCCTGTTTGGACAGCTGCCTTGTCTGTCCGTCTGTTTGGGAGATGCTGG | |
| | CTGATAGATGGGGATGGGCGGACTGTTAACCCCTCGTTGCCTGCACTGCTATGTGC | |
| | TTCCTGCCTCATCCATGGGGTAGAAGGTAGCCAGAAGGTGGTCCTGGCTGTGCCCC | |
| | CAGCTCCTCTCTAGGGGGGAAACCTCTAGTTCTGAGTCAGGGACAGAGTGAGGAGG | |
| | GCTCCAGGGCATCAAGAGCTTGCTCCTCCCCGCACCAGGGAGCCAAGGACAGAGGA | |
| | GAAGGGGGTCTTCCCCAGTGGTGACTAGGGGCAGAATATGTCTCTGAGTGAGTGTC | |
| | TGGAGCCCTCCTCACCCCAACACCATGGGGCTGGGCATAAAAGTCAGGGCAGAGCC | |
| | ATCTATTGCTTACATTTGCTTCTG | |
| CNS-2 | GGTGTGTGGAAGGGTGAGAGGCACACACACAGACACTGAAAGAATCCTAGGCCTGG | 709 |
| (SEQ ID | TAGGCACTTAACAAATGTCTGTTACAGACCAGAATTTTATTGCTGTTAGAGACCCA | |
| NO: | AGCCCCTCATAGGAACAGTGAGAAACAGGTGCAGAAAGGCGGAGTAACTTTATCTA | |
| 113) | AAGTCATAGGCTCCCTGAATAGCAGAGCTGACACCTACAAGGAAGCGTTGGAGACC | |
| | AGATCTACCAGCTAGCCTCCCTGAGACCACGAGGTGGCGCCGCAGCACCGGCTGTG | |
| | GCCGATGCCAGCCAGGTAGCCGGTTTCCCACGTCCCCCGCACGCACGCACCTCTTT | |
| | GCTGCAGGAATCCCGGGCTGCCCCGACCTGGAGTAGGGGGGGTGGTGAGTGGGACT | |
| | GAGTCCCTAGAAGCCTGGACCCTCACTTCGTTCCTGTACATCCAGCTCGCCTGTAG | |
| | ACAGTGGGGGAGGATGAAGGGAAGAGGACTCAAGCGCAACTTTGAATCATCACGCC | |
| | TTCGACAGTCCGCGCACGTTTATTTCATTTATCTTTGAAAACGAGGGAGGGGAAGC | |
| | CTGGAGAAGGCGGGATGGGCCAAGGGTGAGTTGGCCCCCGGGGAGCTGGTCCCTGT | |
| | TCCTGGCTTTAGTCCCAGGGGCGCGGTCTGTGTGTAGGGCGGGCTGGGCATAAAAG | |
| | TCAGGGCAGAGCCATCTATTGCTTACATTTGCTTCTG | |
| CNS-3 | TGCTACCAGAGCCGGGAGAGCTGCTCGGAGACGCCTCCGGGGTGCGGGCTGGACAT | |
| (SEQ ID | GAGCAGCGGCTGCCGGTCCTGGGACTAGGCCCCGCCATTTTGGATCCGCTGACAGG | |
| NO: | TCAGCGAAGTCTCTTCCTAGAGTTCCGGTGTCGTGAAGGCCGCCCTGACATCGCAA | |
| 114) | TAGGGAATTAGTGGGAAGGGCCCTTAAATTGGGCGAGCCAAGGTGGGGGGAGGATT | |
| | GGAACAGAGACAAAAGGGAGGAGAGACGGACAGCGACAAGTGGAGAAAATCGGCGA | |
| | AACTTGAGTGGCAGAGAAGTCTGAGCGCTGAGACCCGGCGGCCCCGTGCGCCTTCC | |
| | CACCTGGCGCCGATCCACTTTCCTCGGGGTAGCGGCCCAACCCACTTCGCTGCCAG | |
| | CCGATCCCTTTTACCCGTGGCTACCGGGACCACTCTACTCTCGCCCACTTGGCTCT | |
| | GCCTAAGCGTCCTAGCCGGAGCGCGGTCTCTGCCACGTGGGGAGGGGCGCGGCCGA | |
| | GTTGCTGAAGAGCGCTTCTGATTGGCCAGAGGGCGGGGTTCTTGGCGTCTCGCCGG | |
| | CCAGACCCCTCCCTCAAAGGCGGGGCCTGGAGATCCACAGCTGGAAAGGGCGGAGC | |
| | CCCAGCAGGGCAGCTGGAAAGGGGCGGGGCCTGACGCGCGCGGCTCGCCGCGGCGG | |
| | GCTGGGGGCGCCCTGGTCTGCCATAAAGTGAATGGGCGCCGGCTGGGGGTGGCAGT | |
| | ACGCGGTGAGGCTCACTCCCTCCGAGAGTCCAGGAGCGCC | |
| CNS-4 | AAGGAGAATGGTAAACAGCAGGAGCGAAGCGGCTGAGGAGAAAGAAGAGGAAAGAA | |
| (SEQ ID | AGGCGAGACGTGGGAGGATTGGAACAGAGACAAAAGGGAGGAGAGACGGACAGCGA | |
| NO: | CAAGTGGAGAAAATCGGCGAAACTTGAGTGGCAGAGAAGTCTGAGCGCTGAGACCC | |
| 115) | GGCGGCCCCGTGCGCCTTCCCACCTGGCGCCGATCCACTTTCCTCGGGGTAGCGGC | |
| | CCAACCCACTTCGCTGCCAGCCGATCCCTTTTACCCGTGGCTACCGGGACCACTCT | |
| | ACTCTCGCCCACTTGGCTCTGCCTAAGCGTCCTAGCCGGAGCGCGGTCTCTGCCAC | |
| | GTGGGGAGGGGCGCGGCCGAGTTGCTGAAGAGCGCTTCTGATTGGCCAGAGGGCGG | |
| | GGTTCTTGGCGTCTCGCCGGCCAGACCCCTCCCTCAAAGGCGGGGCCTGGAGATCC | |
| | ACAGCTGGAAAGGGCGGAGCCCCAGCAGGGCAGCTGGAAAGGGGCGGGGCCTGACG | |
| | CGCGCGGCTCGCCGCGGCGGGCTGGGGGCGCCCTGGTCTGCCATAAAGTGAATGGG | |
| | CGCCGGCTGGGGGTGGCAGTACGCGGTGAGGCTCACTCCCTCCGAGAGTCCAGGAG | |
| | CGCCCGAGCGGAGAGGCGGCCCGGGAGCAGGGGGGCGGCCCCCACTCCGGCCGGGT | |
| | GCCCGGCCCCTGGCCCCTGCCTGCCCTCTAGATCGCCGCCGCAGCCGCCGCTACTG | |
| | GGAGTCTG | |
| CNS- | ttgtAATGGGAATAAGGGCAGGACtcctgggtataagtagctcagctgatcccacc | 1744 |
| 5_v2 | ctgcttctatgtgttaattcatttattcattcattcaacaagcatttgttgaaatg | |
| (SEQ ID | ctctttgtgtcaggctcagcaggaagcagtggcaataaaatggtgaacaagaaaga | |
| NO: | ctcggggtttcttcatctatgttgatgtctgcagagaacagtatcagccttctagg | |
| 116) | aagtttgtaatcagatacattgttagagagatacttatctagtaaattcctactca | |
| | tcctataaggctcaaaacaaatgcctctatgaaaccttccgtgattccctcaggca | |
| | gagttaagagcttcctttcctgggcctctatctccttccattagtattataactgt | |
| | ttaccagtttcccctctagactaaatttctcaaaagagagaatgaggtctctttca | |
| | gtcttctttgcatctttaaactagcctgggtcccagcctgtttgatgaaagaaaca | |
| | agaacactgatacaagccacagccccttggcaaaaaagatacccaatagcaatggc | |
| | aatgtaaaatcagttttagtaaatgaatcaagaattctgatgctttagggaaagta | |
| | atgtgaacctggcaccattaacaaattcagaactcttcttcttaggagctctctaa | |
| | ctgaacagacagagggatgtcaacccctaattcagcttgatcgtatctcagcaact | |
| | acatttaatgagacagtgggaaaaagagagctgtccacttttaaatcagcatattt | |
| | ctaactaaacaatggcaatggctaaatctttaaaatgcctatttctctcaagaaca | |
| | ctgcaatggaacatttagactttgggaaagagattagtgatttacattgctatctc | |
| | actgatttaatttaaatgctcttccaaaccaaacacacatgtgccgaagaggctac | |
| | taagaaacccaacatgcagagttctctataagtgcagccgacagtgttgactgaaa | |
| | ctaaacttggaaatccagggcactaatgcacaatatcaagcaataaaacggcatct | |
| | ctttggcaatatttaatttaaaaaagaagaaagagacaggcgaagatcaggcactg | |
| | tctgttttggaggatcaaccattctgcatttcaaagcattggtccctgcaatatcc | |
| | aggttactgtgctagaatctcgactattatatcgcagttgtgagagggagggcaaa | |
| | gatgtgtttactcagtgattaggcccttagaataagcctctagctcctagagagac | |
| | agctcaccacttattcatttgggccaattcacaaagcctaggaagattaaacatcc | |
| | atgctgagaagacaagcgaatgcagacggtgaaaaagaaataaaaattctttaaaa | |
| | actctgagatgacttcattatttttccacaaggaaactttaggaaagtgtttagtt | |
| | agagaaaaacccacattgacctctctctaaaccettaatctttcctttgtggtggc | |
| | actgctttgtggtaagcgactggctcgcctcgcccctcttttcactggaagctgag | |
| | agaaaaaagactctggagaaacagttttcgttccagggacacaaacccctgacact | |
| | gttaaacatgagatgccaggaaaacacacttaaaaaaaaaattcccactttaagct | |
| | ttagactgaatgtgagaaaggagatgataaaaagagtatcacaaGAGAATCTTCAG | |
| | GCTGTGGG | |
| CNS- | TTTGGCACTGTGAGCAGTTTACttgacaaattctgtcaaatatttgctttctgaaa | 1104 |
| 6_v2 | tctcgagaattggttgaatataattgtacttaatgtttgcaaaataaataaatatg | |
| (SEQ ID | ggactaaggacgttctatcattaatttgtcagaaaagagagttgtcatttctgaaa | |
| NO: | atttaatgtcattgaagctctatttccaatagcaaaggagcactattgctaataga | |
| 117) | cttcagagcttgaaataaataaatctttggaatcctgttgcatctcttggggtgtg | |
| | acatttgacagtcttttatagcacagaacgaaacaagtttgtgagctggaattcaa | |
| | ttgtggcgtattgattccttgcatcagtcattattccctgctgattgacaggtgaa | |
| | aattggttacgttaagtatttcatatgttatattggctgacatttgcttgcctgct | |
| | cttgtgtcaatattgttgtaaagatctccagctttatgagatagcaatagacactg | |
| | actgtggcttttgtgtgatgttccagtgtttttcctgacataatttaagacatatt | |
| | aaaaaccagcagcatcttccctcttgagaagcttaatgccaatattattgtcttcc | |
| | aggggaagatcatgtatgctcataatcgggtgctaatttccaccagtacgctcatg | |
| | tttaggcattaggcactataactgtaaaattgagccttcttgattgattcatgtca | |
| | agcctcatctcggctcctgcaggggaagtcatccggctgaccctttttacactaaa | |
| | agaagagatttgtgttcctttctttcacctggaaccatcaaattgactgaataatc | |
| | tgtaatacattagtgctgacatttgttagggagaattaaacaagacacagtaatca | |
| | ttccccagaataaaaattgtgtttgatttccagcagagttctattaaagggaggac | |
| | agaatctgtctcttccaaggtggaaaatcgtgaatattccctgcattaatgaacca | |
| | agttaacactttaattgcttatagaaccgagttctccaatgacagcattaaaagat | |
| | agggaggctctgatttaTGGTCAACACAGATTTGTAACCC | |
| CNS- | TCAACATGGATAACCAAAGTTCTtaaaactacgctttcaatgaacacatatccttt | 1941 |
| 7_v2 | gagcaagactaataatgaggaatgggagccagctcctgtgatatttatgcaactac | |
| (SEQ ID | taaattctcactgaagtcaatgggagtttgcttacgtaagggctgcaaactttagc | |
| NO: | ctccagagattaaaggggaaaaaaatccttaaactctttcaacattaatattgcct | |
| 118) | gtaaggaatccagccatgacctaagccatggagctttctgaacctagcaagtagaa | |
| | gggtaaacagtaaacaccagttattttaagcacaatctaatcagagttcaatgaga | |
| | agcaatattatatttgatctctaaggtattaatacttgtatatcactattagacat | |
| | ctttatgtagtccattatccaaacaatggcttaagtctgtggtatttaataaatca | |
| | agtttccatggccgtgagactgagtgggagtggggatgaagccttttttcttcatt | |
| | tttttttcctcaggtgcaattctgtgttaatataagagaagtgtggccttccttct | |
| | catagcactaaaagtgagataatccctgtgtaagaaatcagtaagtacggtctgct | |
| | taatctagtcccagtgtgaaactgttgacatttgttcttttttctatcattatgtg | |
| | actgggcctgttttgtgctggattaggcacaaatctcctatgcagcacatttggca | |
| | tgttactagtagtttaacttcattaataatgtatgaagaaaatgtaatccatgaca | |
| | aggaagcaaagaaaagtatttttttttttttttgcttctcccaaatcctttggaat | |
| | gagtaattattcaacattttatgtttgatgttatattttacaattcaacttccata | |
| | gtgatatttaaaaaagaaactttggcaaatgcttgcaaaaaacacaccttttacaa | |
| | ttttaaatgtgatttactgatggccagaacttgttaaacatagtaggaaattaaat | |
| | atttattcatcttatttcattttcagggccgtaaacgctccttctgagtcattccc | |
| | aataacaagaatttctaccagtaaagctattaacaggcatcaaaataggggagtgc | |
| | taaattaagatgagattgtaaaagcaaataagaacatacgcagactcgcataggag | |
| | tgcaaatgatcgtttctgattgaaatgtttatagctaaatgagtttggctgaatta | |
| | aacacaaatgttccaaaagataagccgtagctggtgcttcttttttctgtttttta | |
| | agctgctttacagacgaaaatggaactatatttggaacaatgctttctgtttttcc | |
| | atactattgatatttgtggaaagtcacaaaatggcctaaggaagctaagctcgccc | |
| | caagcagtggtcacttacaagtacttttgtactctgtactcctgtcacatttgggc | |
| | gatcagagcaacagctggggagactttttcaacaaagatgagtgtcagataatcct | |
| | gatgagattccacatccaacatcttttgtaattatgtcacattcagctgtaatgga | |
| | ataattcaagctgaaagaacaagctttgatcctttcttaaacctttccctgtggac | |
| | tggctatctaaaagatttaaagatatttctgttacaagatctagtgtttcctcaga | |
| | gaagtcatgcttctgaagcatcgtgatctacaagaacaatatcaagtttgccaaac | |
| | acatttctgaaagcatcgtgttttggggggaggggttgtatttaatgaagatatca | |
| | ataatatgctatgcttcaattttcatctaggtgatcaagattcattttcttgttct | |
| | gtcatccaaataggcagacagaaaagtgattgaaatacattaTGGAGATGTGTCAT | |
| | TGCACA | |
| CNS- | GCTGGTGCTTCTTTTTTCTGTTTTTTAAGCTGCTTTACAGACGAAAATGGAACTAT | 540 |
| 8_v2 | ATTTGGAACAATGCTTTCTGTTTTTCCATACTATTGATATTTGTGGAAAGTCACAA | |
| (SEQ ID | AATGGCCTAAGGAAGCTAAGCTCGCCCCAAGCAGTGGTCACTTACAAGTACTTTTG | |
| NO | TACTCTGTACTCCTGTCACATTTGGGCGATCAGAGCAACAGCTGGGGAGACTTTTT | |
| 119) | CAACAAAGATGAGTGTCAGATAATCCTGATGAGATTCCACATCCAACATCTTTTGT | |
| | AATTATGTCACATTCAGCTGTAATGGAATAATTCAAGCTGAAAGAACAAGCTTTGA | |
| | TCCTTTCTTAAACCTTTCCCTGTGGACTGGCTATCTAAAAGATTTAAAGATATTTC | |
| | TGTTACAAGATCTAGTGTTTCCTCAGAGAAGTCATGCTTCTGAAGCATCGTGATCT | |
| | ACAAGAACAATATCAAGTTTGCCAAACACATTTCTGAAAGCATCGTGTTTTGGGGG | |
| | GAGGGGTTGTATTTAATGAAGATATCAATAATATGC | |
| CNS-1 + | CTGGGCAGAGAGGGGGCATCGGGGGCATGGCTAGGGGCCAGCACTGTGCTTCCTGG | 982 |
| CMV-IE | GCGCCTCACCTCCTCCCTGACTCCTGGAGACTCCCAGCCCCTGTCTGGGAGATGAG | |
| UTR and | CATTTAGGAATCTGCTTGTGCAGGGGTGGTGGGAGGGGCCGGGGTGGAGGGCGCAT | |
| intron | CCCCACGGGGAGATTGGATGGAAATGGCCTGCCAGTGTGTGTGTGAGTGTGCGCCT | |
| (SEQ ID | GTGGCAGCAGCAGAGTAAACAGCCGCTGCCCTGTCCTCTCTGCGGCCGTGGCCAGG | |
| NO: | TACACAGGCCTGTTTGGACAGCTGCCTTGTCTGTCCGTCTGTTTGGGAGATGCTGG | |
| 120) | CTGATAGATGGGGATGGGCGGACTGTTAACCCCTCGTTGCCTGCACTGCTATGTGC | |
| | TTCCTGCCTCATCCATGGGGTAGAAGGTAGCCAGAAGGTGGTCCTGGCTGTGCCCC | |
| | CAGCTCCTCTCTAGGGGGGAAACCTCTAGTTCTGAGTCAGGGACAGAGTGAGGAGG | |
| | GCTCCAGGGCATCAAGAGCTTGCTCCTCCCCGCACCAGGGAGCCAAGGACAGAGGA | |
| | GAAGGGGGTCTTCCCCAGTGGTGACTAGGGGCAGAATATGTCTCTGAGTGAGTGTC | |
| | TGGAGCCCTCCTCACCCCAACACCATGGGGCTGGGCATAAAAGTCAGGGCAGAGCC | |
| | ATCTATTGCTTACATTTGTCAGATCGCCTGGAGACGCCATCCACGCTGTTTTGACC | |
| | TCCATAGAAGACACCGGGACCGATCCAGCCTCCGCGGCCGGGAACGGTGCATTGGA | |
| | ACGCGGATTCCCCGTGCCAAGAGTGACGTAAGTACCGCCTATAGACTCTATAGGCA | |
| | CACCCCTTTGGCTCTTATGCATGAACGGTGGAGGGCAGTGTAGTCTGAGCAGTACT | |
| | CGTTGCTGCCGCGCGCGCCACCAGACATAATAGCTGACAGACTAACAGACTGTTCC | |
| | TTTCCATGGGTCTTTTCTGCAGATGCCACC | |
| CNS-4 + | AAGGAGAATGGTAAACAGCAGGAGCGAAGCGGCTGAGGAGAAAGAAGAGGAAAGAA | 876 |
| CMV-IE | AGGCGAGACGTGGGAGGATTGGAACAGAGACAAAAGGGAGGAGAGACGGACAGCGA | |
| UTR and | CAAGTGGAGAAAATCGGCGAAACTTGAGTGGCAGAGAAGTCTGAGCGCTGAGACCC | |
| intron | GGCGGCCCCGTGCGCCTTCCCACCTGGCGCCGATCCACTTTCCTCGGGGTAGCGGC | |
| (SEQ ID | CCAACCCACTTCGCTGCCAGCCGATCCCTTTTACCCGTGGCTACCGGGACCACTCT | |
| NO: | ACTCTCGCCCACTTGGCTCTGCCTAAGCGTCCTAGCCGGAGCGCGGTCTCTGCCAC | |
| 121) | GTGGGGAGGGGCGCGGCCGAGTTGCTGAAGAGCGCTTCTGATTGGCCAGAGGGCGG | |
| | GGTTCTTGGCGTCTCGCCGGCCAGACCCCTCCCTCAAAGGCGGGGCCTGGAGATCC | |
| | ACAGCTGGAAAGGGCGGAGCCCCAGCAGGGCAGCTGGAAAGGGGCGGGGCCTGACG | |
| | CGCGCGGCTCGCCGCGGCGGGCTGGGGGCGCCCTGGTCTGCCATAAAGTGAATGGG | |
| | CGCCGGCTGGGGGTGGCAGTACGCTCAGATCGCCTGGAGACGCCATCCACGCTGTT | |
| | TTGACCTCCATAGAAGACACCGGGACCGATCCAGCCTCCGCGGCCGGGAACGGTGC | |
| | ATTGGAACGCGGATTCCCCGTGCCAAGAGTGACGTAAGTACCGCCTATAGACTCTA | |
| | TAGGCACACCCCTTTGGCTCTTATGCATGAACGGTGGAGGGCAGTGTAGTCTGAGC | |
| | AGTACTCGTTGCTGCCGCGCGCGCCACCAGACATAATAGCTGACAGACTAACAGAC | |
| | TGTTCCTTTCCATGGGTCTTTTCTGCAGATGCCACC | |
| CNS-5 | GGAACATTTAGACTTTGGGAAAGAGATTAGTGATTTACATTGCTATCTCACTGATT | 835 |
| (SEQ ID | TAATTTAAATGCTCTTCCAAACCAAACACACATGTGCCGAAGAGGCTACTAAGAAA | |
| NO: | CCCAACATGCAGAGTTCTCTATAAGTGCAGCCGACAGTGTTGACTGAAACTAAACT | |
| 122) | TGGAAATCCAGGGCACTAATGCACAATATCAAGCAATAAAACGGCATCTCTTTGGC | |
| | AATATTTAATTTAAAAAAGAAGAAAGAGACAGGCGAAGATCAGGCACTGTCTGTTT | |
| | TGGAGGATCAACCATTCTGCATTTCAAAGCATTGGTCCCTGCAATATCCAGGTTAC | |
| | TGTGCTAGAATCTCGACTATTATATCGCAGTTGTGAGAGGGAGGGCAAAGATGTGT | |
| | TTACTCAGTGATTAGGCCCTTAGAATAAGCCTCTAGCTCCTAGAGAGACAGCTCAC | |
| | CACTTATTCATTTGGGCCAATTCACAAAGCCTAGGAAGATTAAACATCCATGCTGA | |
| | GAAGACAAGCGAATGCAGACGGTGAAAAAGAAATAAAAATTCTTTAAAAACTCTGA | |
| | GATGACTTCATTATTTTTCCACAAGGAAACTTTAGGAAAGTGTTTAGTTAGAGAAA | |
| | AACCCACATTGACCTCTCTCTAAACCCTTAATCTTTCCTTTGTGGTGGCACTGCTT | |
| | TGTGGTAAGCGACTGGCTCGCCTCGCCCCTCTTTTCACTGGAAGCTGAGAGAAAAA | |
| | AGACTCTGGAGAAACAGTTTTCGTTCCAGGGACACAAACCCCTGACACTGTTAAGG | |
| | GCTGGGCATAAAAGTCAGGGCAGAGCCATCTATTGCTTACATTTGCTTCTG | |
| CNS-6 | GAAAATTTAATGTCATTGAAGCTCTATTTCCAATAGCAAAGGAGCACTATTGCTAA | 812 |
| (SEQ ID | TAGACTTCAGAGCTTGAAATAAATAAATCTTTGGAATCCTGTTGCATCTCTTGGGG | |
| NO: | TGTGACATTTGACAGTCTTTTATAGCACAGAACGAAACAAGTTTGTGAGCTGGAAT | |
| 123) | TCAATTGTGGCGTATTGATTCCTTGCATCAGTCATTATTCCCTGCTGATTGACAGG | |
| | TGAAAATTGGTTACGTTAAGTATTTCATATGTTATATTGGCTGACATTTGCTTGCC | |
| | TGCTCTTGTGTCAATATTGTTGTAAAGATCTCCAGCTTTATGAGATAGCAATAGAC | |
| | ACTGACTGTGGCTTTTGTGTGATGTTCCAGTGTTTTTCCTGACATAATTTAAGACA | |
| | TATTAAAAACCAGCAGCATCTTCCCTCTTGAGAAGCTTAATGCCAATATTATTGTC | |
| | TTCCAGGGGAAGATCATGTATGCTCATAATCGGGTGCTAATTTCCACCAGTACGCT | |
| | CATGTTTAGGCATTAGGCACTATAACTGTAAAATTGAGCCTTCTTGATTGATTCAT | |
| | GTCAAGCCTCATCTCGGCTCCTGCAGGGGAAGTCATCCGGCTGACCCTTTTTACAC | |
| | TAAAAGAAGAGATTTGTGTTCCTTTCTTTCACCTGGAACCATCAAATTGACTGAAT | |
| | AATCTGTAATACATTAGTGCTGACATTTGTTAGGGAGAATTAAACAAGACACAGTA | |
| | ATCATTCCCCAGAATAAAAATTGTGTTTGATGGGCTGGGCATAAAAGTCAGGGCAG | |
| | AGCCATCTATTGCTTACATTTGCTTCTG | |
| CNS-7 | TGAGACTGAGTGGGAGTGGGGATGAAGCCTTTTTTCTTCATTTTTTTTTCCTCAGG | 487 |
| (SEQ ID | TGCAATTCTGTGTTAATATAAGAGAAGTGTGGCCTTCCTTCTCATAGCACTAAAAG | |
| NO: | TGAGATAATCCCTGTGTAAGAAATCAGTAAGTACGGTCTGCTTAATCTAGTCCCAG | |
| 124) | TGTGAAACTGTTGACATTTGTTCTTTTTTCTATCATTATGTGACTGGGCCTGTTTT | |
| | GTGCTGGATTAGGCACAAATCTCCTATGCAGCACATTTGGCATGTTACTAGTAGTT | |
| | TAACTTCATTAATAATGTATGAAGAAAATGTAATCCATGACAAGGAAGCAAAGAAA | |
| | AGTATTTTTTTTTTTTTTTGCTTCTCCCAAATCCTTTGGAATGAGTAATTATTCAA | |
| | CATTTTATGTTTGATGTTATATTTTACAATTCAACTTCCATAGGGCTGGGCATAAA | |
| | AGTCAGGGCAGAGCCATCTATTGCTTACATTTGCTTCTG | |
| CNS-8 | GCTGGTGCTTCTTTTTTCTGTTTTTTAAGCTGCTTTACAGACGAAAATGGAACTAT | 593 |
| (SEQ ID | ATTTGGAACAATGCTTTCTGTTTTTCCATACTATTGATATTTGTGGAAAGTCACAA | |
| NO: | AATGGCCTAAGGAAGCTAAGCTCGCCCCAAGCAGTGGTCACTTACAAGTACTTTTG | |
| 125) | TACTCTGTACTCCTGTCACATTTGGGCGATCAGAGCAACAGCTGGGGAGACTTTTT | |
| | CAACAAAGATGAGTGTCAGATAATCCTGATGAGATTCCACATCCAACATCTTTTGT | |
| | AATTATGTCACATTCAGCTGTAATGGAATAATTCAAGCTGAAAGAACAAGCTTTGA | |
| | TCCTTTCTTAAACCTTTCCCTGTGGACTGGCTATCTAAAAGATTTAAAGATATTTC | |
| | TGTTACAAGATCTAGTGTTTCCTCAGAGAAGTCATGCTTCTGAAGCATCGTGATCT | |
| | ACAAGAACAATATCAAGTTTGCCAAACACATTTCTGAAAGCATCGTGTTTTGGGGG | |
| | GAGGGGTTGTATTTAATGAAGATATCAATAATATGCGGGCTGGGCATAAAAGTCAG | |
| | GGCAGAGCCATCTATTGCTTACATTTGCTTCTG | |
| CNS-10 | CCTGAGGGCAGCACTCACCTCCACAGGGGTCCAGGGGCCTCCCCAGCCTCAGCACC | 848 |
| (SEQ ID | TGGCCTGGGCTCCTGCCTCCAGAGAGCCTGGCCCCAAGGAAGAGTCTAGTAAGCTT | |
| NO: | AGTTCCCATCGGGCTTCCATGAAAGCACAACTGGCCCGGCAGGAAACCGAATTAAA | |
| 187) | AAGCAATATTTGTATCAGTGGAAGACATTTGCTGAAAGGTTAAATCCACATCCGGC | |
| | AGTGTGGGCCATGAGCCTCCGGCGTGGTGTTCATCAGGCATGTCTCTCCTCCTGGC | |
| | CTGGGCACCTGAGCACTGGGGCCGCCCTGGGCAGAGCTGGGGGGGGGTGCTGGGGG | |
| | GCCTGGAGCTGCCTCACCGAGGGATCCTCAGCAGCCGACCCTGGGGGAGGCAAATG | |
| | AGACTCTTTCTGGGGACCTTGAGGGGAGCTCGGGGGAGCCATGCAGAGCTTCACCA | |
| | GGCCTGGACACTGGGCATGGAGGCTGGGCCACCCAAGGGCCATCACCAGGGACTCA | |
| | GGTGGGTGGGCCTCAGCCCTGGGTGACAGAAGCTCACGGGCCGCAGGGCGAGGCCA | |
| | GAGGCTGAGCCTTCAGGCTGAGGTCTTGGAGGCAAATCCCTCCAACGCCCTTCTGA | |
| | GCAGGCACCCAGACCTACTGTGGGCAGGACCCACAGGAGGTGGAGGCCTTTGGGGA | |
| | ACACTGTGGAGGGGCATAGCATCTCCGAGAGAGGACAGGGTCTGCACTGGGTGCTG | |
| | AGAGACAGCAGGGGCCGAGCGGTAGGCTTCCCTGCCCCCAGGGATGTTCCAGAGGA | |
| | GCGCAAGGGAGGGGCTGGGCATAAAAGTCAGGGCAGAGCCATCTATTGCTTACATT | |
| | TGCTTCTG | |
| CNS-11 | TAACTTAGCATAGTGAGACAGGAAGCAGCCACTGTCAGTGGTGGCCGAGAAGGGGA | 734 |
| (SEQ ID | CTTCACGCGGTCATCCTGAACCATAGATTCTGGGAGTCTTGGAGCCTGGTGCAATT | |
| NO: | ATTCTCTCATCTGTGTGCAGGAGGGAGGGCTCTATAATGAGTGTAGGTGGCCGCTC | |
| 188) | GAGAACGAGGTATCCAGGGGAGGGAATCCACCCACGGTCGGTCTTCCATCTCTGAT | |
| | ACTTAAATGTCAACAGAGCCAAATTGGCTGCTCAGCCAGCCCTTCCTCCACCTCTG | |
| | CTGCTAAGGCTCAGCAAACTTGCCGCTGTGCCCGCCAAGAGGGGACACAGCCTTGA | |
| | GTCATCTCAGACCACTTCCTGCTCATGCACTGGGTGACCTTGAACAGGTGATTCAG | |
| | CCAGCCTGTGAGTCAGCTGCTCTTGGAACCTCACCCTGGGGGCAGGTGAAGAGACA | |
| | AGTCCTCGACCAAGATCATGGATATGGAGTGGATGGGTGGATGGATGGACAGACAA | |
| | GACAGATGGACAGGGTAGAGAGACAGGGCAGAAGGGATACACAGGTAAACATGTCA | |
| | TTTCTCCTTTTGCACCTGTGTGATCACTATTTTATCGATCATAAAGACCTTGAGGA | |
| | CCTACTGTGTGTTGTGCAGGGAACAGTGCCTACATTTTGACACACCTACTCCTGGT | |
| | GGTGCAGGAGGGCTGGGCATAAAAGTCAGGGCAGAGCCATCTATTGCTTACATTTG | |
| | CTTCTG | |
| CNS-13 | CCTGAGGGCAGCACTCACCTCCACAGGGGTCCAGGGGCCTCCCCAGCCTCAGCACC | 1132 |
| (SEQ ID | TGGCCTGGGCTCCTGCCTCCAGAGAGCCTGGCCCCAAGGAAGAGTCTAGTAAGCTT | |
| NO: | AGTTCCCATCGGGCTTCCATGAAAGCACAACTGGCCCGGCAGGAAACCGAATTAAA | |
| 189) | AAGCAATATTTGTATCAGTGGAAGACATTTGCTGAAAGGTTAAATCCACATCCGGC | |
| | AGTGTGGGCCATGAGCCTCCGGCGTGGTGTTCATCAGGCATGTCTCTCCTCCTGGC | |
| | CTGGGCACCTGAGCACTGGGGCCGCCCTGGGCAGAGCTGGGGGGGGGTGCTGGGGG | |
| | GCCTGGAGCTGCCTCACCGAGGGATCCTCAGCAGCCGACCCTGGGGGAGGCAAATG | |
| | AGACTCTTTCTGGGGACCTTGAGGGGAGCTCGGGGGAGCCATGCAGAGCTTCACCA | |
| | GGCCTGGACACTGGGCATGGAGGCTGGGCCACCCAAGGGCCATCACCAGGGACTCA | |
| | GGTGGGTGGGCCTCAGCCCTGGGTGACAGAAGCTCACGGGCCGCAGGGCGAGGCCA | |
| | GAGGCTGAGCCTTCAGGCTGAGGTCTTGGAGGCAAATCCCTCCAACGCCCTTCTGA | |
| | GCAGGCACCCAGACCTACTGTGGGCAGGACCCACAGGAGGTGGAGGCCTTTGGGGA | |
| | ACACTGTGGAGGGGCATAGCATCTCCGAGAGAGGACAGGGTCTGCACTGGGTGCTG | |
| | AGAGACAGCAGGGGCCGAGCGGTAGGCTTCCCTGCCCCCAGGGATGTTCCAGAGGA | |
| | GCGCAAGGGAGTCCCTGTGTGCTCTCTTGAGGGCTGTGGGGCCAAGGGGACCCTGG | |
| | CTGTCTCAGCCCCCCGCAGAGCACGAGCCCCTGGTCCCCGCAAGCCCGCGGGCTGA | |
| | GGATGATTCAGACAGGGCTGGGGAGTGAAGGCAATTAGATTCCACGGACGAGCCCT | |
| | TTCTCCTGCGCCTCCCTCCTTCCTCACCCACCCCCGCCTCCATCAGGCACAGCAGG | |
| | CAGGGGTGGGGGATGTAAGGAGGGGAAGGTGGGGGACCCAGAGGGGGCTTTGACGT | |
| | CAGCTCAGCTTATAAGAGGCTGCTGGGCCAGGGCTGTGGAGACGGAGCCCGGACCT | |
| | CCACACTGAGCC | |

TABLE 12
Minimal/Proximal Promoters comprised in the promoters of Table 11

| Name | SEQUENCE |
|---|---|
| SYNP CRE151 | GGGCTGGGCATAAAAGTCAGGGCAGAGCCATCTATTGCTTACATTTGCTTCTG |
| (SEQ ID NO: | |
| 126) | |
| CRE0001v1_Pitx3 | GGGAGGATTGGAACAGAGACAAAAGGGAGGAGAGACGGACAGCGACAAGTGGAGAAA |
| (SEQ ID NO: | ATCGGCGAAACTTGAGTGGCAGAGAAGTCTGAGCGCTGAGACCCGGCGGCCCCGTGC |
| 127) | GCCTTCCCACCTGGCGCCGATCCACTTTCCTCGGGGTAGCGGCCCAACCCACTTCGC |
| | TGCCAGCCGATCCCTTTTACCCGTGGCTACCGGGACCACTCTACTCTCGCCCACTTG |
| | GCTCTGCCTAAGCGTCCTAGCCGGAGCGCGGTCTCTGCCACGTGGGGAGGGGCGCGG |
| | CCGAGTTGCTGAAGAGCGCTTCTGATTGGCCAGAGGGCGGGGTTCTTGGCGTCTCGC |
| | CGGCCAGACCCCTCCCTCAAAGGCGGGGCCTGGAGATCCACAGCTGGAAAGGGCGGA |
| | GCCCCAGCAGGGCAGCTGGAAAGGGGCGGGGCCTGACGCGCGCGGCTCGCCGCGGCG |
| | GGCTGGGGGCGCCCTGGTCTGCCATAAAGTGAATGGGCGCCGGCTGGGGGTGGCAGT |
| | ACGCGGTGAGGCTCACTCCCTCCGAGAGTCCAGGAGCGCC |

TABLE 13
Synthetic CNS-specific promoter overview

| | Minimal/proximal | | |
|---|---|---|---|
| Promoter name | promoter | CRE | UTR |
| CNS-1 | SYNP_CRE151 | CRE0004_Lmx1b | |
| CNS-2 | SYNP_CRE151 | CRE0003_Pitx3 | |
| CNS-3 | CRE0001v1_Pitx3 | CRE0002_Gbf1 | |
| CNS-4 | CRE0001_Pitx3 | | |
| CNS-5_v2 | CRE0005_faf1 | | |
| CNS-6_v2 | CRE0006_Pitx2 | | |
| CNS-7_v2 | CRE0007_Pitx2 | | |
| CNS-8_v2 | CRE0008_Pitx2 | | |
| CNS-1 + CMV-IE | SYNP_CRE151 | CRE0004_Lmx1b | CMV-IE UTR and |
| UTR and intron | | | intron |
| CNS-4 + CMV-IE | CRE0001_Pitx3 | | CMV-IE UTR and |
| UTR and intron | | | intron |
| CNS-5 | SYNP_CRE151 | CRE0005_faf1_short | |
| CNS-6 | SYNP_CRE151 | CRE0006_Pitx2_short | |
| CNS-7 | SYNP_CRE151 | CRE0007_Pitx2_short | |
| CNS-8 | SYNP_CRE151 | CRE0008_Pitx2_short | |
| CNS 10 | CRE151 | CRE00010 | |
| CNS 11 | CRE151 | CRE00011 | |
| CNS 13 | CRE527 | CRE00010 | |

TABLE 14
Exemplary CREs

| Name | SEQUENCE |
|---|---|
| CRE0006_GFAP | AACATATCCTGGTGTGGAGTAGGGGACGCTGCTCTGACAGAGGCTCGGGGGCCTGAGCT |
| (SEQ ID | GGCTCTGTGAGCTGGGGAGGAGGCAGACAGCCAGGCCTTGTCTGCAAGCAGACCTGGCA |
| NO: 99) | GCATTGGGCTGGCCGCCCCCCAGGGCCTCCTCTTCATGCCCAGTGAATGACTCACCTTG |
| | GCACAGACACAATGTTCGGGGTGGGCACAGTGCCTGCTTCCCGCCGCACCCCAGCCCCC |
| | CTCAAATGCCTTCCGAGAAGCCCATTGAGCAGGGGGCTTGCATTGCACCCCAGCCTGAC |
| | AGCCTGGCATCTTGGGATAAAAGCAGCACAGCCCCCTAGGGGCTGCCCTTGCTGTGTGG |
| | CGCCA |
| CRE0008_GFAP | AACATATCCTGGTGTGGAGTAGGGGACGCTGCTCTGACAGAGGCTCGGGGGCCTGAGCT |
| (SEQ ID | GGCTCTGTGAGCTGGGGAGGAGGCAGACAGCCAGGCCTTGTCTGCAAGCAGACCTGGCA |
| NO: 100) | GCATTGGGCTGGCCGCCCCCCAGGGCCTCCTCTTCATGCCCAGTGAATGACTCACCTTG |
| | GCACAGACACAATGTTCGGGGTGGGCACAGTGCCTGCTTCCCGCCGCACCCCAGCCCCC |
| | CTCAAATGCCTTCCGAGAAGCCCATTGAGCAGGGGGCTTGCATTGCACCCCAGCCTGAC |
| | AGCCTGGCATCTTGGGATAAAAGCACCGGCGGTGGAGAACAAGGCTCTATTCAGCCTGT |
| | GCCCAGGAAAGGGGATCAGGGGATGCCCAGGCATGGACAGTGGGTGGCAGGGGGGGAGA |
| | GGAGGGCTGTCTGCTTCCCAGAAGTCCAAGGACACAAATGGGTGAGGGGA |
| CRE0006_AQP4 | CTAGCCCACAGGAAATGTCTGTCTATATCCAGGCAAGTACCTTGCTCATTGGACCAACC |
| (SEQ ID NO: | CGAAACTGTTCAGGGAAGATCAGGGAAATCAACTCAGTTACAAATGGGATAATCATGCC |
| 101) | CAGTAAAAACTACCTGTGGTGAATAAAGAGTTAACCCCTGTTCCATCTTAGGTCACTAT |
| | GCAGAGTACCAATGAGTACAAGAGATGGTGCCAAAGAGGGTGGCCCCTCCCTAGCTGGG |
| | AACAGTCAACCCTTAGGAACTAGACTGTCAACACATCAGCCAGCCAGAGACAAGGGAAA |
| | CCGTGGCAACCAAGTGTTGCTGGCACATTGTGAGGTGGTGATGGGAACTGCAGAGGCCC |
| | TGCACAGCATGCTAATGAGCCCAGGCAAACAT |
| CRE0008_AQP4 | TCCAAAGAAAAGCCAGATAAGTAGCTGATTATTGCATAGAGCTGACAGTATCACAGGAA |
| (SEQ ID | GATCAGTAGTAGCAGCTCAAGTACAAAAAGGTTAATTAGCAATACTTAATAAGAAAAAC |
| NO: 102) | TACCTCTGGCAGGTGAAGAGTTAATCCCTGGTCAATTTTAAGCTACTCTGCTGAGAGTA |
| | CTAATAAGTGTAGGGGTTGGAGCCAATGAGGGTGACCCCTTCCTTGATGGGAACAGTCA |
| | TCCCTTAGGAACTGCCCTGGAAAGCATCAGCCAGCCAGAAAACAGGGAAAGAGGCTGAG |
| | AAACCGTGGTAACCAAGTTTTGCTGGCACTTTGTAAAATGGTAACTGCAACTGCCGAGG |
| | CTGTGCAGAGAATGCTAATAAGCCTAGGACAACCTGTAAAGAGTGGACCTAGAAAATGT |
| | CCACC |
| CRE0005_GFAP | AACATATCCTGGTGTGGAGTAGGGGACGCTGCTCTGACAGAGGCTCGGGGGCCTGAGCT |
| | GGCTCTGTGAGCTGGGGAGGAGGCAGACAGCCAGGCCTTGTCTGCAAGCAGACCTGGCA |
| (SEQ ID NO: | GCATTGGGCTGGCCGCCCCCCAGGGCCTCCTCTTCATGCCCAGTGAATGACTCACCTTG |
| 103) | GCACAGACACAATGTTCGGGGTGGGCACAGTGCCTGCTTCCCGCCGCACCCCAGCCCCC |
| | CTCAAATGCCTTCCGAGAAGCCCATTGAGCAGGGGGCTTGCATTGCACCCCAGCCTGAC |
| | AGCCTGGCATCTTGGGATAAAAGCAGCACAGCCCCCTAGGGGCTGCCCTTGCTGTGTGG |
| | CGCCACCGGCGGTGGAGAACAAGGCTCTATTCAGCCTGTGCCCAGGAAAGGGGATCAGG |
| | GGATGCCCAGGCATGGACAGTGGGTGGCAGGGGGGGAGAGGAGGGCTGTCTGCTTCCCA |
| | GAAGTCCAAGGACACAAATGGGTGAGGGGA |
| CRE0007_GFAP | AACATATCCTGGTGTGGAGTAGGGGACGCTGCTCTGACAGAGGCTCGGGGGCCTGAGCT |
| (SEQ ID NO: | GGCTCTGTGAGCTGGGGAGGAGGCAGACAGCCAGGCCTTGTCTGCAAGCAGACCTGGCA |
| 104) | GCATTGGGCTGGCCGCCCCCCAGGGCCTCCTCTTCATGCCCAGTGAATGACTCACCTTG |
| | GCACAGACACAATGTTCGGGGTGGGCACAGTGCCTGCTTCCCGCCGCACCCCAGCCCCC |
| | CTCAAATGCCTTCCGAGAAGCCCATTGAGCAGGGGGCTTGCATTGCACCCCAGCCTGAC |
| | AGCCTGGCATCTTGGGATAAAAGCAGCAC |
| CRE0012_ArC | AGCGCGCAGAGTCTGCATGCGTGAGGAAGCTCCTGGGCGCGTCACAGCCGCGCTATTCT |
| (SEQ ID NO: | CAGCGTCTCTCCTTTTATGGCTCCGGAAGTGAGCTGGGGTTGCTGGCAGCCTGGCTGGC |
| 105) | ACT |
| CRE0001_S100B | CTTCCTCTTATATTTCACCAAGACTCAGTTCCTGAGCAAGAAACCACAGGCACAGCAAG |
| (SEQ ID NO: | TGCCATGAAAAGCGGCTTTGTGTGGGGTGGGCTCTTCACACTCCAATCTCCACTTCCTT |
| 106) | CTCAAGGCCTCAAAAAAAGTTGAAAAATGAAAACAAAAGCCCTGCTGTGTTGAGCTGGG |
| | CTCTGGCGTTGCCATGGACCCAGGGCAAACAGCGGTGCTCCTGCTCTGCCCCCGGCTCA |
| | GCTCATGCTGGGCCTGCACTTCTGGAAGGGAGCATGGACTTTGGAATGACTGGTTAGAA |
| | CCCAAATGAATTAATGGAATTTGACATAGTTCAAAAATAATAAAATGTGATACCCATGA |
| | AATGCTGATATTCTGCCTTAATTTGCCAGATTGGGGGCC |
| CRE0009_S100B | TTCAGAGGTCGGATCTGAATCCAGCTCCAAGGCCCCAGCACCCAAGCCCTGACCCCATG |
| (SEQ ID NO: | GCTGCCTGCTGGCTGGGAGTGGCATTCTTTAGAACAGGAAAGGCAGTGAGTTCTCATTG |
| 107) | CATCAATACTTGCATTTGCTACAACAGAAGCTTTTTGTCTCTTCCTCACATTCCTTTAG |
| | AACACAAGCCTCCTTTTCTGCCTGTGGCCATCGGTCACTGGAGTCAGCCTCGTGAGTGG |
| | CTTGGTGGCGGATGGCACCGTCCTTTGTGGGAGAAAACAATGTTGCTGCCCAGGCCTTT |
| | CTGGAATGACCCCTTCCCACCCCCTGACCAGCCCCAGCAAGGCCCGGGGCTGGCTGCCT |
| | AGTTGATTGGAGCCAAGAGTTTGCTGAATGGATGAAGGGAGAAGGGACATCCAA |
| CRE0002_S100B | TCAGGGGTGCAGCTTTTTTTCTGTCTTTTACTCAGCCTGAGAAAGGTTGTCGTTTGACA |
| (SEQ ID NO: | AGGTTTGTTCAGAGGTCGGATCTGAATCCAGCTCCAAGGCCCCAGCACCCAAGCCCTGA |
| 108) | CCCCATGGCTGCCTGCTGGCTGGGAGTGGCATTCTTTAGAACAGGAAAGGCAGTGAGTT |
| | CTCATTGCATCAATACTTGCATTTGCTACAACAGAAGCTTTTTGTCTCTTCCTCACATT |
| | CCTTTAGAACACAAGCCTCCTTTTCTGCCTGTGGCCATCGGTCACTGGAGTCAGCCTCG |
| | TGAGTGGCTTGGTGGCGGATGGCACCGTCCTTTGTGGGAGAAAACAATGTTGCTGCCCA |
| | GGCCTTTCTGGAATGACCCCTTCCCACCCCCTGACCAGCCCCAGCAAGGCCCGGGGCTG |
| | GCTGCCTAGTTGATTGGAGCCAAGAGTTTGCTGAATGGATGAAGGGAGAAGGGACATCC |
| | AAGTGACTGAAGAAGATCTTAACAGAA |

TABLE 15
Cis-regulatory elements (CRE) comprised in the promoters of Table 11

| Name | SEQUENCE |
|---|---|
| CRE0004_Lmx1b | CTGGGCAGAGAGGGGGCATCGGGGGCATGGCTAGGGGCCAGCACTGTGCTTCCTG |
| (SEQ ID NO: 128) | GGCGCCTCACCTCCTCCCTGACTCCTGGAGACTCCCAGCCCCTGTCTGGGAGATG |
| | AGCATTTAGGAATCTGCTTGTGCAGGGGTGGTGGGAGGGGCCGGGGTGGAGGGCG |
| | CATCCCCACGGGGAGATTGGATGGAAATGGCCTGCCAGTGTGTGTGTGAGTGTGC |
| | GCCTGTGGCAGCAGCAGAGTAAACAGCCGCTGCCCTGTCCTCTCTGCGGCCGTGG |
| | CCAGGTACACAGGCCTGTTTGGACAGCTGCCTTGTCTGTCCGTCTGTTTGGGAGA |
| | TGCTGGCTGATAGATGGGGATGGGCGGACTGTTAACCCCTCGTTGCCTGCACTGC |
| | TATGTGCTTCCTGCCTCATCCATGGGGTAGAAGGTAGCCAGAAGGTGGTCCTGGC |
| | TGTGCCCCCAGCTCCTCTCTAGGGGGGAAACCTCTAGTTCTGAGTCAGGGACAGA |
| | GTGAGGAGGGCTCCAGGGCATCAAGAGCTTGCTCCTCCCCGCACCAGGGAGCCAA |
| | GGACAGAGGAGAAGGGGGTCTTCCCCAGTGGTGACTAGGGGCAGAATATGTCTCT |
| | GAGTGAGTGTCTGGAGCCCTCCTCACCCCAACACCATG |
| CRE0003_Pitx3 | GGTGTGTGGAAGGGTGAGAGGCACACACACAGACACTGAAAGAATCCTAGGCCTG |
| (SEQ ID NO: 129) | GTAGGCACTTAACAAATGTCTGTTACAGACCAGAATTTTATTGCTGTTAGAGACC |
| | CAAGCCCCTCATAGGAACAGTGAGAAACAGGTGCAGAAAGGCGGAGTAACTTTAT |
| | CTAAAGTCATAGGCTCCCTGAATAGCAGAGCTGACACCTACAAGGAAGCGTTGGA |
| | GACCAGATCTACCAGCTAGCCTCCCTGAGACCACGAGGTGGCGCCGCAGCACCGG |
| | CTGTGGCCGATGCCAGCCAGGTAGCCGGTTTCCCACGTCCCCCGCACGCACGCAC |
| | CTCTTTGCTGCAGGAATCCCGGGCTGCCCCGACCTGGAGTAGGGGGGGTGGTGAG |
| | TGGGACTGAGTCCCTAGAAGCCTGGACCCTCACTTCGTTCCTGTACATCCAGCTC |
| | GCCTGTAGACAGTGGGGGAGGATGAAGGGAAGAGGACTCAAGCGCAACTTTGAAT |
| | CATCACGCCTTCGACAGTCCGCGCACGTTTATTTCATTTATCTTTGAAAACGAGG |
| | GAGGGGAAGCCTGGAGAAGGCGGGATGGGCCAAGGGTGAGTTGGCCCCCGGGGAG |
| | CTGGTCCCTGTTCCTGGCTTTAGTCCCAGGGGCGCGGTCTGTGTGTAGGGC |
| CRE0002_Gbf1 | TGCTACCAGAGCCGGGAGAGCTGCTCGGAGACGCCTCCGGGGTGCGGGCTGGACA |
| (SEQ ID NO: 130) | TGAGCAGCGGCTGCCGGTCCTGGGACTAGGCCCCGCCATTTTGGATCCGCTGACA |
| | GGTCAGCGAAGTCTCTTCCTAGAGTTCCGGTGTCGTGAAGGCCGCCCTGACATCG |
| | CAATAGGGAATTAGTGGGAAGGGCCCTTAAATTGGGCGAGCCAAGGTGGG |
| CRE0005_faf1_ | GGAACATTTAGACTTTGGGAAAGAGATTAGTGATTTACATTGCTATCTCACTGAT |
| short | TTAATTTAAATGCTCTTCCAAACCAAACACACATGTGCCGAAGAGGCTACTAAGA |
| (SEQ ID NO: 131) | AACCCAACATGCAGAGTTCTCTATAAGTGCAGCCGACAGTGTTGACTGAAACTAA |
| | ACTTGGAAATCCAGGGCACTAATGCACAATATCAAGCAATAAAACGGCATCTCTT |
| | TGGCAATATTTAATTTAAAAAAGAAGAAAGAGACAGGCGAAGATCAGGCACTGTC |
| | TGTTTTGGAGGATCAACCATTCTGCATTTCAAAGCATTGGTCCCTGCAATATCCA |
| | GGTTACTGTGCTAGAATCTCGACTATTATATCGCAGTTGTGAGAGGGAGGGCAAA |
| | GATGTGTTTACTCAGTGATTAGGCCCTTAGAATAAGCCTCTAGCTCCTAGAGAGA |
| | CAGCTCACCACTTATTCATTTGGGCCAATTCACAAAGCCTAGGAAGATTAAACAT |
| | CCATGCTGAGAAGACAAGCGAATGCAGACGGTGAAAAAGAAATAAAAATTCTTTA |
| | AAAACTCTGAGATGACTTCATTATTTTTCCACAAGGAAACTTTAGGAAAGTGTTT |
| | AGTTAGAGAAAAACCCACATTGACCTCTCTCTAAACCCTTAATCTTTCCTTTGTG |
| | GTGGCACTGCTTTGTGGTAAGCGACTGGCTCGCCTCGCCCCTCTTTTCACTGGAA |
| | GCTGAGAGAAAAAAGACTCTGGAGAAACAGTTTTCGTTCCAGGGACACAAACCCC |
| | TGACACTGTTAA |
| CRE0006_Pitx2_ | GAAAATTTAATGTCATTGAAGCTCTATTTCCAATAGCAAAGGAGCACTATTGCTA |
| short (SEQ ID NO: | ATAGACTTCAGAGCTTGAAATAAATAAATCTTTGGAATCCTGTTGCATCTCTTGG |
| 132) | GGTGTGACATTTGACAGTCTTTTATAGCACAGAACGAAACAAGTTTGTGAGCTGG |
| | AATTCAATTGTGGCGTATTGATTCCTTGCATCAGTCATTATTCCCTGCTGATTGA |
| | CAGGTGAAAATTGGTTACGTTAAGTATTTCATATGTTATATTGGCTGACATTTGC |
| | TTGCCTGCTCTTGTGTCAATATTGTTGTAAAGATCTCCAGCTTTATGAGATAGCA |
| | ATAGACACTGACTGTGGCTTTTGTGTGATGTTCCAGTGTTTTTCCTGACATAATT |
| | TAAGACATATTAAAAACCAGCAGCATCTTCCCTCTTGAGAAGCTTAATGCCAATA |
| | TTATTGTCTTCCAGGGGAAGATCATGTATGCTCATAATCGGGTGCTAATTTCCAC |
| | CAGTACGCTCATGTTTAGGCATTAGGCACTATAACTGTAAAATTGAGCCTTCTTG |
| | ATTGATTCATGTCAAGCCTCATCTCGGCTCCTGCAGGGGAAGTCATCCGGCTGAC |
| | CCTTTTTACACTAAAAGAAGAGATTTGTGTTCCTTTCTTTCACCTGGAACCATCA |
| | AATTGACTGAATAATCTGTAATACATTAGTGCTGACATTTGTTAGGGAGAATTAA |
| | ACAAGACACAGTAATCATTCCCCAGAATAAAAATTGTGTTTGAT |
| CRE0007_Pitx2_ | TGAGACTGAGTGGGAGTGGGGATGAAGCCTTTTTTCTTCATTTTTTTTTCCTCAG |
| short (SEQ ID NO: | GTGCAATTCTGTGTTAATATAAGAGAAGTGTGGCCTTCCTTCTCATAGCACTAAA |
| 133) | AGTGAGATAATCCCTGTGTAAGAAATCAGTAAGTACGGTCTGCTTAATCTAGTCC |
| | CAGTGTGAAACTGTTGACATTTGTTCTTTTTTCTATCATTATGTGACTGGGCCTG |
| | TTTTGTGCTGGATTAGGCACAAATCTCCTATGCAGCACATTTGGCATGTTACTAG |
| | TAGTTTAACTTCATTAATAATGTATGAAGAAAATGTAATCCATGACAAGGAAGCA |
| | AAGAAAAGTATTTTTTTTTTTTTTTGCTTCTCCCAAATCCTTTGGAATGAGTAAT |
| | TATTCAACATTTTATGTTTGATGTTATATTTTACAATTCAACTTCCATA |
| CRE0008_Pitx2_ | GCTGGTGCTTCTTTTTTCTGTTTTTTAAGCTGCTTTACAGACGAAAATGGAACTA |
| short (SEQ ID NO: | TATTTGGAACAATGCTTTCTGTTTTTCCATACTATTGATATTTGTGGAAAGTCAC |
| 134) | AAAATGGCCTAAGGAAGCTAAGCTCGCCCCAAGCAGTGGTCACTTACAAGTACTT |
| | TTGTACTCTGTACTCCTGTCACATTTGGGCGATCAGAGCAACAGCTGGGGAGACT |
| | TTTTCAACAAAGATGAGTGTCAGATAATCCTGATGAGATTCCACATCCAACATCT |
| | TTTGTAATTATGTCACATTCAGCTGTAATGGAATAATTCAAGCTGAAAGAACAAG |
| | CTTTGATCCTTTCTTAAACCTTTCCCTGTGGACTGGCTATCTAAAAGATTTAAAG |
| | ATATTTCTGTTACAAGATCTAGTGTTTCCTCAGAGAAGTCATGCTTCTGAAGCAT |
| | CGTGATCTACAAGAACAATATCAAGTTTGCCAAACACATTTCTGAAAGCATCGTG |
| | TTTTGGGGGGAGGGGTTGTATTTAATGAAGATATCAATAATATGC |
| CRE151 (SEQ ID | GGGCTGGGCATAAAAGTCAGGGCAGAGCCATCTATTGCTTACATTTGCTTCTG |
| NO: 190) | |
| CRE00010 (SEQ | CCTGAGGGCAGCACTCACCTCCACAGGGGTCCAGGGGCCTCCCCAGCCTCAGCAC |
| ID NO: 191) | CTGGCCTGGGCTCCTGCCTCCAGAGAGCCTGGCCCCAAGGAAGAGTCTAGTAAGC |
| | TTAGTTCCCATCGGGCTTCCATGAAAGCACAACTGGCCCGGCAGGAAACCGAATT |
| | AAAAAGCAATATTTGTATCAGTGGAAGACATTTGCTGAAAGGTTAAATCCACATC |
| | CGGCAGTGTGGGCCATGAGCCTCCGGCGTGGTGTTCATCAGGCATGTCTCTCCTC |
| | CTGGCCTGGGCACCTGAGCACTGGGGCCGCCCTGGGCAGAGCTGGGGCGGGGTGC |
| | TGGGGGGCCTGGAGCTGCCTCACCGAGGGATCCTCAGCAGCCGACCCTGGGGGAG |
| | GCAAATGAGACTCTTTCTGGGGACCTTGAGGGGAGCTCGGGGGAGCCATGCAGAG |
| | CTTCACCAGGCCTGGACACTGGGCATGGAGGCTGGGCCACCCAAGGGCCATCACC |
| | AGGGACTCAGGTGGGTGGGCCTCAGCCCTGGGTGACAGAAGCTCACGGGCCGCAG |
| | GGCGAGGCCAGAGGCTGAGCCTTCAGGCTGAGGTCTTGGAGGCAAATCCCTCCAA |
| | CGCCCTTCTGAGCAGGCACCCAGACCTACTGTGGGCAGGACCCACAGGAGGTGGA |
| | GGCCTTTGGGGAACACTGTGGAGGGGCATAGCATCTCCGAGAGAGGACAGGGTCT |
| | GCACTGGGTGCTGAGAGACAGCAGGGGCCGAGCGGTAGGCTTCCCTGCCCCCAGG |
| | GATGTTCCAGAGGAGCGCAAGGGAG |
| CRE00011 (SEQ | TAACTTAGCATAGTGAGACAGGAAGCAGCCACTGTCAGTGGTGGCCGAGAAGGGG |
| ID NO: 192) | ACTTCACGCGGTCATCCTGAACCATAGATTCTGGGAGTCTTGGAGCCTGGTGCAA |
| | TTATTCTCTCATCTGTGTGCAGGAGGGAGGGCTCTATAATGAGTGTAGGTGGCCG |
| | CTCGAGAACGAGGTATCCAGGGGAGGGAATCCACCCACGGTCGGTCTTCCATCTC |
| | TGATACTTAAATGTCAACAGAGCCAAATTGGCTGCTCAGCCAGCCCTTCCTCCAC |
| | CTCTGCTGCTAAGGCTCAGCAAACTTGCCGCTGTGCCCGCCAAGAGGGGACACAG |
| | CCTTGAGTCATCTCAGACCACTTCCTGCTCATGCACTGGGTGACCTTGAACAGGT |
| | GATTCAGCCAGCCTGTGAGTCAGCTGCTCTTGGAACCTCACCCTGGGGGCAGGTG |
| | AAGAGACAAGTCCTCGACCAAGATCATGGATATGGAGTGGATGGGTGGATGGATG |
| | GACAGACAAGACAGATGGACAGGGTAGAGAGACAGGGCAGAAGGGATACACAGGT |
| | AAACATGTCATTTCTCCTTTTGCACCTGTGTGATCACTATTTTATCGATCATAAA |
| | GACCTTGAGGACCTACTGTGTGTTGTGCAGGGAACAGTGCCTACATTTTGACACA |
| | CCTACTCCTGGTGGTGCAGGA |
| CRE527 (SEQ ID | TCCCTGTGTGCTCTCTTGAGGGCTGTGGGGCCAAGGGGACCCTGGCTGTCTCAGC |
| NO: 211) | CCCCCGCAGAGCACGAGCCCCTGGTCCCCGCAAGCCCGCGGGCTGAGGATGATTC |
| | AGACAGGGCTGGGGAGTGAAGGCAATTAGATTCCACGGACGAGCCCTTTCTCCTG |
| | CGCCTCCCTCCTTCCTCACCCACCCCCGCCTCCATCAGGCACAGCAGGCAGGGGT |
| | GGGGGATGTAAGGAGGGGAAGGTGGGGGACCCAGAGGGGGCTTTGACGTCAGCTC |
| | AGCTTATAAGAGGCTGCTGGGCCAGGGCTGTGGAGACGGAGCCCGGACCTCCACA |
| | CTGAGCC |

In a further aspect of the present invention, there is provided a synthetic central nervous system (CNS)-specific promoter comprising or consisting of a sequence according to any one of SEQ ID NOs: 187, 188 or 189 or a functional variant thereof. In some embodiments, the synthetic CNS-specific promoter comprises or consists of a sequence which is at least 60%, 65%, 70%, 75%, 80%, 85%, 90%, 91%, 92%, 93%, 94%, 95%, 96%, 97%, 98% or 99% identical to any one of SEQ ID NOs: 187, 188 or 189.

The present invention thus provides various synthetic CNS-specific promoters and functional variants thereof. It is generally preferred that a promoter according to the present invention which is a variant of any one of SEQ TD NO: 187, 188 or 189 retains at least 25%, at least 50%, at least 75%, at least 80%, at least 85%, at least 90%, at least 91%, at least 92%, at least 93%, at least 94%, at least 95%, at least 96%, at least 97%, at least 98%, at least 99% or at least 100% of the activity of the reference CNS-specific promoter (any one of SEQ ID NO: 187, 188 or 189).

In some embodiments of the present invention, the CNS-specific promoter has a length of 1200 or fewer nucleotides, preferably 1175 or fewer, 1150 or fewer, 1125 or fewer, 1100 or fewer, 1075 or fewer, 1050 or fewer, 1025 or fewer, 1000 or fewer, 975 or fewer, 950 or fewer, 925 or fewer, 900 or fewer, 875 or fewer, 850 or fewer, 825 or fewer, 800 or fewer, 775 or fewer, 750 or fewer, 740 or fewer, 730 or fewer, 720 or fewer, 710 or fewer, 700 or fewer nucleotides.

In another aspect of the present invention there is provided a synthetic CNS-specific promoter comprising a cis-regulatory element (CRE) according to any one of SEQ ID NO: 191 or 192, or a functional variant thereof. In some embodiments, the CRE may be operably linked to a promoter element. In some embodiments, the promoter element may be a minimal or a proximal promoter. Preferably, the proximal promoter is a CNS-specific proximal promoter. In some embodiments, the minimal promoter is a CNS-specific minimal promoter. In some embodiments, the functional variant of the CRE according to any one of SEQ ID NO: 191 or 192 comprises or consists of a sequence which is at least 60%, 65%, 70%, 75%, 80%, 85%, 90%, 91%, 92%, 93%, 94%, 95%, 96%, 97%, 98% or 99% identical to SEQ ID NO: 191 or 192.

In some embodiments, the synthetic CNS-specific promoter comprises or consists of a CRE00010 (SEQ ID NO: 191), or a functional variant thereof, operably linked to CRE151 (SEQ ID NO: 190), or a functional variant thereof.

In another embodiment, the synthetic CNS-specific promoter comprises or consists of CRE00011 (SEQ ID NO: 192), or a functional variant thereof, operably linked to CRE151 (SEQ ID NO: 190), or a functional variant thereof.

The element CRE151 is a promoter element. It functions in combination with one or more CNS-specific CREs to provide CNS-specific transcription from the promoter in which they are comprised. Its sequence and functional variants thereof are discussed further below.

The elements CRE00010 and CRE00011 are CNS-specific CREs. They function in combination with a promoter element to modulate, typically enhance, CNS-specific transcription from the promoter in which they are comprised. Their sequences and functional variants thereof are discussed further below.

In embodiments wherein CRE00010 is operably linked to CRE151, CRE00010 can be contiguous (i.e. positioned immediately adjacent to) with the adjacent CRE151, or it can be separated by a spacer or other sequence.

In embodiments wherein CRE00011 is operably linked to CRE151, CRE00011 can be contiguous (i.e. positioned immediately adjacent to) with the adjacent CRE151, or it can be separated by a spacer or other sequence.

In another embodiment, the synthetic CNS-specific promoter comprises or consists of CRE00010 (SEQ ID NO: 191), or a functional variant thereof, operably linked to CRE527 (SEQ ID NO: 211), or a functional variant thereof.

The element CRE527 is a promoter element, suitably a minimal promoter element. In some embodiments, the element CRE527 is a CNS-specific promoter element.

In embodiments wherein CRE00010 is operably linked to CRE527, CRE00010 can be contiguous (i.e. positioned immediately adjacent to) with the adjacent CRE527, or it can be separated by a spacer or other sequence.

In one embodiment, the synthetic CNS-specific promoter comprises or consists of CRE00010 (SEQ ID NO: 191), or a functional variant thereof, operably linked to CRE527 (SEQ ID NO: 211) or CRE151 (SEQ ID NO: 190), or a functional variants thereof.

In a further aspect of the present invention, there is provided a minimal promoter comprising or consisting of a sequence according to SEQ ID NO: 211 or a functional variant thereof. Suitably a functional variant of the minimal promoter comprises a sequence which is at least 60%, 65%, 70%, 75%, 80%, 85%, 90%, 95%, 96%, 97%, 98%, 99% identical to SEQ ID NO: 211.

In another aspect of the present invention, there is provided a synthetic promoter comprising a minimal promoter according to SEQ ID NO: 211 or a functional variant thereof. Suitably a functional variant of the minimal promoter according to SEQ ID NO: 211 comprises a sequence which is at least 60%, 65%, 70%, 75%, 80%, 85%, 90%, 95%, 96%, 97%, 98%, 99% identical to SEQ ID NO: 211.

In a further aspect of the present invention, there is provided a CRE comprising or consisting of a sequence according to any one of SEQ ID NO: 191 or SEQ ID NO: 192, or a functional variant thereof.

The CRE according to the present invention may be combined with additional CREs to form a cis-regulatory module (CRM). Suitably, the additional CREs may be CREs according to SEQ ID NO: 191 or 192 or functional variants thereof, or they can be other CREs. Suitably, the additional CREs are CNS-specific.

Suitably, in another aspect there is provided a CRM comprising a CRE of the present invention. Suitably, in some embodiments, the CRM comprises a CRE according to SEQ ID NO: 191 and/or SEQ ID NO: 192 or a functional variant thereof. In one embodiment, the CRM comprises a CRE according to SEQ ID NO: 191 or a functional variant thereof. In another embodiment, the CRM comprises a CRE according to SEQ ID NO: 192 or a functional variant thereof. In another embodiment, the CRM comprises a CRE according to SEQ ID NO: 191 or a functional variant thereof and a CRE according to SEQ ID NO: 192 or a functional variant thereof. In some embodiments, the functional variant of CRE according to any one of SEQ ID NO: 191 and/or 192 comprises or consists of a sequence which is at least 60%, 65%, 70%, 75%, 80%, 85%, 90%, 91%, 92%, 93%, 94%, 95%, 96%, 97%, 98% or 99% identical to SEQ ID NO: 191 or 192.

In one aspect of the present invention, there is provided a synthetic central nervous system (CNS)-specific promoter comprising a CRM of the present invention.

The CREs, CRMs, minimal promoters or synthetic CNS-specific promoters of the present invention can be active in one or more specific regions of the CNS, preferably in a specific region in the brain, or in a specific brain cell type or cell types or in a combination of both.

The CREs, CRMs, minimal promoters or synthetic CNS-specific promoters of the present invention can be active in one or more of the various parts of the CNS. The CNS consists primarily of the brain and the spinal cord. The retina, optic nerve, olfactory nerves, and olfactory epithelium are sometimes considered to be part of the CNS alongside the brain and spinal cord. This is because they connect directly with brain tissue without intermediate nerve fibers. Suitably, the CREs, CRMs, minimal promoters or synthetic CNS-specific promoters of the present invention may be active in the brain and the spinal cord. Suitably, the CREs, minimal promoters or synthetic CNS-specific promoters of the present invention may be active in the brain but not in the spinal cord or any other part of the CNS. Suitably, the CREs, CRMs, minimal promoters or synthetic CNS-specific promoters of the present invention may be active in the spinal cord but not in the brain. Preferably the CREs, CRMs, minimal promoters or synthetic CNS-specific promoters of the present invention may be active in the brain.

Suitably the CREs, CRMs, minimal promoters or synthetic CNS-specific promoters of the present invention may be active in one or more of the various areas within the brain. Non-limiting examples of brain areas include: frontal lobe, pariental lobe, occipital lobe, temporal lobe (which includes the hippocampus and amygdala), cerebellum, midbrain, pons, medulla and the diencephalon (which includes the thalamus and hypothalamus). Non-limiting examples of spinal cord areas include: cervical vertebrae, thoracic vertebrae, lumbar vertebrae, sacrum vertebrae and coccyx vertebrae. In some embodiments, it may be desirable that the CREs, CRMs, minimal promoters or synthetic CNS-specific promoters of the present invention shows widespread activity in the brain. In some embodiments, the CREs, CRMs, minimal promoters or synthetic CNS-specific promoters of the present invention are active in all parts of the brain or CNS (pan-CNS), preferably in all areas of the brain. In some embodiments the CREs, CRMs, minimal promoters or synthetic CNS-specific promoters of the present invention are active in the brain but not in other parts of the CNS e.g. the spinal cord. In some embodiments, the CREs, CRMs, minimal promoters or synthetic CNS-specific promoters of the present invention are active in 1, 2, 3, 4, 5, 6, 7, 8 or 9 of the areas of the brain recited above. In some embodiments, the CREs, CRMs, minimal promoters or synthetic CNS-specific promoters of the present invention are active in the majority of the areas in the brain, i.e. at least 5, at least 6, at least 7, at least 8 or all 9 of the 9 areas of the brain recited above. In some embodiments, the CREs, CRMs, minimal promoters or synthetic CNS-specific promoters of the present invention are active in from 4 to 6 areas of the brain recited above. In some embodiments, the CREs, CRMs, minimal promoters or synthetic CNS-specific promoters of the present invention are active in from 2 to 4 areas of the brain recited above, such as for example the midbrain, temporal lobe and diencephalon. In some embodiments, the CREs, CRMs, minimal promoters or synthetic CNS-specific promoters of the present invention may be active in the abovementioned areas of the brain and the spinal cord. In some embodiments the CREs, CRMs, minimal promoters or synthetic CNS-specific promoters of the present invention are active in the spinal cord but not in other parts of the CNS, e.g., the brain. In some embodiments, the CREs, CRMs, minimal promoters or synthetic CNS-specific promoters of the present invention are active in 1, 2, 3, 4, or 5 of the areas of the spinal cord recited above. In some embodiments, the CREs, CRMs, minimal promoters or synthetic CNS-specific promoters of the present invention are active in the majority of the areas in the spinal cord, i.e. at least 3, at least 4 or all 5 of the 5 areas of the spinal cord recited above.

In some embodiments, it may be desirable that the CREs, CRMs, minimal promoters or synthetic CNS-specific promoters of the present invention show predominant activity in one area of the CNS, suitably in one area of the brain. Suitably, it may be desirable that the CREs, CRMs, minimal promoters or synthetic CNS-specific promoters of the present invention show activity in one area of the brain but no, or only minimal, activity in the rest of the brain or CNS. In some embodiments, the CREs, CRMs, minimal promoters or synthetic CNS-specific promoters of the present invention are active in only one area of the CNS areas of the brain recited above, such as the midbrain. In some preferred embodiments, the CREs, CRMs, minimal promoters or synthetic CNS-specific promoters of the present invention are specifically active in the midbrain (midbrain-specific). In one preferred embodiment, the CREs, CRMs, minimal promoters or synthetic CNS-specific promoters of the present invention are specifically active in the midbrain (midbrain-specific) but show no or only minimal activity in other areas of the brain. The CREs, CRMs, minimal promoters or synthetic CNS-specific promoters of the present invention can be active in various cells of the CNS. The predominant cell types in the brain are neurons, astrocytes, oligodendrocytes, microglia, and ependymal cells. Other cell types may be present, particularly in inflammatory condition. In some embodiments, it may be desirable for the promoter to be active in many different cell types. In some embodiments, the CREs, CRMs, minimal promoters or synthetic CNS-specific promoters of the present invention are active in substantially all cells of the CNS (e.g. neurons, astrocytes, oligodendrocytes, microglia, ependymal cells). In some embodiments, the CREs, CRMs, minimal promoters or synthetic CNS-specific promoters of the present invention are active in at least four CNS cell types from the CNS cell types listed above, such as neurons, astrocytes, microglia and oligodendrocytes. In some embodiments, the CREs, CRMs, minimal promoters or synthetic CNS-specific promoters of the present invention are active in at least three CNS cell types from the CNS cell types listed above, such as neurons, astrocytes and oligodendrocytes. In some embodiments, it may be desirable for the promoter to be active in a limited number of CNS cell types, or in not more than one CNS cell type. In some embodiments, the CREs, CRMs, minimal promoters or synthetic CNS-specific promoters of the present invention are active in no more than 4, 3, 2 or 1 of CNS cell types from the CNS cell types listed above. In some embodiments, the CREs, CRMs, minimal promoters or synthetic CNS-specific promoters of the present invention are active in no more than two CNS cell types from the CNS cell types listed above, such as neurons, and oligodendrocytes. In some embodiments, the CREs, CRMs, minimal promoters or synthetic CNS-specific promoters of the present invention are active in only one CNS cell type from the CNS cell types listed above, such as neurons. In some embodiments, the CREs, CRMs, minimal promoters or synthetic CNS-specific promoters of the present invention are active in specific subtypes of CNS cell, such as for example dopaminergic neurons. In some specifically preferred embodiments, the CREs, CRMs, minimal promoters or synthetic CNS-specific promoters of the present invention are active in dopaminergic neurons. In some preferred embodiments, the CREs, CRMs, minimal promoters or synthetic CNS-specific promoters of the present invention are active in dopaminergic neurons but not in other CNS cell types or other CNS cell subtypes. In some preferred embodiments, the CREs, CRMs, minimal promoters or synthetic CNS-specific promoters of the present invention are active in GABAergic or glutamatergic neurons. In some embodiments, the CREs, CRMs, minimal promoters or synthetic CNS-specific promoters of the present invention are active in a specific type of CNS cell or subtype of CNS cell, and in a specific area of the brain. The CREs, CRMs, minimal promoters or synthetic CNS-specific promoters of the present invention may or may not be active in tissues outside the CNS. Non-limiting examples of tissues outside the CNS are: the heart, the liver, the kidney, skeletal muscles and the spleen. Suitably, in some embodiments, the CREs, CRMs, minimal promoters or synthetic CNS-specific promoters of the present invention are not or are minimally active in tissues or cells outside of the CNS. Suitably, the CREs, CRMs, minimal promoters or synthetic CNS-specific promoters of the present invention are active in no more than 1,2, 3, 4 tissues out of the tissues outside of the CNS described above in ICV delivery. Suitably, the CREs, CRMs, minimal promoters or synthetic CNS-specific promoters of the present invention are active in no more than 1, 2, 3, 4 tissues out of the tissues outside of the CNS described above in IV delivery.

Suitably, in some embodiments, it may be desirable for the CREs, CRMs, minimal promoters or synthetic CNS-specific promoters of the present invention to be active in the CNS but to also have activity in other tissues outside of the CNS. Suitably, the CREs, CRMs, minimal promoters or synthetic CNS-specific promoters of the present invention may be active in at least 1, 2, 3, 4 or 5 of the tissues outside of the CNS described above in ICV delivery. Suitably, the CREs, CRMs, minimal promoters or synthetic CNS-specific promoters of the present invention may be active in at least 1, 2, 3, 4 or 5 of the tissues outside of the CNS described above in IV delivery.

In some embodiments, the CREs, CRMs, minimal promoters or synthetic CNS-specific promoters of the present invention may be active in the CNS and in the peripheral nervous system (PNS). If the CREs, CRMs, minimal promoters or synthetic CNS-specific promoters of the present invention are active in the CNS and PNS, the CREs, CRMs, minimal promoters or synthetic CNS-specific promoters of the present invention may be called nervous system-specific (NS-specific). The PNS refers to the parts of the nervous system which are outside the brain and spinal cord. Non-limiting examples of peripheral nervous system include cranial nerves, brachial plexus, thoracoabdominal nerves, lumbar plexus, sacral plexus and neuromuscular junctions. In some embodiments, it may be desirable that the CREs, CRMs, minimal promoters or synthetic CNS-specific promoters of the present invention show widespread activity in the PNS. In some embodiments, the CREs, CRMs, minimal promoters or synthetic CNS-specific promoters of the present invention are active in 1, 2, 3, 4, 5, or 6, of the areas of the PNS recited above. In some embodiments, the CREs, CRMs, minimal promoters or synthetic CNS-specific promoters of the present invention are active in the majority of the areas in the PNS, i.e. at least 4, at least 5, or all 6 of the 6 areas of the PNS recited above.

A CNS-specific promoter can be expressed in other non-CNS cells. However, it has higher degree of expression in CNS cells such as neuronal cells in the brain and spinal cord as well as non-neuronal cells or neuronal supporting cells located in the brain and spinal cord. For example, a CNS-specific promoter expresses a gene at least 25%, or at least 35%, or at least 45%, or at least 55%, or at least 65%, or at least 75%, or at least 80%, or at least 90%, or at least 95%, or any integer between 25%-95% higher in cells located in the CNS, including neuronal and non-neuronal cells located in the brain and spinal cord as compared to cells located outside the CNS.

In a further aspect of the invention, there is provided an expression cassette comprising a synthetic CNS-specific promoter according to any of the aspects of the present invention operably linked to a sequence encoding an expression product, suitably a gene, e.g. a transgene. In one embodiment, there is provided an expression cassette comprising any one of CNS-10, CNS-11 or CNS-13 or functional variants thereof, operably linked to a sequence encoding an expression product, suitably a gene, e.g. a transgene.

In a further aspect of the invention, there is provided an expression cassette comprising CNS-10 or a functional variant thereof, operably linked to a sequence encoding an expression product, suitably a gene, e.g. a transgene.

In another aspect of the invention, there is provided an expression cassette comprising CNS-11 or a functional variant thereof, operably linked to a sequence encoding an expression product, suitably a gene, e.g. a transgene.

In another aspect of the invention, there is provided an expression cassette comprising CNS-13 or a functional variant thereof, operably linked to a sequence encoding an expression product, suitably a gene, e.g. a transgene.

In some embodiments, the transgene is any one of the genes selected from the group consisting of NPC1, EAAT2, NPY, CYP46A1, GLB1, APOE (or APOE2), HEX, CLN1, CLN2, CLN3, CLN4, CLN5, CLN6, SUMF1, DCTN1, PRPH, SOD1, NEFH, GBA, IDUA, NAGLU, GUSB, ARSA, MANB, AADC, GDNF, NTN, ASP, MECP2, PTCHD1, GJB1, UBE3A, HEXA, FXN, MOG and SLC6A3. Suitably, in some embodiments, the expression product is a therapeutic expression product. In some embodiments, the expression product is a reporter gene such as, but not limited to, GFP, RFP, YFP or luciferase. In some embodiments, where the transgene is GDNF, the expression product is GDNF protein. In some particularly preferred embodiments, the transgene is codon optimized GDNF. In some embodiments, the transgene is CpG depleted GDNF. More preferably, in some embodiments, the transgene is CpG depleted and codon optimized GDNF.

In a further aspect, there is provided a vector comprising a synthetic CNS-specific promoter or an expression cassette according to the invention. In some embodiments, the vector is an expression vector. In some embodiments, the vector is a viral vector. In some embodiments, the vector is a gene therapy vector, suitably an AAV vector, an adenoviral vector, a retroviral vector or a lentiviral vector. In a preferred embodiment, the vector is an AAV vector.

In a further aspect, there is provided a virion (a viral particle) comprising a viral vector, according to the present invention.

In a further aspect, there is provided a pharmaceutical composition comprising a synthetic CNS-specific promoter, expression cassette, vector or virion according to the present invention.

In a further aspect, there is provided a synthetic CNS-specific promoter, expression cassette, vector, virion or pharmaceutic composition according to the present invention for use in therapy, i.e. the prevention or treatment of a medical condition or disease. Suitably, the condition or disease is associated with aberrant gene expression, optionally aberrant gene expression in the CNS. Suitably, the use is for gene therapy, preferably for use in treatment of a disease involving aberrant gene expression. In some embodiments the disease is a CNS-related disease. CNS-related diseases, include but are not limited to dopamine transporter deficiency syndrome (DTDS), Alzheimer's disease, Epilepsy, Parkinson's disease, Multiple sclerosis, Motor Neurone Disease and Huntington's disease. In a preferred embodiment, the disease effects dopaminergic neurons in the CNS.

Suitably, the gene therapy involves expression of a therapeutic expression product in the CNS.

In a further aspect, there is provided a cell comprising a synthetic CNS-specific promoter, expression cassette, vector or virion as described herein. In some embodiments, the cell is a eukaryotic cell, optionally a mammalian cell, optionally a human cell. In some embodiments, the cell is a CNS cell, optionally wherein the cell is human CNS cell. Suitably, in some embodiments the CNS cell is a neurone, astrocyte, oligodendrocyte, microglial cell or an ependymal cell. In a preferred embodiment, the CNS cell is a neurone, even more preferably a dopaminergic neurone. The synthetic CNS-promoter or expression cassette can be in a vector or can be in the genome of the cell.

In a further aspect, there is provided a synthetic CNS-specific promoter, expression cassette, vector or virion according to the present invention for use in the manufacture of a pharmaceutical composition for the treatment of a medical condition or disease as discussed herein. In some embodiments, the disease is a CNS-related disease.

In a further aspect, there is provided a method of expressing a therapeutic transgene in a cell, preferably a CNS cell, wherein the method comprises introducing into the cell a synthetic CNS-specific expression cassette, vector or virion as described herein. In some preferred embodiments the therapeutic transgene is GDNF.

In a further aspect, there is provided a method of therapy of a subject, preferably a human in need thereof, the method comprising: administering to the subject in need thereof, an expression cassette, vector, virion, or pharmaceutical composition as described herein, which comprises a sequence encoding a therapeutic product operably linked to a promoter according to the present invention; and expressing a therapeutic amount of the therapeutic product in the CNS of said subject.

In one embodiment, therapeutic product is GDNF protein.

In some embodiments, the expression cassette, vector, virion or pharmaceutical composition as described herein, is administered directly into the CNS of the subject.

In some embodiments, the method further comprises introducing into the CNS of the subject an expression cassette, vector, virion or pharmaceutical composition of the present invention which comprises a gene encoding a therapeutic product. In some embodiments, the vector is a viral gene therapy vector, preferably an AAV vector.

In a further aspect, there is provided a method for producing an expression product, the method comprising: introducing a synthetic CNS-specific expression cassette of the present invention into a CNS cell; and expressing the gene present in the synthetic CNS-specific expression cassette.

The method suitably comprises maintaining said CNS cell under suitable conditions for expression of the gene. In culture this may comprise incubating the cell, or tissue comprising the cell, under suitable culture conditions. The expression may of course be in vitro, ex vivo or in vivo, e.g. in one or more cells in the CNS of a subject.

Suitably the method comprises the step of introducing the synthetic CNS-specific expression cassette into the CNS cell. A wide range of methods of transfecting CNS cells are well-known in the art. A preferred method of transfecting CNS cells is transducing the cells with a viral vector comprising the synthetic CNS-specific expression cassette, e.g. an AAV vector.

### CREs and Functional Variants Thereof

Disclosed herein are CREs that can be used in construction of CNS-specific promoters. Suitably, the CREs are CNS-specific. These CREs are generally derived from genomic promoter and enhancer sequences, but they are used herein in contexts quite different from their native genomic environment. Generally, the CREs constitute small parts of much larger genomic regulatory domains, which control expression of the genes with which they are normally associated. It has been surprisingly found that these CREs, many of which are very small, can be isolated form their normal environment and retain CNS specific regulatory activity. This is surprising because the removal of a regulatory sequence from the complex and “three dimensional” natural context in the genome often results in a significant loss of activity, so there is no reason to expect a given CRE to retain the levels of activity observed once removed from their natural environment. It is even more surprising when a CRE retain CNS-specific activity in an AAV vector. This is particularly the case as an AAV vector comprises Inverted Terminal Repeat (ITR) and has a different DNA structure compared to the genome and both ITRs and the DNA structure are known to influence the activity of CREs. It should be noted that the sequences of the CREs of the present invention can be altered without causing a substantial loss of activity. Functional variants of the CREs can be prepared by modifying the sequence of the CREs, provided that modifications which are significantly detrimental to activity of the CRE are avoided. In view of the information provided in the present disclosure, modification of CREs to provide functional variants is straightforward. Moreover, the present disclosure provides methodologies for simply assessing the functionality of any given CRE variant (see e.g. Example 3).

The size of certain CREs according to the present invention is advantageous because it allows for the CREs, more specifically promoters containing them, to be provided in vectors while taking up the minimal amount of the payload of the vector. This is particularly important when a CRE is used in a vector with limited capacity, such as an AAV based vector.

CREs of the present invention comprise certain CNS-specific TFBS. It is generally desired that in functional variants of the CREs these CNS-specific TFBS remain functional. The skilled person is well aware that TFBS sequences can vary yet retain functionality. In view of this, the sequence for a TFBS is typically illustrated by a consensus sequence from which some degree of variation is typically present. Further information about the variation that occurs in a TFBS can be illustrated using a positional weight matrix (PWM), which represents the frequency with which a given nucleotide is typically found at a given location the consensus sequence. Details of TF consensus sequences and associated positional weight matrices can be found in, for example, the Jaspar or Transfac databases http://jaspar.genereg.net/ and http://gene-regulation.com/pub/databases.html). This information allows the skilled person to modify the sequence in any given TFBS of a CRE in a manner which retains, and in some cases even increases, CRE functionality. In view of this the skilled person has ample guidance on how the TFBS for any given TF can be modified, while maintaining ability to bind the desired TF; the Jaspar system will, for example, score a putative TFBS based on its similarity to a given PWM. Furthermore, CREs can be scanned against all PWM from JASPAR database to identify/analyse all TFBS. The skilled person can of course find additional guidance in the literature, and, moreover, routine experimentation can be used to confirm TF binding to a putative TFBS in any variant CRE. It will be apparent that significant sequence modification in a CRE, even within TFBS in a CRE, can be made while retaining function. CREs of the present invention can be used in combination with a wide range of suitable minimal promoters or CNS-specific proximal promoters. Functional variants of a CRE include sequences which vary from the reference CRE element, but which substantially retain activity as CNS-specific CREs. It will be appreciated by the skilled person that it is possible to vary the sequence of a CRE while retaining its ability to recruit suitable CNS-specific transcription factors (TFs) and thereby enhance expression. A functional variant of a CRE can comprise substitutions, deletions and/or insertions compared to a reference CRE, provided they do not render the CRE substantially non-functional.

In some embodiments, a functional variant of a CRE can be viewed as a CRE which, when substituted in place of a reference CRE in a promoter, substantially retains its activity. For example, a CNS-specific promoter which comprises a functional variant of a given CRE preferably retains at least 80% of its activity, more preferably at least 90% of its activity, more preferably at least 95% of its activity, and yet more preferably 100% of its activity (compared to the reference promoter comprising the unmodified CRE). Suitably, functional variants of a CRE retain a significant level of sequence identity to a reference CRE. Suitably functional variants comprise a sequence that is at least 70% identical to the reference CRE, more preferably at least 80%, 90%, 91%, 92%, 93%, 94%, 95%, 96%, 97%, 98% or 99% identical to the reference CRE.

### Promoter Elements and Functional Variants Thereof

The one or more promoter elements as disclosed herein may be minimal promoters or proximal promoters. Suitably, in some embodiments, the minimal promoters or proximal promoters may be non-tissue specific (or ubiquitous) minimal or proximal promoters. In another embodiment, the minimal promoters or proximal promoters may be a CNS-specific minimal promoter or a CNS-specific proximal promoter. While CREs of the present invention can be used in combination with a range of suitable minimal promoters or CNS-specific proximal promoters, some promoter elements act synergistically with the one or more CREs to contribute significantly to the activity of the CNS-specific promoter.

Functional variants of a promoter element include sequences which vary from the reference promoter element, but substantially retain their activity as promoter elements. The skilled person will understand that it is possible to vary the sequence of a promoter element while retaining the ability to recruit RNA polymerase II, and where relevant bind CNS-specific TFs to enhance expression. A functional variant of a promoter element can comprise substitutions, deletions and/or insertions compared to a reference promoter element, provided said substitutions, deletions and/or insertions do not render the promoter element non-functional.

In some embodiments, a functional variant of CRE151 can be viewed as a promoter element which, when substituted in place of the reference promoter element in a promoter, substantially retains its activity. Suitable assays for assaying CNS-specific promoter activity are disclosed herein, e.g. in Example 3.

Suitably, functional variants of a promoter element retain a significant level of sequence identity to a reference promoter element. Suitably, functional elements of CRE151 comprise a sequence that is at least 70% identical to SEQ ID NO: 190, more preferably at least 80%, 90%, 91%, 92%, 93%, 94%, 95%, 96%, 97%, 98% or 99% identical to SEQ ID NO: 190. As discussed above, functional variants of CRE151 substantially retain the ability of the reference promoter element to act as a promoter element. For example, when a functional variant of CRE151 is substituted into a promoter comprising the reference promoter element, the modified promoter retains at least 80% of its activity, more preferably at least 90% of its activity, more preferably at least 95% of its activity, and yet more preferably 100% of its activity. Suitably, the functional variant of CRE151 comprises a sequence which has at least 70%, 80%, 90%, 95%, 96%, 97%, 98% or 99% identity to SEQ ID NO: 190.

In some embodiments, a functional variant of CRE527 can be viewed as a promoter element which, when substituted in place of the reference promoter element in a promoter, substantially retains its activity. Suitable assays for assaying CNS-specific promoter activity are disclosed herein, e.g. in Example 1.

Suitably, functional variants of a promoter element retain a significant level of sequence identity to a reference promoter element. Suitably, functional elements of CRE527 comprise a sequence that is at least 70% identical to SEQ ID NO: 211, more preferably at least 80%, 90%, 91%, 92%, 93%, 94%, 95%, 96%, 97%, 98% or 99% identical to SEQ ID NO: 211. As discussed above, functional variants of CRE527 substantially retain the ability of the reference promoter element to act as a promoter element. For example, when a functional variant of CRE527 is substituted into a promoter comprising the reference promoter element, the modified promoter retains at least 80% of its activity, more preferably at least 90% of its activity, more preferably at least 95% of its activity, and yet more preferably 100% of its activity. Suitably, the functional variant of CRE527 comprises a sequence which has at least 70%, 80%, 90%, 95%, 96%, 97%, 98% or 99% identity to SEQ ID NO: 211.

Retention of activity can be assessed by comparing expression of a suitable reporter under the control of the reference promoter with an otherwise identical promoter comprising the substituted promoter element under equivalent conditions. Suitable assays for assessing CNS-specific promoter activity are disclosed herein, e.g. in Example 3.

Promoter elements used in the present invention can be natural (i.e. obtained or derived from a naturally occurring gene promoter) or can be synthetic (i.e. non-naturally occurring).

### Synthetic CNS-Specific Promoters and Functional Variants Thereof

A functional variant of a reference synthetic CNS-specific promoter is a promoter which comprises a sequence which varies from the reference synthetic CNS-specific promoter, but which substantially retains CNS-specific promoter activity. It will be appreciated by the skilled person that it is possible to vary the sequence of a synthetic CNS-specific promoter while retaining its ability to recruit suitable CNS-specific transcription factors (TFs) and to recruit RNA polymerase II to provide CNS-specific expression of an operably linked sequence (e.g. open reading frame). A functional variant of a synthetic CNS-specific promoter can comprise substitutions, deletions and/or insertions compared to a reference promoter, provided such substitutions, deletions and/or insertions do not render the synthetic CNS-specific promoter substantially non-functional compared to the reference promoter (e.g. CNS-10, CNS-11 or CNS-13).

Accordingly, in some embodiments, a functional variant of a promoter according to the present invention can be viewed as a variant which substantially retains the CNS-specific promoter activity of the reference promoter. For example, a functional variant of a promoter according to the present invention preferably retains at least 70% of the activity of the reference promoter, more preferably at least 80% of its activity, more preferably at least 90% at least 91%, at least 92%, at least 93%, at least 94%, at least 95%, at least 96%, at least 97%, at least 98%, at least 99% of its activity, more preferably 100% of its activity.

In some embodiments, a functional variant of CNS-10 can be viewed as a variant which substantially retains the CNS-specific promoter activity of the reference promoter. For example, a functional variant of CNS-10 preferably retains at least 70% of the activity of the reference promoter, more preferably at least 80% of its activity, more preferably at least 90% at least 91%, at least 92%, at least 93%, at least 94%, at least 95%, at least 96%, at least 97%, at least 98%, at least 99% of its activity, more preferably 100% of its activity.

In some embodiments, a functional variant of CNS-11 can be viewed as a variant which substantially retains the CNS-specific promoter activity of the reference promoter. For example, a functional variant of CNS-11 preferably retains at least 70% of the activity of the reference promoter, more preferably at least 80% of its activity, more preferably at least 90% of its activity, more preferably at least 95% of its activity, and yet more preferably 100% of its activity.

In some embodiments, a functional variant of CNS-13 can be viewed as a variant which substantially retains the CNS-specific promoter activity of the reference promoter. For example, a functional variant of CNS-13 preferably retains at least 70% of the activity of the reference promoter, more preferably at least 80% of its activity, more preferably at least 90% of its activity, more preferably at least 95% of its activity, and yet more preferably 100% of its activity.

Functional variants of a synthetic CNS-specific promoter often retain a significant level of sequence similarity to a reference synthetic CNS-specific promoter.

Accordingly, in some embodiments, a functional variant of a synthetic CNS-specific promoter according to the present invention can be viewed as a variant which substantially retains the CNS-specific promoter activity of the reference promoter. For example, a functional variant of a synthetic CNS-specific promoter according to the present invention preferably retains at least 70% of the activity of the reference promoter, more preferably at least 80% of its activity, more preferably at least 90% at least 91%, at least 92%, at least 93%, at least 94%, at least 95%, at least 96%, at least 97%, at least 98%, at least 99% of its activity, more preferably 100% of its activity.

Suitably, in one embodiment, functional variants of any one of CNS-10, CNS-11 or CNS-13 are at least 60%, 65%, 70%, 75%, 80%, 85%, 90%, 91%, 92%, 93%, 94%, 95%, 96%, 97%, 98% or 99% identical to any one of SEQ ID NOs: 187, 188 or 189.

In some embodiments, functional variants of CNS-10 comprise a sequence that is at least 70% identical to the reference synthetic CNS-specific promoter, more preferably at least 80%, 90%, 91%, 92%, 93%, 94%, 95%, 96%, 97%, 98% or 99% identical to the reference synthetic CNS-specific promoter. Suitably, functional variants of CNS-10 comprise a sequence that is at least 70% identical to SEQ ID NO: 187, more preferably at least 80%, 90%, 95%, 96%, 97%, 98% or 99% identical to SEQ ID NO: 187.

In some embodiments, functional variants of CNS-11 comprise a sequence that is at least 70% identical to the reference synthetic CNS-specific promoter, more preferably at least 80%, 90%, 91%, 92%, 93%, 94%, 95%, 96%, 97%, 98% or 99% identical to the reference synthetic CNS-specific promoter. Suitably, functional variants of CNS-11 comprise a sequence that is at least 70% identical to SEQ ID NO: 188, more preferably at least 80%, 90%, 95%, 96%, 97%, 98% or 99% identical to SEQ ID NO: 188.

In some embodiments, functional variants of CNS-13 comprise a sequence that is at least 70% identical to the reference synthetic CNS-specific promoter, more preferably at least 80%, 90%, 91%, 92%, 93%, 94%, 95%, 96%, 97%, 98% or 99% identical to the reference synthetic CNS-specific promoter. Suitably, functional variants of CNS-11 comprise a sequence that is at least 70% identical to SEQ ID NO: 189 more preferably at least 80%, 90%, 91%, 92%, 93%, 94%, 95%, 96%, 97%, 98% or 99% identical to SEQ ID NO: 189.

Activity in a functional variant can be assessed by comparing expression of a suitable reporter under the control of the reference synthetic CNS-specific promoter with the putative functional variant under equivalent conditions. Suitable assays for assessing CNS-specific promoter activity are disclosed herein, e.g. in Example 3.

Functional variants of a given synthetic CNS-specific promoter can comprise functional variants of one or more CREs and/or functional variants of the promoter element present in the reference synthetic CNS-specific promoter. Functional variants of CNS-10 can comprise functional variants of CRE00010 and/or functional variants of CRE151. Functional variants of CNS-11 can comprise functional variants of CRE00011 and/or functional variants of CRE151. Functional variants of CNS-13 can comprise functional variants of CRE00010 and/or functional variants of CRE527.

Functional variants of a given synthetic CNS-specific promoter can comprise one or more additional CREs to those present in a reference synthetic CNS-specific promoter. The additional CREs can be CREs disclosed herein, or they can be other CREs.

Functional variants of a given synthetic CNS-specific promoter can comprise one or more additional regulatory elements compared to a reference synthetic CNS-specific promoter. For example, they may comprise an inducible element, an intronic element, a boundary control element, an insulator, a locus control region, a response element, a binding site, a segment of a terminal repeat, a responsive site, a stabilizing element, a de-stabilizing element, and a splicing element, etc., provided that they do not render the promoter substantially non-functional. Functional variants can also include a 5′ UTR sequence.

Functional variants of a given synthetic CNS-specific promoter can comprise additional spacers between adjacent CREs and/or between CRE and promoter element.

Preferred synthetic CNS-specific promoters of the present invention exhibit CNS-specific promoter activity which is at least 15%, 20%, 25%, 30%, 40%, 50%, 60%, 70%, 80%, 90%, 100%, 125%, 150%, 175%, 200%, 300% or 400% of the activity of Synapsin-1, Camk2a, GFAP, MBP, IBA1 or NSE promoters in CNS cells. In some embodiments, the synthetic CNS-specific promoters of the present invention exhibit CNS-specific promoter activity which is at least 15%, 20% 25%, 30%, 40%, 50%, 60%, 70%, 80%, 90%, 100%, 125%, 150%, 175% or 200% of the activity of the human TH promoter. In some embodiments, the synthetic CNS-specific promoters of the invention are suitable for promoting CNS-specific transgene expression at a level at least 100% of the activity of Synapsin-1, Camk2a, GFAP, MBP, IBA1 or NSE promoters in CNS cells, preferably 150%, 200%, 300% or 500% of the activity of any one Synapsin-1, Camk2a, GFAP, MBP, IBA1 or NSE promoters.

In some embodiments, the synthetic CNS-specific promoters of the invention are suitable for promoting CNS-specific transgene expression at a level at least 100% of the activity of the human TH promoter, preferably 150% or 200% of the activity of the human TH promoter. In many cases higher levels of promoter activity is preferred, but this is not always the case; thus, in some cases more moderate levels of expression may be preferred. In some cases, more moderate levels of expression may be preferred, e.g. to prevent toxicity or accumulation of protein. Activity of a given synthetic CNS-specific promoter of the present invention compared to a reference promoter (e.g. a control promoter) can be assessed by comparing CNS-specific expression of a reporter gene under control of the synthetic CNS-specific promoter with expression of the same reporter under control of the reference promoter, when the two promoters are provided in otherwise equivalent expression constructs and under equivalent conditions.

In some embodiments a synthetic CNS-specific promoter of the invention is able to increase expression of a gene (e.g. a therapeutic gene or gene of interest) in the CNS of a subject or in a CNS cell by at least 20%, at least 40%, at least 60%, at least 80%, at least 100%, at least 200%, at least 300%, at least 500%, at least 1000% or more relative to a known CNS-specific promoter, suitably, Synapsin-1, Camk2a, GFAP, MBP, IBA1 or NSE. In another embodiment, a synthetic CNS-specific promoter of the invention is able to increase expression of a gene (e.g. a therapeutic gene or gene of interest) in neurons, suitably dopaminergic neurons, of a subject by at least 20%, at least 40%, at least 60%, at least 80%, at least 100%, at least 200%, at least 300%, or more relative to the human TH promoter. In a preferred embodiment, a synthetic CNS-specific promoter of the present invention is able to increase the expression of a gene, preferably a transgene, in neurons, optionally dopaminergic neurons.

Preferred synthetic CNS-specific promoters of the present invention exhibit activity in non-CNS cells (e.g. HEK293 cells) which is 50% or less when compared to CMV-IE, preferably 25% or less than CMV-IE, more preferably 10% or less than CMV-IE, and in some cases 5% or less than CMV-IE, or 1% or less than CMV-IE. In some embodiments, a promoter is CNS-specific if it is more active in the CNS than in non-CNS cells or tissues. The promoters of the present invention may be active in all types of CNS cells (neurons, astrocytes, oligodendrocytes, microglial cells and ependymal cells). In some embodiments, the synthetic CNS-specific promoters of the present invention are more active in neurons than in other cells of the CNS (e.g. microglia, astrocytes, ependymal and oligodendrocytes). In a preferred embodiment, the synthetic CNS-specific promoter is more active in dopaminergic neurons than in other cells of the CNS (e.g. microglia, astrocytes, ependymal and oligodendrocytes.

The skilled person will understand that the activity of a synthetic CNS-specific promoter of the present invention may vary depending on the route of administration to a subject. Suitably, in some embodiments, the synthetic CNS-specific promoter is more active in dopaminergic neurons than in other cells of the CNS when delivered intracerebroventricularly (ICV). Alternatively, in some embodiments, the synthetic CNS-specific promoter is more active in dopaminergic neurons than in other cells of the CNS when delivered intravenously (IV). Suitably, in one embodiment, a CNS-specific promoter comprising or consisting of a sequence according to any one of SEQ ID NOs: 187 or a functional variant thereof is active in dopaminergic neurons when administered by either ICV or IV delivery. In one embodiment, a CNS-specific promoter comprising or consisting of a sequence according to any one of SEQ ID NOs: 188 or a functional variant thereof is active in dopaminergic neurons when administered by either ICV or IV delivery. Alternatively, in one embodiment, a CNS-specific promoter comprising or consisting of a sequence according to any one of SEQ ID NOs: 189 or a functional variant thereof is active in dopaminergic neurons when administered by ICV delivery.

The synthetic CNS-specific promoters of the present invention may be active in specific areas of the CNS, in specific CNS cells or CNS cell subtypes or both. In some embodiments, the synthetic CNS-specific promoters of the present invention may be active in a specific CNS cell type, such as neurons, within all areas of the CNS. In other embodiments, the synthetic CNS-specific promoters of the present invention may be active in a specific CNS cell type, such as neurons, within no more than one area of the CNS, such as midbrain. In some embodiments, the synthetic CNS-specific promoters of the present invention may be active in all CNS cells in all areas of the CNS. In some embodiments, the synthetic CNS-specific promoters of the present invention may be active in no more than one area of the CNS, such as the midbrain. Suitably, in some embodiments, the synthetic CNS-specific promoters of the present invention may be active only in dopaminergic neurons in the midbrain.

It is expected that CNS-10 and CNS-13 are particularly active in dopaminergic neurons. It is expected that CNS-11 is particularly active in the medial entorhinal cortex. This has been demonstrated in preliminary in vivo experiments whereby predominantly neuronal tropism was observed with CNS-10, CNS-11 and CNS-13. In particular, CNS-10 and CNS-13 showed mixed neuronal and minimal astroglial expression.

Aspects of the disclosure relate to an isolated nucleic acid comprising more than one promoter (e.g., 2, 3, 4, 5, or more promoters). For example, in the context of a construct having a transgene comprising a first region encoding a protein and an second region encoding an inhibitory RNA (e.g., miRNA), it may be desirable to drive expression of the protein coding region using a first promoter sequence (e.g., a first promoter sequence operably linked to the protein coding region), and to drive expression of the inhibitory RNA encoding region with a second promoter sequence (e.g., a second promoter sequence operably linked to the inhibitory RNA encoding region). Generally, the first promoter sequence and the second promoter sequence can be the same promoter sequence or different promoter sequences. In some embodiments, the first promoter sequence (e.g., the promoter driving expression of the protein coding region) is a RNA polymerase III (polIII) promoter sequence. Non-limiting examples of polIII promoter sequences include U6 and HI promoter sequences. In some embodiments, the second promoter sequence (e.g., the promoter sequence driving expression of the inhibitory RNA) is a RNA polymerase II (polII) promoter sequence. Non-limiting examples of polII promoter sequences include T7, T3, SP6, RSV, and cytomegalovirus promoter sequences. In some embodiments, a polIII promoter sequence drives expression of an inhibitory RNA (e.g., miRNA) encoding region. In some embodiments, a polII promoter sequence drives expression of a protein coding region.

In some embodiments, the nucleic acid comprises a transgene that encodes a protein. The protein can be a therapeutic protein (e.g., a peptide, protein, or polypeptide useful for the treatment or prevention of disease states in a mammalian subject) or a reporter protein. In some embodiments, the protein is CYP46A1. In some embodiments, the protein is human CYP46A1. In some embodiments, the protein encodes SEQ ID NO; 2 or a protein comprising SEQ ID NO: 2. In some embodiments, the protein encodes a protein with a sequence identity of at least 80%, at least 85%, at least 90%, at least 95%, at least 98% to SEQ ID NO: 2. In some embodiments, the therapeutic protein is useful for treatment or prevention of Huntington's disease, for example Polyglutamine binding peptide 1 (QBP1), PTD-QBP1, ED 11, C4 intrabody, VL12.3 intrabody, MW7 intrabody, Happ1 antibodies, Happ3 antibodies, mEM48 intrabody, certain monoclonal antibodies (e.g., 1C2), and peptide P42 and variants thereof, as described in Marelli et al. (2016) Orphanet Journal of Rare Disease 11:24; doi: 10.1186/s 13023-016-0405-3. In some embodiments, the therapeutic protein is wild-type huntingtin protein (e.g., huntingtin protein having a PolyQ repeat region comprising less than 36 repeats).

### CYP46A1

Cholesterol 24-hydroxylase is a neuronal enzyme that is coded by the CYP46A1 gene. It converts cholesterol into 24-hydroxycholesterol and has a critical role in the efflux of cholesterol from the brain (Dietschy, J. M. et al., 2004). Brain cholesterol is essentially produced—but cannot be degraded-in situ, and intact blood-brain barrier restricts direct transportation of cholesterol from the brain (Dietschy, J. M. et al., 2004). 24-hydroxycholesterol is able to cross the plasma membrane and the blood-brain barrier and reaches the liver where it is degraded.

CYP46A1 is neuroprotective in a cellular model of HD (see, e.g., WO2012/049314). Moreover, there is a reduction of CYP46A1 mRNAs in the striatum, the more vulnerable brain structure in the disease, of the R6/2 transgenic HD mouse model.

During the early stages of AD, 24-hydroxycholesterol concentrations are high in CSF and in peripheral circulation. In later stages of AD, concentrations of 24-hydroxycholesterol may fall likely reflecting neuronal loss (Kolsch, H. et al., 2004). CYP46A1 is expressed around the amyloid core of the neuritic plaques in the brain of AD patients (Brown, J., 3rd et al., 2004).

Agonism of cholesterol 24-hydroxylase, encoded by CYP46A1, provided marked decrease of neuropathology and an improvement of cognitive deficits in mouse models of CNS disease. For example, co-expression of CYP46A1 with ExpHtt in a Huntington's disease model promoted a strong and significant decrease of ExpHtt aggregates formation (58% versus 27.5%)) (WO2012/049314). (see also, International Patent Publication WO2009/034127; which is incorporated by reference herein in its entirety). The methods described herein relate to agonism of CYP46A1 in combination with the administration of miRNAs targeting certain other targets. For example, the methods can relate to administration of a viral vector for the treatment of a neurological disease or disorder, wherein the vector expresses CYP46A1 in cells of the central nervous system.

In some embodiments, described herein is a viral vector for treating a neurological disease or disorder, which vector comprises a cholesterol 24-hydroxylase encoding nucleic acid. In some embodiments, the viral vector comprises a nucleic acid sequence that encodes the amino acid sequence SEQ ID NO:2. In some embodiments, the viral vector comprises a nucleic acid sequence that encodes an amino acid sequence with at least 80%, at least 85%, at least 90%, at least 95%, at least 98%, or more sequence identity to SEQ ID NO:2. In some embodiments, the viral vector comprises a sequence with at least 80%, at least 85%, at least 90%, at least 95%, at least 98%, or more sequence identity to SEQ ID NO: 1. In some embodiments, the viral vector comprises the sequence of SEQ ID NO: 1. In some embodiments, the viral vector may be an Adeno-Associated-Virus (AAV) vector.

Further description of CY46A1 and its therapeutic uses (e.g., for Alzheimer's disease, ALS, and ataxia) are described in the art, e.g., in WO 2012/049314, WO 2009/034127, WO 2018/138371, and WO2020/089154. The sequences, methods, and compositions described therein can be utilized in the methods and compositions described herein. The foregoing references are incorporated by reference herein in their entireties. The term “gene” refers to a polynucleotide containing at least one open reading frame that is capable of encoding a particular polypeptide or protein after being transcribed or translated.

The terms “coding sequence” or “a sequence which encodes a particular protein”, denotes a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences.

A cDNA sequence for CYP46A1 is disclosed in Genbank Access Number NM_006668 (SEQ ID NO: 1). The amino acid sequence is shown in SEQ ID NO:2. The invention makes use of a nucleic acid construct comprising sequence SEQ ID NO:1 or a variant thereof for the treatment of a neurological disease or disorder. The variants include, for instance, naturally-occurring variants due to allelic variations between individuals (e.g., polymorphisms), alternative splicing forms, etc. The term variant also includes CYP46A1 gene sequences from other sources or organisms. Variants are preferably substantially homologous to SEQ ID NO:1 and/or 2, i.e., exhibit a nucleotide sequence identity of typically at least about 75%, preferably at least about 85%, more preferably at least about 90%, more preferably at least about 95% with SEQ ID NO:1 or 2. In some embodiments, the nucleic acid construct comprises a sequence with at least 95% sequence identity to SEQ ID NO: 1 and which retains the activity of SEQ ID NO: 1 or 2 (e.g., the ability to convert cholesterol into 24-hydroxycholesterol). Variants of a CYP46A1 gene also include nucleic acid sequences, which hybridize to a sequence as defined above (or a complementary strand thereof) under stringent hybridization conditions. Typical stringent hybridization conditions include temperatures above 30° C., preferably above 35° C., more preferably in excess of 42° C., and/or salinity of less than about 500 mM, preferably less than 200 mM. Hybridization conditions may be adjusted by the skilled person by modifying the temperature, salinity and/or the concentration of other reagents such as SDS, SSC, etc.

In one embodiment, the CYP46A1 is a codon optimized CYP46A1. An exemplary codon-optimized CYP46A1 contemplated for use herein is provided in SEQ ID NOs: 109 and 110. In some embodiments, the viral vector comprises a nucleic acid sequence that encodes the amino acid sequence SEQ ID NO: 109. In some embodiments, the viral vector comprises a nucleic acid sequence that encodes an amino acid sequence with at least 80%, at least 85%, at least 90%, at least 95%, at least 98%, or more sequence identity to SEQ ID NO: 109. In some embodiments, the viral vector comprises the nucleic acid sequence of SEQ ID NO:110. In some embodiments, the viral vector comprises a nucleic acid sequence with at least 80%, at least 85%, at least 90%, at least 95%, at least 98%, or more sequence identity to the sequence of SEQ ID NO: 110.

In one embodiment, the CYP46A1 nucleic acid sequence is codon-optimized, i.e., a codon-optimized variant. In one embodiment, the wild-type CYP46A1 nucleic acid sequence (e.g., SEQ ID NO: 1) is codon-optimized, i.e., a codon-optimized variant. Exemplary methods for codon optimization are known in the art are described herein below. In one embodiment, more than more method for codon-optimization is utilized. For example, the CYP46A1 nucleic acid sequence is modified to have reduced CpG motifs and increased GC content.

For example, at least one or more CpG islands (a cytosine base followed immediately by a guanine base (a CpG) in which the cytosines in such arrangement tend to be methylated) that typically occur at, or near the transcription start site in the CYP46A1 nucleic acid sequence are deleted and/or substituted. In one embodiment, deletion or reduction in the number of CpG islands can enhance expression in vivo or reduce the innate immune response of the transgene. In one embodiment, at least 1 CpG motif is deleted and/or substituted, e.g., at least 1, 2, 3, 4, 5, or more CpG motifs are deleted and/or substituted. In one embodiment, the wild-type CYP46A1 nucleic acid sequence is optimized to comprise 0% CpGs. In one embodiment, the wild-type CYP46A1 nucleic acid sequence is optimized to comprise no more than 10% CpGs, or no more than 20% CpGs, or no more than 30% CpGs, or no more than 40% CpGs, or no more than 50% CpGs, or no more than 60% CpGs, or no more than 70% CpGs, or no more than 80% CpGs, or no more than 90% CpGs present in the wild type CYP46A1 nucleic acid sequence. Exemplary modifications to the wild-type CYP46A1 nucleic acid sequence (SEQ ID NO: 1) that result in the removal of CpG islands are described in Table 23 below.

Alternatively, codon-optimization can be achieved by editing the nucleic acid to remove alternative reading frames. In one embodiment, deletion of alternative reading frames can enhance expression in vivo or reduce the innate immune response of the transgene. In one embodiment, at least 1 alternative reading frame is deleted, e.g., at least 2, at least 3, or more alternative reading frames. Exemplary modifications to the wild-type CYP46A1 nucleic acid sequence (SEQ ID NO: 1) that result in the removal of an alternative reading frame are described in Table 23 below.

In various embodiments, the CYP46A1 nucleic acid sequence is codon-optimized via increasing the total GC content, i.e., the percent of guanines and cytosines in the entire coding sequence; removing cryptic splice donor or acceptor sites; and/or adding or removing ribosomal entry and/or initiation sites, such as Kozak sequences.

In one embodiment, the wild-type CYP46A1 nucleic acid (e.g., SEQ ID NO: 1) is codon-optimized by including at least one modification provided in Table 23 below. The “new elements” described in Table 23 are meant to be exemplary, and not limiting. For example, T222 can be replaced with “A” as described in Table 23. However, T22 can also be replaced with “G,” or “C,” and each modification can result in removal of an alternative reading frame. In one embodiment, the wild-type CYP46A1 nucleic acid (e.g., SEQ ID NO: 1) is codon-optimized by including at least two or more modifications provided in Table 23 below.

TABLE 23
Modifications for codon-optimizing wild-type CYP461

| NT# based on | | | | |
|---|---|---|---|---|
| SEQ ID NO: 1 | element | action | new element | impact |
| 27 | C | replaced | T | remove CpG |
| 193-195 | TTG | replaced | CTA | remove alternative reading frame |
| 222 | T | replaced | A | remove alternative reading frame |
| 420 | C | replaced | G | remove alternative reading frame |
| 477 | G | replaced | A | remove CpG |
| 531-536 | AGTGTC | replaced | TGTGAG | remove alternative reading frame |
| 1002-1005 | GGTC | replaced | AGTG | remove alternative reading frame |
| 1114-1116 | CGC | replaced | AGG | remove alternative reading frame |
| 1149 | C | replaced | G | remove alternative reading frame |
| 1191 | C | replaced | G | remove alternative reading frame |

In one embodiment, the wild-type CYP46A1 nucleic acid (e.g., SEQ ID NO: 1) is codon-optimized by modifying the untranslated regions of the wild-type CYP46A1 nucleic acid.

In one embodiment, the CYP46A1 protein is encoded by a codon optimized CYP46A1 nucleic acid sequence, for example, for any one or more of: (1) enhanced expression in vivo, (2) to reduce CpG islands or (3) reduce the innate immune response.

In some embodiments, the codon-optimized CYP46A1 variant exhibits enhanced long term expression relative to the wild type CYP46A1, such as for a period of at least 1 month, at least 2 months, at least 3 months, at least 4 months, at least 5 months, at least 6 months, at least 7 months, at least 8 months, at least 9 months, at least 10 months, at least 11 months, at least 1 year, at least 2 years, at least 3 years, at least 4 years, or at least 5 years.

In some embodiments, the codon-optimized CYP46A1 variant exhibits a reduced immune response relative to the wild type CYP46A1.

In some aspects, the rAAV encoding CYP46A1 and/or CYP46A1 variants is produced using closed linear DNA molecules that lack bacterial backbone sequences.

In one aspect, provided herein is a composition comprising an isolated nucleic acid comprising a sequence at least 80% identical, e.g., at least 85%, at least 90%, at least 95%, at least 98%, at least 99% or 100% identical, to SEQ ID NO: 110. In one aspect, provided herein is a composition comprising a recombinant viral vector comprising an isolated nucleic acid comprising a sequence at least 80% identical, e.g., at least 85%, at least 90%, at least 95%, at least 98%, at least 99% or 100% identical, to SEQ ID NO: 110. In some embodiments, an isolated nucleic acid encoding a CYP46A1 protein comprises at least 1, at least 2, at least 3, at least 4, at least 5, at least 6, at least 7, at least 8, at least 9, at least 10, at least 11, at least 12, at least 13, at least 14, or at least 15 of the mutations of SEQ ID NO: 110 as compared to SEQ ID NO: 1. The foregoing compositions can be used, e.g., in the absence of an administered miRNA to treat a neurological disease or disorder as described herein.

| SEQ ID NO: 1 | |
|---|---|
| CYP46A1 mRNA | |
| atg agc ccc ggg ctg ctg ctg ctc ggc agc gcc gtc ctg ctc gcc ttc | 48 |
| Met Ser Pro Gly Leu Leu Leu Leu Gly Ser Ala Val Leu Leu Ala Phe | |
| 1 5 10 15 | |
| ggc ctc tgc tgc acc ttc gtg cac cgc gct cgc agc cgc tac gag cac | 96 |
| Gly Leu Cys Cys Thr Phe Val His Arg Ala Arg Ser Arg Tyr Glu His | |
| 20 25 30 | |
| atc ccc ggg ccg ccg cgg ccc agt ttc ctt cta gga cac ctc ccc tgc | 144 |
| Ile Pro Gly Pro Pro Arg Pro Ser Phe Leu Leu Gly His Leu Pro Cys | |
| 35 40 45 | |
| ttt tgg aaa aag gat gag gtt ggt ggc cgt gtg ctc caa gat gtg ttt | 192 |
| Phe Trp Lys Lys Asp Glu Val Gly Gly Arg Val Leu Gln Asp Val Phe | |
| 50 55 60 | |
| ttg gat tgg gct aag aag tat gga cct gtt gtg cgg gtc aac gtc ttc | 240 |
| Leu Asp Trp Ala Lys Lys Tyr Gly Pro Val Val Arg Val Asn Val Phe | |
| 65 70 75 80 | |
| cac aaa acc tca gtc atc gtc acg agt cct gag tcg gtt aag aag ttc | 288 |
| His Lys Thr Ser Val Ile Val Thr Ser Pro Glu Ser Val Lys Lys Phe | |
| 85 90 95 | |
| ctg atg tca acc aag tac aac aag gac tcc aag atg tac cgt gcg ctc | 336 |
| Leu Met Ser Thr Lys Tyr Asn Lys Asp Ser Lys Met Tyr Arg Ala Leu | |
| 100 105 110 | |
| cag act gtg ttt ggt gag aga ctc ttc ggc caa ggc ttg gtg tcc gaa | 384 |
| Gln Thr Val Phe Gly Glu Arg Leu Phe Gly Gln Gly Leu Val Ser Glu | |
| 115 120 125 | |
| tgc aac tat gag cgc tgg cac aag cag cgg aga gtc ata gac ctg gcc | 432 |
| Cys Asn Tyr Glu Arg Trp His Lys Gln Arg Arg Val Ile Asp Leu Ala | |
| 130 135 140 | |
| ttc agc cgg agc tcc ttg gtt agc tta atg gaa aca ttc aac gag aag | 480 |
| Phe Ser Arg Ser Ser Leu Val Ser Leu Met Glu Thr Phe Asn Glu Lys | |
| 145 150 155 160 | |
| gct gag cag ctg gtg gag att cta gaa gcc aag gca gat ggg cag acc | 528 |
| Ala Glu Gln Leu Val Glu Ile Leu Glu Ala Lys Ala Asp Gly Gln Thr | |
| 165 170 175 | |
| cca gtg tcc atg cag gac atg ctg acc tac acc gcc atg gac atc ctg | 576 |
| Pro Val Ser Met Gln Asp Met Leu Thr Tyr Thr Ala Met Asp Ile Leu | |
| 180 185 190 | |
| gcc aag gca gct ttt ggg atg gag acc agt atg ctg ctg ggt gcc cag | 624 |
| Ala Lys Ala Ala Phe Gly Met Glu Thr Ser Met Leu Leu Gly Ala Gln | |
| 195 200 205 | |
| aag cct ctg tcc cag gca gtg aaa ctt atg ttg gag gga atc act gcg | 672 |
| Lys Pro Leu Ser Gln Ala Val Lys Leu Met Leu Glu Gly Ile Thr Ala | |
| 210 215 220 | |
| tcc cgc aac act ctg gca aag ttc ctg cca ggg aag agg aag cag ctc | 720 |
| Ser Arg Asn Thr Leu Ala Lys Phe Leu Pro Gly Lys Arg Lys Gln Leu | |
| 225 230 235 240 | |
| cgg gag gtc cgg gag agc att cgc ttc ctg cgc cag gtg ggc agg gac | 768 |
| Arg Glu Val Arg Glu Ser Ile Arg Phe Leu Arg Gln Val Gly Arg Asp | |
| 245 250 255 | |
| tgg gtc cag cgc cgc cgg gaa gcc ctg aag agg ggc gag gag gtt cct | 816 |
| Trp Val Gln Arg Arg Arg Glu Ala Leu Lys Arg Gly Glu Glu Val Pro | |
| 260 265 270 | |
| gcc gac atc ctc aca cag att ctg aaa gct gaa gag gga gcc cag gac | 864 |
| Ala Asp Ile Leu Thr Gln Ile Leu Lys Ala Glu Glu Gly Ala Gln Asp | |
| 275 280 285 | |
| gac gag ggt ctg ctg gac aac ttc gtc acc ttc ttc att gct ggt cac | 912 |
| Asp Glu Gly Leu Leu Asp Asn Phe Val Thr Phe Phe Ile Ala Gly His | |
| 290 295 300 | |
| gag acc tct gcc aac cac ttg gcg ttc aca gtg atg gag ctg tct cgc | 960 |
| Glu Thr Ser Ala Asn His Leu Ala Phe Thr Val Met Glu Leu Ser Arg | |
| 305 310 315 320 | |
| cag cca gag atc gtg gca agg ctg cag gcc gag gtg gat gag gtc att | 1008 |
| Gln Pro Glu Ile Val Ala Arg Leu Gln Ala Glu Val Asp Glu Val Ile | |
| 325 330 335 | |
| ggt tct aag agg tac ctg gat ttc gag gac ctg ggg aga ctg cag tac | 1056 |
| Gly Ser Lys Arg Tyr Leu Asp Phe Glu Asp Leu Gly Arg Leu Gln Tyr | |
| 340 345 350 | |
| ctg tcc cag gtc ctc aaa gag tcg ctg agg ctg tac cca cca gca tgg | 1104 |
| Leu Ser Gln Val Leu Lys Glu Ser Leu Arg Leu Tyr Pro Pro Ala Trp | |
| 355 360 365 | |
| ggc acc ttt cgc ctg ctg gaa gag gag acc ttg att gat ggg gtc aga | 1152 |
| Gly Thr Phe Arg Leu Leu Glu Glu Glu Thr Leu Ile Asp Gly Val Arg | |
| 370 375 380 | |
| gtc ccc ggc aac acc ccg ctc ttg ttc agc acc tat gtc atg ggg cgg | 1200 |
| Val Pro Gly Asn Thr Pro Leu Leu Phe Ser Thr Tyr Val Met Gly Arg | |
| 385 390 395 400 | |
| atg gac aca tac ttt gag gac ccg ctg act ttc aac ccc gat cgc ttc | 1248 |
| Met Asp Thr Tyr Phe Glu Asp Pro Leu Thr Phe Asn Pro Asp Arg Phe | |
| 405 410 415 | |
| ggc cct gga gca ccc aag cca cgg ttc acc tac ttc ccc ttc tcc ctg | 1296 |
| Gly Pro Gly Ala Pro Lys Pro Arg Phe Thr Tyr Phe Pro Phe Ser Leu | |
| 420 425 430 | |
| ggc cac cgc tcc tgc atc ggg cag cag ttt gct cag atg gag gtg aag | 1344 |
| Gly His Arg Ser Cys Ile Gly Gln Gln Phe Ala Gln Met Glu Val Lys | |
| 435 440 445 | |
| gtg gtc atg gca aag ctg ctg cag agg ctg gag ttc cgg ctg gtg ccc | 1392 |
| Val Val Met Ala Lys Leu Leu Gln Arg Leu Glu Phe Arg Leu Val Pro | |
| 450 455 460 | |
| ggg cag cgc ttc ggg ctg cag gag cag gcc aca ctc aag cca ctg gac | 1440 |
| Gly Gln Arg Phe Gly Leu Gln Glu Gln Ala Thr Leu Lys Pro Leu Asp | |
| 465 470 475 480 | |
| ccc gtg ctg tgc acc ctg cgg ccc cgc ggc tgg cag ccc gca ccc cca | 1488 |
| Pro Val Leu Cys Thr Leu Arg Pro Arg Gly Trp Gln Pro Ala Pro Pro | |
| 485 490 495 | |
| cca ccc ccc tgc tga gggggcctcc aggcaggacg agactcctcg ggcaagggcc | 1543 |
| Pro Pro Pro Cys | |
| 500 | |
| gtgcccgccc acctctgctg cccacggcca cccacccttc tcccctgccc cgtcccctgg | 1603 |
| gccacccttc acgctggctt ccagggggcc ctctgccgac cgcctgcttc acacccctca | 1663 |
| gcgctccctg tcgcctgcgg actccatggc ccttcctgga ctggcccttg cccaactccc | 1723 |
| agccaccacc actgtcccta ccactgagcc cttgcacagg ccacttgctc agacgagaca | 1783 |
| ccctaactct tgctcactcc ctaaagccct cttcaggggt cacctcctcc aagaagccct | 1843 |
| ccttgccacc ccccgccggc aggggcccct cctctgtgct ccctcggtca cctgtgctac | 1903 |
| ctctaacacc acactgacca cactgtatcg tgagtgtccg ttgacgtgac caattgccct | 1963 |
| gccaggctgt cagcgcctca agggtagggt ctgcgtgtga tttgtctctg agccccctgt | 2023 |
| gcccacccag ggcccggcac agagtcgatg ctcaataaat gtgtgttgac tgcaaaaaaa | 2083 |
| aaaaaaaaaa aaaaaaaaaa aaaaaaaaaa aaaaaaaaaa aaaaaaaaaa aaaaa | 2138 |

```
SEQ ID NO: 2
CYP46A1 amino acid sequence
Met Ser Pro Gly Leu Leu Leu Leu Gly Ser Ala Val Leu Leu Ala Phe
1 5 10 15
Gly Leu Cys Cys Thr Phe Val His Arg Ala Arg Ser Arg Tyr Glu His
                        20 25 30
Ile Pro Gly Pro Pro Arg Pro Ser Phe Leu Leu Gly His Leu Pro Cys
                35 40 45
Phe Trp Lys Lys Asp Glu Val Gly Gly Arg Val Leu Gln Asp Val Phe
        50 55 60
Leu Asp Trp Ala Lys Lys Tyr Gly Pro Val Val Arg Val Asn Val Phe
65 70 75 80
His Lys Thr Ser Val Ile Val Thr Ser Pro Glu Ser Val Lys Lys Phe
                                85 90 95
Leu Met Ser Thr Lys Tyr Asn Lys Asp Ser Lys Met Tyr Arg Ala Leu
                        100 105 110
Gln Thr Val Phe Gly Glu Arg Leu Phe Gly Gln Gly Leu Val Ser Glu
                115 120 125
Cys Asn Tyr Glu Arg Trp His Lys Gln Arg Arg Val Ile Asp Leu Ala
        130 135 140
Phe Ser Arg Ser Ser Leu Val Ser Leu Met Glu Thr Phe Asn Glu Lys
145 150 155 160
Ala Glu Gln Leu Val Glu Ile Leu Glu Ala Lys Ala Asp Gly Gln Thr
                                165 170 175
Pro Val Ser Met Gln Asp Met Leu Thr Tyr Thr Ala Met Asp Ile Leu
                        180 185 190
Ala Lys Ala Ala Phe Gly Met Glu Thr Ser Met Leu Leu Gly Ala Gln
                195 200 205
Lys Pro Leu Ser Gln Ala Val Lys Leu Met Leu Glu Gly Ile Thr Ala
        210 215 220
Ser Arg Asn Thr Leu Ala Lys Phe Leu Pro Gly Lys Arg Lys Gln Leu
225 230 235 240
Arg Glu Val Arg Glu Ser Ile Arg Phe Leu Arg Gln Val Gly Arg Asp
                                245 250 255
Trp Val Gln Arg Arg Arg Glu Ala Leu Lys Arg Gly Glu Glu Val Pro
                        260 265 270
Ala Asp Ile Leu Thr Gln Ile Leu Lys Ala Glu Glu Gly Ala Gln Asp
                275 280 285
Asp Glu Gly Leu Leu Asp Asn Phe Val Thr Phe Phe Ile Ala Gly His
        290 295 300
Glu Thr Ser Ala Asn His Leu Ala Phe Thr Val Met Glu Leu Ser Arg
305 310 315 320
Gln Pro Glu Ile Val Ala Arg Leu Gln Ala Glu Val Asp Glu Val Ile
                                325 330 335
Gly Ser Lys Arg Tyr Leu Asp Phe Glu Asp Leu Gly Arg Leu Gln Tyr
                        340 345 350
Leu Ser Gln Val Leu Lys Glu Ser Leu Arg Leu Tyr Pro Pro Ala Trp
                355 360 365
Gly Thr Phe Arg Leu Leu Glu Glu Glu Thr Leu Ile Asp Gly Val Arg
        370 375 380
Val Pro Gly Asn Thr Pro Leu Leu Phe Ser Thr Tyr Val Met Gly Arg
385 390 395 400
Met Asp Thr Tyr Phe Glu Asp Pro Leu Thr Phe Asn Pro Asp Arg Phe
                                405 410 415
Gly Pro Gly Ala Pro Lys Pro Arg Phe Thr Tyr Phe Pro Phe Ser Leu
                        420 425 430
Gly His Arg Ser Cys Ile Gly Gln Gln Phe Ala Gln Met Glu Val Lys
                435 440 445
Val Val Met Ala Lys Leu Leu Gln Arg Leu Glu Phe Arg Leu Val Pro
        450 455 460
Gly Gln Arg Phe Gly Leu Gln Glu Gln Ala Thr Leu Lys Pro Leu Asp
465 470 475 480
Pro Val Leu Cys Thr Leu Arg Pro Arg Gly Trp Gln Pro Ala Pro Pro
                                485 490 495
Pro Pro Pro Cys
                          500
```

### Vectors

Without wishing to be bound by any particular theory, allele-specific silencing of a pathogenic gene, e.g., mutant huntingtin (HTT), may provide an improved safety profile in a subject compared to non-allele specific silencing (e.g., silencing of both wild-type and mutant HTT alleles) because wild-type expression and function is preserved in the cells. For example, aspects of the invention relate to the inventors' recognition and appreciation that isolated nucleic acids and vectors that incorporate one or more inhibitory RNA (e.g., miRNA) sequences targeting the HTT gene in a non-allele-specific manner while driving the expression of hardened wild-type HTT gene (a wild-type HTT gene that is not targeted by the miRNA) are capable of achieving concomitant mutant HTT knockdown e.g., in the CNS tissue, with increased expression of wildtype HTT. Generally, the sequence of the nucleic acid encoding endogenous wild-type and mutant HTT mRNAs, and the nucleic acid of the transgene encoding the “hardened” wild-type HTT mRNA are sufficiently different such that the “hardened” wild-type HTT transgene mRNA is not targeted by the one or more inhibitory RNAs (e.g., miRNAs). This may be accomplished, for example, by introducing one or more silent mutations into the HTT transgene sequence such that it encodes the same protein as the endogenous wild-type HTT gene but has a different nucleic acid sequence. In this case, the exogenous mRNA may be referred to as “hardened.” Alternatively, the inhibitory RNA (e.g., miRNA) can target the 5′ and/or 3′ untranslated regions of the endogenous wild-type HTT mRNA. These 5′ and/or 3′ regions can then be removed or replaced in the transgene mRNA such that the transgene mRNA is not targeted by the one or more inhibitory RNAs.

Reporter sequences (e.g., nucleic acid sequences encoding a reporter protein) that may be provided in a transgene include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for D-galactosidase activity. Where the transgene is green fluorescent protein or luciferase, the vector carrying the signal may be measured visually by color or light production in a luminometer. Such reporters can, for example, be useful in verifying the tissue-specific targeting capabilities and tissue specific promoter regulatory activity of a nucleic acid. Recombinant adeno-associated viruses (rAAVs).

In some embodiments, the vector is adeno-associated virus (AAV) or recombinant AAV. In some aspects, the disclosure provides isolated AAVs. As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a nuclease and/or transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected.

AAV vectors are preferably used as self-complementary, double-stranded AAV vectors (scAAV) in order to overcome one of the limiting steps in AAV transduction (i.e. single-stranded to double-stranded AAV conversion), although the use of single-stranded AAV vectors (ssAAV) is also encompassed herein. In some embodiments of the invention, the AAV vector is chimeric, meaning it comprises components from at least two AAV serotypes, such as the ITRs of an AAV2 and the capsid protein of an AAV5. AAV9 is known to effectively transduce CNS cells and tissue particularly effectively, and thus AAV9 and derivatives thereof are of particular interest for targeting CNS cells and tissue. AAV2g9 is known to effectively transduce CNS cells and tissue particularly effectively, and thus AAV2g9 and derivatives thereof are of particular interest for targeting CNS cells and tissue. AAVrh10 is known to effectively transduce CNS cells and tissue particularly effectively, and thus AAVrh10 and derivatives thereof are of particular interest for targeting CNS cells and tissue. AAVrh10 is particularly preferred as systemic or intravenous delivery of AAVrh10 has been found to provide high transgene expression in the central nervous system as described in (Tanguy et al., 2015) which is incorporated herein by reference. AAVDJ8 is known to effectively transduce CNS cells and tissue particularly effectively, and thus AAVDJ8 and derivatives thereof are of particular interest for targeting CNS cells and tissue. AAVDJ8 is preferred as it has been shown to effectively target multiple regions of the brain and to effectively target astrocytes as described in (Hammond et al., 2017) which is incorporated herein by reference. AAV1, AAV2, AAV4, AAV5 and AAV8 are also known to target CNS cells and tissue, and thus these AAV serotypes and derivates thereof are also of particular interest for targeting CNS cells and tissue. AAV-PHP.eB is also of particular interest as it is known to effectively cross the blood brain barrier (Mathiesen et al., 2020).

Methods for obtaining recombinant AAVs having a desired capsid protein are well known in the art. (See, for example, US 2003/0138772), the contents of which are incorporated herein by reference in their entirety). Typically, the methods involve culturing a host cell which contains a nucleic acid sequence encoding an AAV capsid protein; a functional rep gene; a recombinant AAV vector composed of, AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, a recombinant AAV (rAAV) capsid protein is of an AAV serotype selected from the group consisting of AAV2, AAV3, AAV4, AAV5, AAV6, AAV8, AAVrh8, AAVrh10, AAV 2G9, AAV 2.5G9, AAV9, AAVDJ8, AAV-PHP.eB, and AAV10. In some embodiments, an AAV capsid protein is of a serotype derived from a non-human primate, for example AAVrh10 serotype. In some embodiments, an AAV capsid protein is of an AAV9 serotype. In some embodiments, the capsid protein is an AAV1, AAV2, AAV3b, AAV4, AAV5, AAV6, AAV7, AAV8, AAV9, AAV10, AAVrh10, AAV11, AAV12, or AAV13 capsid protein or, a chimera thereof. In some embodiments, the rAAV comprises a capsid protein from serotype AAV1, AAV2, AAV3a, AAV3b, AAV4, AAV5, AAV6, AAV7, AAV8, AAV9, AAV 2G9, AAV 2.5G9, AAV rh8, AAV rh10, AAV rh74, AAV10, or, AAV11 or, a chimera thereof. In certain embodiments, the rAAV comprises a chemically modified capsid as disclosed in WO 2017/212019 e.g., mannose ligand is chemically coupled to AAV2. The rAAVs with chemically modified capsids disclosed in WO 2017/212019 is incorporated herein by reference in its entirety. As a further embodiment, the AAV capsid proteins and virus capsids of this invention can be polyploid (also referred to as rational haploid) in that they can comprise different combinations of VP1, VP2 and VP3 AAV serotypes in a single AAV capsid as described in PCT/US18/22725, PCT/US2018/044632, or U.S. Pat. No. 10,550,405 which are incorporated by reference.

In one embodiment, the AAV is AAV9 or derivatives thereof.

In one embodiment, the AAV is AAVrh10 or derivatives thereof.

In another embodiment, the AAV is AAV-PHP.eB or derivatives thereof.

The components to be cultured in the host cell to package a rAAV vector in an AAV capsid may be provided to the host cell in trans. Alternatively, any one or more of the required components {e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain El helper functions under the control of a constitutive promoter), but which contain the rep and/or cap proteins under the control of inducible promoters. Still other stable host cells may be generated by one of skill in the art. In some embodiments, the instant disclosure relates to a host cell containing a nucleic acid that comprises a coding sequence encoding a protein (e.g., wild-type huntingtin protein, optionally “hardened” wild-type huntingtin protein). In some embodiments, the instant disclosure relates to a composition comprising the host cell described above. In some embodiments, the composition comprising the host cell above further comprises a cryopreservative.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the triple transfection method uses closed linear DNA molecules that lack bacterial backbone sequences, for example, as described in PCT/US2021/013689, published as WO/2021/146591, which is incorporated herein by reference in its entirety.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13: 197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. One type of vector is a “plasmid,” which refers to a circular double stranded DNA loop into which additional DNA segments are ligated. Another type of vector is a viral vector, wherein additional DNA segments are ligated into the viral genome. Certain vectors are capable of autonomous replication in a host cell into which they are introduced (e.g., bacterial vectors having a bacterial origin of replication and episomal mammalian vectors). Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” is used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

A cloning vector is one which is able to replicate autonomously or integrated in the genome in a host cell, and which is further characterized by one or more endonuclease restriction sites at which the vector may be cut in a determinable fashion and into which a desired DNA sequence can be ligated such that the new recombinant vector retains its ability to replicate in the host cell. In the case of plasmids, replication of the desired sequence can occur many times as the plasmid increases in copy number within the host cell such as a host bacterium or just a single time per host before the host reproduces by mitosis. In the case of phage, replication can occur actively during a lytic phase or passively during a lysogenic phase.

An expression vector is one into which a desired DNA sequence can be inserted by restriction and ligation such that it is operably joined to regulatory sequences and can be expressed as an RNA transcript. Vectors can further contain one or more marker sequences suitable for use in the identification of cells which have or have not been transformed or transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). In certain embodiments, the vectors used herein are capable of autonomous replication and expression of the structural gene products present in the DNA segments to which they are operably joined.

The present invention further provides a vector comprising a synthetic CNS-specific promoter, or expression cassette of the present invention. In some embodiments of the invention, the vector is a plasmid. Such a plasmid may include a variety of other functional nucleic acid sequences, such as one or more selectable markers, one or more origins of replication, multiple cloning sites and the like. In some embodiments of the invention, the vector is a viral vector.

In some embodiments of the invention, the vector is an expression vector for expression in eukaryotic cells. Examples of eukaryotic expression vectors include, but are not limited to, pW-LNEO, pSV2CAT, pOG44, pXTI and pSG available from Stratagene; pSVK3, pBPV, pMSG and pSVL available from Amersham Pharmacia Biotech; and pCMVDsRed2-express, pIRES2-DsRed2, pDsRed2-Mito, pCMV-EGFP available from Clontech. Many other vectors are well-known and commercially available. For mammalian cells adenoviral vectors, the pSV and the pCMV series of vectors are particularly well-known non-limiting examples. There are many well-known yeast expression vectors including, without limitation, yeast integrative plasmids (YIp) and yeast replicative plasmids (YRp). For plants the Ti plasmid of agrobacterium is an exemplary expression vector, and plant viruses also provide suitable expression vectors, e.g. tobacco mosaic virus (TMV), potato virus X, and cowpea mosaic virus.

In some preferred embodiments, the vector is a gene therapy vector. Various gene therapy vectors are known in the art, and mention can be made of AAV vectors, adenoviral vectors, retroviral vectors and lentiviral vectors. Where the vector is a gene therapy vector the vector preferably comprises a nucleic acid sequence operably linked to the synthetic CNS-specific promoter of the invention that encodes a therapeutic product, suitably a therapeutic protein. The therapeutic protein may be a secretable protein. Non-limiting examples of secretable proteins are discussed above, and exemplary secretable therapeutic proteins, include clotting factors, such as factor VIII or factor IX, insulin, erythropoietin, lipoprotein lipase, antibodies or nanobodies, growth factors, cytokines, chemokines, plasma factors, toxic proteins, etc.

In some embodiments, useful vectors are contemplated to be those vectors in which the nucleic acid segment to be transcribed is positioned under the transcriptional control of a promoter. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

A “promoter” refers to a DNA sequence recognized by the synthetic machinery of the cell, or introduced synthetic machinery, required to initiate the specific transcription of a gene. When the nucleic acid molecule that encodes any of the polypeptides described herein is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The term “synthetic promoter” as used herein relates to a promoter that does not occur in nature. In the present context it typically comprises a CRE of the present invention operably linked to a promoter element (e.g. minimal (or core) promoter or CNS-specific proximal promoter). The one or more CREs of the present invention serve to enhance CNS-specific transcription of a gene operably linked to the synthetic promoter. Parts of the synthetic promoter may be naturally occurring (e.g. the minimal promoter or one or more CREs in the promoter), but the synthetic promoter as a complete entity is not naturally occurring.

The precise nature of the regulatory sequences needed for gene expression can vary between species or cell types, but in general can include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences can also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA). That heterologous DNA (RNA) is placed under operable control of transcriptional elements to permit the expression of the heterologous DNA in the host cell.

The phrases “operatively positioned,” “under control” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or functional RNA (e.g., guide RNA) from a transcribed gene.

The foregoing methods for packaging recombinant vectors in desired AAV capsids to produce the rAAVs of the disclosure are not meant to be limiting and other suitable methods will be apparent to the skilled artisan.

In some embodiments, any one or more thymidine (T) nucleotides or uridine (U) nucleotides in a sequence provided herein, including a sequence provided in the sequence listing, may be replaced with any other nucleotide suitable for base pairing (e.g., via a Watson-Crick base pair) with an adenosine nucleotide. For example, in some embodiments, any one or more thymidine (T) nucleotides in a sequence provided herein, including a sequence provided in the sequence listing, may be suitably replaced with a uridine (U) nucleotide or vice versa.

In some embodiments of any of the aspects, a nucleic acid (e.g., miRNA) is chemically modified to enhance stability or other beneficial characteristics. The nucleic acids described herein may be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of nucleic acid compounds useful in the embodiments described herein include, but are not limited to nucleic acids containing modified backbones or no natural internucleoside linkages. nucleic acids having modified backbones include, among others, those that do not have a phosphorus atom in the backbone. For the purposes of this specification, and as sometimes referenced in the art, modified nucleic acids that do not have a phosphorus atom in their internucleoside backbone can also be considered to be oligonucleosides. In some embodiments of any of the aspects, the modified nucleic acid will have a phosphorus atom in its internucleoside backbone.

Modified nucleic acid backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified nucleic acid backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2--NH—CH2--, —CH2--N(CH3)--O—CH2-- [known as a methylene (methylimino) or MMI backbone], —CH2--O—N(CH3)--CH2--, —CH2--N(CH3)--N(CH3)--CH2-- and —N(CH3)--CH2--CH2-- [wherein the native phosphodiester backbone is represented as —O—P—O—CH2--].

In other nucleic acid mimetics, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

The nucleic acid can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, OR. et al., (2007) Mol. Canc. Ther. 6(3):833-843; Grunweller, A. et al., (2003) Nucleic Acids Research 31(12):3185-3193).

Modified nucleic acids can also contain one or more substituted sugar moieties. The nucleic acids described herein can include one of the following at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C10 alkyl or C₂ to C10 alkenyl and alkynyl. Exemplary suitable modifications include O[(CH2)nO]mCH3, O(CH2)nOCH3, O(CH2)nNH2, O(CH2) nCH3, O(CH2)nONH2, and O(CH2)nON[(CH2)nCH3)]2, where n and m are from 1 to about 10. In some embodiments of any of the aspects, nucleic acids include one of the following at the 2′ position: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleic acid, or a group for improving the pharmacodynamic properties of a nucleic acid, and other substituents having similar properties. In some embodiments of any of the aspects, the modification includes a 2′ methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O--CH2--O—CH2--N(CH2)2, also described in examples herein below.

Other modifications include 2′-methoxy (2′-OCH3), 2′-aminopropoxy (2′-OCH2CH2CH2NH2) and 2′-fluoro (2′-F). Similar modifications can also be made at other positions on the nucleic acid, particularly the 3′ position of the sugar on the 3′ terminal nucleotide or in 2′-5′ linked dsRNAs and the 5′ position of 5′ terminal nucleotide. Nucleic acids may also have sugar mimetics such as cyclobutyl moieties in place of the pentofuranosyl sugar.

A nucleic acid can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases can include other synthetic and natural nucleobases including but not limited to as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications. In some embodiments of any of the aspects, modified nucleobases can include d5SICS and dNAM, which are a non-limiting example of unnatural nucleobases that can be used separately or together as base pairs (see e.g., Leconte et. al. J. Am. Chem. Soc. 2008, 130, 7, 2336-2343; Malyshev et. al. PNAS. 2012. 109 (30) 12005-12010). In some embodiments of any of the aspects, oligonucleotide tags (e.g., Oligopaint) comprise any modified nucleobases known in the art, i.e., any nucleobase that is modified from an unmodified and/or natural nucleobase.

The preparation of the modified nucleic acids, backbones, and nucleobases described above are well known in the art.

Another modification of a nucleic acid featured in the invention involves chemically linking to the nucleic acid to one or more ligands, moieties or conjugates that enhance the activity, cellular distribution, pharmacokinetic properties, or cellular uptake of the nucleic acid. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In some embodiments of any of the aspects, the vector is pEMBL. In some embodiments of any of the aspects, the vector is pEMBL-D(+)Syn1. In some embodiments of any of the aspects, the vector is pEMBL-D(+)Syn1-hCG intron only. In some embodiments of any of the aspects, the vector is pEMBL-D(+)Syn1-hCGin-2× control pre-miR. In some embodiments of any of the aspects, the vector is pEMBL-D(+)Syn1-hCGin-2× artificial pre-miR. In some embodiments of any of the aspects, the vector is pEMBL-D(+)Syn1-CYP46A1-hCGin-2× artificial pre-miR. In some embodiments of any of the aspects, the vector is pEMBL-D(+)Syn1-luc-HTT-3′UTR/mutant. In some embodiments of any of the aspects, the vector comprises at least one of the following: at least one (e.g., 2) ITRs; Syn1 promoter; at least one (e.g., 2) hCG intron; at least one (e.g., 2) copy of a premiR (e.g., control pre-miR; artificial pre-miR; SEQ ID NO: 6-17, 40-44, 50-66, 158-185, or 217-260); small polyA; CYP46A1; luciferase; HTT targeting sequences; and/or HTT-3′UTR/mutant. In some embodiments, the vector comprises a neuron specific synthetic promoter selected from Tables 10-13, and/or a CRE selected from Tables 13-15. In certain aspects of embodiments, the miRNA targets wild type HTT allele. In other aspects of the embodiments, the miRNA targets mutant HTT allele. In yet another embodiment, the miRNA targets both wild type and mutant HTT alleles. In yet another embodiment, the miRNA targets any HTT mRNA.

In some embodiments, one or more of the recombinantly expressed gene can be integrated into the genome of the cell.

A nucleic acid molecule that encodes the enzyme of the claimed invention can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding the enzymes of the claimed invention also may be accomplished by integrating the nucleic acid molecule into the genome.

In some embodiments, the promoter is a synapsin (Syn1) promoter (see e.g., SEQ ID NO: 152). In one aspect, the promoter comprises a nucleic acid sequence at least 80% identical, e.g., at least 85%, at least 90%, at least 95%, at least 98%, at least 99% or 100% identical, to SEQ ID NO: 152. In one aspect, provided herein is a composition comprising a recombinant viral vector comprising a promoter comprising a nucleic acid sequence at least 80% identical, e.g., at least 85%, at least 90%, at least 95%, at least 98%, at least 99% or 100% identical, to SEQ ID NO: 152.

Synapsin-1 (SEQ ID NO: 152)
(SEQ ID NO: 152)
GAGGGCCCTGCGTATGAGTGCAAGTGGGTTTTAGGACCAGGATGAGGCGG
GGTGGGGGTGCCTACCTGACGACCGACCCCGACCCACTGGACAAGCACCC
AACCCCCATTCCCCAAATTGCGCATCCCCTATCAGAGAGGGGGAGGGGAA
ACAGGATGCGGCGAGGCGCGTGCGCACTGCCAGCTTCAGCACCGCGGACA
GTGCCTTCGCCCCCGCCTGGCGGCGCGCGCCACCGCCGCCTCAGCACTGA
AGGCGCGCTGACGTCACTCGCCGGTCCCCCGCAAACTCCCCTTCCCGGCC
ACCTTGGTCGCGTCCGCGCCGCCGCCGGCCCAGCCGGACCGCACCACGCG
AGGCGCGAGATAGGGGGGCACGGGCGCGACCATCTGCGCTGCGGCGCCGG
CGACTCAGCGCTGCCTCAGTCTGCGGTGGGCAGCGGAGGAGTCGTGTCGT
GCCTGAGAGCGCAGTCG

In one aspect, provided herein is a composition comprising an isolated nucleic acid comprising a sequence at least 80% identical, e.g., at least 85%, at least 90%, at least 95%, at least 98%, at least 99% or 100% identical, to SEQ ID NO: 111. In one aspect, provided herein is a composition comprising a recombinant viral vector comprising an isolated nucleic acid comprising a sequence at least 80% identical, e.g., at least 85%, at least 90%, at least 95%, at least 98%, at least 99% or 100% identical, to SEQ ID NO: 111. In some embodiments, the vector (e.g., rAAV) comprises a promoter (e.g., a synthetic nervous system specific promoter; see e.g., Tables 10-13) or fragments thereof, an enhancer, and/or cis-regulatory elements (CREs; see e.g., Tables 13-15). In some embodiments, the enhancer is a CMV enhancer. In some embodiments, the promoter is an ACTB proximal promoter. In some embodiments, the vector further comprises an intron. In some embodiments, the intron comprises an ACTB intron/chimeric ACTB-HBB2 intron. See e.g., SEQ ID NO: 111, Table 16. The foregoing compositions can be used, e.g., in the absence of an administered miRNA to treat a neurological disease or disorder as described herein.

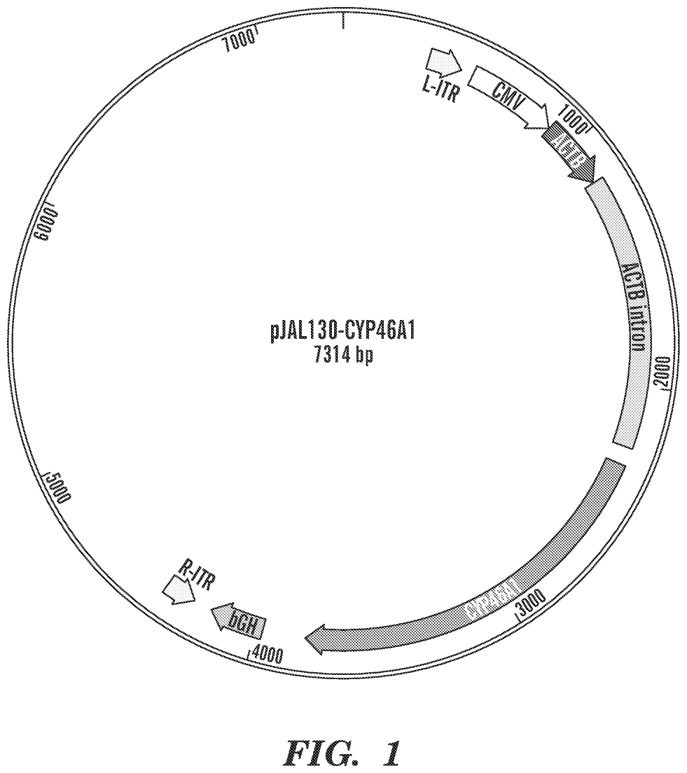

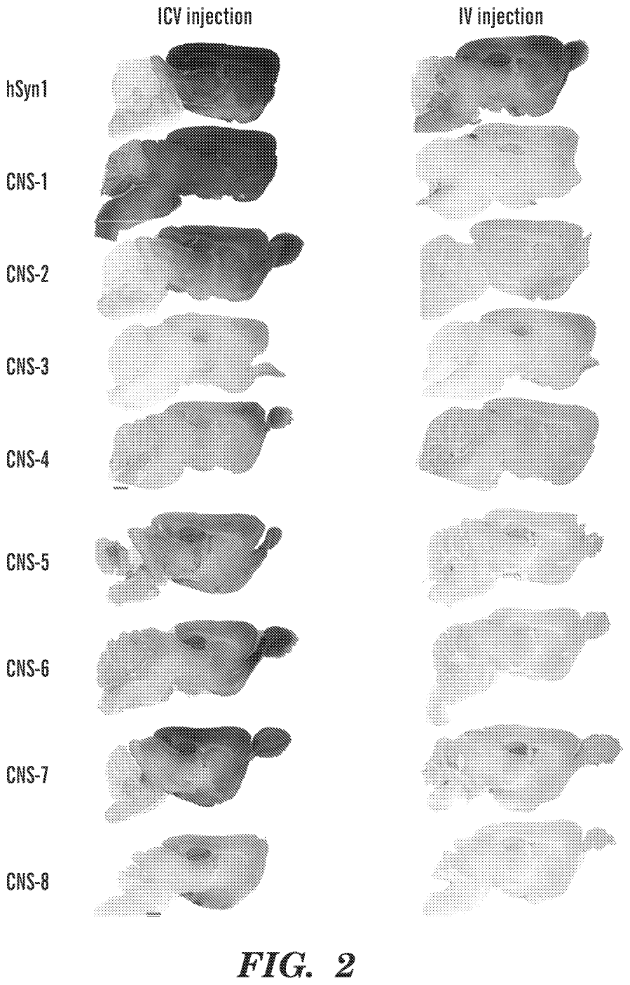

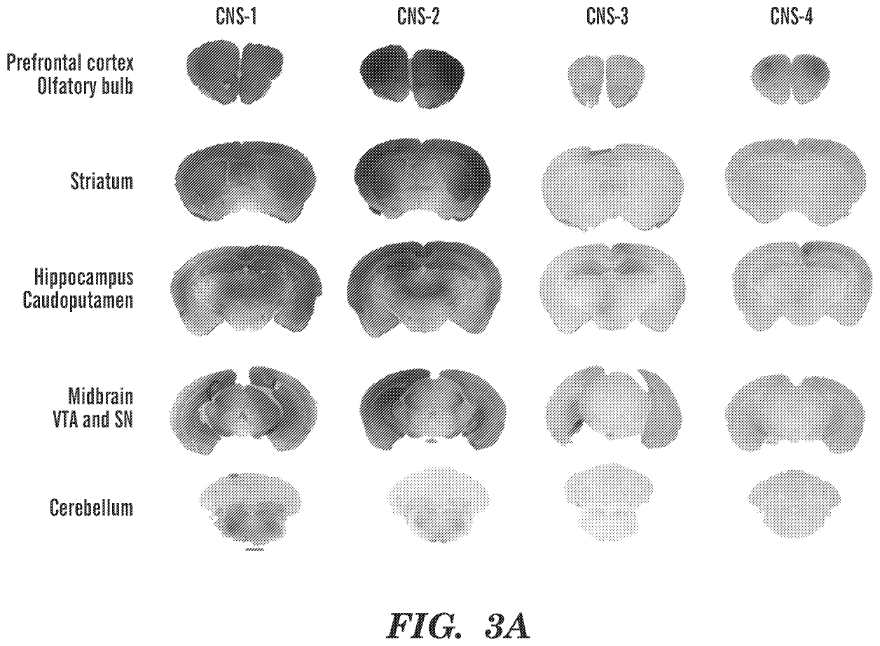

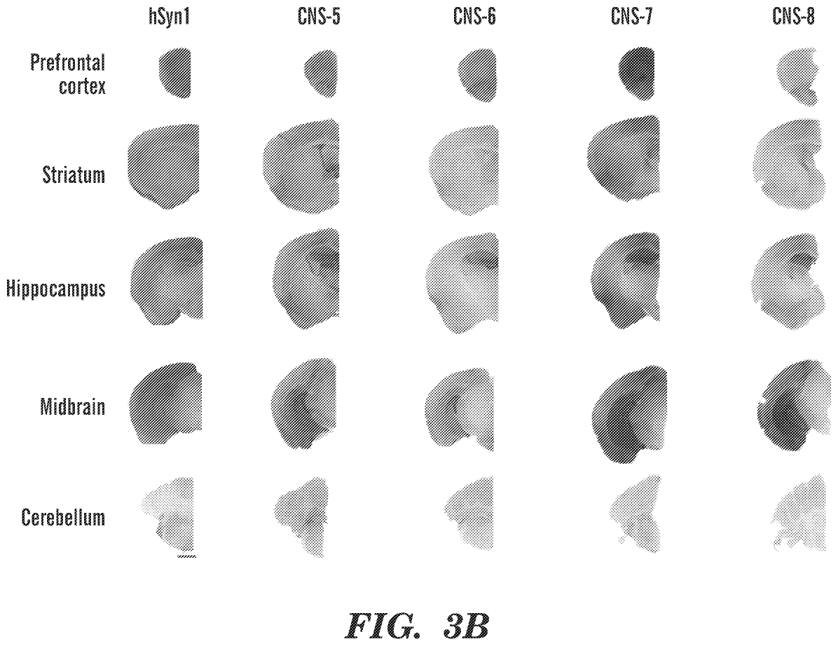

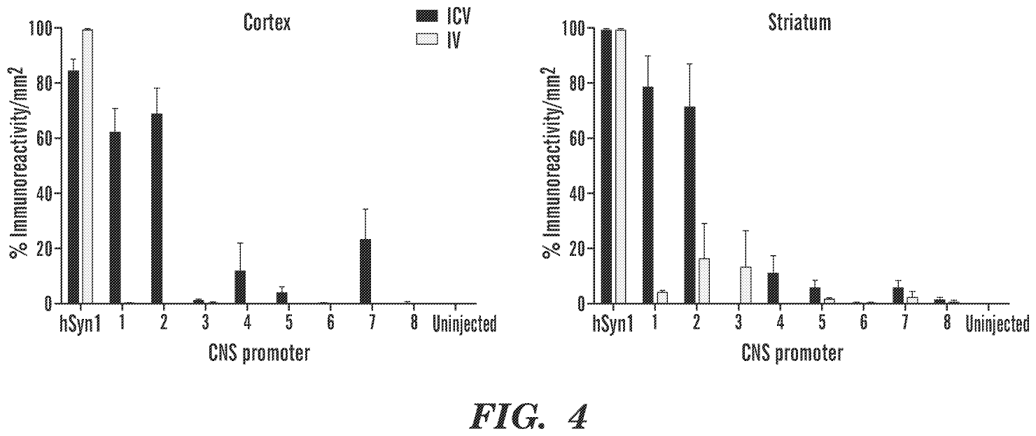

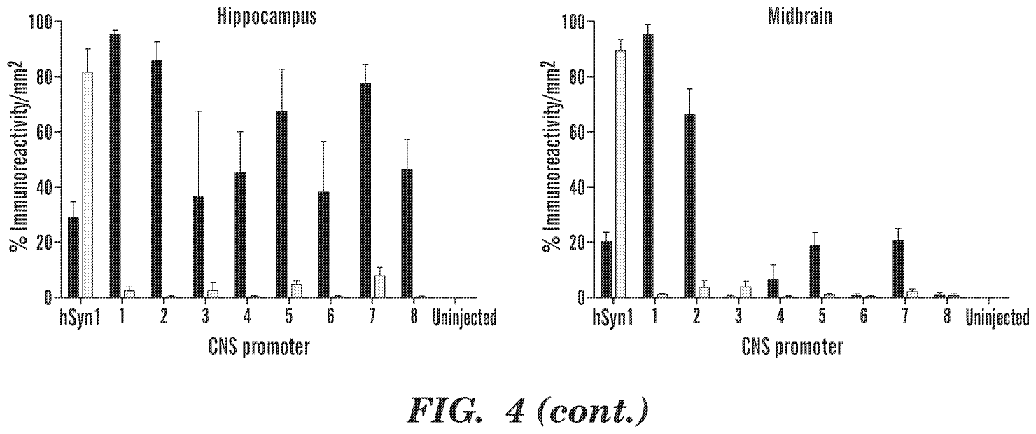

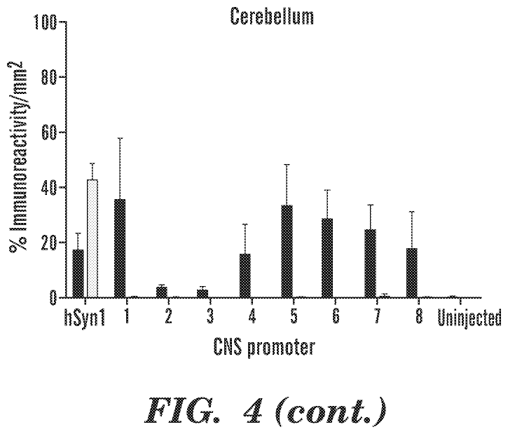

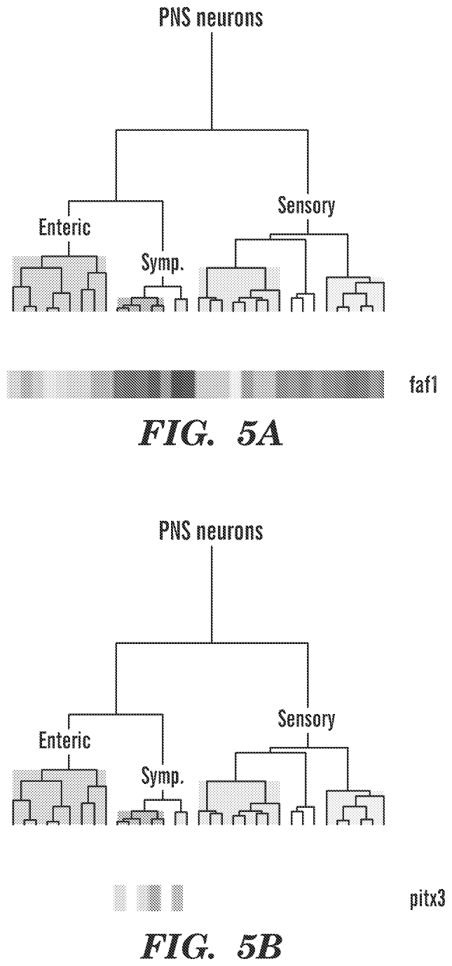

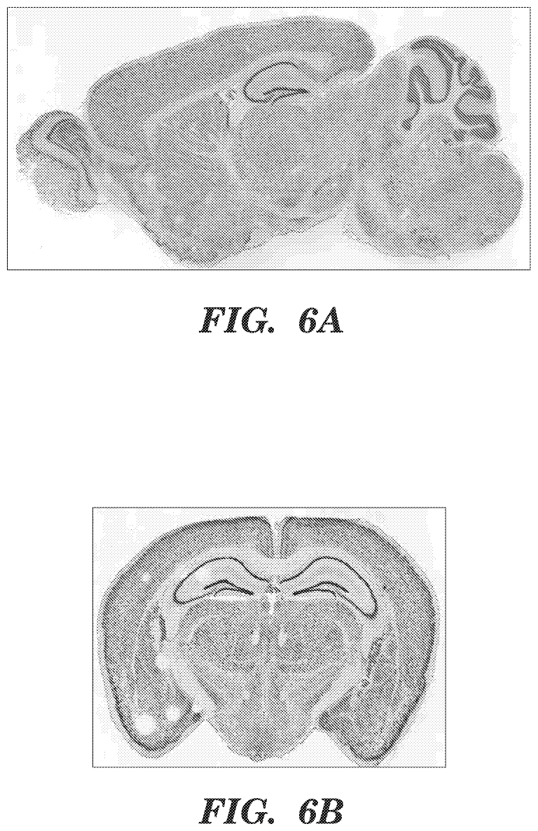

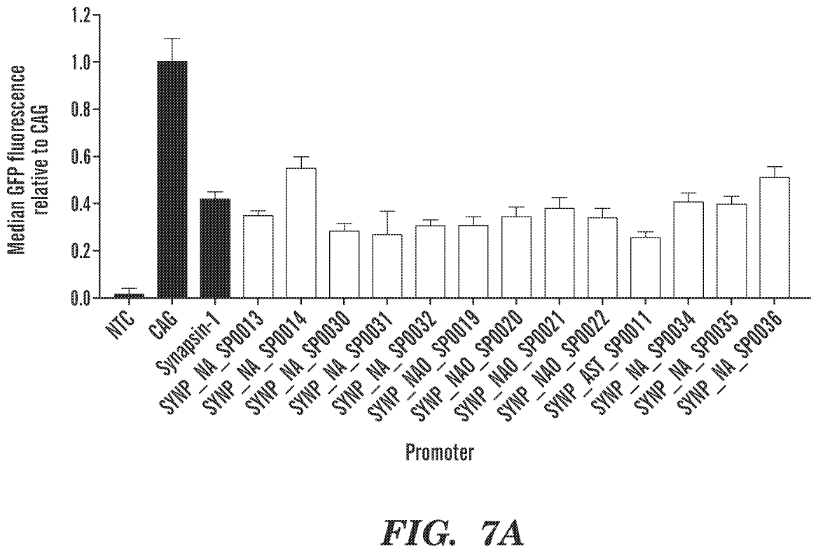

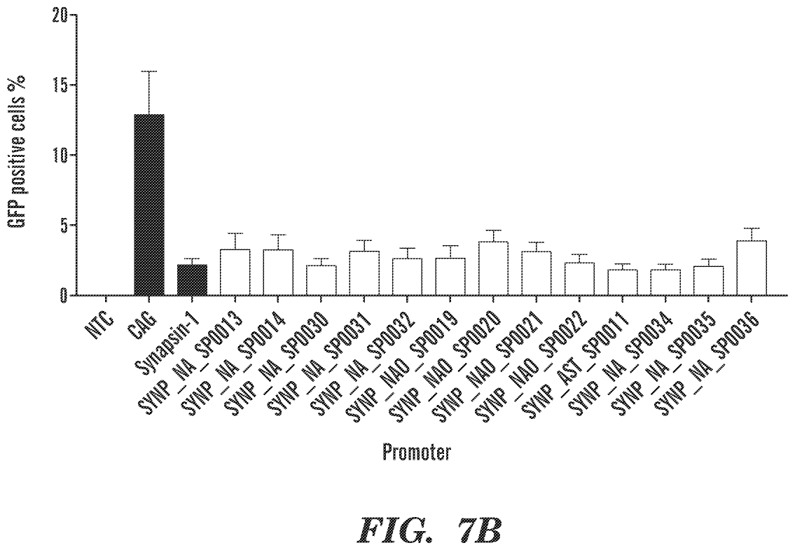

SEQ ID NO: 111, pJAL130-CYP46A1, 4036 bp, ITR to ITR sequence of the codon-optimized CYP46 sequence (see e.g., SEQ ID NO: 110; see e.g., FIG. 1).

Bolded text (e.g., nucleotides (nt) 1-130 of SEQ ID NO: 111) indicates the left ITR.

Italicized text (e.g., nt 182-436 of SEQ ID NO: 111) indicates the enhancer.

Bold italicized text (e.g., nt 550-804 of SEQ ID NO: 111) indicates the promoter.

Double underlined text (e.g., nt 824-1892 of SEQ ID NO: 111) indicates the intron.

Bolded double underlined text (e.g., nt 1966-3465 of SEQ ID NO: 111) indicates the coding sequence (CDS) of codon-optimized CYP46A1 (see e.g., SEQ ID NO: 110).

Italicized double underlined text (e.g., nt 3629-3853 of SEQ ID NO: 111) indicates the polyA.

Bolded italicized double underlined text (e.g., nt 3907-4036 of SEQ ID NO: 111) indicates the right ITR.

(SEQ ID NO: 111)
CTGCGCGCTCGCTCGCTCACTGAGGCCGCCCGGGCAAAGCCCGGGCGTCGGGCGACCTTTGGTCGCCCGGCCTCA
GTGAGCGAGCGAGCGCGCAGAGAGGGAGTGGCCAACTCCATCACTAGGGGTTCCTTGTAGTTAATGATTAACCCG
CCATGCTACTTATCTACGGCGCGCCacgcgtgactagttattaatagtaatcaattacggggtcattagttcata
gcccatatatggagttccgcgttacataacttacggtaaatggcccgcctggctgaccgcccaacgacccccgcc
cattgacgtcaataatgacgtatgttcccatagtaacgccaatagggactttccattgacgtcaatgggtggagt
atttacggtaaactgcccacttggcagtacatcaagtgtatcatatgccaagtacgccccctattgacgtcaatg
acggtaaatggcccgcctggcattatgcccagtacatgaccttatgggactttcctacttggcagtacatctacg
tattagtcatcgctattaccatggtcgaggtgagccccacgttctgcttcactctccccatctcccccccctccc
cacccccaattttgtatttatttattttttaattattttgtgcagcgatgggggcgggggggggggggggcgcgc
gccaggcggggcggggcggggcgaggggcggggcggggcgaggcggagaggtgcggcggcagccaatcagagcgg
cgcgctccgaaagtttccttttatggcgaggcggcggcggcggcggccctataaaaagcgaagcgcgcggcgggc
gggagtcgctgcgcgctgccttcgccccgtgccccgctccgccgccgcctcgcgccgcccgccccggctctgact
gaccgcgttactcccacaggtgagcgggcgggacggcccttctcctccgggctgtaattagcgcttggtttaatg
acggcttgtttcttttctgtggctgcgtgaaagccttgaggggctccgggagggccctttgtgcggggggagcgg
ctcggggggtgcgtgcgtgtgtgtgtgcgtggggagcgccgcgtgcggctccgcgctgcccggcggctgtgagcg
ctgcgggcgcggcgcggggctttgtgcgctccgcagtgtgcgcgaggggagcgcggccgggggcggtgccccgcg
gtgcggggggggctgcgaggggaacaaaggctgcgtgcggggtgtgtgcgtgggggggtgagcagggggtgtggg
cgcgtcggtcgggctgcaaccccccctgcacccccctccccgagttgctgagcacggcccggcttcgggtgcggg
gctccgtacggggcgtggcgcggggctcgccgtgccgggcggggggtggcggcaggtgggggtgccgggcggggc
ggggccgcctcgggccggggagggctcgggggaggggcgcggcggcccccggagcgccggcggctgtcgaggcgc
ggcgagccgcagccattgccttttatggtaatcgtgcgagagggcgcagggacttcctttgtcccaaatctgtgc
ggagccgaaatctgggaggcgccgccgcaccccctctagcgggcgcggggcgaagcggtgcggcgccggcaggaa
ggaaatggggggggagggccttcgtgcgtcgccgcgccgccgtccccttctccctctccagcctcggggctgtcc
gcggggggacggctgccttcgggggggacggggcagggcggggttcggcttctggcgtgtgaccggcggctctag
agcctctgctaaccatgttcatgccttcttctttttcctacagctcctgggcaacgtgctggttattgtgctgtc
tcatcattttggcaaagaattgattaattcgagcgaacgcgtcgagtcgctcggtacgatttaaattgaattcct
taagctatcatagga

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

tgagtgatagcttggtaccgagctcgatccaattgcaatgatcatcatgacagatctgcg
cgcgatcgatatcagcgctttaaatttgcgcatgcagctatagttctagagggccctattctatagtgtcaccta
aatgctagagctcgctgatcagcctcgactgtgccttctagttgccagccatctgttgtttgcccctcccccgtg
ccttccttgaccctggaaggtgccactcccactgtcctttcctaataaaatgaggaaattgcatcgcattgtctg
agtaggtgtcattctattctggggggtggggtggggcaggacagcaagggggaggattgggaagacaatagcagg
catgctggggatgcggtgggctctatgggtttaaacGCGGCCGCGTAGATAAGTAGCATGGCGGGTTAATCATTA
ACTACA

[symbol]

[symbol]

[symbol]

[symbol]

[symbol]

[symbol]

TABLE 16

| | Location in | | |
|---|---|---|---|
| | SEQ ID | | |
| Feature | NO: 111 | | Qualifier |
| misc_feature | 1 | 130 | /label=L-ITR |
| enhancer | 182 | 548 | /label=CMVe |
| | | | /label=CMVenhancer |
| | | | /note=“GenBank: KX529075.1” |
| promoter | 550 | 823 | /label=ACTBp |
| | | | /label=ACTB proximal promoter |
| misc_feature | 824 | 1892 | /label=chimeric ACTB-HBB2 intron |
| CDS | 1966 | 3468 | /codon_start=1 /product=“>NM_006668.2 Homo sapiens |
| | | | cytochrome P450 family 46 subfamily A member 1 (CYP46A1), |
| | | | mRNA” |
| polyA_signal | 3629 | 3853 | /label=bGH polyA /label=bGH\poly(A)\signal |
| | | | /note=“Bovine growth hormone gene, complete cds. GenBank: |
| | | | M57764.1 ncbi.nlm.nih.gov/nuccore/M57764.1” |
| misc_feature | 3907 | 4036 | /label=R-ITR |
| source | 182 | 3853 | /organism=“recombinant plasmid” |
| | | | /mol_type=“other DNA” |
| | | | /note=“/vntifkey=98” |
| source | 550 | 1794 | /label=Gallus gallus sequence |
| | | | /note=“/Transferred_From=CBA-Promoter UNC-AskBio_JMI |
| | | | edited” |
| | | | /note=“/Transferred_Similarity=99.36%” |
| | | | /note=“/created_by=User” |
| source | 1803 | 1892 | /label=Oryctolagus cuniculus |
| | | | /note=“/Transferred_From=CBA Promoter UNC-AskBio_JMI |
| | | | edited” |
| | | | /note=“/Transferred_Similarity=100.00%”/note=“/created_by=User” |
| | | | /note=“/modified_by=User” |

In one aspect, provided herein is a composition comprising an isolated nucleic acid comprising a sequence at least 80% identical, e.g., at least 85%, at least 90%, at least 95%, at least 98%, at least 99% or 100% identical, to SEQ ID NO: 194. In one aspect, provided herein is a composition comprising a recombinant viral vector comprising an isolated nucleic acid comprising a sequence at least 80% identical, e.g., at least 85%, at least 90%, at least 95%, at least 98%, at least 99% or 100% identical, to SEQ ID NO: 194. In some embodiments, the vector (e.g., rAAV) comprises a promoter (e.g., a synthetic nervous system specific promoter; see e.g., Tables 10-13) or fragments thereof, an enhancer, and/or cis-regulatory elements (CREs; see e.g., Tables 13-15). In some embodiments, the enhancer is a CMV enhancer. In some embodiments, the promoter is an ACTB proximal promoter. In some embodiments, the promoter is a CAG promoter. In some embodiments, the vector further comprises an intron. In some embodiments, the intron comprises an ACTB intron/chimeric ACTB-HBB2 intron. The foregoing compositions can be used, e.g., in the absence of an administered miRNA to treat a neurological disease or disorder as described herein.

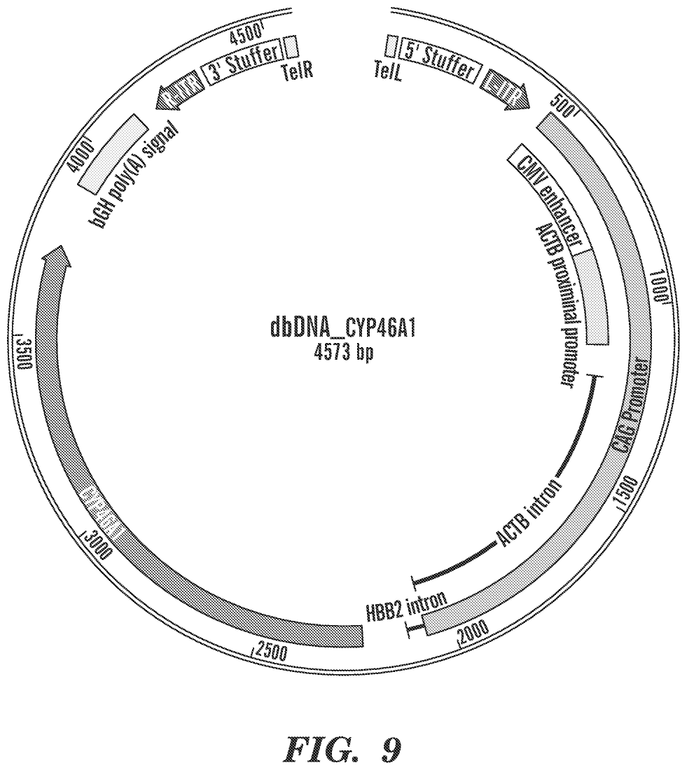

SEQ ID NO: 194, dbDNA-CYP46A1, 4,573 bp, ITR to ITR sequence of the codon-optimized CYP46 sequence (see e.g., SEQ ID NO: 212; see e.g., FIG. 9). SEQ ID NO: 212 is the sequence of SEQ ID NO: 110 with an additional three base pairs at the c-terminus that translate a stop codon.

Base pairs 1-28 of SEQ ID NO: 194 describe a TelN protelomerase recognition sequence; i.e., SEQ ID NO: 195.

Base pairs 29-37 of SEQ ID NO: 194 describe a linker sequence; i.e., SEQ ID NO: 196.

Base pairs 38-249 of SEQ ID NO: 194 describe a 5′ Stuffer sequence; i.e., SEQ ID NO: 197.

Base pairs 250-271 of SEQ ID NO: 194 describe a linker sequence; i.e., SEQ ID NO: 198.

Base pairs 272-401 of SEQ ID NO: 194 describe a L-ITR sequence; i.e., SEQ ID NO: 199.

Base pairs 402-452 of SEQ ID NO: 194 describe a linker sequence; i.e., SEQ ID NO: 200.

Base pairs 453-2073 of SEQ ID NO: 194 describe a CAG Promoter sequence; i.e., SEQ ID NO: 193.

Base pairs 2074-2114 of SEQ ID NO: 194 describe a HBB2 intron sequence; i.e., SEQ ID NO: 201.

Base pairs 2115-2236 of SEQ ID NO: 194 describe a linker sequence; i.e., SEQ ID NO: 202.

Base pairs 2237-2379 of SEQ ID NO: 194 describe a CYP46A1 sequence; i.e., SEQ ID NO: 212 (SEQ ID NO: 212 is the sequence of SEQ ID NO: 110 with an additional three base pairs at the c-terminus that translate a stop codon.)

Base pairs 2380-3899 of SEQ ID NO: 194 describe a linker sequence; i.e., SEQ ID NO: 203.

Base pairs 3900-4124 of SEQ ID NO: 194 describe a bGH poly(A) signal sequence; i.e., SEQ ID NO: 204.

Base pairs 4125-4177 of SEQ ID NO: 194 describe a linker sequence; i.e., SEQ ID NO: 205.

Base pairs 4178-4307 of SEQ ID NO: 194 describe a R-ITR sequence; i.e., SEQ ID NO: 206.

Base pairs 4308-4321 of SEQ ID NO: 194 describe a linker sequence; i.e., SEQ ID NO: 207.

Base pairs 4322-4533 of SEQ ID NO: 194 describe a 3′ Stuffer sequence; i.e., SEQ ID NO: 208.

Base pairs 4534-4545 of SEQ ID NO: 194 describe a linker sequence; i.e., TD NO: 209.

Base pairs 4546-4573 of SEQ TD NO: 194 describe a TelN protelomerase recognition sequence; i.e., SEQ TD NO: 210.

TABLE 25
Sequences comprised within dbDNA-CYP46A1, see SEQ ID NO: 194.

| SEQ | |
|---|---|
| ID | |
| NO: | Nucleic Acid Sequence |
| 195 | gcgtataatggactattgtgtgctgata |
| 196 | tctgtacac |
| 197 | ttaagggctagatcttagcttacgtcactagagggtccacgtttagtttttaagatccattgatctccta |
| | aacgctgcaagattcgcaacctggtatacttagcctaggcgctaggtcctagtgcagcgggacttttttt |
| | ctaaagtcgttgagaggaggagtcgtcagaccagatagctttgatgtcctgatcggaaggatcgttggcc |
| | cc |
| 198 | CCTGCAGGCAGCTGTTAATTAA |
| 199 | CTGCGCGCTCGCTCGCTCACTGAGGCCGCCCGGGCAAAGCCCGGGCGTCGGGCGACCTTTGGTCGCCCGG |
| | CCTCAGTGAGCGAGCGAGCGCGCAGAGAGGGAGTGGCCAACTCCATCACTAGGGGTTCCT |
| 200 | TGTAGTTAATGATTAACCCGCCATGCTACTTATCTACGGCGCGCCacgcgt |
| 201 | Cctctgctaaccatgttcatgccttcttctttttcctacag |
| 202 | Ctcctgggcaacgtgctggttattgtgctgtctcatcattttggcaaagaattgattaattcgagcgaac |
| | gcgtcgagtcgctcggtacgatttaaattgaattccttaagctatcatagga |
| 203 | gtgatagcttggtaccgagctcgatccaattgcaatgatcatcatgacagatctgcgcgcgatcgatatc |
| | agcgctttaaatttgcgcatgcagctatagttctagagggccctattctatagtgtcacctaaatgctag |
| | agctcgctgatcagcctcga |
| 204 | ctgtgccttctagttgccagccatctgttgtttgcccctcccccgtgccttccttgaccctggaaggtgc |
| | cactcccactgtcctttcctaataaaatgaggaaattgcatcgcattgtctgagtaggtgtcattctatt |
| | ctggggggtggggtggggcaggacagcaagggggaggattgggaagacaatagcaggcatgctggggatg |
| | cggtgggctctatgg |
| 205 | gtttaaacGCGGCCGCGTAGATAAGTAGCATGGCGGGTTAATCATTAACTACA |
| 206 | AGGAACCCCTAGTGATGGAGTTGGCCACTCCCTCTCTGCGCGCTCGCTCGCTCACTGAGGCCGGGCGACC |
| | AAAGGTCGCCCGACGCCCGGGCTTTGCCCGGGCGGCCTCAGTGAGCGAGCGAGCGCGCAG |
| 207 | TTAATTAAGGCGCC |
| 208 | ctaggccgacccttagactctgtactcagttctataaacgagccattggatacgagatccgtagattga |
| | aagggacacggaatatccccggacgcaatagacaccggtggacagcttggtatcctgagcacagtcgcgc |
| | gtccgaatctagctctactttagaggccccggattctgatggtcgtagaccgcagaaccgattgggggga |
| | tg |
| 209 | agatctactagt |
| 210 | tatcagcacacaattgcccattatacgc |

### Synthetic CNS-Specific Expression Cassettes

The present invention also provides a synthetic CNS-specific expression cassette comprising a synthetic CNS-specific promoter of the present invention operably linked to a sequence encoding an expression product, suitably a gene (e.g. a transgene).

The gene typically encodes a desired gene expression product such as a polypeptide (protein) or RNA. The gene may be full length cDNA or a genomic DNA sequence, or any fragment, subunit or mutant thereof that has at least some desired biological activity.

Where the gene encodes a protein, it can be essentially any type of protein. By way of non-limiting example, the protein may be an enzyme, an antibody (e.g. a monoclonal antibody), an antibody fragment, a viral protein (e.g. REP-CAP, REV, VSV-G or RD 114), a therapeutic protein (e.g. FVIII), or a toxic protein (e.g. Caspase 3, 8 or 9).

Various expression products suitable for treating the above conditions have been described in the art. Suitably, the nucleic acid encoding an expression product operably linked to the CRE, minimal/proximal promoter or promoter according to the invention may be one of the genes selected from the group consisting of: NPC1, EAAT2, NPY, CYP46A1, GLB1, APOE (e.g. ApoE2, ApoE3 or ApoE4), HEX, CLN1, CLN2, CLN3, CLN4, CLN5, CLN6, SUMF1, DCTN1, PRPH, SOD1, NEFH, GBA, IDUA, NAGLU, GUSB, ARSA, MANB, AADC, GDNF, NTN, ASP, MECP2, PTCHD1, GIB1, UBE3A, HEXA, MOG or SLC6A3. Additionally, or alternatively, expression product operably linked to the CRE, minimal/proximal promoter or promoter according to the invention may the miRNA/CRISPR Cas9 directed to the disease allele.

In some embodiments, useful expression products include dystrophins (including micro-dystrophins), beta 1,4-n-acetylgalactosamine galactosyltransferase (GALGT2), carbamoyl synthetase I, alpha-1 antitrypsin, ornithine transcarbamylase, arginosuccinate synthetase, arginosuccinate lyase, arginase, fumarylacetacetate hydrolase, phenylalanine hydroxylase, glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) and a dopaminergic transporter (hDAT).

Still other useful expression products include enzymes useful in enzyme replacement therapy, and which are useful in a variety of conditions resulting from deficient activity of enzyme. For example, enzymes containing mannose-6-phosphate may be utilized in therapies for lysosomal storage diseases (e.g., a suitable gene includes that encoding β-glucuronidase (GUSB)).

In some embodiments, exemplary polypeptide expression products include neuroprotective polypeptides and anti-angiogenic polypeptides. Suitable polypeptides include, but are not limited to, glial derived neurotrophic factor (GDNF), fibroblast growth factor 2 (FGF-2), nurturin, ciliary neurotrophic factor (CNTF), nerve growth factor (NGF; e.g., nerve growth factor-. beta.), brain derived neurotrophic factor (BDNF), neurotrophin-3 (NT-3), neurotrophin-4 (NT-4), neurotrophin-6 (NT-6), epidermal growth factor (EGF), pigment epithelium derived factor (PEDF), a Wnt polypeptide, soluble Fit-1, angiostatin, endostatin, VEGF, an anti-VEGF antibody, a soluble VEGFR, Factor VIII (FVIII), Factor IX (FIX), and a member of the hedgehog family (sonic hedgehog, Indian hedgehog, and desert hedgehog, etc.).

In some embodiments, useful therapeutic expression product include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor alpha superfamily, including TGFa., activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

In some embodiments, useful expression products include proteins that regulate the immune system including, without limitation, cytokines and lymphokines such as thrombopoietin (TPO), interleukins (IL) IL-1 through IL-25 (including IL-2, IL-4, IL-12 and IL-18), monocyte chemoattractant protein, leukemia inhibitory factor, granulocyte-macrophage colony stimulating factor, Fas ligand, tumor necrosis factors alpha and beta., interferons (alpha, beta, and gamma), stem cell factor, flk-2/flt3 ligand. Gene products produced by the immune system are also useful in the present invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

In some embodiments, useful expression product includes any one of the receptors for the hormones, growth factors, cytokines, lymphokines, regulatory proteins and immune system proteins. Useful heterologous nucleic acid sequences also include receptors for cholesterol regulation and/or lipid modulation, including the low-density lipoprotein (LDL) receptor, high density lipoprotein (HDL) receptor, the very low density lipoprotein (VLDL) receptor, and scavenger receptors. The invention also encompasses the use of gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP-2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

In some embodiments, useful expression products include non-naturally occurring polypeptides, such as chimeric or hybrid polypeptides having a non-naturally occurring amino acid sequence containing insertions, deletions or amino acid substitutions.

Further suitable expression products include micro RNA (miRNA), interfering RNA, antisense RNA, ribozymes, and aptamers.

In some embodiments of the invention, the synthetic CNS-specific expression cassette comprises a gene useful for gene editing, e.g. a gene encoding a site-specific nuclease, such as a meganuclease, zinc finger nuclease (ZFN), transcription activator-like effector-based nuclease (TALEN), or the clustered regularly interspaced short palindromic repeats system (CRISPR-Cas). Suitably the site-specific nuclease is adapted to edit a desired target genomic locus by making a cut (typically a site-specific double-strand break) which is then repaired via non-homologous end-joining (NHEJ) or homology dependent repair (HDR), resulting in a desired edit. The edit can be the partial or complete repair of a gene that is dysfunctional, or the knock-down or knock-out of a functional gene. Alternatively, the edit can be via base editing or prime editing, using suitable systems which are known in the art.

Suitably the synthetic CNS-specific expression cassette comprises sequences providing or coding for one or more of, and preferably all of, a ribosomal binding site, a start codon, a stop codon, and a transcription termination sequence. Suitably the expression cassette comprises a nucleic acid encoding a post-transcriptional regulatory element. Suitably the expression cassette comprises a nucleic acid encoding a polyA element.

### Modified Capsids

In one embodiment, the capsid described herein is further modified to increase tropism for the CNS. Provided herein is a composition comprising a modified viral capsid comprising a payload, wherein the payload comprises a regulatory sequence and a nucleic acid sequence flanked by inverted terminal repeats (ITRs) that target a central nervous system disorder, and wherein the modification is a chemical, non-chemical or amino acid modification. In some embodiments, the nucleic acid sequence of the payload comprises (a) an isolated nucleic acid encoding a transgene encoding one or more miRNAs; and (b) an isolated nucleic acid encoding a CYP46A1 protein. In some embodiments, the nucleic acid sequence of the payload comprises an isolated nucleic acid encoding a transgene encoding one or more miRNAs. In some embodiments, the nucleic acid sequence of the payload comprises an isolated nucleic acid encoding a CYP46A1 protein.

Further provided herein is a composition comprising (a) a first modified viral capsid comprising a first payload, and (b) at least a second modified viral capsid comprising a second payload, wherein the payload comprises a regulatory sequence and a nucleic acid sequence flanked by inverted terminal repeats (ITRs) that target a central nervous system disorder, wherein the first and at least second modified viral capsids are the same, and the first and second payloads are different, and wherein the modification is a chemical, non-chemical or amino acid modification. In some embodiments, the nucleic acid sequence of the first or second payload comprises an isolated nucleic acid encoding a transgene encoding one or more miRNAs. In some embodiments, the nucleic acid sequence of the first or second payload comprises an isolated nucleic acid encoding a CYP46A1 protein.

Further provided herein is a composition comprising (a) a first modified capsid comprising a first payload, and (b) at least a second modified capsid comprising a second payload, wherein the payload comprises a regulatory sequence and a nucleic acid sequence flanked by inverted terminal repeats (ITRs) that target a central nervous system disorder, wherein the first and at least second modified capsids are different, and the first and second payloads can be the same or different, and wherein the modification is a chemical, non-chemical or amino acid modification. In some embodiments, the nucleic acid sequence of the first or second payload comprises an isolated nucleic acid encoding a transgene encoding one or more miRNAs. In some embodiments, the nucleic acid sequence of the first or second payload comprises an isolated nucleic acid encoding a CYP46A1 protein.

Another aspect described herein provides a composition or combination comprising at least one of (a) an isolated nucleic acid encoding a transgene encoding one or more miRNAs; and (b) an isolated nucleic acid encoding a CYP46A1 protein. In one aspect, described herein is a composition or combination comprising: (a) a recombinant viral vector comprising an isolated nucleic acid comprising (i) a first region comprising a first adeno-associated virus (AAV) inverted terminal repeat (ITR), or a variant thereof, and (ii) a second region comprising a transgene encoding one or more miRNAs; and (b) a recombinant viral vector comprising an isolated nucleic acid encoding the CYP46A1 protein.

Another aspect described herein provides a composition or combination comprising (a) an isolated nucleic acid encoding a transgene encoding an isolated nucleic acid encoding a CYP46A1 protein. In some aspects, the composition or combination further comprises (b) an isolated nucleic acid encoding a transgene encoding one or more miRNAs. In other aspects, the composition or combination does not comprise (b) an isolated nucleic acid encoding a transgene encoding one or more miRNAs. In one aspect, described herein is a composition or combination comprising: (a) a recombinant viral vector comprising an isolated nucleic acid comprising (i) a first region comprising a first adeno-associated virus (AAV) inverted terminal repeat (ITR), or a variant thereof, and (ii) a second region comprising a transgene encoding a CYP46A1 protein. In some aspects, the composition or combination further comprises: (b) a recombinant viral vector comprising an isolated nucleic acid encoding one or more miRNAs. In other aspects, the composition or combination does not comprise (b) a recombinant viral vector comprising an isolated nucleic acid encoding one or more miRNAs.

Another aspect described herein provides a composition or combination comprising (a) an isolated nucleic acid encoding a transgene encoding an isolated nucleic acid encoding one or more miRNAs. In some aspects, the composition or combination further comprises (b) an isolated nucleic acid encoding a transgene encoding a CYP46A1 protein. In other aspects, the composition or combination does not comprise (b) an isolated nucleic acid encoding a transgene encoding a CYP46A1 protein. In one aspect, described herein is a composition or combination comprising: (a) a recombinant viral vector comprising an isolated nucleic acid comprising (i) a first region comprising a first adeno-associated virus (AAV) inverted terminal repeat (ITR), or a variant thereof, and (ii) a second region comprising a transgene encoding one or more miRNAs. In some aspects, the composition or combination further comprises: (b) a recombinant viral vector comprising an isolated nucleic acid encoding a CYP46A1 protein. In other aspects, the composition or combination does not comprise (b) a recombinant viral vector comprising an isolated nucleic acid encoding a CYP46A1 protein.

In certain embodiments, the modified viral capsid comprises modification that results in its preferential targeting of the CNS or PNS. For example, the modified viral capsid has increased tropism for the CNS, and/or decreased tropism for at least a second location, e.g., the liver. Preferential targeting of the CNS does not exclude targeting to other sites, but rather indicates that it is more highly targeted to the CNS as compared to another site.

In one embodiment, the modified viral capsid comprises modification that results in its targeting of the CNS or PNS. For example, a modification to a capsid that typically targets a non-CNS site (e.g., the liver) can redirect the capsid to now target both the CNS and the non-CNS site. In such embodiment, the CNS-targeting does not need to be preferential.

In one embodiment, the modification to the capsid is an amino acid modification, e.g., an amino acid deletion, insertion, or substitute. In one embodiment, the amino acid modification increases tropism for the CNS or PNS. In one embodiment, the amino acid modification targets the modified capsid to the CNS or PNS.

In one embodiment, the modified viral capsid has or consists of, or consists essentially of a nucleic acid sequence that is 90% identical to SEQ ID NOs 1-4 of U.S. patent application Ser. No. 16/511,913, the contents of which are incorporated herein by references in its entirety. This US Patent application describes chimeric AAV capsid sequences that exhibit a dominant tropism for oligodendrocytes, and can be used to create AAV vectors that transduce oligodendrocytes in the CNS of subject.

In one embodiment, the modified viral capsid is an AAV capsid protein comprising one or more amino acids substitutions, wherein the substitutions introduce a new glycan binding site into the AAV capsid protein. In some embodiments, the amino acid substitutions are in amino acid 266, amino acids 463-475 and amino acids 499-502 in AAV2 or the corresponding amino acid positions in AAV1, AAV3, AAV4, AAV5, AAV6, AAV7, AAV8 or AAV10. Such AAV capsid protein is further described in, e.g., U.S. patent application Ser. No. 16/110,773; the contents of which are incorporated herein by references in its entirety.

In one embodiment, the modified viral capsid is an AAV capsid protein that comprises, consists of, or consists essentially of an AAV 2.5 capsid protein (SEQ ID NO: 1 of International Patent Application No. PCT/US2020/029493; the contents of which are incorporated herein by references in its entirety) comprising one or more amino acid substitutions that introduce a new glycan binding site. Such amino acid substitutions can target the capsid to neurons and glial cells, such as astrocytes. In embodiments of the capsid proteins, capsids, viral vectors and methods described in the International Patent Application No. PCT/US2020/029493, the one or more amino acid substitutions comprise A267S, SQAGASDIRDQSR464-476SX₁AGX₂SX₃X₄X₅X₆QX₇R (SEQ ID NOS 153 and 154, respectively), wherein X₁₋₇ can be any amino acid, and EYSW 500-503 (SEQ ID NO: 155) EX₈X₉W, wherein X₈₋₉ can be any amino acid. In embodiments of the capsid proteins, capsids, viral vectors and methods described herein, X₁ is V or a conservative substitution thereof, X₂ is P or a conservative substitution thereof, X₃ is N or a conservative substitution thereof, X₄ is M or a conservative substitution thereof, X₅ is A or a conservative substitution thereof; X₆ is V or a conservative substitution thereof, X₇ is G or a conservative substitution thereof, X₈ is F or a conservative substitution thereof, and/or X₉ is A or a conservative substitution thereof. In embodiments of the capsid proteins, capsids, viral vectors and methods described herein, X₁ is V, X₂ is P, X₃ is N, X₄ is M, X₅ is A, X₆ is V, X₇ is G, X₈ is F, and X₉ is A, wherein the new glycan binding site is a galactose binding site. Such AAV capsid protein is further described in, e.g., International Patent Application No. PCT/US2020/029493; the contents of which are incorporated herein by references in its entirety.

In one embodiment, the modified viral capsid is an AAV capsid protein particle comprising a surface-bound peptide, wherein the peptide bound to the surface of the AAV particle is Angiopep-2, GSH, HIV-1 TAT (48-60), ApoE (159-167)2, Leptin 30 (61-90), THR, PB5-3, PB5-5, PB5-14, or any combination thereof, as described in, e.g., U.S. patent application Ser. No. 16/956,306; the contents of which are incorporated herein by references in its entirety. Such AAV capsid permits delivery, e.g., of a payload, across the blood brain barrier.

In one embodiment, the modified viral capsid is AAV capsid protein (e.g., an AAV1, AAV5, or AAV6 capsid protein), wherein the VP3 region of the capsid protein comprises modifications (e.g., replacement of a tyrosine residue with a non-tyrosine residue and/or a threonine residue with a non-threonine residue) at positions corresponding to: one or more of, or each of Y705, Y731, and T492 of a wild-type AAV1 capsid protein (e.g., SEQ ID NO: 1 of U.S. patent application Ser. No. 16/565,191; the contents of which are incorporated herein by references in its entirety); one or more of, or each of Y436, Y693, and Y719 of a wild-type AAV5 capsid protein (e.g., SEQ ID NO: 2 of U.S. patent application Ser. No. 16/565,191); or one or more of, or each of Y705, Y731, and T492 of a wild-type AAV6 capsid protein (e.g., SEQ ID NO: 3 of U.S. patent application Ser. No. 16/565,191). Such AAV capsids target neurons and astrocytes.

In one embodiment, the modified viral capsid is AAV capsid protein (e.g., an AAV1, AAV5, or AAV6 capsid protein) comprising Y to F (tyrosine to phenylalanine) modifications or T to V (threonine to valine) modifications in the VP3 region of the capsid at positions corresponding to: one or more of or each of Y705F, Y731F, and T492V of a wild-type AAV1 capsid protein (e.g., SEQ ID NO: 1 of U.S. patent application Ser. No. 16/565,191); one or more of or each of Y436F, Y693F, and Y719F of a wild-type AAV5 capsid protein (e.g., SEQ ID NO: 2 of U.S. patent application Ser. No. 16/565,191); or one or more of or each of Y705F, Y731F, and T492V of a wild-type AAV6 capsid protein (e.g., SEQ ID NO: 3 of U.S. patent application Ser. No. 16/565,191). Such AAV capsids target neurons and astrocytes.

In one embodiment, the modified viral capsid is AAV capsid protein (e.g., an AAV1, AAV5, or AAV6 capsid protein), wherein a VP3 region of the capsid protein comprises modifications (e.g., replacement of a tyrosine residue with a non-tyrosine residue and/or a threonine residue with a non-threonine residue) at positions corresponding to: one or more of or each of Y705, Y731, and T492 of a wild-type AAV1 capsid protein (e.g., SEQ ID NO: 1 of U.S. patent application Ser. No. 16/565,191); one or more of or each of Y436, Y693, and Y719 of a wild-type AAV5 capsid protein (e.g., SEQ ID NO: 2 of U.S. patent application Ser. No. 16/565,191); or one or more of or each of Y705, Y731, and T492 of a wild-type AAV6 capsid protein (e.g., SEQ ID NO: 3 of U.S. patent application Ser. No. 16/565,191). Such AAV capsids target neurons and astrocytes.

In one embodiment, the modified viral capsid is AAV capsid protein (e.g., an AAV1, AAV5, or AAV6 capsid protein) comprising Y to F (tyrosine to phenylalanine) modifications or T to V (threonine to valine) modifications in the VP3 region of the capsid protein at positions corresponding to: one or more of or each of Y705F, Y731F, and T492V of a wild-type AAV1 capsid protein (e.g., SEQ ID NO: 1 of U.S. patent application Ser. No. 16/565,191); one or more of or each of Y436F, Y693F, and Y719F of a wild-type AAV5 capsid protein (e.g., SEQ ID NO: 2 of U.S. patent application Ser. No. 16/565,191); or one or more of or each of Y705F, Y731F, and T492V of a wild-type AAV6 capsid protein (e.g., SEQ ID NO: 3 of U.S. patent application Ser. No. 16/565,191). Such AAV capsids target neurons and astrocytes.

In one embodiment, the amino acid modification permits the modified capsid to evade neutralizing antibodies, for example, that are generated against a viral vector, e.g., of the same serotype. In one embodiment, the amino acid modification permits the modified capsid to be used for repeat administration, for example, the modification will enable the capsid to have a therapeutic effect upon re-administration.

In one embodiment, the modified viral capsid is a chimeric capsid. A “chimeric” capsid protein as used herein means an AAV capsid protein (e.g., any one or more of VP1, VP2 or VP3) that has been modified by substitutions in one or more (e.g., 2, 3, 4, 5, 6, 7, 8, 9, 10, etc.) amino acid residues in the amino acid sequence of the capsid protein relative to wild type, as well as insertions and/or deletions of one or more (e.g., 2, 3, 4, 5, 6, 7, 8, 9, 10, etc.) amino acid residues in the amino acid sequence relative to wild type. In some embodiments, complete or partial domains, functional regions, epitopes, etc., from one AAV serotype can replace the corresponding wild type domain, functional region, epitope, etc. of a different AAV serotype, in any combination, to produce a chimeric capsid protein of this invention. Production of a chimeric capsid protein can be carried out according to protocols well known in the art and a significant number of chimeric capsid proteins are described in the literature as well as herein that can be included in the capsid of this invention.

In one embodiment, the modified viral capsid is a haploid capsid. As used herein, the term “haploid AAV” shall mean that AAV as described in International Application WO2018/170310, or US Application US2018/037149, which are incorporated herein in their entirety by reference. In some embodiments, a population of virions is a haploid AAV population where a virion particle can be constructed wherein at least one viral protein from the group consisting of AAV capsid proteins, VP1, VP2 and VP3, is different from at least one of the other viral proteins, required to form the virion particle capable of encapsulating an AAV genome. For each viral protein present (VP1, VP2, and/or VP3), that protein is the same type (e.g., all AAV2 VP1). In one instance, at least one of the viral proteins is a chimeric viral protein and at least one of the other two viral proteins is not a chimeric. In one embodiment VP1 and VP2 are chimeric and only VP3 is non-chimeric. For example, only the viral particle composed of VP1/VP2 from the chimeric AAV2/8 (the N-terminus of AAV2 and the C-terminus of AAV8) paired with only VP3 from AAV2; or only the chimeric VP1/VP2 28m-2P3 (the N-terminal from AAV8 and the C-terminal from AAV2 without mutation of VP3 start codon) paired with only VP3 from AAV2. In another embodiment only VP3 is chimeric and VP1 and VP2 are non-chimeric. In another embodiment at least one of the viral proteins is from a completely different serotype. For example, only the chimeric VP1/VP2 28m-2P3 paired with VP3 from only AAV3. In another example, no chimeric protein is present.

In some embodiments of the technology described herein, a modified viral capsid comprises one or more modifications, e.g., a chemical modification, a non-chemical modification, or an amino acid modification to the capsid. Such modifications can, for example, modify the tissue-type tropism or cell-type tropism of the modified capsid, among other things.

Modifications can alter the properties of the capsid, including biochemical properties such as receptor binding, directly, such that the modification itself alters the behavior of the capsid, or can permit further modification, such as the attachment of a ligand which in turn modifies behavior of the capsid in a desired manner.

In one embodiment, chemical modification of cysteine residues, which may be naturally present or introduced by genetic modification of a capsid polypeptide coding sequence, permits the covalent attachment of a ligand via disulfide bond formation (see, e.g., WO 2005/106046, the contents of which are incorporated herein by reference).

Various ligands are contemplated, including but not limited to antibodies or antigen-binding fragments thereof that, for example, target a cell-surface protein expressed by a target cell (see, e.g., WO 2000/002654, which is incorporated herein by reference).

WO2015/062516, the contents of which are also incorporated herein by reference, describes the insertion of an amino acid comprising an azido group by genetic modification of the capsid gene, followed by chemical conjugation of a ligand via the azido group.

The modification of AAV capsid tropism by glycation, or chemical conjugation of sugar moieties, is described by Horowitz et al., Bioconjugate Chem. 22: 529-532 (2011). That approach, and similar approaches are contemplated for modification of capsids as described herein.

In other embodiments, the coating of a viral capsid with a polymer, such as polyethylene glycol (PEG) or poly-(N-hydroxypropyl)methacrylamide (pHPMA) is specifically contemplated. Such modification can, for example, reduce specific and nonspecific interactions with non-target tissues.

In other embodiments, carbodiimide coupling is specifically contemplated. See, e.g., Joo et al. ACS Nano 5, titled “Enhanced Real-time Monitoring of Adeno-Associated Virus Trafficking by Virus-Quantum Dot Conjugates” (2011).

In other embodiments, the viral capsid can be modified, e.g., as described in WO 2017/212019, see also U.S. National Phase U.S. Ser. No. 16/308,740, the contents of which are each incorporated herein by reference. The approach described therein couples a viral capsid to a ligand via bonds comprising —CSNH— and an aromatic moiety. While genetically modified viral capsids can be further modified by this approach, the modifications described therein do not require genetic modification of the viral capsid. Ligands described therein include, for example, a targeting agent, a steric shielding agent for avoiding neutralizing antibody interactions, a labeling agent or a magnetic agent. Targeting ligands described therein include, for example, a cell-type specific ligand, a protein, a mono- or polysaccharide, a steroid hormone, an RGD motif peptide (e.g., Arg-Gly-Asp, a cell adhesion motif which can mimic cell adhesion proteins and bind to integrins), a vitamin, and a small molecule.

In one embodiment, the chemical modification of the invention is a modification described in International patent application PCT/EP2017/064089, the content of which is incorporated herein by reference in its entirety.

In one embodiment, the chemical modification of the invention is a modification described in International patent application PCT/EP2020/069554, the content of which is incorporated herein by reference in its entirety.

In one embodiment, the capsid has at least one chemically-modified tyrosine residue in its capsid, wherein said chemically-modified tyrosine residue is of formula (I):

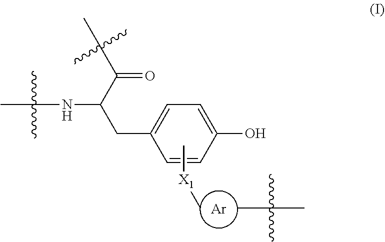

wherein:
X1 is selected from the group consisting of

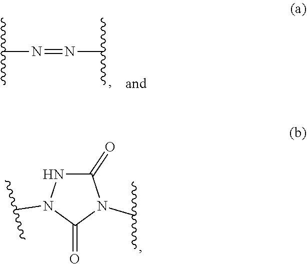

and
Ar is an aryl or a heteroaryl moiety optionally substituted.

In one embodiment, the capsid has at least one chemically-modified tyrosine residue is of formula (Ia):

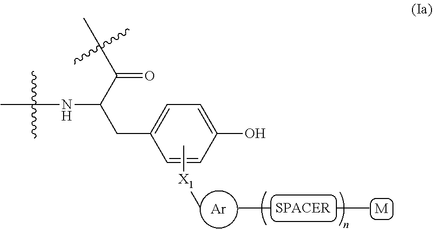

wherein:
Xi, and Ar are as defined herein above,
Spacer is a group for linking the “Ar” group to the functional moiety “M” which preferably comprises up to 1000 carbon atoms and which is preferably in the form of a chemical chain which optionally comprises heteroatoms and/or cyclic moieties,
n is 0 or 1; and
M is a functional moiety comprising a steric agent, a labelling agent, cell-types specific ligand or a drug moiety.

In one embodiment, Xi is of formula (a) and/or “Ar” is selected from substituted or unsubstituted phenyl, pyridyl, naphthyl, and anthracenyl.

In one embodiment, the capsid has at least one chemically-modified tyrosine is of formula (Ic):

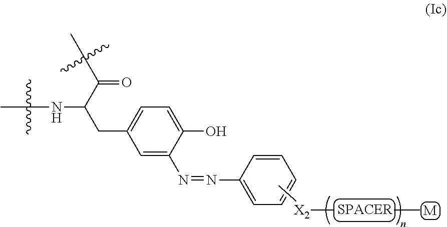

wherein:
X2 is —C(═O)—NH, —C(═O)—O, —C(═O)—O—C(═O)—, O—(C═O)—, NH—C(═O)—, NH—C(═O)—NH, —O—C═O—O—, O, NH, —NH(C═S)—, or —(C=S)—NH—, preferably —(C=O)—NH—or —(C=O)—O—
X2 is at position para, meta or ortho, preferably at position para of the phenyl group,
Spacer, n and M are as defined herein above.

In one embodiment, “Spacer”, when present, is selected from the group consisting of saturated or unsaturated, linear or branched C2-C40 hydrocarbon chains, optionally substituted, polyethylene glycol, polypropylene glycol, pHPMA (polymer of N-(2-Hydroxypropyl)methacrylamide), Poly Lactic-co-Glycolic Acid (PLGA), polymers of alkyl diamines and combinations thereof, and/or

“M” comprises, or consists of, cell-type targeting ligand, preferably selected from a mono- or a polysaccharide, a hormone, including a steroid hormone, a peptide such as RGD peptide (e.g., Arg-Gly-Asp, a cell adhesion motif which can mimic cell adhesion proteins and bind to integrins), a muscle targeting peptide (MTP) or Angiopep-2, a protein or a fragment thereof, a membrane receptor or a fragment thereof, an aptamer, an antibody including heavy-chain antibody, and fragments thereof such as antigen-binding fragment (Fab), Fab′ (which is the antigen-binding fragment further comprising a free sulfhydryl group), and VHH, a single-chain fragment variable (ScFv), a spiegelmer, a peptide aptamer, vitamins and drugs such as Cannabinoid receptor 1 (CB1) and/or Cannabinoid receptor 2 (CB2) ligands.

In one embodiment, “Spacer” (when present) is selected from the group consisting of linear or branched C2-C20 alkyl chains, polyethylene glycol, polypropylene glycol, pHPMA, PLGA, polymer of alkyl diamine and combinations thereof, said polymers having from 2 to 20 monomers and/or “M” comprises, or consists of, a cell-type specific ligand derived from a protein selected from transferrin, Epidermal Growth Factor (EGF), and basic Fibroblast Growth Factor 13FGF, a mono- or a polysaccharide comprising one or several galactose, mannose, N-acetylgalactosamine residues, bridge GalNac, or mannose-6-phosphate, MTP selected from SEQ ID NO:1 to SEQ ID NO:7, and vitamins such as folic acid.

In one embodiment, the capsid further has at least one additional chemically modified amino acid residue in the capsid, which is different from a tyrosine residue, said amino acid residue preferably bearing an amino group chemically modified with a group of formula (V):

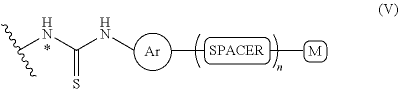

wherein:
N* being the nitrogen of the amino group of an amino acid residue, e.g. of a lysine residue or arginine residue, and
Ar, Spacer, n and M has the same definition as Ar, Spacer, n and M of formula (II) of claim 2.

In one embodiment, the capsid is incubated a chemical reagent bearing a reactive group selected from an aryl diazonium, and a 4-phenyl-1,2,4-triazole-3,5-dione (PTAD) moiety in conditions conducive for reacting said reactive group with a tyrosine residue present in the capsid so as to form a covalent bound.

In one embodiment, the capsid is incubated with a chemical reagent of formula VId to obtain the at least one chemically-modified tyrosine residue in the capsid of formula Ic.

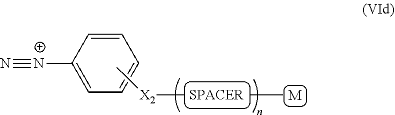

### Pharmaceutical Compositions

The expression cassettes, vectors or virions of the present invention may be formulated in a pharmaceutical composition with a pharmaceutically acceptable excipient i.e. one or more pharmaceutically acceptable carrier substances and/or additives, e.g. buffers, carriers, excipients, stabilizers, etc. The pharmaceutical composition may be provided in the form of a kit.

Accordingly, a further aspect of the present invention provides a pharmaceutical composition comprising an expression cassette, a vector or virion as described herein.

In various aspects, the pharmaceutical composition comprises a phosphate buffer. The phosphate buffer comprises from about 1 mM to about 50 mM phosphate, such as phosphate at a concentration of about 1 mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM, or about 50 mM. The phosphate is prepared from a combination of dibasic phosphate (e.g., Na₂HPO₄, K₂HPO₄) and monobasic phosphate (e.g., NaH₂PO₄, KH₂PO₄) at a dibasic phosphate: monobasic phosphate molar ratio of from about 1:10 to about 10:1. For example, in various exemplary embodiments, the 10 mM phosphate comprises 9.5 mM dibasic phosphate and 0.5 mM monobasic phosphate, 9 mM dibasic phosphate and 1 mM monobasic phosphate, 8.5 mM dibasic phosphate and 1.5 mM monobasic phosphate, or 8 mM dibasic phosphate and 2 mM monobasic phosphate. The pH of the phosphate buffer is from about 6.5 to about 7.5, such as a pH of 6.5, 6.6, 6.7, 6.8, 6.9, 6.95, 7.0, 7.05, 7.1, 7.2, 7.3, 7.4, or 7.5. The phosphate buffer can also include NaCl at a concentration of from about 50 mM to about 200 mM, such as at a concentration of about 50 mM, about 60 mM, about 70 mM, about 80 mM, about 90 mM, about 100 mM, about 110 mM, about 120 mM, about 130 mM, about 135 mM, about 136 mM, about 137 mM, about 138 mM, about 139 mM, about 140 mM, about 150 mM, about 160 mM, about 170 mM, about 180 mM, about 190 mM, or about 200 mM. The phosphate buffer can also include KCl at a concentration of from about 0.5 mM to about 10 mM, such as at a concentration of about 0.5 mM, about 0.6 mM, about 0.7 mM, about 0.8 mM, about 0.9 mM, about 1 mM, about 2 mM, about 2.5 mM, about 2.6 mM, about 2.7 mM, about 2.8 mM, about 2.9 mM, about 3 mM, about 4 mM, about 5 mM, about 6 mM, about 7 mM, about 8 mM, about 9 mM, or about 10 mM. The phosphate buffer can also include CaCl₂ at a concentration of from about 0.20 mM to about 10 mM, such as at a concentration of about 0.2 mM, about 0.3 mM, about 0.4 mM, about 0.5 mM, about 0.6 mM, about 0.7 mM, about 0.8 mM, about 0.81 mM, about 0.82 mM, about 0.83 mM, about 0.84 mM, about 0.85 mM, about 0.86 mM, about 0.87 mM, about 0.88 mM, about 0.89 mM, about 0.9 mM, about 1 mM, about 2 mM, about 3 mM, about 4 mM, about 5 mM, about 6 mM, about 7 mM, about 8 mM, about 9 mM, or about 10 mM. The phosphate buffer can also include MgCl₂ at a concentration of from about 0.10 mM to about 1 mM, such as at a concentration of about 0.1 mM, about 0.2 mM, about 0.3 mM, about 0.4 mM, 0.41 mM, 0.42 mM, 0.43 mM, 0.44 mM, 0.45 mM, 0.46 mM, 0.47 mM, 0.48 mM, 0.49 mM, about 0.5 mM, about 0.6 mM, about 0.7 mM, about 0.8 mM, about 0.9 mM, or about 1 mM. The phosphate buffer can also include Poloxamer 188 (e.g., Pluronic™ F-68 non-ionic surfactant) at a concentration of from about 0.0001 wt. % to about 0.005 wt. %, such as at a concentration of about 0.0001 wt. %, about 0.0002 wt. %, about 0.0003 wt. %, about 0.0004 wt. %, about 0.0005 wt. %, about 0.0006 wt. %, about 0.0007 wt. %, about 0.0008 wt. %, about 0.0009 wt. %, about 0.001 wt. %, about 0.0015 wt. %, about 0.002 wt. %, about 0.0025 wt. %, about 0.003 wt. %, about 0.0035 wt. %, about 0.004 wt. %, about 0.0045 wt. %, or about 0.005 wt. %. The phosphate buffer can also include sorbitol at a concentration of from about 0.005 wt. % to about 10 wt. %, such as at a concentration of about 0.005 wt. %, about 0.075 wt. %, about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 2 wt. %, about 3 wt. %, about 4 wt. %, about 5 wt. %, about 6 wt. %, about 7 wt. %, about 8 wt. %, about 9 wt. %, or about 10 wt. %. The rAAV comprising the nucleic acid (for example, AAVrh.10 comprising the CAG promoter and the nucleic acid comprising a sequence at least 80% identical, e.g., at least 85%, at least 90%, at least 95%, at least 98%, at least 99% or 100% identical, to SEQ ID NO: 194; AAVrh10.CAG.hCYP46A1) can have a titer in the phosphate buffer of from about 0.1×10⁹ vg/μl to about 5×10¹⁰ vg/μl, such as a titer of about 0.1×10⁹ vg/μl, about 0.2×10⁹ vg/μl, about 0.3×10⁹ vg/μl, about 0.4×10⁹ vg/μl, about 0.5×10⁹ vg/μl, about 0.6×10⁹ vg/μl, about 0.7×10⁹ vg/μl, about 0.8×10⁹ vg/μl, about 0.9×10⁹ vg/μl, about 1×10⁹ vg/μl, about 2×10⁹ vg/μl, about 3×10⁹ vg/μl, about 4×10⁹ vg/μl, about 5×10⁹ vg/μl, about 6×10⁹ vg/μl, about 7×10⁹ vg/μl, about 8×10⁹ vg/μl, about 9×10⁹ vg/μl, about 1×10¹⁰ vg/μl, about 1×10¹⁰ vg/μl, about 1×10¹⁰ vg/μl, about 4×10¹⁰ vg/μl, or about 5×10¹⁰ vg/μl.

In some embodiments, the pharmaceutical composition comprises 10 mM phosphate, 137 mM NaCl, 2.7 mM KCl, 0.88 mM CaCl₂, 0.49 mM MgCl₂, 0.001 wt. % Poloxamer 188, pH 7.2, and 0.4×10⁹ vg/μl AAVrh10.CAG.hCYP46A1.

In some embodiments, the pharmaceutical composition comprises 10 mM phosphate (9 mM Na₂HPO₄ and 1 mM KH₂PO₄), 137 mM NaCl, 2.7 mM KCl, 0.88 mM CaCl₂, 0.49 mM MgCl₂, 0.001 wt. % Poloxamer 188, pH 7.2, and 0.4×10⁹ vg/μl AAVrh10.CAG.hCYP46A1.

In some embodiments, the pharmaceutical composition comprises 10 mM phosphate, 137 mM NaCl, 2.7 mM KCl, 0.88 mM CaCl₂, 0.49 mM MgCl₂, 0.001 wt. % Poloxamer 188, pH 7.2, and 1.1×10⁹ vg/μl AAVrh10.CAG.hCYP46A1.

In some embodiments, the pharmaceutical composition comprises 10 mM phosphate (9 mM Na₂HPO₄ and 1 mM KH₂PO₄), 137 mM NaCl, 2.7 mM KCl, 0.88 mM CaCl₂, 0.49 mM MgCl₂, 0.001 wt. % Poloxamer 188, pH 7.2, and 1.1×10⁹ vg/μl AAVrh10.CAG.hCYP46A1.

In some embodiments, the pharmaceutical composition comprises 10 mM phosphate, 137 mM NaCl, 2.7 mM KCl, 0.88 mM CaCl₂, 0.49 mM MgCl₂, 0.001 wt. % Poloxamer 188, pH 7.2, and 3×10⁹ vg/μl AAVrh10.CAG.hCYP46A1.

In some embodiments, the pharmaceutical composition comprises 10 mM phosphate (9 mM Na₂HPO₄ and 1 mM KH₂PO₄), 137 mM NaCl, 2.7 mM KCl, 0.88 mM CaCl₂, 0.49 mM MgCl₂, 0.001 wt. % Poloxamer 188, pH 7.2, and 3×10⁹ vg/μl AAVrh10.CAG.hCYP46A1.

In some embodiments, the pharmaceutical composition comprises 10 mM phosphate, 137 mM NaCl, 2.7 mM KCl, 0.88 mM CaCl₂, 0.49 mM MgCl₂, 0.001 wt. % Poloxamer 188, pH 7.2, and 7×10⁹ vg/ul AAVrh10.CAG.hCYP46A1.

In some embodiments, the pharmaceutical composition comprises 10 mM phosphate (9 mM Na₂HPO₄ and 1 mM KH₂PO₄), 137 mM NaCl, 2.7 mM KCl, 0.88 mM CaCl₂, 0.49 mM MgCl₂, 0.001 wt. % Poloxamer 188, pH 7.2, and 7×10⁹ vg/μl AAVrh10.CAG.hCYP46A1.

In some embodiments, the pharmaceutical composition comprises 10 mM phosphate, 137 mM NaCl, 2.7 mM KCl, 0.88 mM CaCl₂, 0.49 mM MgCl₂, 0.001 wt. % Poloxamer 188, pH 7.2, and 8.2×10⁹ vg/μl AAVrh10.CAG.hCYP46A1.

In some embodiments, the pharmaceutical composition comprises 10 mM phosphate (9 mM Na₂HPO₄ and 1 mM KH₂PO₄), 137 mM NaCl, 2.7 mM KCl, 0.88 mM CaCl₂, 0.49 mM MgCl₂, 0.001 wt. % Poloxamer 188, pH 7.2, and 8.2×10⁹ vg/μl AAVrh10.CAG.hCYP46A1.

In some embodiments, the pharmaceutical composition comprises 10 mM phosphate, 137 mM NaCl, 2.7 mM KCl, 0.88 mM CaCl₂, 0.49 mM MgCl₂, 0.001 wt. % Poloxamer 188, pH 7.2, and 3×10¹⁰ vg/μl AAVrh10.CAG.hCYP46A1.

In some embodiments, the pharmaceutical composition comprises 10 mM phosphate (9 mM Na₂HPO₄ and 1 mM KH₂PO₄), 137 mM NaCl, 2.7 mM KCl, 0.88 mM CaCl₂, 0.49 mM MgCl₂, 0.001 wt. % Poloxamer 188, pH 7.2, and 3×10¹⁰ vg/μl AAVrh10.CAG.hCYP46A1.

In alternative embodiments, the pharmaceutical composition is equivalent to any of the above exemplified compositions, but with a phosphate concentration of about 9.53 mM comprising about 8.06 mM Na₂HPO₄ and about 1.47 mM KH₂PO₄.

In alternative embodiments, the pharmaceutical composition is equivalent to any of the above exemplified compositions, but with a sorbitol concentration of from about 0.005 wt. % to about 10 wt. %.

### Administration

The rAAVs of the disclosure may be delivered to a subject in compositions according to any appropriate methods known in the art. For example, an rAAV, preferably suspended in a physiologically compatible carrier (i.e., in a composition), may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus and/or putamen of the dorsal striatum), spinal cord and neuromuscular junction, or cerebellar lobule, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000). In some embodiments, rAAV as described in the disclosure are administered by intravenous injection. In some embodiments, the rAAV are administered by intracerebral injection. In some embodiments, the rAAV are administered by intrathecal injection. In some embodiments, the rAAV are administered by intrastriatal injection. In some embodiments, the rAAV are delivered by intracranial injection. In some embodiments, the rAAV are delivered by cisterna magna injection. In some embodiments, the rAAV are delivered by cerebral lateral ventricle injection.

In some embodiments, the rAAV are administered by a plurality of injections along a single trajectory. For example, a cannula can be positioned through the brain to a first position in a target structure (e.g., a putamen) and a first injection is made at the first position. The cannula is then extended or retracted along the same single trajectory to a second position within the target structure and a second injection is made at the second position. Further injections can be made at additional positions within the target structure by extending or retracting the cannula along the same single trajectory. Targeting structures of the brain can be performed by positioning a cannula through the brain at a trajectory guided MRI. The trajectory can be anterior, for example, through a frontal bone, or posterior, for example, through an occipital bone. In other embodiments, a plurality of trajectories are used to target a single tissue or structure.

Aspects of the instant disclosure relate to compositions comprising a recombinant AAV comprising a capsid protein and a nucleic acid encoding a transgene, wherein the transgene comprises a nucleic acid sequence encoding one or more miRNAs. In some embodiments, each miRNA comprises a sequence set forth in any one of SEQ ID NOs: 6-17, 40-44, 50-66, 158-185, or 217-260. In some embodiments, the nucleic acid further comprises AAV ITRs. In some embodiments, the ITR is an AAV1, AAV2, AAV3b, AAV4, AAV5, AAV6, AAV7, AAV8, AAV9, AAV10, AAVrh10, AAV11, AAV12, or AAV13 ITR. In some embodiments, a composition further comprises a pharmaceutically acceptable carrier. The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired. In some embodiments, all or, at least one of the nucleic acid sequences disclosed herein are delivered via non-viral DNA constructs comprising at least one DD-ITR. For example, the non-viral DNA constructs as described in WO 2019/246554 can be utilized to deliver one or more of the nucleic acids described herein. WO 2019/246554 is incorporated herein by reference in its entirety.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An effective amount of an rAAV is an amount sufficient to target infect an animal, target a desired tissue. In some embodiments, an effective amount of an rAAV is an amount sufficient to produce a stable somatic transgenic animal model. The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 10⁹ to 10¹⁶ genome copies. In some cases, a dosage between about 10¹¹ to 1013 rAAV genome copies is appropriate. In certain embodiments, 10¹² or 1013 rAAV genome copies is effective to target CNS tissue. In some cases, stable transgenic animals are produced by multiple doses of an rAAV.

In other cases, a dose administered is dependent on an rAAV titer and a volume of a tissue to be injected. For example, the rAAV (or rAAV composition) may have a titer of from about 0.1×10⁹ vg/μL to about 1.5×10⁹ vg/μL, such as a titer of about 0.1×10⁹ vg/μL, about 0.2×10⁹ vg/μL, about 0.3×10⁹ vg/μL, about 0.4×10⁹ vg/μL, about 0.5×10⁹ vg/μL, about 0.6×10⁹ vg/μL, about 0.7×10⁹ vg/μL, about 0.8×10⁹ vg/μL, about 0.9×10⁹ vg/μL, about 1×10⁹ vg/μL, about 1.1×10⁹ vg/μL, about 1.2×10⁹ vg/μL, about 1.3×10⁹ vg/μL, about 1.4×10⁹ vg/μL, or about 2.5×10⁹ vg/μL. The amount of the titer to be injected may independently depend on a volume of the at least one target tissue or structure as determined by MRI. For example, the volume of a tissue to be injected can be from about 1% to about 90% of the total volume of the tissue or structure, such as a tissue or structure volume of about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90%. Similarly, the volume of the tissue or structure to be injected can be at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or more of the tissue's or structure's volume. When more than one tissue or structure is targeted (i.e., injected), the volume of each tissue or structure to be injected may the same or different. For example, in some embodiments, a titer is injected into the left and right putamen at independent volumes equivalent to, for example, about 25% of the total volume of each putamen. In other embodiments, a titer is injected into the left and right caudate at independent volumes equivalent to, for example, about 25% of the total volume of each caudate. In yet other embodiments, both the left and right putamens and both the left and right caudates receive injections of the titer, for example, about 25%, based on the total volumetric size of each putamen and caudate.

In some embodiments, a dose of rAAV is administered to a subject no more than once per calendar day (e.g., a 24-hour period). In some embodiments, a dose of rAAV is administered to a subject no more than once per 2, 3, 4, 5, 6, or 7 calendar days. In some embodiments, a dose of rAAV is administered to a subject no more than once per calendar week (e.g., 7 calendar days). In some embodiments, a dose of rAAV is administered to a subject no more than bi-weekly (e.g., once in a two calendar week period). In some embodiments, a dose of rAAV is administered to a subject no more than once per calendar month (e.g., once in 30 calendar days). In some embodiments, a dose of rAAV is administered to a subject no more than once per six calendar months. In some embodiments, a dose of rAAV is administered to a subject no more than once per calendar year (e.g., 365 days or 366 days in a leap year).

In some embodiments, rAAV compositions are formulated to reduce aggregation of AAV particles in the composition, particularly where high rAAV concentrations are present (e.g., −10¹³ GC/ml or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1% or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intrapancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringeability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 m. Sonication of MLVs results in the formation of small unilamellar vesicles (SUVs) with diameters in the range of 200 to 500 A, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 m) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

In some embodiments, the methods described herein relate to treating a subject having or diagnosed as having a neurological disease or disorder, e.g., Huntington's disease with a nucleic acid described herein. Subjects having a neurological disease or disorder, e.g., Huntington's disease can be identified by a physician using current methods of diagnosing such diseases and disorders. For example, symptoms and/or complications of Huntington's disease which characterize these conditions and aid in diagnosis are well known in the art and include but are not limited to, depression and anxiety and with characteristic movement disturbances and chorea. Tests that may aid in a diagnosis of Huntington's disease, e.g. include, but are not limited to, genetic tests. A family history of Huntington's disease can also aid in determining if a subject is likely to have Huntington's disease or in making a diagnosis of Huntington's disease.

The compositions and methods described herein can be administered to a subject having or diagnosed as having a neurological disease or disorder. In some embodiments, the methods described herein comprise administering an effective amount of compositions described herein, e.g. a nucleic acid described herein to a subject in order to alleviate a symptom of a neurological disease or disorder. As used herein, “alleviating a symptom” is ameliorating any condition or symptom associated with a neurological disease or disorder. As compared with an equivalent untreated control, such reduction is by at least 5%, 10%, 20%, 40%, 50%, 60%, 80%, 90%, 95%, 99% or more as measured by any standard technique.

Effective amounts, toxicity, and therapeutic efficacy can be determined by standard pharmaceutical procedures in cell cultures or experimental animals, e.g., for determining the minimal effective dose and/or maximal tolerated dose. The dosage can vary depending upon the dosage form employed and the route of administration utilized. A therapeutically effective dose can be estimated initially from cell culture assays. Also, a dose can be formulated in animal models to achieve a dosage range between the minimal effective dose and the maximal tolerated dose. The effects of any particular dosage can be monitored by a suitable bioassay, e.g., assay for neuronal degradation or functionality among others. The dosage can be determined by a physician and adjusted, as necessary, to suit observed effects of the treatment.

The present invention also provides a synthetic CNS-specific promoter, expression cassette, vector, virion or pharmaceutical composition according to various aspects of the present invention for use in the treatment of a disease, preferably a disease associated with aberrant gene expression, optionally in the CNS (e.g. a genetic CNS disease). Relevant conditions, diseases and therapeutic expression products are discussed above.

The present invention also provides a synthetic CNS-specific promoter, expression cassette, vector, virion according to the various aspects of the present invention for use as medicament.

The present invention also provides a synthetic CNS-specific promoter, expression cassette, vector, virion according to the various aspects of the present invention for use the manufacture of a pharmaceutical composition for treatment of any condition or disease mentioned herein.

The present invention further provides a cell comprising a synthetic CNS-specific promoter, expression cassette, vector, virion according to the various aspects of the invention. Suitably the cell is a eukaryotic cell. The eukaryotic cell can suitably be an animal (metazoan) cell (e.g. a mammalian cell). Suitably, the cell is a human cell.

In some embodiments of the invention, the cell is ex vivo, e.g. in cell culture. In other embodiments of the invention the cell may be part of a tissue or multicellular organism.

In a preferred embodiment, the cell is a CNS cell, which may be ex vivo or in vivo. The CNS cell may be a primary neuron, astrocyte, oligodendrocyte, microglial cell or an ependymal cell. Alternatively, the CNS cell may be a CNS-derived cell line, e.g. immortalized cell line.

In a most preferred embodiment, the CNS cell is a neuron, suitably a dopaminergic neuron.

The cell may be present within a CNS tissue environment (e.g. within the CNS of an animal) or may be isolated from CNS tissue, e.g. it may be in cell culture. Suitably the primary cell or the cell line is a human cell.

The synthetic CNS-specific promoter, expression cassette, or vector, according to the invention may be inserted into the genome of the cell, or it may be episomal (e.g. present in an episomal vector).

It will be evident to the skilled person that a synthetic CNS-specific promoter, expression cassette, vector or virion according to various aspects of the invention may be used for gene therapy. Accordingly, the use of such nucleic acid constructs in gene therapy forms part of the present invention.

The invention thus provides, in some embodiments, an expression cassette, vector or virion according to the present invention for use in gene therapy in a subject, preferably gene therapy through CNS-specific expression of a therapeutic gene. The therapy may involve treatment of a disease through secretion of a therapeutic product from CNS cells, suitably a disease involving aberrant gene expression in the CNS, as discussed above.

The present invention also provides a method of expressing a therapeutic transgene in a CNS cell, the method comprising introducing into the CNS cell an expression cassette or vector according to the present invention. The CNS cell can be in vivo or ex vivo.

The present invention also provides a method of gene therapy of a subject, preferably a human, in need thereof, the method comprising: administering to the subject (suitably introducing into the CNS of the subject) a synthetic CNS-specific expression cassette, vector, virion or pharmaceutical composition of the present invention, which comprises a gene encoding a therapeutic product.

The method suitably comprises expressing a therapeutic amount of the therapeutic product from the gene in the CNS of said subject. Various conditions and diseases that can be treated are discussed above. Genes encoding suitable therapeutic products are discussed above.

The method suitably comprises administering a vector or virion according to the present invention to the subject. Suitably the vector is a viral gene therapy vector, for example an AAV vector.

In some embodiments, the method comprises administering the gene therapy vector systemically. Systemic administration may be enteral (e.g. oral, sublingual, and rectal) or parenteral (e.g. injection). Preferred routes of injection include intravenous, intramuscular, subcutaneous, intra-arterial, intra-articular, intrathecal, and intradermal injections. In one embodiment, the gene therapy vector may be delivered by injection into the cerebrospinal fluid (CSF) pathway. Non-limiting examples of delivery to the CSF pathway include intrathecal and intracerebroventricular administration.

Particularly preferred route of administration of AAV vector or virion comprising the synthetic CNS-specific promoter or expression cassette according to this invention is intravascular. Suitably, the AAV vector or virion comprising the synthetic CNS-specific promoter or expression cassette according to this invention may be administered in the veins of the dorsal hand or the veins of the anterior forearm. Suitable veins in the anterior forearm are the cephalic, median or basilic veins. This is because this administration route is generally safe for the patient while still allowing some penetration into the CNS.

In some embodiments, the viral gene therapy vector may be administered concurrently or sequentially with one or more additional therapeutic agents or with one or more saturating agents designed to prevent clearance of the vectors by the reticular endothelial system.

Where the vector is an AAV vector, the dosage of the vector may be from 1×10¹⁰ gc/kg to 1×10⁵ gc/kg or more, suitably from 1×10² gc/kg to 1×10⁴ gc/kg, suitably from 5×10¹² gc/kg to 5×10¹³ gc/kg.

In general, the subject in need thereof will be a mammal, and preferably a primate, more preferably a human. Typically, the subject in need thereof will display symptoms characteristic of a disease. The method typically comprises ameliorating the symptoms displayed by the subject in need thereof, by expressing the therapeutic amount of the therapeutic product. In one embodiment, the therapeutic methods of the present invention may be used to reduce the decline of functional capacity and activities of daily living as measured by a standard evaluation system such as, but not limited to, the total functional capacity (TFC) scale. In one embodiment, the methods of the present invention may be used to improve performance on any assessment used to measure symptoms of neurological disease. Such assessments include, but are not limited to ADAS-cog (Alzheimer Disease Assessment Scale—cognitive), MMSE (Mini-Mental State Examination), GDS (Geriatric Depression Scale), FAQ (Functional Activities Questionnaire), ADL (Activities of Daily Living), GPCOG (General Practitioner Assessment of Cognition), Mini-Cog, AMTS (Abbreviated Mental Test Score), Clock-drawing test, 6-CIT (6-item Cognitive Impairment Test), TYM (Test Your Memory), MoCa (Montreal Cognitive Assessment), ACE-R (Addenbrookes Cognitive Assessment), MIS (Memory Impairment Screen), BADLS (Bristol Activities of Daily Living Scale), Barthel Index, Functional Independence Measure, Instrumental Activities of Daily Living, IQCODE (Informant Questionnaire on Cognitive Decline in the Elderly), Neuropsychiatric Inventory, The Cohen-Mansfield Agitation Inventory, BEHAVE-AD, EuroQol, Short Form-36 and/or MBR Caregiver Strain Instrument, or any of the other tests as described in Sheehan B (Ther Adv Neurol Disord. 5(6):349-358 (2012)), the contents of which are herein incorporated by reference in their entirety.

Gene therapy protocols for therapeutic gene expression in target cells in vitro and in vivo, are well-known in the art and will not be discussed in detail here. Briefly, they include intravenous or intraarterial administration (e.g. intra-corotid artery, intra-hepatic artery, intra-hepatic vein), intracranial administration, intramuscular injection, interstitial injection, instillation in airways, application to endothelium and intra-hepatic parenchyme, of plasmid DNA vectors (naked or in liposomes) or viral vectors. Various devices have been developed for enhancing the availability of DNA to the target cell. While a simple approach is to contact the target cell physically with catheters or implantable materials containing the relevant vector, more complex approaches can use jet injection devices and suchlike. Gene transfer into mammalian CNS cells can been performed using both ex vivo and in vivo procedures. The ex vivo approach typically requires harvesting of the CNS cells, in vitro transduction with suitable expression vectors, followed by reintroduction of the transduced CNS cells into the CNS. This approach is generally less preferred due to the difficulty and danger of harvesting and reintroducing CNS cells in the brain. In vivo gene transfer has been achieved by injecting DNA or viral vectors directly into the CNS, e.g. by intracranial injection, or by intravenous or intraarterial injection of viral vectors.

In one embodiment, the gene therapy vector may be administered to a subject (e.g., to the CNS of a subject) in a therapeutically effective amount to reduce the symptoms of neurological disease of a subject (e.g., determined using a known evaluation method). In some embodiments, the gene therapy vector and compositions comprising the gene therapy vector may be administered in a way which allows them to cross the blood-brain barrier, vascular barrier, or other epithelial barrier.

The gene therapy vectors may be used in combination with one or more other therapeutic, prophylactic, research or diagnostic agents. By “in combination with,” it is not intended to imply that the agents must be administered at the same time and/or formulated for delivery together, although these methods of delivery are within the scope of the present invention. Compositions can be administered concurrently with, prior to, or subsequent to, one or more other desired therapeutics or medical procedures. 53 parker Compounds which may be used in combination with the AAV particles described herein include, but are not limited to, cholinesterase inhibitors (donepezil, rivastigmine, galantamine), NMDA receptor antagonists such as memantine, anti-psychotics, anti-depressants, anti-convulsants (e.g., sodium valproate and levetiracetam for myoclonus), secretase inhibitors, amyloid aggregation inhibitors, copper or zinc modulators, BACE inhibitors, inhibitors of tau aggregation, such as Methylene blue, phenothiazines, anthraquinones, n-phenylamines or rhodamines, microtubule stabilizers such as NAP, taxol or paclitaxel, kinase or phosphatase inhibitors such as those targeting GSK3 (lithium) or PP2A, immunization with ? beta peptides or tau phospho-epitopes, anti-tau or anti-amyloid antibodies, dopamine-depleting agents (e.g., tetrabenazine for chorea), benzodiazepines (e.g., clonazepam for myoclonus, chorea, dystonia, rigidity, and/or spasticity), amino acid precursors of dopamine (e.g., levodopa for rigidity), skeletal muscle relaxants (e.g., baclofen, tizanidine for rigidity and/or spasticity), inhibitors for acety choline release at the neuromuscular junction to cause muscle paralysis (e.g., botulinum toxin for bruxism and/or dystonia), atypical neuroleptics (e.g., olanzapine and quetiapine for psychosis and/or irritability, risperidone, sulpiride and haloperidol for psychosis, chorea and/or irritability, clozapine for treatment-resistant psychosis, aripiprazole for psychosis with prominent negative symptoms), selective serotonin reuptake inhibitors (SSRIs) (e.g., citalopram, fluoxetine, paroxetine, sertraline, mirtazapine, venlafaxine for depression, anxiety, obsessive compulsive behavior and/or irritability), hypnotics (e.g., xopiclone and/or Zolpidem for altered sleep-wake cycle), anticonvulsants (e.g., sodium valproate and carbamazepine for mania or hypomania) and mood stabilizers (e.g., lithium for mania or hypomania).

According to some preferred embodiments, the methods set out above may be used for the treatment of a subject with a CNS-related disease as discussed above, e.g. dopamine transporter deficiency syndrome.

### Immune Modulators

In some embodiments, the compositions described herein include an immune modulator and the methods further comprise administering the immune modulator. The immune modulator can be administered at the time of administration, before the administration or, after the administration. In the case in which a subject is re-administered at least a second composition, the immune modulator can be administered prior to, with, or after the at least second administration.

In a preferred embodiment, the immune modulator is administered prior to administration of a recombinant viral vector. In various embodiments, the immune modulator is administered at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or more prior to administration of a recombinant viral vector. In one embodiment, the immune modulator is administered no more than 24 hours prior to administration of a recombinant viral vector.

In one embodiment, the immune modulator is administered at substantially the same time as the recombinant viral vector, e.g., slightly before administration of the recombinant viral vector as disclosed herein (i.e., within 6-hours, or 5-hours, or 4-hours, or 3-hours, or 2-hours, or 1-hour). In some embodiments, the immune modulator is administered simultaneously, or within 6 hours after, administration of the viral vector, (i.e., within 1-hour, or within 2-hours, or within 3-hours, or within 4-hours, or within 5-hours or within 6-hours, or about 6-hours after administration of a viral vector composition as disclosed.

In some embodiments, the immune modulator allows for the administration of a recombinant viral vector to a subject who would otherwise not be a good candidate to receive such vector. A subject who would otherwise not be a good candidate to receive such a vector is, for example, a subject who has previously received administration of a recombinant viral vector and/or who was previously exposed to the recombinant viral vector and has subsequently developed an antibody response to the vector. Typically, a subject is considered to be a candidate, i.e., a good candidate, for administration of a recombinant viral vector when they have a titer for viral vector binding antibodies that is less than 1:5 (e.g., 1:1, 1:2, 1:3, or 1:4). In contrast, a subject is considered not to be a suitable candidate for administration of a recombinant viral vector when they have a titer for viral vector binding antibodies that is 1:5 or greater (e.g., 1:6, 1:6, 1:7, 1:8, 1:9, 1:10, 1:20, 1:30, 1:50, 1:100, 1:1,000 or more). One skilled in the art can assess the antibody titer of a subject using standard techniques in the art, e.g., by taking a biological sample from a subject, e.g., the subject's blood, challenging the biological sample with known antigens, and detecting the presence of the viral binding antibodies to the known antigens. An antibody titer is a measure of how much a sample can be diluted before a 50% viral vector neutralization can be detected in the sample. Antibody titers are usually expressed as ratios, such as 1:100, meaning that one-part serum to 100 parts saline solution (i.e., dilutant) results in 50% antibody neutralization in the sample, i.e., a reciprocal dilution of serum required to inhibit viral infection by 50% can be designated as neutralizing antibody titer at 50% inhibition. A titer of 1:10 of viral vector antibody is, therefore, an indication of lower level of viral vector antibodies than a 1:100 titer.

Accordingly, in one embodiment, the subject is assessed for the presence of anti-AAV antibodies to the AAV vector of a gene therapy prior to administration of the gene therapy. In one embodiment, the subject is assessed for the presence of neutralizing anti-AAV antibodies to the AAV vector of a gene therapy prior to administration of the gene therapy. Methods for detecting neutralizing anti-AAV antibodies is further described in, e.g., Kasprzyk T., et al. Mol Therapy. Methods & Clinical Dev. Jan. 6, 2022, the contents of which are incorporated herein in its entirety by reference.

In one embodiment, the immune modulator is administered to a subject having a titer of viral vector binding antibodies present in the biological sample, e.g., a blood sample, from the subject that is less than about 1:5 (e.g., 1:1, 1:2, 1:3, or 1:4), where 1 part of the biological sample diluted in 10,000 parts of buffer results in 50% viral vector neutralization.

In one embodiment, the immune modulator is administered to a subject having a titer of viral vector binding antibodies present in the biological sample or blood product from the subject that is greater than or equal to 1:5 and less than about 1:10 (e.g., 1:6, 1:7, 1:8, or 1:9), where 1 part of the biological sample or blood product diluted in 10,000 parts of buffer results in 50% viral vector neutralization, to enlarge the pool of subjects that can effectively be treated with AAV gene therapy. Currently, prospective patients with viral neutralizing antibody levels 1:5 or higher are excluded from such treatment, i.e., they are not good candidates. Administration of the immune modulator to a subject having an antibody titer greater than or equal to 1:5 but less than 1:10 is expected to decrease the antibody titer present in the subject to less that 1:5, thereby qualifying the subject as a candidate for administration of the recombinant viral vector (e.g., a gene therapy vector).

In one embodiments, the immune modulator is administered to a subject that was found to have a titer of viral vector binding antibodies present in the biological sample, e.g., a blood sample, from the subject that is greater than or equal to 1:5 and less than about 1:25 (e.g., 1:6, 1:7, 1:8, 1:9, 1:10, 1:11, 1:12, 1:13, 1:14, 1:15, 1:16, 1:17, 1:18, 1:19, 1:20, 1:21, 1:22, 1:23 and 1:24), where 1 part of the biological sample or blood product diluted in 10,000 parts of buffer results in 50% viral vector neutralization. For example, administration of the immune modulator to a subject having a titer greater than or equal to 1:5 but less than 1:15 is expected to decrease the antibody titer present in the subject to less that 1:5, thereby qualifying the subject as a candidate for administration of the recombinant viral vector (e.g., a gene therapy vector). In one embodiment, the immune modulator is administered to a subject having an antibody titer of viral vector binding antibodies present in the biological sample from the subject that is greater than or equal to 1:5 and less than about 1:100 (e.g., 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:30, 1:35, 1:40, 1:45, 1:50, 1:55, 1:60, 1:65, 1:70, 1:75, 1:80, 1:85, 1:90, 1:95, and 1:99), where 1 part of the biological sample or blood product diluted in 10,000 parts of buffer results in 50% viral vector neutralization. Administration of the immune modulator to a subject having an antibody titer greater than or equal to 1:5 but less than 1:25 is expected to decrease the antibody titer present in the subject to less that 1:5, thereby qualifying the subject as a candidate for administration of the recombinant viral vector (e.g., a gene therapy vector).

In some embodiments, the immune modulator enables repeated dosages, or repeat administration of an AAV vector as disclosed herein. For example, administration of the viral vector, e.g., a AAV vector disclosed herein, with an immune modulator (at substantially the same time, or before or after the administration of the AAV vector) can be administered multiple times (i.e., greater than one time) over a defined time period. For exemplary purposes, the AAV vector can be administered several times, i.e., more than once, over a several weeks (e.g., 2-weeks) to several months (e.g., 2-months). Without wishing to be limited to theory, administration of the AAV vector with the immune modulator according to the methods as disclosed herein can be, as non-limiting examples, every month over a period of 6-months, 3-4 times over a period of 6-weeks, every week over a period of 1-month (or about 4 weeks) or 2-months (or about 8-weeks). In some embodiments, where a AAV vector as disclosed herein is administered with an immune modulator (at substantially the same time, or before, or after the administration of the AAV vector) multiple times (e.g., a repeat dose), the dose of the viral vector, e.g., AAV vector is lower than typically used in a single-dose regimen, for example, at a dose lower than a single-dose regimen as described herein. For example, the dose of the AAV vector can be less than or equal to about 10¹², or lower than about 10¹², for example, the dose can be about 10⁷, 10⁸, 10⁹, 10¹⁰, 10¹¹, or 10¹², or any dose between 10⁷ and 10¹². In some embodiments, where repeat doses of a AAV vector as disclosed herein are administered to a subject according to the methods as disclosed herein with an immune modulator, the immune modulator can be changed between the doses, i.e., the same or different immune modulators can be used in repeat doses. For example, when the first dose of AAV is co-administered with an immune modulator A, a second immune modulator administered with the second or third dose of AAV is different than immune modulator A, e.g., the second immune modulator is immune modulator B. For exemplary purposes, a dosing regimen according to the methods as disclosed can be administration of a AAV vector at a concentration of 10¹² or less than 10¹², where the immune modulator is administered as A-B-C-D, or A-A-B-C, or A-B-A-C, where A, B, C and D are different immune modulators as disclosed herein. Put another way, the dosing regimen can include a plurality of doses of immune modulator over the time period, wherein each dose of the plurality includes an immune modulator independently selected from immune modulator A, immune modulator B, immune modulator C, immune modulator D, and combinations thereof (i.e., each dose can include more than one immune modulator). Such varying immune modulators enables repeated doses of the AAV vector as disclosed herein. In some embodiments, for example, an immune modulator “A” can be imlifidase (such as IdeS), and an immune modulator “B” can be ImmTOR™, as disclosed herein. In some embodiments, an AAV vector as disclosed herein? is administered at a first timepoint with an IdeS immune modulator, and an AAV vector as disclosed herein is administered at a second timepoint with a different immune modulator, such as immunoglobulin degrading protein or a small molecule, e.g., ImmTOR™, or vice versa. For example, an AAV vector as disclosed here? can be administered at a first timepoint with an ImmTOR™ immune modulator, and an AAV vector as disclosed herein can be administered at a second timepoint with an IdeS.

One aspect herein provides a method for administering a recombinant viral vector (e.g., a gene therapy vector) to a subject who has previously received a recombinant viral vector, for example, the same recombinant viral vector or another viral vector having a similar serotype, the method comprising, prior to administering the recombinant viral vector, administering to the subject an immune modulator. In one embodiment, the previously received recombinant viral vector elicits an immune response resulting in anti-AAV antibodies that target (i.e., recognizes and binds) to the recombinant viral vector administered.

Another aspect herein provides a method for administering a recombinant viral vector (e.g., a gene therapy vector) to a subject who was previously exposed to a viral vector, wherein the exposure elicits an immune response resulting in anti-AAV antibodies that target the recombinant viral vector to be administered, and wherein the subject has anti-AAV antibody titer of at least 1:5-1:15, at least 1:5-1:25, at least 1:5-1:50, or at least 1:5-1:100, the method comprising the steps of, prior to administering the recombinant viral vector, administering to the subject an immune modulator.

In one embodiment, the immune modulator is administered systemically.

In some embodiments, the immune modulator crosses the blood brain barrier. In alternative embodiments, the immune modulator does not cross the blood brain barrier.

In one embodiment, the immune modulator is administered locally. For example, when the recombinant viral vector is to be administer locally to the brain tissue and the immune modulator does not cross the blood brain barrier, it is preferred to administer the immune modulator locally to the brain tissue, e.g., via an appropriate catheter, either directly to the brain tissue or indirectly to the brain tissue through cerebrospinal fluid circulating about the spinal cord (i.e., a spinal tap).

In one embodiment, the immune modulator is administered locally to central nervous system (CNS) tissue (e.g., brain tissue, spinal cord tissue, cerebrospinal fluid (CSF)). CNS tissue also includes, but is not limited to, neuronal cells, glial cells, astrocytes, cerebrospinal fluid (CSF), interstitial spaces, bone, cartilage and the like. Any composition described herein may be delivered directly to the CNS or brain by injection into, e.g., the ventricular region, as well as to the striatum (e.g., the caudate nucleus or putamen of the striatum), spinal cord and neuromuscular junction, or cerebellar lobule.

In one embodiment, the immune modulator is administered locally to any of the following: neural pathways, somatosensory systems, visual systems, auditory systems, nerves, neuro endocrine systems, neuro vascular systems, brain neurotransmitter systems, dural meningeal system, or combinations thereof.

In one embodiment, the immune modulator is administered locally to the eye, e.g., the vitreous, the retina, or the sclera.

In one embodiment, the immune modulator is administered systemically.

In some embodiments, the immune modulator is an immunoglobulin degrading enzyme such as IdeS, IdeZ, IdeS/Z, Endo S, or, their functional variant. Non-limiting examples of such immunoglobulin degrading enzymes and their uses are described in U.S. Pat. Nos. 7,666,582, 8,133,483, US 20180037962, US 20180023070, US 20170209550, U.S. Pat. No. 8,889,128, WO 2010057626, U.S. Pat. Nos. 9,707,279, 8,323,908, US 20190345533, US 20190262434, US 20210246469 and WO 2020016318, each of which are incorporated in their entirety herein by reference.

In some embodiments, an immune modulator disclosed herein can be administered to a subject at any suitable dose, such as a suitable dose determined by a medical professional. For example, a suitable dosage may be from about 0.05 mg/kg to about 5 mg/kg body weight of a subject, or from about 0.1 mg/kg to about 4 mg/kg body weight of a subject.

In some embodiments, an immune modulator disclosed herein, e.g., IdeZ, is administered at a dosage of about 0.01 mg/kg to about 10 mg/kg body weight of a subject. For example, a suitable dosage may be from about 0.05 mg/kg to about 5 mg/kg body weight of a subject, or from about 0.1 mg/kg to about 4 mg/kg body weight of a subject.

In some embodiments, an immune modulator disclosed herein, e.g., IdeS, is administered at a dosage of about 0.01 mg/kg to about 10 mg/kg body weight of a subject. For example, a suitable dosage may be from about 0.05 mg/kg to about 5 mg/kg body weight of a subject, or from about 0.1 mg/kg to about 4 mg/kg body weight of a subject.

In some embodiments, an immune modulator disclosed herein, e.g., EndoS, is administered at a dosage of about 0.01 mg/kg to about 10 mg/kg body weight of a subject. For example, a suitable dosage may be from about 0.05 mg/kg to about 5 mg/kg body weight of a subject, or from about 0.1 mg/kg to about 4 mg/kg body weight of a subject.

Further, reference to a numerical range, such as “0.01 to 10” includes 0.011, 0.012, 0.013, etc., as well as 9.5, 9.6, 9.7, 9.8, 9.9, 10, etc., and so forth. For example, a dosage of about “0.01 mg/kg to about 10 mg/kg” body weight of a subject includes 0.011 mg/kg, 0.012 mg/kg, 0.013 mg/kg, 0.014 mg/kg, 0.015 mg/kg etc., as well as 9.5 mg/kg, 9.6 mg/kg, 9.7 mg/kg, 9.8 mg/kg, 9.9 mg/kg etc., and so forth.

In various embodiments, administration of a recombinant viral vector to a subject is preceded by administration of a protease and/or glycosidase to inhibit, reduce, or prevent an immune response (e.g., a humoral immune response) against the recombinant viral vector or antibodies that bind to the heterologous polynucleotide or a protein or peptide encoded by the heterologous polynucleotide encapsidated by the viral vector. For example, administration of the viral vector can be preceded by administration of a protease and/or glycosidase by at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours; or at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days; or by at least 1 month, 2 months, 3 months, 4 months, 5 months, 6 months, 7 months, 8 months, 9 months, 10 months, 11 months; or by at least 1 year, 2 years, 3 years, 4 years, 5 years, or more.

In one embodiments, administration of a recombinant viral vector to a subject is performed concurrently with administration of a protease and/or glycosidase to inhibit, reduce, or prevent an immune response (e.g., a humoral immune response) against the recombinant viral vector or antibodies that bind to the heterologous polynucleotide or a protein or peptide encoded by the heterologous polynucleotide encapsidated by the viral vector.

In certain embodiments, a protease and/or glycosidase is administered to a subject before an immune response (e.g., a humoral immune response), such as before development of neutralizing antibodies or development of antibodies that bind to the heterologous polynucleotide, protein, or peptide encoded by the heterologous polynucleotide encapsidated by the viral vector. In one embodiment, an immune response occurs within 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours; or within 1 day, 2 days, 3 days, 4 days, 5 days, or more following administration of a recombinant viral vector.

In some embodiments, the immune modulator is a proteasome inhibitor. In some embodiments, the immune modulator is a protease or glycosidase. In certain aspects, the proteasome inhibitor is Bortezomib. In some aspects of the embodiment, the immune modulator comprises bortezomib and an anti-CD20 antibody, such as Rituximab. In other aspects of the embodiment, the immune modulator comprises bortezomib, Rituximab, methotrexate, and intravenous gamma globulin. Non-limiting examples of proteasome inhibitors and their combinations with Rituximab, methotrexate and intravenous gamma globulin are described in U.S. Pat. Nos. 10,028,993, 9,592,247, and 8,809,282, each of which is incorporated in its entirety herein by reference.

In alternative embodiments, the immune modulator is an inhibitor of the NF-kB pathway. In certain aspects of the embodiment, the immune modulator is Rapamycin or a functional variant thereof. Non-limiting examples of uses of rapamycin are described in U.S. Pat. No. 10,071,114, US 20160067228, US 20160074531, US 20160074532, US 20190076458, U.S. Pat. No. 10,046,064, which are each incorporated herein by reference in their entirety. In other aspects of the embodiment, the immune modulator is synthetic nanocarriers comprising an immunosuppressant. Non limiting examples of immunosuppressants, immunosuppressants coupled to synthetic nanocarriers, synthetic nanocarriers comprising rapamycin, and/or, tolerogenic synthetic nanocarriers, their doses, administration and use are described in US20150320728, US 20180193482, US 20190142974, US 20150328333, US20160243253, U.S. Pat. No. 10,039,822, US 20190076522, US 20160022650, U.S. Pat. Nos. 10,441,651, 10,420,835, US 20150320870, US 2014035636, U.S. Pat. Nos. 10,434,088, 10,335,395, US 20200069659, U.S. Pat. No. 10,357,483, US 20140335186, U.S. Pat. Nos. 10,668,053, 10,357,482, US 20160128986, US 20160128987, US 20200038462, US 20200038463, each of which is incorporated in its entirety herein by reference.

In some embodiments, the immune modulator comprises synthetic nanocarriers comprising rapamycin (i.e., ImmTOR™ nanoparticles) as disclosed in Kishimoto, et al., 2016, Nat Nanotechnol, 11(10): 890-899; Maldonado, et al., 2015, PNAS, 112(2): E156-165) and in US20200038463 and U.S. Pat. No. 9,006,254, each of which is incorporated herein by reference in its entirety. In some embodiments, the immune modulator is an engineered cell, e.g., an immune cell that has been modified using SQZ technology as described in WO2017192786, which is incorporated herein in its entirety by reference.

In some embodiments, the immune modulator is selected from the group consisting of poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, Aquila's QS21 stimulon, and combinations thereof. In another further embodiment, the immunomodulator or poly-ICLC.

In some embodiments, the immune modulator is a small molecule that inhibits the innate immune response in cells, such as chloroquine (a TLR signaling inhibitor) and/or 2-aminopurine (a PKR inhibitor), which can also be administered in combination with the composition comprising at least one rAAV as disclosed herein. Some non-limiting examples of commercially available TLR-signaling inhibitors include BX795, chloroquine, CLI-095, OxPAPC, polymyxin B, and rapamycin (all available for purchase from INVIVOGEN). In addition, inhibitors of pattern recognition receptors (PRR) (which are involved in innate immunity signaling) such as 2-aminopurine, BX795, chloroquine, and H-89, can also be used in the compositions and methods comprising at least one rAAV vector as disclosed herein for in vivo protein expression as disclosed herein.

In some embodiments, the immune modulator is photopheresis, also known as extracorporeal photochemotherapy, or ECP. Photopheresis treatment is performed on a subject's blood. Using either an IV or a catheter, blood is routed from the subject through a device which separates out a portion of white blood cells (leukocytes). The separated white blood cells are treated with naturally occurring photosensitizing chemicals called 8-methoxypsoralen (8-MOP) and then exposed to specific wavelengths of ultraviolet (UVA) light. Following exposure to the UVA light, the blood is administered back to the subject. Photopheresis can be performed at least once daily. In one embodiment, photopheresis is performed at least 1, 2, 3, 4, 5, 6, 7 times a week prior to administration of the recombinant viral vector. In one embodiment, photopheresis is performed at least 1, 2, 3, 4, 5, 6, 7 times a week for at least 1, 2, 3, 4, 5, 6, 7, 8, 9, 10, 11, 12 or more weeks, or for at least 1, 2, 3, 4, 5, 6, 7, 8, 9, 10, 11, or 12 months prior to administration of the recombinant viral vector. Therefore, the administering the immune modulator to the subject can include performing photopheresis on the subject. It is understood that the photopheresis can be performed in conjunction with administration of a second immune modulator selected from the enzymes, nanoparticles, and chemical compositions described herein and/or as a portion of a multiple dosing regimen.

In some embodiments, a rAAV vector having the modified viral capsid can also encode a negative regulator of innate immunity such as NLRX1. Accordingly, in some embodiments, a rAAV vector can also optionally encode one or more of NLRX1, NS1, NS3/4A, or A46R. Additionally, in some embodiments, a composition comprising at least one rAAV vector as disclosed herein can also comprise a synthetic, modified-RNA encoding inhibitor of the innate immune system to avoid the innate immune response generated by the tissue or the subject.

In some embodiments, an immune modulator for use in the administration methods as disclosed herein is an immunosuppressive drug or agent. As used herein, the term “immunosuppressive drug or agent” refers to pharmaceutical agents that inhibit or interfere with normal immune function. Examples of immunosuppressive drugs or agents suitable for the methods disclosed herein include agents that inhibit T-cell/B-cell costimulation pathways, such as agents that interfere with the coupling of T-cells and B-cells via the CTLA4 and B7 pathways, as disclosed in U.S. Patent Pub. No 2002/0182211, which is incorporated herein by reference in its entirety. In one embodiment, an immunosuppressive agent is cyclosporine A. Other examples of immunosuppressive agents include myophenylate mofetil, rapamicin, and anti-thymocyte globulin. In various embodiments, the immunosuppressive drug is administered in a composition comprising at least one rAAV vector as disclosed herein, or in a separate composition but simultaneously with, or before or after administration of a composition comprising at least one rAAV vector according to the methods of administration as disclosed herein. An immunosuppressive drug is administered in a formulation which is compatible with the route of administration and is administered to a subject at a dosage sufficient to achieve the desired therapeutic effect. In some embodiments, the immunosuppressive drug is administered transiently for a sufficient time to induce tolerance to the rAAV vector as disclosed herein.

In any embodiment of the methods and compositions as disclosed herein, a subject being administered a composition disclosed herein is also administered an immunosuppressive agent. Various methods are known for achieving immunosuppression of an immune response in a patient being administered AAV. Methods known in the art include administering to the patient an immunosuppressive agent, such as a proteasome inhibitor. One such proteasome inhibitor known in the art, for instance as disclosed in U.S. Pat. No. 9,169,492 and U.S. patent application Ser. No. 15/796,137, both of which are incorporated herein by reference in their entireties, is bortezomib. In some embodiments, the immunosuppressive agent is an antibody, including polyclonal, monoclonal, scfv or other antibody-derived molecule that is capable of suppressing the immune response, for instance, through the elimination or suppression of antibody producing cells. In a further embodiment, the immunosuppressive element is a short hairpin RNA (shRNA). In this embodiment, the coding region of the shRNA is included in the rAAV cassette and is generally located downstream, i.e., 3′, of the poly-A tail. The shRNA can be targeted to reduce, reduce, or eliminate expression of immunostimulatory agents, such as cytokines, growth factors (including transforming growth factors 31 and 02, TNF and others that are known in the art).

The use of such immune modulating agents facilitates the ability to use multiple doses (e.g., multiple administration) over a plurality of months and/or years. This permits using multiple agents as discussed below, e.g., a rAAV vector encoding multiple genes, or multiple administrations to the subject.

### Kits

In one aspect, the instant disclosure relates to a nucleic acid, or recombinant viral vector comprising: (i) one or more inhibitory nucleic acids (e.g., miRNAs); and (ii) a nucleic acid encoding the CYP46A1 protein. In one aspect, the instant disclosure relates to the combination of (i) one or more inhibitory nucleic acids (e.g., miRNAs); and (ii) a nucleic acid encoding the CYP46A1 protein. In a combination of (i) and (ii), the two or more elements can be provided in a mixture or single formulation. Alternatively, the two or more elements can be provided in separate formulations that are packaged or provided as a set or kit.

The agents, e.g., viral vectors, described herein may, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

Exemplary embodiments of the invention will be described in more detail by the following examples. These embodiments are exemplary of the invention, which one skilled in the art will recognize is not limited to the exemplary embodiments.

In some embodiments, the instant disclosure relates to a kit for producing a rAAV, the kit comprising a container housing one or more of:
a) an isolated nucleic acid comprising an miRNA, e.g., comprising or encoded by the sequence set forth in any one of SEQ ID NOs: 6-17, 40-44, 50-66, 158-185, or 217-260 or comprising a seed sequence complementary to SEQ ID NO: 4, 18-39, or 46-49;
b) a recombinant viral vector comprising an isolated nucleic acid comprising a transgene encoding one or more miRNAs,
   e.g., wherein each miRNA comprises a seed sequence complementary to SEQ ID NO: 4, or wherein each miRNA comprises a sequence set forth in any one of SEQ ID NOs: 66-17, 40-44, 50-66, 158-185, or 217-260 flanked by a miRNA backbone sequence;
c) a recombinant viral vector comprising an isolated nucleic acid encoding the CYP46A1 protein; and/or
d) a recombinant viral vector comprising a nucleic acid comprising a transgene encoding one or more miRNAs,
   e.g., wherein each miRNA comprises a seed sequence complementary to SEQ ID NO: 4, or wherein each miRNA comprises a sequence set forth in any one of SEQ ID NOs: 6-17, 40-44, 50-66, 158-185, or 217-260 flanked by a miRNA backbone sequence; and a nucleic acid encoding the CYP46A1 protein.
      In some embodiments, the kit further comprises a container housing an isolated nucleic acid encoding an AAV capsid protein, for example an AAV9 capsid protein.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively, it may be housed in a vial or other container for storage. A second container may have other agents prepared sterilely. Alternatively, the kit may include the active agents premixed and shipped in a syringe, vial, tube, or other container.

Exemplary embodiments of the invention will be described in more detail by the following examples. These embodiments are exemplary of the invention, which one skilled in the art will recognize is not limited to the exemplary embodiments.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of Huntington's disease. A subject can be male or female.

A subject can be one who has been previously diagnosed with or identified as suffering from or having a condition in need of treatment (e.g. Huntington's disease) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA, miRNA.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

In some embodiments of any of the aspects, the miRNA described herein is exogenous. In some embodiments of any of the aspects, the miRNA described herein is ectopic. In some embodiments of any of the aspects, the miRNA described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is a codon-optimized variant, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art. Non-limiting examples of a viral vector of this invention include an AAV vector, an adenovirus vector, a lentivirus vector, a retrovirus vector, a herpesvirus vector, an alphavirus vector, a poxvirus vector a baculovirus vector, and a chimeric virus vector.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. Huntington's disease. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated. The “administration” of an agent to a subject includes any route of introducing or delivering to a subject the agent to perform its intended function. Administration can be carried out by any suitable route, including orally, intranasally, intraocularly, ophthalmically, parenterally (intravenously, intramuscularly, intraperitoneally, or subcutaneously), or topically. Administration includes self-administration and the administration by another.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “CNS cell” or “CNS cells” relates to cells which are found in CNS (CNS tissue) or which are derived from CNS tissue. CNS cells can be primary cells or a cell line (such as SH-Sy5y, Neuro2A, U87-MG). The CNS cells can be in in vivo (e.g. in CNS tissue) or in vitro (e.g. in cell culture). CNS cells comprise of neurons, astrocytes, oligodendrocytes, microglial cells and ependymal cells. Neurons as found in the CNS tissue comprise a cell body, a long axon and a synaptic terminal. A neuron transmits electric signals received in the cell body via its long axon to other cells close to their synaptic terminal. Oligodendrocytes are a type of glial cell in the CNS which produces myelin sheaths which wrap around neuronal axon for faster electrical signal conduction. Astrocytes are starshaped and are the most abundant cell type in the brain. They have multiple roles which aid and regulate transmission of electrical impulses within the brain and neuronal function. Microglia are the resident macrophage cell in the brain and are involved in immune defence. Ependymal cells form the epithelial lining of the ventricles. The term “CNS cell” or “CNS cells” as used herein includes neurons, astrocytes, oligodendrocytes, microglial cells and/or ependymal cells. The promoters of the present invention can be active in any of the CNS cell (e.g. neurons). The promoters of the present invention may be active in more than one type of CNS cell (e.g. neurons and astrocytes). The promoters of the present invention may be active in all types of CNS cells (neurons, astrocytes, oligodendrocytes, microglial cells and ependymal cells). Additionally, synthetic CNS-specific promoters of the present invention may be active in a subtype of a type of CNS cell such as dopaminergic neurons or mature oligodendrocytes. In some embodiments, the synthetic CNS-specific promoters of the present invention may only be active in the subtype of a type of CNS cell such as dopaminergic neurons or mature oligodendrocytes. The CREs, proximal/minimal promoters and promoters of the present invention may be active in specific areas of the CNS, in specific CNS cells or CNS cell subtypes or both. In some embodiments, the CREs, proximal/minimal promoters and promoters of the present invention may be active in a specific CNS cell type, such as neurons, within all areas of the CNS. In other embodiments, the CREs, proximal/minimal promoters and promoters of the present invention may be active in a specific CNS cell type, such as neurons, within no more than one area of the CNS, such as midbrain. In some embodiments, the CREs, proximal/minimal promoters and promoters of the present invention may be active in all CNS cells in all areas of the CNS. In some embodiments, the CREs, proximal/minimal promoters and promoters of the present invention may be active in al CNS cells in no more than one area of the CNS, such as midbrain.

The term “cis-regulatory element” or “CRE”, is a term well-known to the skilled person, and means a nucleic acid sequence such as an enhancer, promoter, insulator, or silencer, that can regulate or modulate the transcription of a neighboring gene (i.e. in cis). CREs are found in the vicinity of the genes that they regulate. CREs typically regulate gene transcription by binding to TFs, i.e. they include TFBS. A single TF may bind to many CREs, and hence control the expression of many genes (pleiotropy). CREs are usually, but not always, located upstream of the transcription start site (TSS) of the gene that they regulate. “Enhancers” in the present context are CREs that enhance (i.e. upregulate) the transcription of genes that they are operably associated with, and can be found upstream, downstream, and even within the introns of the gene that they regulate. Multiple enhancers can act in a coordinated fashion to regulate transcription of one gene. “Silencers” in this context relates to CREs that bind TFs called repressors, which act to prevent or downregulate transcription of a gene. The term “silencer” can also refer to a region in the 3′ untranslated region of messenger RNA, that bind proteins which suppress translation of that mRNA molecule, but this usage is distinct from its use in describing a CRE. Generally, the CREs of the present invention are CNS-specific enhancer elements (often referred to as CNS-specific CREs, or CNS-specific CRE enhancers, or suchlike). In the present context, it is preferred that the CRE is located 2500 nucleotides or less from the transcription start site (TSS), more preferably 2000 nucleotides or less from the TSS, more preferably 1500 nucleotides or less from the TSS, and suitably 1000, 750, 500, 250, 200, 150, or 100 nucleotides or less from the TSS. CREs of the present invention are preferably comparatively short in length, preferably 800 nucleotides or less in length, for example they may be 800, 795, 790, 785, 780, 775, 770, 765, 760, 755, 750, 745, 740, 735, 725, 700, 695, 690, 685, 680, 675, 670, 665, 660, 655, 650, 645, 640, 635, 630, 625, 620, 615, 610, 605 or 600 nucleotides or less in length. The CREs of the present invention are typically provided in combination with an operably linked promoter element, which ca be a minimal promoter or proximal promoter; the CREs of the present invention may enhance CNS-specific activity of the promoter element.

The term “cis-regulatory module” or “CRM” means a functional regulatory nucleic acid module, which usually comprises two or more CREs; in the present invention the CREs are typically CNS-specific enhancers and thus the CRM is a synthetic CNS-specific regulatory nucleic acid. A CRM may comprise a plurality of CNS-specific CREs. Suitably, at least one of the CREs comprised in the CRM is a CRE according to SEQ ID NO: 191 or 192 or a functional variant thereof. Typically, the multiple CREs within the CRM act together (e.g. additively or synergistically) to enhance the transcription of a gene that a promoter comprising the CRM is operably associated with. There is considerable scope to shuffle (i.e. reorder), invert (i.e. reverse orientation), and alter spacing of CREs within a CRM. Accordingly, functional variants of CRMs of the present invention include, inter alia, variants of the referenced CRMs wherein CREs within them have been shuffled and/or inverted, and/or the spacing between CREs has been altered.

As used herein, “minimal promoter” (also known as the “core promoter”) refers to a short DNA segment which is inactive or largely inactive by itself, but can mediate transcription when combined with other transcription regulatory elements. Minimum promoter sequence can be derived from various different sources, including prokaryotic and eukaryotic genes. Examples of minimal promoters include beta-globin minimal promoter, the dopamine beta-hydroxylase gene minimum promoter, cytomegalovirus (CMV) immediate early gene minimum promoter (CMV-MP), and the herpes thymidine kinase minimal promoter (MinTK). A minimal promoter typically comprises the transcription start site (TSS) and elements directly upstream, a binding site for RNA polymerase II, and general transcription factor binding sites (often a TATA box). A minimal promoter may also include some elements downstream of the TSS, but these typically have little functionality absent additional regulatory elements.

As used herein, “proximal promoter” relates to the minimal promoter plus the proximal sequence upstream of the gene that tends to contain primary regulatory elements. It often extends approximately 250 base pairs upstream of the TSS, and includes specific TFBS. A proximal promoter may also include one or more regulatory elements downstream of the TSS, for example a UTR or an intron. In the present case, the proximal promoter may suitably be a naturally occurring CNS-specific proximal promoter that can be combined with one or more CREs of the present invention. For example, the CNS-specific proximal promoter may be the human proximal TH promoter. However, the proximal promoter can be synthetic.

As used herein, “promoter element” refers to either a minimal promoter or proximal promoter as defined above. In the context of the present invention a promoter element may be combined with one or more CREs in order to provide a synthetic CNS-specific promoter of the present invention.

A “functional variant” of a CRE, CRM, promoter element, promoter or other regulatory nucleic acid in the context of the present invention is a variant of a reference sequence that retains the ability to function in the same way as the reference sequence, e.g. as a CNS-specific CRE, CNS-specific CRM or CNS-specific promoter. Alternative terms for such functional variants include “biological equivalents” or “equivalents”. It will be appreciated that the ability of a given CRE, CRM, promoter or other regulatory sequence to function as a CNS-specific enhancer is determined significantly by the ability of the sequence to bind the same CNS-specific TFs that bind to the reference sequence. Accordingly, in most cases, a functional variant of a CRE or CRM will contain TFBS for the most or all of same TFs as the reference CRE, CRM or promoter. It is preferred, but not essential, that the TFBS of a functional variant are in the same relative positions (i.e. order and general position) as the reference CRE, CRM or promoter. It is also preferred, but not essential, that the TFBS of a functional variant are in the same orientation as the reference sequence (it will be noted that TFBS can in some cases be present in reverse orientation, e.g. as the reverse complement vis-á-vis the sequence in the reference sequence). It is also preferred, but not essential, that the TFBS of a functional variant are on the same strand as the reference sequence. Thus, in preferred embodiments, the functional variant comprises TFBS for the same TFs, in the same order, the same position, in the same orientation and on the same strand as the reference sequence. It will also be appreciated that the sequences lying between TFBS (referred to in some cases as spacer sequences, or suchlike) are of less consequence to the function of the CRE or CRM. Such sequences can typically be varied considerably, and their lengths can be altered. However, in preferred embodiments the spacing (i.e. the distance between adjacent TFBS) is substantially the same (e.g. it does not vary by more than 20%, preferably by not more than 10%, and more preferably it is approximately the same) in a functional variant as it is in the reference sequence. It will be apparent that in some cases a functional variant of a CRE can be present in the reverse orientation, e.g. it can be the reverse complement of a CRE as described above, or a variant thereof.

Levels of sequence identity between a functional variant and the reference sequence can also be an indicator or retained functionality. High levels of sequence identity in the TFBS of the CRE is of generally higher importance than sequence identity in the spacer sequences (where there is little or no requirement for any conservation of sequence). However, it will be appreciated that even within the TFBS, a considerable degree of sequence variation can be accommodated, given that the sequence of a functional TFBS does not need to exactly match the consensus sequence.

The ability of one or more TFs to bind to a TFBS in a given functional variant can determined by any relevant means known in the art, including, but not limited to, electromobility shift assays (EMSA), binding assays, chromatin immunoprecipitation (ChIP), and ChIP-sequencing (ChIP-seq). In a preferred embodiment the ability of one or more TFs to bind a given functional variant is determined by EMSA. Methods of performing EMSA are well-known in the art. Suitable approaches are described in Sambrook et al. cited above. Many relevant articles describing this procedure are available, e.g. Hellman and Fried, Nat Protoc. 2007; 2(8): 1849-1861.

“CNS-specific” or “CNS-specific expression” refers to the ability of a cis-regulatory element, cis-regulatory module or promoter to enhance or drive expression of a gene in CNS cells (or in CNS-derived cells) in a preferential or predominant manner as compared to other tissues (e.g. liver, kidney, spleen, heart, muscle and lung). Expression of the gene can be in the form of mRNA or protein. In preferred embodiments, CNS-specific expression is such that there is negligible expression in other (i.e. non-CNS) tissues or cells, i.e. expression is highly CNS-specific.

The ability of a CRE, CRM or promoter to function as a CNS-specific CRE, CRM or promoter can be readily assessed by the skilled person. The skilled person can thus easily determine whether any variant of the specific CRE, CRM or promoter recited above remains functional (i.e. it is a functional variant as defined above). For example, any given CRM to be assessed can be operably linked to a minimal promoter (e.g. positioned upstream of CMV MP) and the ability of the cis-regulatory element to drive CNS-specific expression of a gene (typically a reporter gene) is measured.

Alternatively, a variant of a CRE or CRM can be substituted into a synthetic CNS-specific promoter in place of a reference CRE or CRM, and the effects on CNS-specific expression driven by said modified promoter can be determined and compared to the unmodified form. Similarly, the ability of a promoter to drive CNS-specific expression can be readily assessed by the skilled person (e.g. as described in the examples below). Expression levels of a gene driven by a variant of a reference promoter can be compared to the expression levels driven by the reference promoter. In some embodiments, where CNS-specific expression levels driven by a variant promoter are at least 50%, at least 60%, at least 70%, at least 80%, at least 90%, or at least 100% of the expression levels driven by the reference promoter, it can be said that the variant remains functional. Suitable nucleic acid constructs and reporter assays to assess CNS-specific expression enhancement can be easily constructed, and the examples set out below gives suitable methodologies.

CNS-specificity can be identified wherein the expression of a gene (e.g. a therapeutic or reporter gene) occurs preferentially or predominantly in CNS-derived cells. Preferential or predominant expression can be defined, for example, where the level of expression is significantly greater in CNS-derived cells than in other types of cells (i.e. non-CNS-derived cells). For example, expression in CNS-derived cells is suitably at least 5-fold higher than in non-CNS cells, preferably at least 10-fold higher than in non-CNS cells, and it may be 50 fold higher or more in some cases. For convenience, CNS-specific expression can suitably be demonstrated via a comparison of expression levels in a different non-CNS cell lines, e.g. primary CNS cells or CNS-derived cell line such as SH-Sy5y, Neuro2A, U87-MG compared with expression level in a muscle-derived cell line such as C2C12 or H2K cells (skeletal muscle) or H9C2 cells (cardiac), in a liver-derived cell line (e.g. Huh7 or HepG2), kidney derived cell line (e.g. HEK-293), a cervical tissue-derived cell line (e.g. HeLa) and/or a lung derived cell line (e.g. A549).

The synthetic CNS-specific promoters of the present invention preferably exhibit reduced expression in non-CNS-derived cells, suitably in C2C12, H9C2, Huh7, HEK-293, HeLa, and/or A549 cells when compared to a non-tissue specific promoter such as CMV-IE. The synthetic CNS-specific promoters of the present invention preferably have an activity of 50% or less than the CMV-IE promoter in non-CNS-derived cells (suitably in C2C12, H9C2, Huh7, HEK-293, HeLa, and/or A549), suitably 25% or less, 20% or less, 15% or less, 10% or less, 5% or less or 1% or less. Generally, it is preferred that expression in non-CNS-derived cells is minimized, but in some cases this may not be necessary. Even if a synthetic CNS-specific promoter of the present invention has higher expression in, e.g., one or two non-CNS cells, as long as it generally has higher expression overall in a range of CNS cells versus non-CNS cell, it can still be a CNS-specific promoter.

The synthetic CNS-specific promoters of the present invention are preferably suitable for promoting expression in the CNS of a subject, e.g. driving CNS-specific expression of a transgene, preferably a therapeutic transgene. Preferred synthetic CNS-specific promoters of the present invention are suitable for promoting CNS-specific transgene expression and have an activity in CNS cells which is at least 15%, 20%, 30%, 40%, 50%, 60%, 70%, 80%, 90%, 100%, 125%, 150%, 175%, 200%, 250%, 300%, 350% or 400% of the activity of any one of Synapsin-1, Camk2a, GFAP, MBP, IBA1 or NSE promoters. In some embodiments, the synthetic CNS-specific promoters of the invention are suitable for promoting CNS-specific transgene expression at a level at least 100% of the activity of the human TH promoter, preferably 150% or 200% of the activity of the human TH promoter. Such CNS-specific expression is suitably determined in CNS-derived cells, e.g. SH-Sy5y, Neuro2A, U87-MG cell lines or primary CNS cells (suitably primary human neurons, astrocytes, oligodendrocytes, microglia and/or ependymal cells). Synthetic CNS-specific promoters of the present invention may also be able to promote CNS-specific expression of a gene at a level at least 50%, 100%, 150% or 200% compared to CMV-IE in CNS-derived cells, e.g. SH-Sy5y, Neuro2A, U87-MG cell lines or primary CNS cells (suitably primary human neurons, astrocytes, oligodendrocytes, microglia and/or ependymal cells).

The term “codon-optimized,” as used herein, refers to a gene coding sequence that has been optimized to increase expression by substituting one or more codons normally present in a coding sequence with a codon for the same (synonymous) amino acid. In this manner, the protein encoded by the gene is identical, but the underlying nucleobase sequence of the gene or corresponding mRNA is different. Codon-optimization can also include substitutions that eliminate CpG islands and/or alternative reading frames, and/or restriction sites. In some embodiments, the optimization substitutes one or more rare codons (that is, codons for tRNA that occur relatively infrequently in cells from a particular species) with synonymous codons that occur more frequently to improve the efficiency of translation. For example, in human codon-optimization one or more codons in a coding sequence are replaced by codons that occur more frequently in human cells for the same amino acid. Codon optimization can also increase gene expression through other mechanisms that can improve efficiency of transcription and/or translation. Desirably, a codon-optimized gene exhibits improved protein expression, for example, the protein encoded thereby is expressed at a detectably greater level in a cell compared with the level of expression of the protein provided by the wildtype gene in an otherwise similar cell. Codon-optimization also provides the ability to distinguish a codon-optimized gene and/or corresponding mRNA from an endogenous gene and/or corresponding mRNA in vitro or in vivo. It is understood that a codon-optimized sequence does not have to be optimized to the fullest extent possible. It is also understood that codon-optimized sequences can include substitutions and/or deletions in untranslated regions of a nucleic acid, for example, to enhance expression, enhance nucleic acid stability, eliminate CpG islands, eliminate restriction sites, and/or to eliminate non-coding RNA.

The terms “identity” and “identical” and the like refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250). Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, MD), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn; Align Sequence Nucleotide BLAST) program may be employed using the default parameters. Nucleic acid sequences with even greater similarity to the reference sequences will show increasing percentage identity when assessed by this method. Typically, the percentage sequence identity is calculated over the entire length of the sequence.

For example, a global optimal alignment is suitably found by the Needleman-Wunsch algorithm with the following scoring parameters: Match score: +2, Mismatch score: −3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100.

The term “transcription factor binding site” (TFBS) is well known in the art. It will be apparent to the skilled person that alternative TFBS sequences can be used, provided that they are bound by the intended TF. Consensus sequences for the various TFBS are known in the art, and the skilled person can readily use this information to determine alternative TFBS. Furthermore, the ability of a TF to bind to a given putative sequence can readily be determined experimentally by the skilled person (e.g. by EMSA and other approaches well known in the art and discussed herein).

The meaning of “consensus sequence” is well-known in the art. In the present application, the following notation is used for the consensus sequences, unless the context dictates otherwise. Considering the following exemplary DNA sequence:

A[CT]N{A}YR

A means that an A is always found in that position; [CT] stands for either C or T in that position; N stands for any base in that position; and {A} means any base except A is found in that position. Y represents any pyrimidine, and R indicates any purine.

“Synthetic” in the present application means a nucleic acid molecule that does not occur in nature. Synthetic nucleic acid expression constructs of the present invention are produced artificially, typically by recombinant technologies. Such synthetic nucleic acids may contain naturally occurring sequences (e.g. promoter, enhancer, intron, and other such regulatory sequences), but these are present in a non-naturally occurring context. For example, a synthetic gene (or portion of a gene) typically contains one or more nucleic acid sequences that are not contiguous in nature (chimeric sequences), and/or may encompass substitutions, insertions, and deletions and combinations thereof.

“Complementary” or “complementarity”, as used herein, refers to the Watson-Crick base-pairing of two nucleic acid sequences. For example, for the sequence 5′-AGT-3′ binds to the complementary sequence 3′-TCA-5′. Complementarity between two nucleic acid sequences may be “partial”, in which only some of the bases bind to their complement, or it may be complete as when every base in the sequence binds to its complementary base. The degree of complementarity between nucleic acid strands has significant effects on the efficiency and strength of hybridization between nucleic acid strands.

As used herein, the phrase “transgene” refers to an exogenous nucleic acid sequence. In one example, a transgene is a gene encoding an industrially or pharmaceutically useful compound, or a gene encoding a desirable trait. In yet another example, the transgene encodes an antisense nucleic acid sequence, wherein expression of the antisense nucleic acid sequence inhibits expression of a target nucleic acid sequence. The transgene preferably encodes a therapeutic product, e.g. a protein.

The term “specifically active in an area or in a tissue” refers to a promoter which is predominantly active in that area or tissue, i.e. more active in that area or tissue than in other areas or tissues.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), WO 2018/057855A, U.S. Pat. No. 10,457,940, the contents of each of which are all incorporated by reference herein in their entireties.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:

1. A method for treating a neurological disease or disorder in a subject in need thereof, the method comprising administering to a subject having or at risk of developing the neurological disease or disorder a therapeutically effective amount of
(a) an isolated nucleic acid encoding a transgene encoding one or more miRNAs; and
(b) an isolated nucleic acid encoding a CYP46A1 protein.

2. A method for treating a neurological disease or disorder in a subject in need thereof, the method comprising administering to a subject having or at risk of developing the neurological disease or disorder a therapeutically effective amount of
(a) a recombinant viral vector comprising an isolated nucleic acid comprising (i) a first region comprising a first adeno-associated virus (AAV) inverted terminal repeat (ITR), or a variant thereof, and (ii) a second region comprising a transgene encoding one or more miRNAs; and
(b) a recombinant viral vector comprising an isolated nucleic acid encoding the CYP46A1 protein.

3. The method of any of the preceding paragraphs, wherein the neurological disease or disorder is Alzheimer's disease, Parkinson's disease, Huntington's disease, Canavan disease, Leigh's disease, spinal cerebral ataxia, polyglutamine repeat spinocerebellar ataxia, Krabbe's disease, Batten's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, neuropathic pain, trauma due to spinal cord or head injury, ophthalmic diseases and disorders, Tay-Sachs disease, Lesch-Nyhan disease, epilepsy, cerebral infarcts, depression, bipolar affective disorder, persistent affective disorder, secondary mood disorder, schizophrenia, drug dependency, neuroses, psychosis, dementia, paranoia, attention deficit disorder, psychosexual disorders, sleeping disorders, pain disorders, eating or weight disorders.

4. The method of any of the preceding paragraphs, wherein the neurological disease or disorder is a central nervous system (CNS) disease or disorder.

5. The method of any of the preceding paragraphs, wherein the CNS disease or disorder is selected from Huntington's disease, Alzheimer's disease, Polyglutamine repeat spinocerebellar ataxias, Amyotrophic lateral sclerosis and Parkinson's disease.

6. The method of any of the preceding paragraphs, wherein the CNS disease or disorder is Alzheimer's disease and the at least one miRNA comprises a seed sequence complementary to Amyloid Precursor Protein (APP), Presenilin 1, Presenilin 2, ABCA7, SORL1, and disease-associated alleles thereof.

7. The method of any of the preceding paragraphs, wherein the CNS disease or disorder is Parkinson's disease and the at least one miRNA comprises a seed sequence complementary to SNCA, LRRK2/PARK8, PRKN, PINK1, DJ1/PARK7, VPS35, EIF4G1, DNAJC13, CHCHD2, UCHL1, GBA1, and disease-associated alleles thereof.

8. The method of any of the preceding paragraphs, wherein the CNS disease is Huntington's disease and at least one miRNA comprises a seed sequence complementary to SEQ ID NO: 4, or wherein at least one miRNA comprises the sequence of any one of SEQ ID NOs: 6-17, 40-44, 50-66, 158-185, or 217-260 flanked by a miRNA backbone sequence.

9. The method of any of the preceding paragraphs, wherein the CNS disease is Huntington's disease and at least one miRNA comprises the sequence of any one of SEQ ID NOs6-17, 40-44, 50-66, 158-185, or 217-260.

10. The method of any of the preceding paragraphs, wherein at least one of the miRNAs hybridizes with and inhibits expression of human huntingtin.

11. The method of any of the preceding paragraphs, wherein the subject comprises a huntingtin gene having more than 36 CAG repeats, more than 40 repeats, or more than 100 repeats.

12. The method of any of the preceding paragraphs, wherein the subject is less than 20 years of age.

13. The method of any of the preceding paragraphs, wherein the recombinant viral vector is selected from the group consisting of: an AAV vector, an adenovirus vector, a lentivirus vector, a retrovirus vector, a herpesvirus vector, an alphavirus vector, a poxvirus vector, a baculovirus vector, and a chimeric virus vector.

14. The method of any of the preceding paragraphs, wherein the recombinant viral vector comprising (a) is the same as the recombinant viral vector comprising (b).

15. The method of any of the preceding paragraphs, wherein the isolated nucleic acid of (a) and (b) are comprised in separate recombinant viral vectors.

16. The method of any of the preceding paragraphs, wherein the isolated nucleic acid of (a) and (b) are comprised in the same recombinant viral vector.

17. The method of any of the preceding paragraphs, wherein (a) and (b) are administered at substantially the same time.

18. The method of any of the preceding paragraphs, wherein (a) and (b) are administered at different time points.

19. The method of any of the preceding paragraphs, wherein the different time points are spaced by at least 1 min, at least 1 hour, at least 1 day, at least 1 week, at least 1 month, at least 1 year, or more.

20. The method of any of the preceding paragraphs, wherein (a) is administered prior to the administration of (b).

21. The method of any of the preceding paragraphs, wherein (b) is administered prior to the administration of (a).

22. The method of any of the preceding paragraphs, wherein the administration of (a), (b), or (a) and (b) is repeated at least once.

23. The method of any of the preceding paragraphs, wherein the transgene comprises two miRNAs in tandem that are flanked by introns.

24. The method of any of the preceding paragraphs, wherein the flanking introns are identical

25. The method of any of the preceding paragraphs, wherein the flanking introns are from the same species.

26. The method of any of the preceding paragraphs, wherein the flanking introns are hCG introns.

27. The method of any of the preceding paragraphs, wherein the transgene comprises a promoter.

28. The method of any of the preceding paragraphs, wherein the promoter is a synapsin (Syn1) promoter, or a promoter of Tables 10-13.

29. The method of any of the preceding paragraphs, wherein the one or more miRNAs are located in an untranslated portion of the transgene.

30. The method of any of the preceding paragraphs, wherein the untranslated portion is an intron.

31. The method of any of the preceding paragraphs, wherein the untranslated portion is between the last codon of the nucleic acid sequence encoding a protein and a poly-A tail sequence, or between the last nucleotide base of a promoter sequence and a poly-A tail sequence.

32. The method of any of the preceding paragraphs, further comprising a third region comprising a second adeno-associated virus (AAV) inverted terminal repeat (ITR), or a variant thereof.

33. The method of any of the preceding paragraphs, wherein the ITR variant lacks a functional terminal resolution site (TRS), optionally wherein the ITR variant is a ATRS ITR.

34. The method of any of the preceding paragraphs, wherein the administration results in delivery of the viral vector or isolated nucleic acid to the central nervous system (CNS) of the subject.

35. The method of any of the preceding paragraphs, wherein the administration is via injection, optionally intravenous injection or intrastriatal injection.

36. The method of any of the preceding paragraphs, wherein the viral vector is AAV1, AAV2, AAV3b, AAV4, AAV5, AAV6, AAV7, AAV8, AAV9, AAV10, AAVrh10, AAV11, or, AAV12, or a chimera thereof.

37. The method of any of the preceding paragraphs, the viral vector comprises a capsid protein from AAV serotype AAV1, AAV2, AAV3b, AAV4, AAV5, AAV6, AAV7, AAV8, AAV9, AAV10, AAVrh10, AAV11, or, AAV12, or a chimera thereof.

38. The method of any of the preceding paragraphs, wherein the capsid protein is an AAV9 capsid protein.

39. The method of any of the preceding paragraphs, wherein the viral vector is a self-complementary AAV (scAAV).

40. The method of any of the preceding paragraphs, wherein the viral vector is formulated for delivery to the central nervous system (CNS).

41. A composition or combination comprising:
(a) an isolated nucleic acid encoding a transgene encoding one or more miRNAs; and
(b) an isolated nucleic acid encoding a CYP46A1 protein.

42. A composition or combination comprising:
(a) a recombinant viral vector comprising an isolated nucleic acid comprising (i) a first region comprising a first adeno-associated virus (AAV) inverted terminal repeat (ITR), or a variant thereof, and (ii) a second region comprising a transgene encoding one or more miRNAs; and
(b) a recombinant viral vector comprising an isolated nucleic acid encoding the CYP46A1 protein.

43. The composition or combination of any of the preceding paragraphs, for use in a method for treating a neurological disease or disorder in a subject in need thereof, the method comprising administering to a subject having or at risk of developing the neurological disease or disorder a therapeutically effective amount of the composition or combination.

44. The composition or combination of any of the preceding paragraphs, wherein the neurological disease or disorder is Alzheimer's disease, Parkinson's disease, Huntington's disease, Canavan disease, Leigh's disease, spinal cerebral ataxia, Krabbe's disease, polyglutamine repeat spinocerebellar ataxia, Batten's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, neuropathic pain, trauma due to spinal cord or head injury, ophthalmic diseases and disorders, Tay-Sachs disease, Lesch-Nyhan disease, epilepsy, cerebral infarcts, depression, bipolar affective disorder, persistent affective disorder, secondary mood disorder, schizophrenia, drug dependency, neuroses, psychosis, dementia, paranoia, attention deficit disorder, psychosexual disorders, sleeping disorders, pain disorders, eating or weight disorders.

45. The composition or combination of any of the preceding paragraphs, wherein the neurological disease or disorder is a central nervous system (CNS) disease or disorder.

46. The composition or combination of any of the preceding paragraphs, wherein the CNS disease or disorder is selected from Huntington's disease, Alzheimer's disease, Polyglutamine repeat spinocerebellar ataxias, Amyotrophic lateral sclerosis and Parkinson's disease.

47. The composition or combination of any of the preceding paragraphs, wherein the at least one miRNA comprises a seed sequence complementary to Amyloid Precursor Protein (APP), Presenilin 1, Presenilin 2, ABCA7, SORL1, and disease-associated alleles thereof.

48. The composition or combination of any of the preceding paragraphs, wherein the at least one miRNA comprises a seed sequence complementary to SNCA, LRRK2/PARK8, PRKN, PINK1, DJ1/PARK7, VPS35, EIF4G1, DNAJC13, CHCHD2, UCHL1, GBA1, and disease-associated alleles thereof.

49. The composition or combination of any of the preceding paragraphs, wherein the at least one miRNA comprises a seed sequence complementary to SEQ ID NO: 4, or wherein the at least one miRNA comprises the sequence of any one of SEQ ID NOs: 6-17, 40-44, 50-66, 158-185, or 217-260 flanked by a miRNA backbone sequence.

50. The composition or combination of any of the preceding paragraphs, wherein the at least one miRNA comprises the sequence of any one of SEQ ID NOs: 6-17, 40-44, 50-66, 158-185, or 217-260. 51. The composition or combination of any of the preceding paragraphs, wherein at least one of the miRNAs hybridizes with and inhibits expression of human huntingtin.

52. The composition or combination of any of the preceding paragraphs, wherein the subject comprises a huntingtin gene having more than 36 CAG repeats, more than 40 repeats, or more than 100 repeats.

53. The composition or combination of any of the preceding paragraphs, wherein the subject is less than 20 years of age.

54. The composition or combination of any of the preceding paragraphs, wherein the recombinant viral vector is selected from the group consisting of: an AAV vector, an adenovirus vector, a lentivirus vector, a retrovirus vector, a herpesvirus vector, an alphavirus vector, a poxvirus vector a baculovirus vector, and a chimeric virus vector.

55. The composition or combination of any of the preceding paragraphs, wherein the recombinant viral vector comprising (a) is the same as the recombinant viral vector comprising (b).

56. The composition or combination of any of the preceding paragraphs, wherein the isolated nucleic acid of (a) and (b) are comprised in separate recombinant viral vectors.

57. The composition or combination of any of the preceding paragraphs, wherein the isolated nucleic acid of (a) and (b) are comprised in the same recombinant viral vector.

58. The composition or combination of any of the preceding paragraphs, wherein (a) and (b) are administered at substantially the same time.

59. The composition or combination of any of the preceding paragraphs, wherein (a) and (b) are administered at different time points.

60. The composition or combination of any of the preceding paragraphs, wherein the different time points are spaced by at least 1 min, at least 1 hour, at least 1 day, at least 1 week, at least 1 month, at least 1 year, or more.

61. The composition or combination of any of the preceding paragraphs, wherein (a) is administered prior to the administration of (b).

62. The composition or combination of any of the preceding paragraphs, wherein (b) is administered prior to the administration of (a).

63. The composition or combination of any of the preceding paragraphs, wherein the administration of (a), (b), or (a) and (b) is repeated at least once.

64. The composition or combination of any of the preceding paragraphs, wherein the transgene comprises two miRNAs in tandem that are flanked by introns.

65. The composition or combination of any of the preceding paragraphs, wherein the flanking introns are identical.

66. The composition or combination of any of the preceding paragraphs, wherein the flanking introns are from the same species.

67. The composition or combination of any of the preceding paragraphs, wherein the flanking introns are hCG introns.

68. The composition or combination of any of the preceding paragraphs, wherein the transgene comprises a promoter.

69. The composition or combination of any of the preceding paragraphs, wherein the promoter is a synapsin (Syn1) promoter or a promoter of Tables 10-13.

70. The composition or combination of any of the preceding paragraphs, wherein the one or more miRNAs are located in an untranslated portion of the transgene.

71. The composition or combination of any of the preceding paragraphs, wherein the untranslated portion is an intron.

72. The composition or combination of any of the preceding paragraphs, wherein the untranslated portion is between the last codon of the nucleic acid sequence encoding a protein and a poly-A tail sequence, or between the last nucleotide base of a promoter sequence and a poly-A tail sequence.

73. The composition or combination of any of the preceding paragraphs, further comprising a third region comprising a second adeno-associated virus (AAV) inverted terminal repeat (ITR), or a variant thereof.

74. The composition or combination of any of the preceding paragraphs, wherein the ITR variant lacks a functional terminal resolution site (TRS), optionally wherein the ITR variant is a ATRS ITR.

75. The composition or combination of any of the preceding paragraphs, wherein the administration results in delivery of the viral vector or isolated nucleic acid to the central nervous system (CNS) of the subject.

76. The composition or combination of any of the preceding paragraphs, wherein the administration is via injection, optionally intravenous injection or intrastriatal injection.

77. The composition or combination of any of the preceding paragraphs, wherein the viral vector is an AAV1, AAV2, AAV3b, AAV4, AAV5, AAV6, AAV7, AAV8, AAV9, AAV10, AAVrh10, AAV11, AAV12, or a chimera thereof.

78. The composition of any of the preceding paragraphs, the viral vector comprises a capsid protein from AAV serotype AAV1, AAV2, AAV3b, AAV4, AAV5, AAV6, AAV7, AAV8, AAV9, AAV10, AAVrh10, AAV11, or, AAV12, or a chimera thereof.

79. The composition or combination of any of the preceding paragraphs, wherein the capsid protein is an AAV9 capsid protein.

80. The composition or combination of any of the preceding paragraphs, wherein the viral vector is a self-complementary AAV (scAAV).

81. The composition or combination of any of the preceding paragraphs, wherein the viral vector is formulated for delivery to the central nervous system (CNS).

82. A composition comprising an isolated nucleic acid encoding a CYP46A1 protein, the nucleic acid comprising a sequence at least 80% identical to SEQ ID NO: 110.

83. A composition comprising a recombinant viral vector comprising an isolated nucleic acid encoding a CYP46A1 protein, the nucleic acid comprising a sequence at least 80% identical to SEQ ID NO: 110.

84. A method for treating a neurological disease or disorder in a subject in need thereof, the method comprising administering to a subject having or at risk of developing the neurological disease or disorder a therapeutically effective amount of a composition of any of the preceding paragraphs.

85. The method of any of the preceding paragraphs, wherein the neurological disease or disorder is Alzheimer's disease, Parkinson's disease, Huntington's disease, Canavan disease, Leigh's disease, spinal cerebral ataxia, polyglutamine repeat spinocerebellar ataxia, Krabbe's disease, Batten's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, neuropathic pain, trauma due to spinal cord or head injury, ophthalmic diseases and disorders, Tay-Sachs disease, Lesch-Nyhan disease, epilepsy, cerebral infarcts, depression, bipolar affective disorder, persistent affective disorder, secondary mood disorder, schizophrenia, drug dependency, neuroses, psychosis, dementia, paranoia, attention deficit disorder, psychosexual disorders, sleeping disorders, pain disorders, eating or weight disorders.

86. The method of any of the preceding paragraphs, wherein the neurological disease or disorder is a central nervous system (CNS) disease or disorder.

87. The method of any of the preceding paragraphs, wherein the CNS disease or disorder is selected from Huntington's disease, Alzheimer's disease, Polyglutamine repeat spinocerebellar ataxias, Amyotrophic lateral sclerosis and Parkinson's disease.

88. The composition or method of any of the preceding paragraphs, wherein the recombinant viral vector is selected from the group consisting of: an AAV vector, an adenovirus vector, a lentivirus vector, a retrovirus vector, a herpesvirus vector, an alphavirus vector, a poxvirus vector a baculovirus vector, and a chimeric virus vector.

89. The method of any of the preceding paragraphs, wherein the administration is repeated at least once.

90. The method of any of the preceding paragraphs, wherein the administration results in delivery of the viral vector or isolated nucleic acid to the central nervous system (CNS) of the subject.

91. The method of any of the preceding paragraphs, wherein the administration is via injection, optionally intravenous injection or intrastriatal injection.

92. The composition or method of any of the preceding paragraphs, wherein the viral vector is AAV1, AAV2, AAV3b, AAV4, AAV5, AAV6, AAV7, AAV8, AAV9, AAV10, AAVrh10, AAV11, or, AAV12, or a chimera thereof.

93. The composition or method of any of the preceding paragraphs, viral vector comprises a capsid protein from AAV serotype AAV1, AAV2, AAV3b, AAV4, AAV5, AAV6, AAV7, AAV8, AAV9, AAV10, AAVrh10, AAV11, or, AAV12, or a chimera thereof.

94. The composition or method of any of the preceding paragraphs, wherein the capsid protein is an AAV9 capsid protein.

95. The composition or method of any of the preceding paragraphs, wherein the viral vector is a self-complementary AAV (scAAV).

96. The composition or method of any of the preceding paragraphs, wherein the viral vector is formulated for delivery to the central nervous system (CNS).

97. The composition or method of any of the preceding paragraphs, wherein the nucleic acid comprises a sequence at least 90% identical to SEQ ID NO: 110.

98. The composition or method of any of the preceding paragraphs, wherein the nucleic acid comprises a sequence at least 95% identical to SEQ ID NO: 110.

99. The composition or method of any of the preceding paragraphs, wherein the nucleic acid comprises a sequence identical to SEQ ID NO: 110.

100. The composition or method of any of the preceding paragraphs, where the viral vector comprises a modified viral capsid.

101. The composition or method of any of the preceding paragraphs, where the viral vector comprises a modification to a viral capsid.

102. The composition or method of any of the preceding paragraphs, wherein the modification is a chemical, non-chemical or amino acid modification of the viral capsid.

103. The composition or method of any of the preceding paragraphs, wherein at least one of the capsid modifications preferentially targets cells in the CNS or PNS.

104. The composition or method of any of the preceding paragraphs, wherein the chemical modification comprises a chemically-modified tyrosine residue modified to comprise a covalently-linked mono- or polysaccharide moiety.

105. The composition or method of any of the preceding paragraphs, wherein the chemically-modified tyrosine residue comprises a mono-saccharide selected from galactose, mannose, N-acetylgalactosamine, bridge GalNac, and mannose-6-phosphate.

106. The composition or method of any of the preceding paragraphs, wherein the chemical modification comprises a ligand covalently linked to a primary amino group of a capsid polypeptide via a —CSNH— bond.

107. The composition or method of any of the preceding paragraphs, wherein the ligand comprises an arylene or heteroarylene radical covalently bound to the ligand.

108. The composition or method of any of the preceding paragraphs, wherein the modified viral capsid is a chimeric capsid or a haploid capsid.

109. The composition or method of any of the preceding paragraphs, wherein the modified viral capsid is a haploid capsid.

110. The composition or method of any of the preceding paragraphs, wherein the modified viral capsid is a chimeric or haploid capsid further comprising a modification.

111. The composition or method of any of the preceding paragraphs, wherein the modified viral capsid is an AAV serotype AAV1, AAV2, AAV3b, AAV4, AAV5, AAV6, AAV7, AAV8, AAV9, AAV10, AAVrh10, AAV11, AAV12, or a mutant modified from, a chimera, a mosaic, or a rational haploid thereof.

112. The composition or method of any of the preceding paragraphs, wherein the modification changes the antigenic profile of the modified viral capsid as compared to the unmodified viral capsid.

113. The composition or method of any of the preceding paragraphs, wherein the modified viral capsid can be used for repeat administration.

114. A synthetic CNS-specific promoter comprising or consisting of a sequence according to any one of SEQ ID NOs: 187-189 or a functional variant thereof.

115. The synthetic CNS-specific promoter of any of the preceding paragraphs, wherein the functional variant is at least 60%, 65%, 70%, 75%, 80%, 85%, 90%, 91%, 92%, 93%, 94%, 95%, 96%, 97%, 98% or 99% identical to any one of SEQ ID NOs: 187-189.

116. A CRE comprising or consisting of a sequence according to any one of SEQ ID NO: 191 or 192 or a functional variant thereof.

117. The CRE of any of the preceding paragraphs, comprising a sequence which is at least 60%, 65%, 70%, 75%, 80%, 85%, 90%, 91%, 92%, 93%, 94%, 95%, 96%, 97%, 98% or 99% identical to any one of SEQ ID NOs: 191 or 192.

118. A CRM comprising a CRE of any of the preceding paragraphs.

119. A minimal promoter comprising or consisting of a sequence according to SEQ ID NO: 211 or a functional variant thereof.

120. A CNS-specific promoter comprising a CRE of any of the preceding paragraphs, a CRM of any of the preceding paragraphs, or a minimal promoter of any of the preceding paragraphs.

121. An expression cassette comprising a synthetic CNS-specific promoter of any of the preceding paragraphs, operably linked to a sequence encoding an expression product.

122. A vector comprising a synthetic CNS-specific promoter of any of the preceding paragraphs or an expression cassette of any of the preceding paragraphs.

123. The vector of any of the preceding paragraphs, wherein the vector is a viral vector.

124. The vector of any of the preceding paragraphs, wherein the viral vector is an AAV vector.

125. A virion comprising a vector of any of the preceding paragraphs.

126. A pharmaceutical composition comprising a synthetic CNS-specific promoter of any of the preceding paragraphs, an expression cassette of any of the preceding paragraphs, a vector of any of the preceding paragraphs, or a virion of any of the preceding paragraphs.

127. A synthetic CNS-specific promoter of any of the preceding paragraphs, an expression cassette of any of the preceding paragraphs, a vector of any of the preceding paragraphs, a virion of any of the preceding paragraphs, or a pharmaceutical composition of any of the preceding paragraphs for use in therapy.

128. The synthetic CNS-specific promoter, the expression cassette, the vector, the virion, or the pharmaceutical composition of any of the preceding paragraphs for use in gene therapy, suitably wherein the gene therapy involves expression of a therapeutic expression product in the CNS.

129. The synthetic CNS-specific promoter, the expression cassette, the vector, the virion, or the pharmaceutical composition of any of the preceding paragraphs for use in gene therapy of a CNS-related disease.

130. A cell comprising a synthetic CNS-specific promoter of any of the preceding paragraphs, an expression cassette of any of the preceding paragraphs, a vector of any of the preceding paragraphs, or a virion of any of the preceding paragraphs.

131. The cell of any of the preceding paragraphs, wherein the cell is a CNS cell, optionally a human CNS cell, preferably a neuron, more preferably a dopaminergic neuron.

132. A synthetic CNS-specific promoter of any of the preceding paragraphs, an expression cassette of any of the preceding paragraphs, a vector of any of the preceding paragraphs, or a virion of any of the preceding paragraphs for use in the manufacture of a pharmaceutical composition for the treatment of a medical condition or disease, optionally wherein the disease is a CNS-related disease.

133. A method for producing an expression product, the method comprising: introducing a synthetic CNS-specific expression cassette of any of the preceding paragraphs into a cell, optionally a CNS cell; and expressing the gene present in the synthetic CNS-specific expression cassette.

134. A method of expressing a therapeutic transgene in a cell, optionally a CNS cell, wherein the method comprises introducing into the cell an expression cassette of any of the preceding paragraphs, a vector of any of the preceding paragraphs, or a virion of any of the preceding paragraphs.

135. A method of therapy of a subject in need thereof, wherein the method comprises: administering to the subject in need thereof, an expression cassette of any of the preceding paragraphs, a vector of any one of any of the preceding paragraphs, or a virion of any of the preceding paragraphs, or a pharmaceutical composition of any of the preceding paragraphs, which comprises a sequence encoding a therapeutic product operably linked to.

### EXAMPLES

### Example 1

In one aspect described herein are inhibitory RNAs that can be used for the treatment of Huntington's disease. In some embodiments of any of the aspects, the nucleic acid sequence of the inhibitory RNA comprises one of SEQ ID NO: 6-17, 40-44, 50-66, 158-185, or 217-260 or a sequence that is at least 95% (e.g., at least 95%, at least 96%, at least 97%, at least 98%, or at least 99%) identical to the sequence of at least one of SEQ ID NO: 6-17, 40-44, 50-66, 158-185, or 217-260 that maintains the same functions as SEQ ID NO: 6-17, 40-44, 50-66, 158-185, or 217-260 (e.g., HTT inhibition).

Described here are constructs comprising artificial miRNAs. pEMBL-D(+)-Syn1-hCG intron is a control vector, which is inserted with empty human chorionic gonadotropin (hCG) intron (hCGin) and driven with synapsin promoter. Two copies of control miRNA precursor (random sequences or non-functional mutation) are inserted into hCGin in the vector pEMBL-D(+)-Syn1-hCGin-2× control pre-miR. Two copies of artificial pre-miR (perfect match with 3′-UTR targeting sequences, including about 100-150 bp flanked upstream and downstream sequences) are cloned into between the hCG introns. The vector pEMBL-D(+)-Syn1-CYP46A1-hCGin-2× artificial pre-miR is a combo construct, which could produce both CYP46A1 and artificial miRNA at the same time. In order to identify whether the pre-miRNA could be processed into mature miRNA and combined with HTT targeting sequences including CAG expansions, which are perfectly complementary with mature miRNA, are inserted behind luciferase gene. For the limit of package size, small poly A is used in the constructs.

The sequences of the following are known in the art: pEMBL; synapsin promoter (Syn1); ITRs (e.g., from AAV1, AAV2, AAV3b, AAV4, AAV5, AAV6, AAV7, AAV8, AAV9, AAV10, AAVrh10, AAV11, AAV12, or AAV13); hCG intron; small polyA; CYP46A1; luciferase; HTT targeting sequences; and/or HTT-3′UTR/mutant.

Synapsin-1 (Syn1) is a member of the synapsin gene family. Synapsins encode neuronal phosphoproteins which associate with the cytoplasmic surface of synaptic vesicles. Family members are characterized by common protein domains, and they are implicated in synaptogenesis and the modulation of neurotransmitter release, suggesting a potential role in several neuropsychiatric diseases. Syn1 plays a role in regulation of axonogenesis and synaptogenesis. Syn1 protein serves as a substrate for several different protein kinases and phosphorylation may function in the regulation of this protein in the nerve terminal. Mutations in this gene may be associated with X-linked disorders with primary neuronal degeneration such as Rett syndrome. Alternatively, spliced transcript variants encoding different isoforms have been identified. In some embodiments of any of the aspects, the Syn1 promoter can comprise a human promoter Syn1 (see e.g., the Syn1 promoter associated with NCBI ref numbers NG_008437.1 RefSeqGene Range 5001-52957; NM_006950.3; NP_008881.2; NM_133499.2; NP_598006.1).

CYP46A1 is a member of the cytochrome P450 superfamily of enzymes. The cytochrome P450 proteins are monooxygenases which catalyze many reactions involved in drug metabolism and synthesis of cholesterol, steroids and other lipids. This endoplasmic reticulum protein is expressed in the brain, where it converts cholesterol to 24S-hydroxycholesterol. While cholesterol cannot pass the blood-brain barrier, 24S-hydroxycholesterol can be secreted in the brain into the circulation to be returned to the liver for catabolism. In some embodiments of any of the aspects, CYP46A1 can comprise a human CYP46A1 (see e.g., NCBI ref numbers NG_007963.1 RefSeqGene Range 4881-47884; NM_006668.2; NP_006659.1). CYP46A1, the rate-limiting enzyme for cholesterol degradation, is neuroprotective in Huntington's disease (see e.g., Boussicault et al., CYP46A1, the rate-limiting enzyme for cholesterol degradation, is neuroprotective in Huntington's disease, Brain. 2016 March, 139(Pt 3):953-70; Kacher et al., CYP46A1 gene therapy deciphers the role of brain cholesterol metabolism in Huntington's disease, Brain. 2019 Aug. 1; 142(8):2432-2450; the contents of each of which are incorporated herein by reference in their entireties).

In some embodiments of any of the aspects, an miRNA comprises a sequence complementary to at least two (e.g., 2, 3, 4, 5, 6, 7, 8, 9, 10, 11, 12, 13, 14, 15, 16, 17, 18, 19, 20, 21, 22, 23, 24, or 25) continuous bases of the sequence set forth in SEQ ID NO: 3 or 4 flanked by a miRNA backbone sequence. In some embodiments of any of the aspects, an miRNA comprises a sequence complementary to at least two (e.g., 2, 3, 4, 5, 6, 7, 8, 9, 10, 11, 12, 13, 14, 15, 16, 17, 18, 19, 20, 21, 22, 23, 24, or 25) continuous bases of the sequence of an untranslated region (e.g., 5′ UTR, 3′UTR), exon, CAG repeat, or CAG jumper (e.g., CAG 5′ jumper, CAG 3′ jumper) associated with HTT (see e.g., NCBI Gene ID: 3064; e.g., SEQ ID NO: 4) flanked by a miRNA backbone sequence.

An isolated nucleic acid encoding a transgene encoding one or more miRNAs and an isolated nucleic acid encoding a CYP46A1 protein, when administered to the same patient can provide an improved therapeutic effect than either administered alone. An isolated nucleic acid encoding a transgene encoding one or more miRNAs and an isolated nucleic acid encoding a CYP46A1 protein, when administered to the same patient can provide a synergistically (rather than an additively) improved therapeutic effect than either administered alone. The isolated nucleic acid encoding a transgene encoding one or more miRNAs and isolated nucleic acid encoding a CYP46A1 protein can be administered sequentially or concurrently to the subject, in accordance with any of the methods described herein.

### Example 2—HDII Exon-Targeting miRNA Analysis

### Materials and Methods

Fibroblasts isolated from a patient with confirmed Huntington's disease (Coriell GM04281) were cultured in MEM (Gibco 11090) supplemented with 15% FBS (Gibco 10270106), 1% glutamax (Gibco 35050), 1% non-essential amino acids (Merck M7145), and 1% Penicillin-Streptomycin (Gibco 15140122). 1E+06 cells were nucleofected with 5 μg of plasmid using the Basic Nucleofector Kit for Primary Mammalian Fibroblasts (Lonza VPI-1002) with nucleofector program U-023 on the Nucleofector 2b device (Lonza). The plasmids contain two copies of a miRNA in the 3′ coding region of the GFP gene, under the control of the CMV promoter. 48 hours post-nucleofection the cells were washed with PBS before dissociation with 0.25% trypsin-EDTA (Gibco 25200056). The cells were collected, resuspended in 90% PBS, 10% FBS solution, and put through a cell strainer (VWR 734-0001). GFP positive cells were isolated by flow cytometry, and the RNA extracted using the RNeasy micro kit (Qiagen 74004). cDNA synthesis was conducted using the M-MLV cDNA synthesis kit (Thermo Fisher 28025013) and the sequence specific primer UAPd18 (sequence GGCCACGCGTCGACTAGTACTTTTTTTTTTTTTTTTTT (SEQ ID NO: 186)). qPCR was used to calculate the relative expression of HTT (HTT exon 1 primer and probe sequences taken from Neueder et al. 2017) relative to housekeeping gene HPRT1 (Hs02800695_m1) using the Tagman Fast Advanced Master Mix (Thermo Fisher 4444554).

### Results

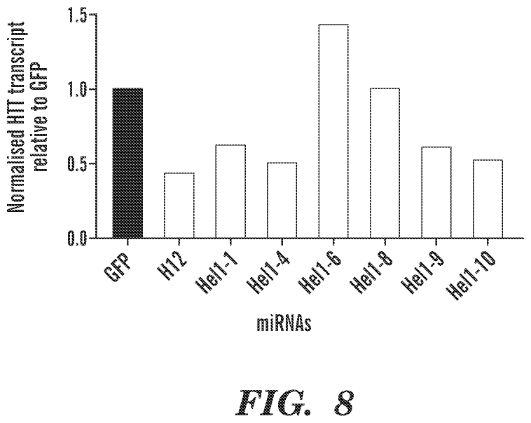

Fibroblasts derived from a patient with Huntington's disease were nucleofected with plasmid constructs expressing miRNAs H12, Hell-1, Hell-4, Hell-6, Hell-8, Hell-9, or Hell-10 in the 3′ end of a GFP coding region, under the control of the ubiquitous CMV promoter. H12 is positive control taken from Miniarikova et al. (2016). Following nucleofection, miRNAs H12, Hell-1, Hell-4, Hell-9, and Hell-10 demonstrate knock down of HTT compared to the GFP control (which contains no miRNAs in the GFP sequence) (see, e.g., FIG. 8).

### REFERENCES

Miniarikova et al. (2016) Design, Characterization, and Lead Selection of Therapeutic miRNAs Targeting Huntingtin for Development of Gene Therapy for Huntington's Disease. Molecular Therapy—Nucleic Acids 5, e297
Neueder et al. (2017) The pathogenic exon 1 HTT protein is produced by incomplete splicing in Huntington's disease patients. Scientific Reports 7(1): 1307

### Example 3—Methods for Assessing Promoter Activity

In order to assess the activity in the CNS of a CRE, CRM, a promoter element or a synthetic CNS-specific promoter, it is desirable to operably link the synthetic CNS-specific promoter or a synthetic CNS-specific promoter comprising the CRE, CRM, a promoter element to a reporter gene (such as GFP, YFP or RFP). The expression construct comprising the above is administered intravenously in a viral vector penetrating the CNS in an animal (such as a mouse or a rat). Following a time period (such as 1 week, 2 weeks, 3 weeks, 1 month, 2 months, 3 months), the animal is sacrificed. The CNS of the animal is processed into tissue sections and immunostaining (fluorescent and colorimetric) may be performed to assess the expression of the reporter throughout the CNS and in different CNS cell types. The skilled person will understand that different CNS cell types may be identified using cell specific markers. For example, microglial cells may be identified using fluorescently tagged antibodies specific to IBA1, astrocytes may be identified using fluorescently tagged antibodies specific to GFAP, oligodendrocytes may be identified using fluorescently tagged antibodies specific to Oligo2 and neurons may be identified using fluorescently tagged antibodies specific to NeuN. If quantification is required, a western blot for the reporter is performed on CNS tissue. Performing these techniques is routine and known.

### Example 4—In Vivo Assessment of the Tolerance of AAV-eGFP-miHTT Constructs at Several Titers in C57BL/6J Mouse Striatum

The goal of the present study is to evaluate the tolerance, at injection site, of AAV-miHTT constructs, after administration at different titers in the striatum of C57BL/6J mice. For this, markers for neurons are analyzed by immunohistochemistry (IHC) and tolerance is evaluated by quantifying local cell loss at the injection site. This study assesses cellular tropism by analyzing eGFP immunoreactivity in the different cell types and vector copy genomes/cell in the striatum.

The present study assesses the tolerance of AVV-GFP-miHTT constructs at the injection site after administration at three different titers in the striatum of C57BL/6J mice: 1.0E9 vg/μL that according to previous studies (BV23) was well tolerated; in addition, two additional titers, 3.5.0E9 vg/μL and 6.5E9 vg/μL, that were not evaluated in vivo using the present cassette (GFP-miHTT), are tested. For this, markers for neurons and glial cells are analyzed by immunohistochemistry (IHC) and tolerance is evaluated by quantifying local cell loss at the injection site. This study also includes an assessment of cellular tropism by analyzing eGFP immunoreactivity in the different cell types in the mouse brain and analysis of vector copy genomes/cell in the striatum.

The day of intracranial injection is designated as Day 1, with subsequent days consecutively numbered. Days in the study prior to the intracranial injection are consecutively numbered. Stereotaxic injections are done at two different days and sacrifice of mice are done on two distinct days to respect the same in-life period (6 weeks).

### Vectors and Vector Preparation

AAVrh10 encoding eGFP and miR-Hell-9 under control of CBA (AAVrh10-CBA-eGFP-miR-Hell-9) (lot-AAV-B367) is provided at a concentration of 6.85E9 vg/μL. AAVrh10 encoding eGFP and miR-Hell-10 under control of CBA (AAVrh10-CBA-eGFP-miR-Hell-10) (lot-AAV-B368) is provided at a concentration of 1.73E10 vg/μL.

A dilution buffer (vehicle) with the same composition as the formulation buffer was used in the manufacturing of the vector (based on the reference from Lonza, #: BE17-513F): dPBS, Ca²⁺/Mg²⁺ supplemented with Pluronic F68 0.001% was prepared by the VVC manufacturing team.

Vectors are diluted in Vehicle (dPBS Ca²⁺/Mg²⁺ Pluronic F68 0.001%) just prior to use under a safety cabinet (BSC; L2 confinement under laminar flow) in an autoclaved 0.5 mL polypropylene Eppendorf tube (Eppendorf, ref. 0030 121.023) to have the appropriate titer, that is maintained at 4° C., on ice, during the period of the stereotaxic procedure. Dose formulations are drawn up into the device of administration: Hamilton syringes (Dutscher; 1701 RN 10 μL; ref: 074493) containing a 32G tip type 3 30 mm length needle (Dutscher; ref: 074753), are filled in the dosing area just prior to each dose administration.

### Experimental Design

A total of 21, 8-week old female C57BL/6J mice, ranging from 15 g to 25 g, are used in this study. Mice are distributed randomly to groups, as indicate in Table 19, without any randomization procedure. Mice receive bilateral striatal injection of the indicated vector in a volume of 4 μL/striatum. The left and right hemispheres of each mouse are bilaterally injected with the vector (4 μL) diluted in the same vehicle. Vectors are administered from 1.0E9 vg/μL (4.0E9 vg/striatum) to 6.5E9 vg/μL (2.6E10 vg/striatum).

TABLE 19
Group assignments

| | | | Dose | | | |
|---|---|---|---|---|---|---|
| | | | Titer | Dose | Volume & injection | |
| Group | Mice | Test Article | (vg/μL) | (vg/striatum) | (μL/animal) | Animals |
| 1 | C57BL/6J | Vehicle | — | | 8 μL in total; 4 μL/site; 1 site/striatum - | 3F |
| | | | | | Bilateral | |
| 2 | C57BL/6J | AAV-B637 | 1.0E9 | 4.0E9 | 8 1.1 L in total; 4 μL/site; 1 site/striatum - | 3F |
| | | | | | Bilateral | |
| 3 | C57BL/6J | AAV-B637 | 3.5E9 | 1.4E10 | 8 μL in total; 4 μL/site; 1 site/striatum - | 3F |
| | | | | | Bilateral | |
| 4 | C57BL/6J | AAV-B637 | 6.5E9 | 2.6E10 | 8 μL in total; 4 μL/site; 1 site/striatum - | 3F |
| | | | | | Bilateral | |
| 5 | C57BL/6J | AAV-B638 | 1.0E9 | 4.0E9 | 8 μL in total; 4 μL/site; 1 site/striatum - | 3F |
| | | | | | Bilateral | |
| 6 | C57BL/6J | AAV-B638 | 3.5E9 | 1.4E10 | 8 μL in total; 4 μL/site; 1 site/striatum - | 3F |
| | | | | | Bilateral | |
| 7 | C57BL/6J | AAV-B638 | 6.5E9 | 2.6E10 | 8 μL in total; 4 μL/site; 1 site/striatum - | 3F |
| | | | | | Bilateral | |

TABLE 20
Vector preparation details

| | | | | | | Total | |
|---|---|---|---|---|---|---|---|
| | | | | Drawn | Drawn | Volume | |
| | Vehicle | | | volume | volume | of | |
| | (internal | | | of | of | diluted | Number of |
| | batch | Initial titer | Final titer | vector | Vehicle | solution | animals to be |
| TA | number) | (vg/μ1) | (vg/μL) | (4) | (μL) | (μL) | injected/group |
| AAVrh10- | 08 | 6.85E9 | 1.0E9 | 14.6 | 85.4 | 100* | 3 |
| CBA-eGFP- | 08 | 6.85E9 | 3.5E9 | 51.1 | 48.9 | 100* | 3 |
| miR-Hel1-9 | 08 | 6.85E9 | 6.5E9 | 94.9 | 5.1 | 100* | 3 |
| AAVrh10- | 08 | 1.73E10 | 1.0E9 | 5.8 | 94.2 | 100* | 3 |
| CBA-eGFP- | 08 | 1.73E10 | 3.5E9 | 20.2 | 79.8 | 100* | 3 |
| miR-Hel1-10 | 08 | 1.73E10 | 6.5E9 | 37.6 | 62.4 | 100* | 3 |

### Summarized Protocol of Intracerebral Delivery in Mouse Striatum:

Vectors are administered via intracerebral stereotaxic delivery in the mouse striatum. Administration consists of a single administration per hemisphere. For each single injection the duration of administration is 20 min, and then the needle is left in place 5 min additionally to avoid flow-back. Total time is 40 minutes of injection+10 min of needle resting in place for each mouse injected bilaterally and 20 minutes of injection+5 min of needle resting in place for each mouse injected unilaterally.

Mice are anesthetized by intraperitoneal injection of ketamine (Imalgene 1000, 100 mg/Kg)/xylazine (Rompun 2%, 10 mg/Kg) and positioned on a stereotaxic frame (Stoelting, Wood Dale, USA). Four microliters of vector preparation is injected into the left striatum and right striatum for mice injected bilaterally, at a rate of 0.2 μL/min by means of a Hamilton syringe containing a 32G tip type 3 30 mm length needle connected to an automatic injector (KD Scientific; Phymep France; ref: 78-8130 INT). For mice injected unilaterally the same protocol is followed, except that the vector preparation is injected only into the left striatum. Stereotaxic coordinates of injection sites from bregma are the following: antero-posterior (+1 mm); medio-lateral (+/−2 mm); dorso-ventral (−3.5 mm); tooth bar: 0. All vector dilutions are prepared immediately prior to dosing.

Daily observation/monitoring of mice are performed to assess food/water consumption, general behavior (general state and movements in the cage), any adverse effects, just after the surgery and then since the first day after the injection. Each animal is weighed at the moment of anesthesia and once a week until the day of necropsy, 6 weeks post-injection.

Whole Brain Tissue is Collected from all Animals Post-Fixed in 4% PFA for IHC and Expression Analysis.

On the day of necropsy, surviving animals have terminal body weight recorded, and anesthesia performed. A lethal dose of Euthasol (Vetcare) (180 mg/Kg) is intraperitoneally administered to mice. Then, animals are euthanized using intracardiac perfusion with 25 mL of ice-cold 4% PFA in PBS 0.1 M.

Immediately after perfusion of mice with ice-cold 4% PFA in PBS 0.1M, the brain is collected from all animals, and post-fixed in 4% PFA solution for 24 h. After that, the 4% PFA solution is replaced, and the brain is cryoprotected by incubation in 20% sucrose/phosphate buffer for 24 h. Subsequently, the post-fixed brain is frozen and coronal 40 μm free-floating brain sections are obtained using a freezing microtome (Leica, Wetzlar, Germany). Brain sections are stored at −20° C. in a solution composed at 1/10 of cryoprotective solution composed at 1/10 of Phosphate buffer (Sigma-Aldrich, ref. P3619-1GA), 3/10 distilled water, 3/10 ethylene glycol (Sigma-Aldrich, ref: 324558), 3/10 glycerol (Life Sciences GE Healthcare).

IHC against the eGFP protein (rabbit anti-eGFP; Abcam, reference: ab6556; dilution: 1:6000), neuronal marker NeuN (Abcam, reference: ab177487; dilution 1/2000), and neuronal marker lba1 (Wako, reference: 019-19741; dilution 1/2000) is performed in serial frozen brain slices containing the whole rostro-caudal sampling of the striatum to evaluate eGFP expression, neuronal expression, and glial expression, respectively, thus allowing to qualitatively estimate the area of expression eGFP, neuronal preservation, and glial activation, respectively, in the overall regions of the striatum.

Images of immunostained sections are acquired with a Zeiss Axio Scan Z1 or a Hamamatsu Nanozoomer S60. During section scanning, light intensity, exposure time, numeric gain and magnification were kept constant between animals.

### Quantification of Frozen Brain Slices

Using ImageJ software, eGFP positive areas, quantification of areas of neuronal loss, and quantification of microglial activation are measured in all frozen brain sections in which the mouse striatum is present. During slice scanning, numeric gain and magnification are kept constant between animals to avoid potential technical artifacts. Images are first converted to 8-bit grey scale and binary thresholded to highlight a positive staining. All evaluated areas are drawn by the same blinded experimenter and measured by the software.

For the analysis the total eGFP transduced area is reported to the total area of the striatum and a percentage of the transduced area per mouse is calculated as follows: (transduced area/total striatal area)×100.

For the analysis the total neuronal depleted area (anti-NeuN immunostaining) is reported to the total area of the striatum and a percentage of depleted area per mouse is calculated as follows: (transduced area in striatum/total striatal area)×100.

For the analysis the total lba1 stained area (anti-lba1 immunostaining) are reported to the total area of the striatum and a percentage of lba1 expressing area per mouse is calculated as follows: (Iba1 expressing area in striatum/total striatal area)×100.

DNA Isolation from Frozen Brain Slices

For 40-μm slices brain, a rostro-caudal series sampling (fixed frozen sections), representative of the whole striatum is dissected for each slice. At the end, all striatal slices from the same hemisphere are pooled in the same polypropylene tube (Sarstedt reference: 72706) for DNA extraction. DNA isolation for brain is performed with the QIAamp® DNA FFPE Tissue Kit (Qiagen reference: 56404) according to manufacturer's instructions.

For each sample, 10 ng of DNA/well is used. Quantitative real-time PCR reactions are performed using LightCycler® 480 SYBR® Green I Master (Roche, ref: 04707516001) according to manufacturer's protocol and run on LightCycler® 480 (Roche Diagnostics, Germany). The mADCK3 housekeeping gene is used to normalize the quantification of hCYP46A1 levels.

Primer sequences:
eGFP (transgene):
(SEQ ID NO: 213)
FW: 5′- GACGACGGCAACTACAAGA-3′;
(SEQ ID NO: 214)
RW: 5′- GCTTGTCGGCCATGATATAGA-3′
mADCK3 (endogenous gene):
(SEQ ID NO: 215)
FW: 5′-CCACCTCTCCTATGGGCAGA-3′;
(SEQ ID NO: 216)
RW: 5′-CCGGG CCTTTTCAATGTCT-3′

Results (vector genome copy number per cell, VGC/cell) are expressed as n-fold differences in the transgene sequence copy number (eGFP) relative to the mADCK3 gene copy (number of viral genomes copy for 2N genome). Results are determined by the formula: NeGFP=2^{2(t)}, where the ΔCₜ value of the sample is determined by subtracting the Cₜ value of the target gene from the Cₜ value of the mACDK3 gene. Positive controls from previous experiments can be used. The striatum and liver are used. The cerebral cortex is collected and conserved at −80° C. and are not immediately processed (optional processing for VGC analysis).

All analyses are performed using GraphPad Prism version 8 (Graph Pad Software, La Jolla, USA). All data are represented as mean±SEM. For the analysis of weight gain, vector genome copies, eGFP transduced area, NeuN depleted area and microglial activated area, statistical significance is assessed using a One-way ANOVA followed by Bonferroni's post-hoc test with treatment as independent factor; a Student's T test may be used to compare one-by-one, groups injected with vectors. For body weight measurements a Two-way ANOVA corrected by Bonferroni's post-hoc is performed with time and treatment as independent factors. Summary statistics (mean and standard error of the mean) of all numerical data from scheduled animals are provided. For all analyses, statistical significance is set to a P-value<0.05.

### Example 5—In Vivo Characterization of Different AAV-eGFP-miHTT Constructs Expressing miRNA targeting Huntingtin sequence after injection in C57BL/6J mouse striatum

The goal of the present study is to assess the in vivo functionality of several AAV-miHTT constructs containing different miRNA sequences targeting human Huntingtin (HTT) (non-allele specific). For this, the expression of the reporter gene, eGFP, is assessed by evaluating total eGFP protein levels by western blot and the number of vector genome copies/cell (vgc/cell) after injection of each vector in C57BL/6J mouse striatum. In addition, eGFP immunoreactivity in mouse brain slices is analyzed by immunohistochemistry (IHC).

The expansion of the polyglutamine (polyQ) tract in HTT protein leads to perturbation of normal functions and induces a toxic gain-of-function causing cellular dysfunction and ultimately neuronal death observed in HD condition. Different studies have shown that lowering mutant HTT in the brain of rodent models can improve molecular, neuropathological and behavioral abnormalities (Caron et al, 2020, Miniarikova et al, 2017 & Spronck et al, 2019). Thus, targeting and reducing the levels of mutant HTT (and normal Huntingtin) has been considered as a promising therapeutic strategy. After selecting different miRNA sequences that target HTT, their in vivo functionality is assessed by injecting several constructs expressing each an miHTT sequence and eGFP, a reporter gene, in C57BL/6J mouse brain. For this, eGFP expression is assessed by evaluating total eGFP protein levels by western blot and the number of vgc/cell after injection of each vector in C57BL/6J mouse striatum. In addition, eGFP immunoreactivity in mouse brain is analyzed by immunohistochemistry (IHC).

The day of intracranial injection is designated as Day 1, with subsequent days consecutively numbered. Days on study prior to the intracranial injection are consecutively numbered. Stereotaxic injections are done at three different days and sacrifice of mice is done on three distinct days to respect the same in-life period (2 weeks).

### Vectors and Vector Preparation

The following vectors are used in the present study:

AAVrh10 encoding eGFP and miR-Hell-9 under control of CBA promoter (AAVrh10-CBA-eGFP-miR-Hell-9 (AAV-B637)) is provided at a concentration of 6.85E9 vg/μL.

AAVrh10 encoding eGFP and miR-Hell-10 under control of CBA promoter (AAVrh10-CBA-eGFP-miR-Hell-10 (AAV-B638)) is provided at a concentration of 1.73E10 vg/μL.

AAVrh10 encoding eGFP and miR-He4 under control of CBA promoter (AAVrh10-CBA-eGFP-miR-He4 (AAV-B639)) is provided at a concentration of 3.73E10 vg/μL.

AAVrh10 encoding eGFP and miR-H12 under control of CBA promoter (AAVrh10-CBA-eGFP-miR-H12 (AAV-B640)) is provided at a concentration of 1.11E10 vg/μL.

AAVrh10 encoding eGFP under control of CBA promoter (AAVrh10-CBA-eGFP (AAV-B641) is provided at a concentration of 1.52E10 vg/μL.

Vectors are prepared and diluted via protocols described in Example 4. Dose formulations are drawn up via protocols described in Example 4.

### Experimental Design

A total of 25, 8-week old female C57BL/6J mice, ranging from 15 g to 25 g, are used in this study. Mice are distributed randomly to groups, as indicate in Table 21, without any randomization procedure. Mice receive bilateral striatal injection of the indicated vector in a volume of 4 μL/striatum. The left and right hemispheres of each mouse are bilaterally injected with the vector (4 μL) diluted in the same vehicle. Vectors are administered from 1.0E9 vg/μL (4.0E9 vg/striatum).

TABLE 21
Group Assignments

| | | | Dose | | | |
|---|---|---|---|---|---|---|
| | | | Titer | Dose | | |
| Group | Mice | Test Article | (vg/μL) | (vg/striatum) | Volume & injection (μL/animal) | Animals |
| 1 | C57BL/6J | AAV-B637 | 1.0E9 | 4.0E9 | 8 μL in total; 4 μL/site; 1 site/striatum - bilateral | 3F |
| | | | | | 4 μL in total; 4 μL/site; 1 site/striatum - Unilateral (LH) | 2F |
| 2 | C57BL/6J | AAV-B638 | 1.0E9 | 4.0E9 | 8 μL in total; 4 μL/site; 1 site/striatum - bilateral | 3F |
| | | | | | 4 μL in total; 4 μL/site; 1 site/striatum - Unilateral (LH) | 2F |
| 3 | C57BL/6J | AAV-B639 | 1.0E9 | 4.0E9 | 8 μL in total; 4 μL/site; 1 site/striatum - Bilateral | 3F |
| | | | | | 4 μL in total; 4 μL/site; 1 site/striatum - Unilateral (LH) | 2F |
| 4 | C57BL/6J | AAV-B640 | 1.0E9 | 4.0E9 | 8 μL in total; 4 μL/site; 1 site/striatum - Bilateral | 3F |
| | | | | | 4 μL in total; 4 μL/site; 1 site/striatum - Unilateral (LH) | 2F |
| 5 | C57BL/6J | AAV-B641 | 1.0E9 | 4.0E9 | 8 μL in total; 4 μL/site; 1 site/striatum - Bilateral | 3F |
| | | | | | 4 μL in total; 4 μL/site; 1 site/striatum - Unilateral (LH) | 2F |

TABLE 22
Test Article preparation details

| | | | | | | Total | |
|---|---|---|---|---|---|---|---|
| | | | | Drawn | Drawn | Volume of | |
| | Vehicle | Initial | Final | volume | volume of | diluted | Number of |
| | (internal batch | titer | titer | of Vector | Vehicle | solution | animals to be |
| TA | number) | (vg/μL) | (vg/μL) | (μL) | (μL) | (μL) | injected/group |
| AAV-B637 | 08 | 6.85E9 | 1.0E9 | 14.6 | 85.4 | 100* | 5 |
| AAV-B638 | 08 | 1.73E10 | 1.0E9 | 5.8 | 94.2 | 100* | 5 |
| AAV-B639 | 08 | 3.73E10 | 1.0E9 | 2.7 | 97.3 | 100* | 5 |
| AAV-B640 | 08 | 1.11E10 | 1.0E9 | 9.0 | 91.0 | 100* | 5 |
| AAV-B641 | 08 | 1.52E10 | 1.0E9 | 6.6 | 93.4 | 100* | 5 |

Vectors are administered via intracerebral delivery in mouse striatum, as described in Example 4. For administration, mice are anesthetized as described in Example 4.

Whole Brain Tissue is Collected from all Animals Post-Fixed in 4% PFA for IHC and Expression Analysis.

Methods and protocols for and after necropsy and euthanasia as described in Example 4 are followed. Animals are euthanized using intracardiac perfusion with 25 mL of ice-cold 4% PFA in PBS 0.1 M. The following tissues are collected from all animals and frozen for eGFP protein levels and vgc/cell analyses, and post-fixed in 4% PFA for IHC analysis: Striatal punch of left and right hemisphere from all cohorts injected bilaterally; cerebral cortex punch of left and right hemisphere from all cohorts injected bilaterally; and whole brain from all cohorts injected unilaterally.

Immediately after euthanasia and perfusion of mice (n=3 mice) with PBS 0.1M, the two hemispheres of the brain are dissected for each animal injected bilaterally and the striatum and cerebral cortex from each hemisphere are collected into four different 1.5 mL Eppendorf tubes (Eppendorf reference: 0030 121.023), that are immediately frozen in dry ice and stored at −80° C. until use (n=6 per condition). Then, DNA and protein are simultaneously extracted from the same sample using AllPrep® DNA/RNA/Protein Mini Kit (Qiagen, ref: 80004), and then stored at −20° C. for genomic DNA that is used in vgc detection and −80° C. for proteins that are used in western blot.

Immediately after perfusion of mice with ice-cold PBS 0.1M, the brain is collected from all animals injected unilaterally (n=2/group), and post-fixed in 4% PFA solution for 24 h. After that, the PFA 4% solution is replaced, and the brain is cryoprotected by incubation in 20% sucrose/phosphate buffer for 24 h. Subsequently, the post-fixed brain is frozen and coronal 40 μm free-floating brain sections are obtained using a freezing microtome (Leica, Wetzlar, Germany). Brain sections are stored at −20° C. in a solution composed at 1/10 of cryoprotective solution composed at 1/10 of Phosphate buffer (Sigma-Aldrich, ref P3619-1GA), 3/10 distilled water, 3/10 ethylene glycol (Sigma-Aldrich, ref: 324558), 3/10 glycerol (Life Sciences GE Healthcare).

Quantitative PCR for vector copy number/cell assessment in mouse striatum is performed as described in Example 4. Electrophoresis is performed on a Bio-rad apparatus with a standard protocol and Bio-rad consumables. Primary antibody rabbit polyclonal anti-eGFP (1:1000, Abcam, reference: ab6556), and mouse monoclonal anti-GAPDH (1:6000, Abcam reference: ab8245), are revealed with appropriate anti-rabbit or anti-mouse peroxidase-conjugated secondary antibodies [1:6000, Invitrogen references: A16023 (anti-rabbit) and A16011 (anti-mouse)] and the ECL chemiluminescent reaction (Bio-rad).

IHC against the eGFP protein (rabbit anti-eGFP; Abcam, reference: ab6556; dilution: 1:6000) is performed in serial frozen brain slices containing the whole rostro-caudal sampling of the striatum to evaluate eGFP expression, thus allowing to qualitatively estimate the area of expression eGFP in the overall regions of the striatum. Quantification of eGFP transduced area on frozen brain slices is performed as described in Example 4.

### Statistical Analyses

All analyses are performed as described in Example 4.

### REFERENCES

Caron, N. S., Southwell, A. L., Brouwers, C. C., Cengio, L. D., Xie, Y., Black, H. F., Anderson, L. M., Ko, S., Zhu, X., van Deventer, S. J., Evers, M. M., Konstantinova, P., & Hayden, M. R. (2020). Potent and sustained huntingtin lowering via AAVS encoding miRNA preserves striatal volume and cognitive function in a humanized mouse model of Huntington disease. Nucleic acids research, 48(1), 36-54. https://cloi.org/10.1093/nar/gkz976
Miniarikova, J., Zimmer, V., Martier, R., Brouwers, C. C., Pythoud, C., Richetin, K., Rey, M., Lubelski, J., Evers, M. M., van Deventer, S. J., Petry, H., DegIon, N., & Konstantinova, P. (2017). AAV5-miHTT gene therapy demonstrates suppression of mutant huntingtin aggregation and neuronal dysfunction in a rat model of Huntington's disease. Gene therapy, 24(10), 630-639. https://doi.org/10.1038/gt.2017.71
Spronck, E. A., Brouwers, C. C., Valles, A., de Haan, M., Petry, H., van Deventer, S. J., Konstantinova, P., & Evers, M. M. (2019). AAV5-miHTT Gene Therapy Demonstrates Sustained Huntingtin Lowering and Functional Improvement in Huntington Disease Mouse Models. Molecular therapy. Methods & clinical development, 13, 334-343. https://doi.org/10.1016nomtm.2019.03.002.

### Example 6—A Study to Evaluate AAVrh10.CAG.hCYP46A1 Striatal Administration in Adults with Early Manifest Huntington's Disease

Described herein in this example is the assessment of safety, tolerability, and preliminary efficacy of striatal administration of the AAVrh10.CAG.hCYP46A1 vector, i.e., having a sequence of SEQ ID NO: 194, in adults with early manifest Huntington's Disease. 18 participants, ages 18-65, are included in the example based on, e.g., the following inclusion and exclusion criteria provided below:

Inclusion Criteria in this Example:
Male or Female subjects between ages 18 and 65 years (both inclusive) at time of consenting, able to provide Informed Consent and able to understand and comply with all study procedures.
Documented genetic confirmation of pathological CAG expansion in the huntingtin gene≥40.
Early manifest HD as defined by a UHDRS total functional capacity (TFC) score of 9 to 13 and a diagnostic classification level (DCL) of 4, or a DCL of 3 if present with cognitive impairment and clear evidence of disease progression.
Striatal MRI volumes per hemisphere: Putamen≥2.3 cm3 (per side); Caudate≥1.7 cm3 (per side) on Screening MRI.
All HD concomitant medications stable for at least 30 days prior to screening at the investigator's discretion.
   Key Exclusion Criteria in this Example:
Prior or ongoing medical condition, physical findings, ECG findings, or laboratory abnormality that, in the investigator's opinion, impact subject's safety and compliance with the study procedures.
Metastatic neoplasms within the five years prior to screening.
Presence of clinically relevant immunologic, hematologic, hepatic, cardiac, or renal disease at the time of screening as per investigator's clinical judgment.
Current untreated and unstable depressive disorder or a serious mood disorder requiring hospitalization.
History of prior suicide attempt or imminent risk of self-harm based on investigator's judgment or with a “yes” answer on item 4 or 5 on the Columbia Suicide Severity Rating Scale (C-SSRS).
Patients with history of confirmed stroke, known intracranial neoplasms, vascular malformations, or intracranial hemorrhage.
Subjects not deemed suitable for the surgical procedure as per the Neurosurgeon's judgment.
Any history of gene therapy, cell transplantation or any other experimental brain surgery.
Any RNA or DNA targeted HD specific investigational agents such as antisense oligonucleotides within 6 months prior to screening.
Subjects unable to tolerate or unwilling to undergo multiple lumbar punctures.
Participation in any clinical trial of an approved or non-approved investigational drug or intervention within 12 weeks or 5 half-lives whichever is longer prior to treatment.

### Vectors and Administration

This example includes two cohorts: a low dose group, “cohort 1,” and a high dose group “cohort 2.”

Cohort 1 receives one-time intracerebral bilateral injections of AAVrh10.CAG.hCYP46A1, an adeno-associated viral vector serotype Rh10 containing the human cholesterol 24-hydroxylase gene, at a first titer of 0.4×10⁹ vg/μl. Cohort 2 receives one-time intracerebral bilateral injections of AAVrh10.CAG.hCYP46A1 at a second titer of 1.1×10⁹ vg/μl. The AAVrh10.CAG.hCYP46A1 compositions comprise 10 mM phosphate (9 mM Na₂HPO₄·7H₂O and 1 mM KH₂PO₄), 137 mM NaCl, 2.7 mM KCl, 0.88 mM CaCl₂, 0.49 mM MgCl₂, 0.001 wt. % Poloxamer 188, pH 7.2, and the respective AAVrh10.CAG.hCYP46A1 titers, and can be prepared by combining the respective amounts of AAVrh10.CAG.hCYP46A1 with 0.02045 mg NaCl, 0.00242 mg Na₂HPO₄·7H₂O, 0.00014 mg KH₂PO₄, 0.00020 mg KCl, 0.050 mg sorbitol, 0.00001 mg Poloxamer 188, and 0.96 mg water.

For each brain hemisphere, injections are performed using one or more cannulas (ClearPoint Cannulas; Solana Beach, CA) and two trajectories: one anterior trajectory for the caudate and one posterior trajectory for the putamen, with two deposits in the putamen positioned along the same posterior trajectory. Injected volume of the virus is calculated for each patient based on volumetric MRI measures of each structure—the two caudates and the two putamens. For example, 25% of the volume of each structure will be injected with the first titer for Cohort 1 and with the second titer for Cohort 2. Two optimal trajectories, one for caudate and one for putamen, for each brain hemisphere is defined by the neurosurgeon based on baseline MRI to ensure safety.

This example consists of 2 parts: Dose-Finding Part and Expansion Part; each part consists of 3 phases: Screening Phase (8 weeks, with extension to 12 weeks to accommodate scheduling if needed), Treatment and Initial Follow-Up Phase (52 weeks) and Long-Term Follow-Up Phase (4 years). In the Dose-Finding Part, 2 dose titers are tested in 3-6 subjects in each cohort. Once a dose is selected based on Dose-Limiting Toxicities, an additional 6 subjects are enrolled into the Dose Expansion Part.

### Primary Outcome Measures

Incidence of Dose-Limiting Toxicities (DLTs), Treatment-Emergent Adverse Events (TEAEs), and Serious Adverse Events (SAEs) are assessed at 52 weeks post-administration. The incidence of DLTs, TEAEs, and SAEs are measured according to protocol specifications know in the art. DLTs, TEAEs, and SAEs are not observed at 52 weeks post-administration.

### Secondary Outcome Measures

The anatomical and volumetric measures of brain regions impacted by HD will be assessed by MRI at 52 weeks post-administration. The magnitude and variability of change from baseline in anatomical and volumetric measures of brain regions impacted by HD as assessed by MRI are measured. The slope of atrophy progression on striatal structures, ventricle dilatation and cortical atrophy are decreased at 52 weeks post-administration.

The composite Unified Huntington Disease Rating Scale (cUHDRS) will be assessed at 52 weeks post-administration. The change from baseline in the cUHDRS is measured; a higher score indicating better functioning. Clinical scores are stabilized or improved as compared to baseline levels at 52 weeks post-administration.

Mutant Huntingtin protein (mHTT) concentration in blood and cerebrospinal fluid (CSF) will be assessed at 52 weeks post-administration. The change from baseline in mHTT in blood and CSF are measured. mHTT concentrations in blood and CSF are decreased as compared to baseline levels at 52 weeks post-administration.

The level of neurofilament light chain (NfL) in blood and CSF will be assessed at 52 weeks post-administration. The change from baseline in blood and CSF NfL is measured. NFL in blood and CSF is decreased as compared to baseline levels, showing neuronal improvement compared to baseline levels at 52 weeks post-administration.

24OH cholesterol concentrations will be assessed in blood and CSF at 52 weeks post-administration. The change from baseline in blood and CSF 24OH cholesterol is measured. The concentration of 24OH cholesterol is increased in blood and CSF as compared to baseline concentrations at 52 weeks post-administration.

Magnetic resonance spectroscopy (MRS) will be used to assess the metabolic profile at 52 weeks post-administration. Change from baseline in MRS metabolic profile is measured. The metabolic profile is improved as compared to baseline at 52 weeks post-administration.

Positron emission tomography (PET) fluoro-deoxyglucose (FDG) striatal profile will be assessed at 52 weeks post-administration. Change from baseline in PET FDG striatal profile is assessed. A glucose profile is improved on PET as compared to baseline levels at 52 weeks post-administration.

### Example 7—Sequences

SEQ ID NO: 3 Exon 1 of human HTT gene
uugcugugug aggcagaacc ugcgggggca ggggcgggcu gguucccugg ccagccauug 60
gcagaguccg caggcuaggg cugucaauca ugcuggccgg cguggccccg ccuccgccgg 120
cgcggccccg ccuccgccgg cgcacgucug ggacgcaagg cgccgugggg gcugccggga 180
cggguccaag auggacggcc gcucagguuc ugcuuuuacc ugcggcccag agccccauuc 240
auugccccgg ugcugagegg cgccgcgagu cggcccgagg ccuccgggga cugccgugcc 300
gggcgggaga ccgccauggc gacccuggaa aagcugauga aggccuucga gucccucaag 360
uccuuccagc agcagcagca gcagcagcag cagcagcage agcagcagca gcagcagcag 420
cagcagcage aacagccgcc accgccgccg ccgccgccgc cgccuccuca gcuuccucag 480
ccgccgccgc aggcacagcc gcugcugccu cagccgcage cgcccccgcc gccgcccccg 540
ccgccacccg gcccggcugu ggcugaggag ccgcugcacc gaccgugagu uugggcccgc 600
ugcagcuccc uguc 614
SEQ ID NO: 4: Human HTT mRNA sequence

| | |
|---|---|
| 1 | gctgccggga cgggtccaag atggacggcc gctcaggttc tgcttttacc tgcggcccag |
| 61 | agccccattc attgccccgg tgctgagcgg cgccgcgagt cggcccgagg cctccgggga |
| 121 | ctgccgtgcc gggcgggaga ccgccatggc gaccctggaa aagctgatga aggccttcga |
| 181 | gtccctcaag tccttccagc agcagcagca gcagcagcag cagcagcagc agcagcagca |
| 241 | gcagcagcag cagcagcagc aacagccgcc accgccgccg ccgccgccgc cgcctcctca |
| 301 | gcttcctcag ccgccgccgc aggcacagcc gctgctgcct cagccgcagc cgcccccgcc |
| 361 | gccgcccccg ccgccacccg gcccggctgt ggctgaggag ccgctgcacc gaccaaagaa |
| 421 | agaactttca gctaccaaga aagaccgtgt gaatcattgt ctgacaatat gtgaaaacat |
| 481 | agtggcacag tctgtcagaa attctccaga atttcagaaa cttctgggca tcgctatgga |
| 541 | actttttctg ctgtgcagtg atgacgcaga gtcagatgtc aggatggtgg ctgacgaatg |
| 601 | cctcaacaaa gttatcaaag ctttgatgga ttctaatctt ccaaggttac agctcgagct |
| 661 | ctataaggaa attaaaaaga atggtgcccc tcggagtttg cgtgctgccc tgtggaggtt |
| 721 | tgctgagctg gctcacctgg ttcggcctca gaaatgcagg ccttacctgg tgaaccttct |
| 781 | gccgtgcctg actcgaacaa gcaagagacc cgaagaatca gtccaggaga ccttggctgc |
| 841 | agctgttccc aaaattatgg cttcttttgg caattttgca aatgacaatg aaattaaggt |
| 901 | tttgttaaag gccttcatag cgaacctgaa gtcaagctcc cccaccattc ggcggacagc |
| 961 | ggctggatca gcagtgagca tctgccagca ctcaagaagg acacaatatt tctatagttg |
| 1021 | gctactaaat gtgctcttag gcttactcgt tcctgtcgag gatgaacact ccactctgct |
| 1081 | gattcttggc gtgctgctca ccctgaggta tttggtgccc ttgctgcagc agcaggtcaa |
| 1141 | ggacacaagc ctgaaaggca gcttcggagt gacaaggaaa gaaatggaag tctctccttc |
| 1201 | tgcagagcag cttgtccagg tttatgaact gacgttacat catacacagc accaagacca |
| 1261 | caatgttgtg accggagccc tggagctgtt gcagcagctc ttcagaacgc ctccacccga |
| 1321 | gcttctgcaa accctgaccg cagtcggggg cattgggcag ctcaccgctg ctaaggagga |
| 1381 | gtctggtggc cgaagccgta gtgggagtat tgtggaactt atagctggag ggggttcctc |
| 1441 | atgcagccct gtcctttcaa gaaaacaaaa aggcaaagtg ctcttaggag aagaagaagc |
| 1501 | cttggaggat gactctgaat cgagatcgga tgtcagcagc tctgccttaa cagcctcagt |
| 1561 | gaaggatgag atcagtggag agctggctgc ttcttcaggg gtttccactc cagggtcagc |
| 1621 | aggtcatgac atcatcacag aacagccacg gtcacagcac acactgcagg cggactcagt |
| 1681 | ggatctggcc agctgtgact tgacaagctc tgccactgat ggggatgagg aggatatctt |
| 1741 | gagccacagc tccagccagg tcagcgccgt cccatctgac cctgccatgg acctgaatga |
| 1801 | tgggacccag gcctcgtcgc ccatcagcga cagctcccag accaccaccg aagggcctga |
| 1861 | ttcagctgtt accccttcag acagttctga aattgtgtta gacggtaccg acaaccagta |
| 1921 | tttgggcctg cagattggac agccccagga tgaagatgag gaagccacag gtattcttcc |
| 1981 | tgatgaagcc tcggaggcct tcaggaactc ttccatggcc cttcaacagg cacatttatt |
| 2041 | gaaaaacatg agtcactgca ggcagccttc tgacagcagt gttgataaat ttgtgttgag |
| 2101 | agatgaagct actgaaccgg gtgatcaaga aaacaagcct tgccgcatca aaggtgacat |
| 2161 | tggacagtcc actgatgatg actctgcacc tcttgtccat tgtgtccgcc ttttatctgc |
| 2221 | ttcgtttttg ctaacagggg gaaaaaatgt gctggttccg gacagggatg tgagggtcag |
| 2281 | cgtgaaggcc ctggccctca gctgtgtggg agcagctgtg gccctccacc cggaatcttt |
| 2341 | cttcagcaaa ctctataaag ttcctcttga caccacggaa taccctgagg aacagtatgt |
| 2401 | ctcagacatc ttgaactaca tcgatcatgg agacccacag gttcgaggag ccactgccat |
| 2461 | tctctgtggg accctcatct gctccatcct cagcaggtcc cgcttccacg tgggagattg |
| 2521 | gatgggcacc attagaaccc tcacaggaaa tacattttct ttggcggatt gcattccttt |
| 2581 | gctgcggaaa acactgaagg atgagtcttc tgttacttgc aagttagctt gtacagctgt |
| 2641 | gaggaactgt gtcatgagtc tctgcagcag cagctacagt gagttaggac tgcagctgat |
| 2701 | catcgatgtg ctgactctga ggaacagttc ctattggctg gtgaggacag agcttctgga |
| 2761 | aacccttgca gagattgact tcaggctggt gagctttttg gaggcaaaag cagaaaactt |
| 2821 | acacagaggg gctcatcatt atacagggct tttaaaactg caagaacgag tgctcaataa |
| 2881 | tgttgtcatc catttgcttg gagatgaaga ccccagggtg cgacatgttg ccgcagcatc |
| 2941 | actaattagg cttgtcccaa agctgtttta taaatgtgac caaggacaag ctgatccagt |
| 3001 | agtggccgtg gcaagagatc aaagcagtgt ttacctgaaa cttctcatgc atgagacgca |
| 3061 | gcctccatct catttctccg tcagcacaat aaccagaata tatagaggct ataacctact |
| 3121 | accaagcata acagacgtca ctatggaaaa taacctttca agagttattg cagcagtttc |
| 3181 | tcatgaacta atcacatcaa ccaccagagc actcacattt ggatgctgtg aagctttgtg |
| 3241 | tcttctttcc actgccttcc cagtttgcat ttggagttta ggttggcact gtggagtgcc |
| 3301 | tccactgagt gcctcagatg agtctaggaa gagctgtacc gttgggatgg ccacaatgat |
| 3361 | tctgaccctg ctctcgtcag cttggttccc attggatctc tcagcccatc aagatgcttt |
| 3421 | gattttggcc ggaaacttgc ttgcagccag tgctcccaaa tctctgagaa gttcatgggc |
| 3481 | ctctgaagaa gaagccaacc cagcagccac caagcaagag gaggtctggc cagccctggg |
| 3541 | ggaccgggcc ctggtgccca tggtggagca gctcttctct cacctgctga aggtgattaa |
| 3601 | catttgtgcc cacgtcctgg atgacgtggc tcctggaccc gcaataaagg cagccttgcc |
| 3661 | ttctctaaca aacccccctt ctctaagtcc catccgacga aaggggaagg agaaagaacc |
| 3721 | aggagaacaa gcatctgtac cgttgagtcc caagaaaggc agtgaggcca gtgcagcttc |
| 3781 | tagacaatct gatacctcag gtcctgttac aacaagtaaa tcctcatcac tggggagttt |
| 3841 | ctatcatctt ccttcatacc tcaaactgca tgatgtcctg aaagctacac acgctaacta |
| 3901 | caaggtcacg ctggatcttc agaacagcac ggaaaagttt ggagggtttc tccgctcagc |
| 3961 | cttggatgtt ctttctcaga tactagagct ggccacactg caggacattg ggaagtgtgt |
| 4021 | tgaagagatc ctaggatacc tgaaatcctg ctttagtcga gaaccaatga tggcaactgt |
| 4081 | ttgtgttcaa caattgttga agactctctt tggcacaaac ttggcctccc agtttgatgg |
| 4141 | cttatcttcc aaccccagca agtcacaagg ccgagcacag cgccttggct cctccagtgt |
| 4201 | gaggccaggc ttgtaccact actgcttcat ggccccgtac acccacttca cccaggccct |
| 4261 | cgctgacgcc agcctgagga acatggtgca ggcggagcag gagaacgaca cctcgggatg |
| 4321 | gtttgatgtc ctccagaaag tgtctaccca gttgaagaca aacctcacga gtgtcacaaa |
| 4381 | gaaccgtgca gataagaatg ctattcataa tcacattcgt ttgtttgaac ctcttgttat |
| 4441 | aaaagcttta aaacagtaca cgactacaac atgtgtgcag ttacagaagc aggttttaga |
| 4501 | tttgctggcg cagctggttc agttacgggt taattactgt cttctggatt cagatcaggt |
| 4561 | gtttattggc tttgtattga aacagtttga atacattgaa gtgggccagt tcagggaatc |
| 4621 | agaggcaatc attccaaaca tctttttctt cttggtatta ctatcttatg aacgctatca |
| 4681 | ttcaaaacag atcattggaa ttcctaaaat cattcagctc tgtgatggca tcatggccag |
| 4741 | tggaaggaag gctgtgacac atgccatacc ggctctgcag cccatagtcc acgacctctt |
| 4801 | tgtattaaga ggaacaaata aagctgatgc aggaaaagag cttgaaaccc aaaaagaggt |
| 4861 | ggtggtgtca atgttactga gactcatcca gtaccatcag gtgttggaga tgttcattct |
| 4921 | tgtcctgcag cagtgccaca aggagaatga agacaagtgg aagcgactgt ctcgacagat |
| 4981 | agctgacatc atcctcccaa tgttagccaa acagcagatg cacattgact ctcatgaagc |
| 5041 | ccttggagtg ttaaatacat tatttgagat tttggcccct tcctccctcc gtccggtaga |
| 5101 | catgctttta cggagtatgt tcgtcactcc aaacacaatg gcgtccgtga gcactgttca |
| 5161 | actgtggata tcgggaattc tggccatttt gagggttctg atttcccagt caactgaaga |
| 5221 | tattgttctt tctcgtattc aggagctctc cttctctccg tatttaatct cctgtacagt |
| 5281 | aattaatagg ttaagagatg gggacagtac ttcaacgcta gaagaacaca gtgaagggaa |
| 5341 | acaaataaag aatttgccag aagaaacatt ttcaaggttt ctattacaac tggttggtat |
| 5401 | tcttttagaa gacattgtta caaaacagct gaaggtggaa atgagtgagc agcaacatac |
| 5461 | tttctattgc caggaactag gcacactgct aatgtgtctg atccacatct tcaagtctgg |
| 5521 | aatgttccgg agaatcacag cagctgccac taggctgttc cgcagtgatg gctgtggcgg |
| 5581 | cagtttctac accctggaca gcttgaactt gcgggctcgt tccatgatca ccacccaccc |
| 5641 | ggccctggtg ctgctctggt gtcagatact gctgcttgtc aaccacaccg actaccgctg |
| 5701 | gtgggcagaa gtgcagcaga ccccgaaaag acacagtctg tccagcacaa agttacttag |
| 5761 | tccccagatg tctggagaag aggaggattc tgacttggca gccaaacttg gaatgtgcaa |
| 5821 | tagagaaata gtacgaagag gggctctcat tctcttctgt gattatgtct gtcagaacct |
| 5881 | ccatgactcc gagcacttaa cgtggctcat tgtaaatcac attcaagatc tgatcagcct |
| 5941 | ttcccacgag cctccagtac aggacttcat cagtgccgtt catcggaact ctgctgccag |
| 6001 | cggcctgttc atccaggcaa ttcagtctcg ttgtgaaaac ctttcaactc caaccatgct |
| 6061 | gaagaaaact cttcagtgct tggaggggat ccatctcagc cagtcgggag ctgtgctcac |
| 6121 | gctgtatgtg gacaggcttc tgtgcacccc tttccgtgtg ctggctcgca tggtcgacat |
| 6181 | ccttgcttgt cgccgggtag aaatgcttct ggctgcaaat ttacagagca gcatggccca |
| 6241 | gttgccaatg gaagaactca acagaatcca ggaatacctt cagagcagcg ggctcgctca |
| 6301 | gagacaccaa aggctctatt ccctgctgga caggtttcgt ctctccacca tgcaagactc |
| 6361 | acttagtccc tctcctccag tctcttccca cccgctggac ggggatgggc acgtgtcact |
| 6421 | ggaaacagtg agtccggaca aagactggta cgttcatctt gtcaaatccc agtgttggac |
| 6481 | caggtcagat tctgcactgc tggaaggtgc agagctggtg aatcggattc ctgctgaaga |
| 6541 | tatgaatgcc ttcatgatga actcggagtt caacctaagc ctgctagctc catgcttaag |
| 6601 | cctagggatg agtgaaattt ctggtggcca gaagagtgcc ctttttgaag cagcccgtga |
| 6661 | ggtgactctg gcccgtgtga gcggcaccgt gcagcagctc cctgctgtcc atcatgtctt |
| 6721 | ccagcccgag ctgcctgcag agccggcggc ctactggagc aagttgaatg atctgtttgg |
| 6781 | ggatgctgca ctgtatcagt ccctgcccac tctggcccgg gccctggcac agtacctggt |
| 6841 | ggtggtctcc aaactgccca gtcatttgca ccttcctcct gagaaagaga aggacattgt |
| 6901 | gaaattcgtg gtggcaaccc ttgaggccct gtcctggcat ttgatccatg agcagatccc |
| 6961 | gctgagtctg gatctccagg cagggctgga ctgctgctgc ctggccctgc agctgcctgg |
| 7021 | cctctggagc gtggtctcct ccacagagtt tgtgacccac gcctgctccc tcatctactg |
| 7081 | tgtgcacttc atcctggagg ccgttgcagt gcagcctgga gagcagcttc ttagtccaga |
| 7141 | aagaaggaca aataccccaa aagccatcag cgaggaggag gaggaagtag atccaaacac |
| 7201 | acagaatcct aagtatatca ctgcagcctg tgagatggtg gcagaaatgg tggagtctct |
| 7261 | gcagtcggtg ttggccttgg gtcataaaag gaatagcggc gtgccggcgt ttctcacgcc |
| 7321 | attgctaagg aacatcatca tcagcctggc ccgcctgccc cttgtcaaca gctacacacg |
| 7381 | tgtgccccca ctggtgtgga agcttggatg gtcacccaaa ccgggagggg attttggcac |
| 7441 | agcattccct gagatccccg tggagttcct ccaggaaaag gaagtcttta aggagttcat |
| 7501 | ctaccgcatc aacacactag gctggaccag tcgtactcag tttgaagaaa cttgggccac |
| 7561 | cctccttggt gtcctggtga cgcagcccct cgtgatggag caggaggaga gcccaccaga |
| 7621 | agaagacaca gagaggaccc agatcaacgt cctggccgtg caggccatca cctcactggt |
| 7681 | gctcagtgca atgactgtgc ctgtggccgg caacccagct gtaagctgct tggagcagca |
| 7741 | gccccggaac aagcctctga aagctctcga caccaggttt gggaggaagc tgagcattat |
| 7801 | cagagggatt gtggagcaag agattcaagc aatggtttca aagagagaga atattgccac |
| 7861 | ccatcattta tatcaggcat gggatcctgt cccttctctg tctccggcta ctacaggtgc |
| 7921 | cctcatcagc cacgagaagc tgctgctaca gatcaacccc gagcgggagc tggggagcat |
| 7981 | gagctacaaa ctcggccagg tgtccataca ctccgtgtgg ctggggaaca gcatcacacc |
| 8041 | cctgagggag gaggaatggg acgaggaaga ggaggaggag gccgacgccc ctgcaccttc |
| 8101 | gtcaccaccc acgtctccag tcaactccag gaaacaccgg gctggagttg acatccactc |
| 8161 | ctgttcgcag tttttgcttg agttgtacag ccgctggatc ctgccgtcca gctcagccag |
| 8221 | gaggaccccg gccatcctga tcagtgaggt ggtcagatcc cttctagtgg tctcagactt |
| 8281 | gttcaccgag cgcaaccagt ttgagctgat gtatgtgacg ctgacagaac tgcgaagggt |
| 8341 | gcacccttca gaagacgaga tcctcgctca gtacctggtg cctgccacct gcaaggcagc |
| 8401 | tgccgtcctt gggatggaca aggccgtggc ggagcctgtc agccgcctgc tggagagcac |
| 8461 | gctcaggagc agccacctgc ccagcagggt tggagccctg cacggcgtcc tctatgtgct |
| 8521 | ggagtgcgac ctgctggacg acactgccaa gcagctcatc ccggtcatca gcgactatct |
| 8581 | cctctccaac ctgaaaggga tcgcccactg cgtgaacatt cacagccagc agcacgtact |
| 8641 | ggtcatgtgt gccactgcgt tttacctcat tgagaactat cctctggacg tagggccgga |
| 8701 | attttcagca tcaataatac agatgtgtgg ggtgatgctg tctggaagtg aggagtccac |
| 8761 | cccctccatc atttaccact gtgccctcag aggcctggag cgcctcctgc tctctgagca |
| 8821 | gctctcccgc ctggatgcag aatcgctggt caagctgagt gtggacagag tgaacgtgca |
| 8881 | cagcccgcac cgggccatgg cggctctggg cctgatgctc acctgcatgt acacaggaaa |
| 8941 | ggagaaagtc agtccgggta gaacttcaga ccctaatcct gcagcccccg acagcgagtc |
| 9001 | agtgattgtt gctatggagc gggtatctgt tctttttgat aggatcagga aaggctttcc |
| 9061 | ttgtgaagcc agagtggtgg ccaggatcct gccccagttt ctagacgact tcttcccacc |
| 9121 | ccaggacatc atgaacaaag tcatcggaga gtttctgtcc aaccagcagc cataccccca |
| 9181 | gttcatggcc accgtggtgt ataaggtgtt tcagactctg cacagcaccg ggcagtcgtc |
| 9241 | catggtccgg gactgggtca tgctgtccct ctccaacttc acgcagaggg ccccggtcgc |
| 9301 | catggccacg tggagcctct cctgcttctt tgtcagcgcg tccaccagcc cgtgggtcgc |
| 9361 | ggcgatcctc ccacatgtca tcagcaggat gggcaagctg gagcaggtgg acgtgaacct |
| 9421 | tttctgcctg gtcgccacag acttctacag acaccagata gaggaggagc tcgaccgcag |
| 9481 | ggccttccag tctgtgcttg aggtggttgc agccccagga agcccatatc accggctgct |
| 9541 | gacttgttta cgaaatgtcc acaaggtcac cacctgctga gcgccatggt gggagagact |
| 9601 | gtgaggcggc agctggggcc ggagcctttg gaagtctgcg cccttgtgcc ctgcctccac |
| 9661 | cgagccagct tggtccctat gggcttccgc acatgccgcg ggcggccagg caacgtgcgt |
| 9721 | gtctctgcca tgtggcagaa gtgctctttg tggcagtggc caggcaggga gtgtctgcag |
| 9781 | tcctggtggg gctgagcctg aggccttcca gaaagcagga gcagctgtgc tgcaccccat |
| 9841 | gtgggtgacc aggtcctttc tcctgatagt cacctgctgg ttgttgccag gttgcagctg |
| 9901 | ctcttgcatc tgggccagaa gtcctccctc ctgcaggctg gctgttggcc cctctgctgt |
| 9961 | cctgcagtag aaggtgccgt gagcaggctt tgggaacact ggcctgggtc tccctggtgg |
| 10021 | ggtgtgcatg ccacgccccg tgtctggatg cacagatgcc atggcctgtg ctgggccagt |
| 10081 | ggctgggggt gctagacacc cggcaccatt ctcccttctc tcttttcttc tcaggattta |
| 10141 | aaatttaatt atatcagtaa agagattaat tttaacgtaa ctctttctat gcccgtgtaa |
| 10201 | agtatgtgaa tcgcaaggcc tgtgctgcat gcgacagcgt ccggggtggt ggacagggcc |
| 10261 | cccggccacg ctccctctcc tgtagccact ggcatagccc tcctgagcac ccgctgacat |
| 10321 | ttccgttgta catgttcctg tttatgcatt cacaaggtga ctgggatgta gagaggcgtt |
| 10381 | agtgggcagg tggccacagc aggactgagg acaggccccc attatcctag gggtgcgctc |
| 10441 | acctgcagcc cctcctcctc gggcacagac gactgtcgtt ctccacccac cagtcaggga |
| 10501 | cagcagcctc cctgtcactc agctgagaag gccagccctc cctggctgtg agcagcctcc |
| 10561 | actgtgtcca gagacatggg cctcccactc ctgttccttg ctagccctgg ggtggcgtct |
| 10621 | gcctaggagc tggctggcag gtgttgggac ctgctgctcc atggatgcat gccctaagag |
| 10681 | tgtcactgag ctgtgttttg tctgagcctc tctcggtcaa cagcaaagct tggtgtcttg |
| 10741 | gcactgttag tgacagagcc cagcatccct tctgcccccg ttccagctga catcttgcac |
| 10801 | ggtgacccct tttagtcagg agagtgcaga tctgtgctca tcggagactg ccccacggcc |
| 10861 | ctgtcagagc cgccactcct atccccaggc caggtccctg gaccagcctc ctgtttgcag |
| 10921 | gcccagagga gccaagtcat taaaatggaa gtggattctg gatggccggg ctgctgctga |
| 10981 | tgtaggagct ggatttggga gctctgcttg ccgactggct gtgagacgag gcaggggctc |
| 11041 | tgcttcctca gccctagagg cgagccaggc aaggttggcg actgtcatgt ggcttggttt |
| 11101 | ggtcatgccc gtcgatgttt tgggtattga atgtggtaag tggaggaaat gttggaactc |
| 11161 | tgtgcaggtg ctgccttgag acccccaagc ttccacctgt ccctctccta tgtggcagct |
| 11221 | ggggagcagc tgagatgtgg acttgtatgc tgcccacata cgtgaggggg agctgaaagg |
| 11281 | gagcccctcc tctgagcagc ctctgccagg cctgtatgag gcttttccca ccagctccca |
| 11341 | acagaggcct cccccagcca ggaccacctc gtcctcgtgg cggggcagca ggagcggtag |
| 11401 | aaaggggtcc gatgtttgag gaggccctta agggaagcta ctgaattata acacgtaaga |
| 11461 | aaatcaccat tccgtattgg ttgggggctc ctgtttctca tcctagcttt ttcctggaaa |
| 11521 | gcccgctaga aggtttggga acgaggggaa agttctcaga actgttggct gctccccacc |
| 11581 | cgcctcccgc ctcccccgca ggttatgtca gcagctctga gacagcagta tcacaggcca |
| 11641 | gatgttgttc ctggctagat gtttacattt gtaagaaata acactgtgaa tgtaaaacag |
| 11701 | agccattccc ttggaatgca tatcgctggg ctcaacatag agtttgtctt cctcttgttt |
| 11761 | acgacgtgat ctaaaccagt ccttagcaag gggctcagaa caccccgctc tggcagtagg |
| 11821 | tgtcccccac ccccaaagac ctgcctgtgt gctccggaga tgaatatgag ctcattagta |
| 11881 | aaaatgactt cacccacgca tatacataaa gtatccatgc atgtgcatat agacacatct |
| 11941 | ataattttac acacacacct ctcaagacgg agatgcatgg cctctaagag tgcccgtgtc |
| 12001 | ggttcttcct ggaagttgac tttccttaga cccgccaggt caagttagcc gcgtgacgga |
| 12061 | catccaggcg tgggacgtgg tcagggcagg gctcattcat tgcccactag gatcccactg |
| 12121 | gcgaagatgg tctccatatc agctctctgc agaagggagg aagactttat catgttccta |
| 12181 | aaaatctgtg gcaagcaccc atcgtattat ccaaattttg ttgcaaatgt gattaatttg |
| 12241 | gttgtcaagt tttgggggtg ggctgtgggg agattgcttt tgttttcctg ctggtaatat |
| 12301 | cgggaaagat tttaatgaaa ccagggtaga attgtttggc aatgcactga agcgtgtttc |
| 12361 | tttcccaaaa tgtgcctccc ttccgctgcg ggcccagctg agtctatgta ggtgatgttt |
| 12421 | ccagctgcca agtgctcttt gttactgtcc accctcattt ctgccagcgc atgtgtcctt |
| 12481 | tcaaggggaa aatgtgaagc tgaaccccct ccagacaccc agaatgtagc atctgagaag |
| 12541 | gccctgtgcc ctaaaggaca cccctcgccc ccatcttcat ggagggggtc atttcagagc |
| 12601 | cctcggagcc aatgaacagc tcctcctctt ggagctgaga tgagccccac gtggagctcg |
| 12661 | ggacggatag tagacagcaa taactcggtg tgtggccgcc tggcaggtgg aacttcctcc |
| 12721 | cgttgcgggg tggagtgagg ttagttctgt gtgtctggtg ggtggagtca ggcttctctt |
| 12781 | gctacctgtg agcatccttc ccagcagaca tcctcatcgg gctttgtccc tcccccgctt |
| 12841 | cctccctctg cggggaggac ccgggaccac agctgctggc cagggtagac ttggagctgt |
| 12901 | cctccagagg ggtcacgtgt aggagtgaga agaaggaaga tcttgagagc tgctgaggga |
| 12961 | ccttggagag ctcaggatgg ctcagacgag gacactcgct tgccgggcct gggcctcctg |
| 13021 | ggaaggaggg agctgctcag aatgccgcat gacaactgaa ggcaacctgg aaggttcagg |
| 13081 | ggccgctctt cccccatgtg cctgtcacgc tctggtgcag tcaaaggaac gccttcccct |
| 13141 | cagttgtttc taagagcaga gtctcccgct gcaatctggg tggtaactgc cagccttgga |
| 13201 | ggatcgtggc caacgtggac ctgcctacgg agggtgggct ctgacccaag tggggcctcc |
| 13261 | ttgtccaggt ctcactgctt tgcaccgtgg tcagagggac tgtcagctga gcttgagctc |
| 13321 | ccctggagcc agcagggctg tgatgggcga gtcccggagc cccacccaga cctgaatgct |
| 13381 | tctgagagca aagggaagga ctgacgagag atgtatattt aattttttaa ctgctgcaaa |
| 13441 | cattgtacat ccaaattaaa ggaaaaaaat ggaaaccatc aaaaaaaaaa aaaaaaaa |

SEQ ID NO: 5 Human HTT protein sequence

| | |
|---|---|
| 1 | matleklmka feslksfqqq qqqqqqqqqq qqqqqqqqqq pppppppppp pqlpqpppqa |
| 61 | qpllpqpqpp ppppppppgp avaeeplhrp kkelsatkkd rvnhcltice nivaqsvrns |
| 121 | pefqkllgia melfllcsdd aesdvrmvad eclnkvikal mdsnlprlql elykeikkng |
| 181 | aprslraalw rfaelahlvr pqkcrpylvn llpcltrtsk rpeesvqetl aaavpkimas |
| 241 | fgnfandnei kvllkafian lksssptirr taagsavsic qhsrrtqyfy swllnvllgl |
| 301 | lvpvedehst llilgvlltl rylvpllqqq vkdtslkgsf gvtrkemevs psaeqlvqvy |
| 361 | eltlhhtqhq dhnvvtgale llqqlfrtpp pellqtltav ggigqltaak eesggrsrsg |
| 421 | siveliaggg sscspvlsrk qkgkvllgee ealeddsesr sdvsssalta svkdeisgel |
| 481 | aassgvstpg saghdiiteq prsqhtlqad svdlascdlt ssatdgdeed ilshsssqvs |
| 541 | avpsdpamdl ndgtqasspi sdssqttteg pdsavtpsds seivldgtdn qylglqigqp |
| 601 | qdedeeatgi lpdeaseafr nssmalqqah llknmshcrq psdssvdkfv lrdeatepgd |
| 661 | qenkpcrikg digqstddds aplvhcvrll sasflltggk nvlvpdrdvr vsvkalalsc |
| 721 | vgaavalhpe sffsklykvp ldtteypeeq yvsdilnyid hgdpqvrgat ailcgtlics |
| 781 | ilsrsrfhvg dwmgtirtlt gntfsladci pllrktlkde ssvtcklact avrncvmslc |
| 841 | sssyselglq liidvltlrn ssywlvrtel letlaeidfr lvsfleakae nlhrgahhyt |
| 901 | gllklqervl nnvvihllgd edprvrhvaa aslirlvpkl fykcdqgqad pvvavardqs |
| 961 | svylkllmhe tqppshfsvs titriyrgyn llpsitdvtm ennlsrviaa vshelitstt |
| 1021 | raltfgccea lcllstafpv ciwslgwhcg vpplsasdes rksctvgmat miltllssaw |
| 1081 | fpldlsahqd alilagnlla asapkslrss waseeeanpa atkqeevwpa lgdralvpmv |
| 1141 | eqlfshllkv inicahvldd vapgpaikaa lpsltnppsl spirrkgkek epgeqasvpl |
| 1201 | spkkgseasa asrqsdtsgp vttskssslg sfyhlpsylk lhdvlkatha nykvtldlqn |
| 1261 | stekfggflr saldvlsqil elatlqdigk cveeilgylk scfsrepmma tvcvqqllkt |
| 1321 | lfgtnlasqf dglssnpsks qgraqrlgss svrpglyhyc fmapythftq aladaslrnm |
| 1381 | vqaeqendts gwfdvlqkvs tqlktnltsv tknradknai hnhirlfepl vikalkqytt |
| 1441 | ttcvqlqkqv ldllaqlvql rvnyclldsd qvfigfvlkq feyievgqfr eseaiipnif |
| 1501 | fflvllsyer yhskqiigip kiiqlcdgim asgrkavtha ipalqpivhd lfvlrgtnka |
| 1561 | dagkeletqk evvvsmllrl iqyhqvlemf ilvlqqchke nedkwkrlsr qiadiilpml |
| 1621 | akqqmhidsh ealgvlntlf eilapsslrp vdmllrsmfv tpntmasvst vqlwisgila |
| 1681 | ilrvlisqst edivlsriqe lsfspylisc tvinrlrdgd ststleehse gkqiknlpee |
| 1741 | tfsrfllqlv gilledivtk qlkvemseqq htfycqelgt llmclihifk sgmfrritaa |
| 1801 | atrlfrsdgc ggsfytldsl nlrarsmitt hpalvllwcq illlvnhtdy rwwaevqqtp |
| 1861 | krhslsstkl lspqmsgeee dsdlaaklgm cnreivriga lilfcdyvcq nlhdsehltw |
| 1921 | livnhiqdli slsheppvqd fisavhrnsa asglfiqaiq srcenlstpt mlkktlqcle |
| 1981 | gihlsqsgav ltlyvdrllc tpfrvlarmv dilacrrvem llaanlqssm aqlpmeelnr |
| 2041 | iqeylqssgl aqrhqrlysl ldrfrlstmq dslspsppvs shpldgdghv sletvspdkd |
| 2101 | wyvhlvksqc wtrsdsalle gaelvnripa edmnafmmns efnlsllapc lslgmseisg |
| 2161 | gqksalfeaa revtlarvsg tvqqlpavhh vfqpelpaep aaywsklndl fgdaalyqsl |
| 2221 | ptlaralaqy lvvvsklpsh lhlppekekd ivkfvvatle alswhliheq iplsldlqag |
| 2281 | ldccclalql pglwsvvsst efvthacsli ycvhfileav avqpgeqlls perrtntpka |
| 2341 | iseeeeevdp ntqnpkyita acemvaemve slqsvlalgh krnsgvpafl tpllrniiis |
| 2401 | larlplvnsy trvpplvwkl gwspkpggdf gtafpeipve flqekevfke fiyrintlgw |
| 2461 | tsrtqfeetw atllgvlvtq plvmeqeesp peedtertqi nvlavqaits lvlsamtvpv |
| 2521 | agnpavscle qqprnkplka ldtrfgrkls iirgiveqei qamvskreni athhlyqawd |
| 2581 | pvpslspatt galishekll lqinperelg smsyklgqvs ihsvwlgnsi tplreeewde |
| 2641 | eeeeeadapa psspptspvn srkhragvdi hscsqfllel ysrwilpsss arrtpailis |
| 2701 | evvrsllvvs dlfternqfe lmyvtltelr rvhpsedeil aqylvpatck aaavlgmdka |
| 2761 | vaepvsrlle stlrsshlps rvgalhgvly vlecdllddt akqlipvisd yllsnlkgia |
| 2821 | hcvnihsqqh vlvmcatafy lienypldvg pefsasiiqm cgvmlsgsee stpsiiyhca |
| 2881 | lrglerllls eqlsrldaes lvklsvdrvn vhsphramaa lglmltcmyt gkekvspgrt |
| 2941 | sdpnpaapds esvivamerv svlfdrirkg fpcearvvar ilpqflddff ppqdimnkvi |
| 3001 | geflsnqqpy pqfmatvvyk vfqtlhstgq ssmvrdwvml slsnftqrap vamatwslsc |
| 3061 | ffvsastspw vaailphvis rmgkleqvdv nlfclvatdf yrhqieeeld rrafqsvlev |
| 3121 | vaapgspyhr lltclrnvhk vttc |

SEQ ID NO: 109; translation of codon-optimized CYP46A1 sequence (SEQ ID NO: 110)
MSPGLLLLGSAVLLAFGLCCTFVHRARSRYEHIPGPPRPSFLLGHLPCFWKKDEVGGRVLQDVFLDWAKKYGPVV
RVNVFHKTSVIVTSPESVKKFLMSTKYNKDSKMYRALQTVFGERLFGQGLVSECNYERWHKORRVIDLAFSRSSL
VSLMETFNEKAEQLVEILEAKADGQTPVSMQDMLTYTAMDILAKAAFGMETSMLLGAQKPLSQAVKLMLEGITAS
RNTLAKFLPGKRKQLREVRESIRFLRQVGRDWVQRRREALKRGEEVPADILTQILKAEEGAQDDEGLLDNFVTFF
IAGHETSANHLAFTVMELSRQPEIVARLQAEVDEVIGSKRYLDFEDLGRLQYLSQVLKESLRLYPPAWGTFRLLE
EETLIDGVRVPGNTPLLFSTYVMGRMDTYFEDPLTFNPDRFGPGAPKPRFTYFPFSLGHRSCIGQQFAQMEVKVV
MAKLLQRLEFRLVPGQRFGLQEQATLKPLDPVLCTLRPRGWQPAPPPPPC
SEQ ID NO: 110; codon-optimized CYP46A1 CDS
atgagccccgggctgctgctgctcggtagcgccgtcctgctcgccttcggcctctgctgcaccttcgtgcaccgc
gctcgcagccgctacgagcacatccccgggccgccgcggcccagtttccttctaggacacctcccctgcttttgg
aaaaaggatgaggttggtggccgtgtgctccaagatgtgtttCtAgattgggctaagaagtatggacctgtAgtg
cgggtcaacgtcttccacaaaacctcagtcatcgtcacgagtcctgagtcggttaagaagttcctgatgtcaacc
aagtacaacaaggactccaagatgtaccgtgcgctccagactgtgtttggtgagagactcttcggccaaggcttg
gtgtccgaatgcaactatgagcgctggcacaagcagcggagagtGatagacctggccttcagccggagctccttg
gttagcttaatggaaacattcaacgaAaaggctgagcagctggtggagattctagaagccaaggcagatgggcag
accccTgtGAGCatgcaggacatgctgacctacaccgccatggacatcctggccaaggcagcttttgggatggag
accagtatgctgctgggtgcccagaagcctctgtcccaggcagtgaaacttatgttggagggaatcactgcgtcc
cgcaacactctggcaaagttcctgccagggaagaggaagcagctccgggaggtccgggagagcattcgcttcctg
cgccaggtgggcagggactgggtccagcgccgccgggaagccctgaagaggggcgaggaggttcctgccgacatc
ctcacacagattctgaaagctgaagagggagcccaggacgacgagggtctgctggacaacttcgtcaccttcttc
attgctggtcacgagacctctgccaaccacttggcgttcacagtgatggagctgtctcgccagccagagatcgtg
gcaaggctgcaggccgaggtggatgaAgtGattggttctaagaggtacctggatttcgaggacctggggagactg
cagtacctgtcccaggtcctcaaagagtcgctgaggctgtacccaccagcatggggcacctttAGGctgctggaa
gaggagaccttgattgatggggtGagagtccccggcaacaccccgctcttgttcagcacctatgtGatggggcgg
atggacacatactttgaggacccgctgactttcaaccccgatcgcttcggccctggagcacccaagccacggttc
acctacttccccttctccctgggccaccgctcctgcatcgggcagcagtttgctcagatggaggtgaaggtggtc
atggcaaagctgctgcagaggctggagttccggctggtgcccgggcagcgcttcgggctgcaggagcaggccaca
ctcaagccactggaccccgtgctgtgcaccctgcggccccgcggctggcagcccgcacccccaccacccccctgc
SEQ ID NO: 194; dbDNA_CYP46A1
gcgtataatggactattgtgtgctgataTCtgtacacttaagggctagatcttagcttacgtcactagagggtcc
acgtttagtttttaagatccattgatctcctaaacgctgcaagattcgcaacctggtatacttagcctaggcgct
aggtcctagtgcagcgggactttttttctaaagtcgttgagaggaggagtcgtcagaccagatagctttgatgtc
ctgatcggaaggatcgttggccccCCTGCAGGCAGCTGTTAATTAACTGCGCGCTCGCTCGCTCACTGAGGCCGC
CCGGGCAAAGCCCGGGCGTCGGGCGACCTTTGGTCGCCCGGCCTCAGTGAGCGAGCGAGCGCGCAGAGAGGGAGT
GGCCAACTCCATCACTAGGGGTTCCTTGTAGTTAATGATTAACCCGCCATGCTACTTATCTACGGCGCGCCacgc
gtgactagttattaatagtaatcaattacggggtcattagttcatagcccatatatggagttccgcgttacataa
cttacggtaaatggcccgcctggctgaccgcccaacgacccccgcccattgacgtcaataatgacgtatgttccc
atagtaacgccaatagggactttccattgacgtcaatgggtggagtatttacggtaaactgcccacttggcagta
catcaagtgtatcatatgccaagtacgccccctattgacgtcaatgacggtaaatggcccgcctggcattatgcc
cagtacatgaccttatgggactttcctacttggcagtacatctacgtattagtcatcgctattaccatggtcgag
gtgagccccacgttctgcttcactctccccatctcccccccctccccacccccaattttgtatttatttattttt
taattattttgtgcagcgatgggggcgggggggggggggggcgcgcgccaggcggggcggggcggggcgaggggc
ggggcggggcgaggcggagaggtgcggcggcagccaatcagagcggcgcgctccgaaagtttccttttatggcga
ggcggcggcggcggcggccctataaaaagcgaagcgcgcggcgggcgggagtcgctgcgcgctgccttcgccccg
tgccccgctccgccgccgcctcgcgccgcccgccccggctctgactgaccgcgttactcccacaggtgagcgggc
gggacggcccttctcctccgggctgtaattagcgcttggtttaatgacggcttgtttcttttctgtggctgcgtg
aaagccttgaggggctccgggagggccctttgtgcggggggagcggctcggggggtgcgtgcgtgtgtgtgtgcg
tggggagcgccgcgtgcggctccgcgctgcccggcggctgtgagcgctgcgggcgcggcgcggggctttgtgcgc
tccgcagtgtgcgcgaggggagcgcggccgggggcggtgccccgcggtgcggggggggctgcgaggggaacaaag
gctgcgtgcggggtgtgtgcgtgggggggtgagcagggggtgtgggcgcgtcggtcgggctgcaaccccccctgc
acccccctccccgagttgctgagcacggcccggcttcgggtgcggggctccgtacggggcgtggcgcggggctcg
ccgtgccgggcggggggtggcggcaggtgggggtgccgggcggggcggggccgcctcgggccggggagggctcgg
gggaggggcgcggcggcccccggagcgccggcggctgtcgaggcgcggcgagccgcagccattgccttttatggt
aatcgtgcgagagggcgcagggacttcctttgtcccaaatctgtgcggagccgaaatctgggaggcgccgccgca
ccccctctagcgggcgcggggcgaagcggtgcggcgccggcaggaaggaaatgggcggggagggccttcgtgcgt
cgccgcgccgccgtccccttctccctctccagcctcggggctgtccgcggggggacggctgccttcgggggggac
ggggcagggcggggttcggcttctggcgtgtgaccggcggctctagagcctctgctaaccatgttcatgccttct
tctttttcctacagctcctgggcaacgtgctggttattgtgctgtctcatcattttggcaaagaattgattaatt
cgagcgaacgcgtcgagtcgctcggtacgatttaaattgaattccttaagctatcataggaatgagccccgggct
gctgctgctcggtagcgccgtcctgctcgccttcggcctctgctgcaccttcgtgcaccgcgctcgcagccgcta
cgagcacatccccgggccgccgcggcccagtttccttctaggacacctcccctgcttttggaaaaaggatgaggt
tggtggccgtgtgctccaagatgtgtttCtAgattgggctaagaagtatggacctgtAgtgcgggtcaacgtctt
ccacaaaacctcagtcatcgtcacgagtcctgagtcggttaagaagttcctgatgtcaaccaagtacaacaagga
ctccaagatgtaccgtgcgctccagactgtgtttggtgagagactcttcggccaaggcttggtgtccgaatgcaa
ctatgagcgctggcacaagcagcggagagtGatagacctggccttcagccggagctccttggttagcttaatgga
aacattcaacgaAaaggctgagcagctggtggagattctagaagccaaggcagatgggcagaccccTgtGAGCat
gcaggacatgctgacctacaccgccatggacatcctggccaaggcagcttttgggatggagaccagtatgctgct
gggtgcccagaagcctctgtcccaggcagtgaaacttatgttggagggaatcactgcgtcccgcaacactctggc
aaagttcctgccagggaagaggaagcagctccgggaggtccgggagagcattcgcttcctgcgccaggtgggcag
ggactgggtccagcgccgccgggaagccctgaagaggggcgaggaggttcctgccgacatcctcacacagattct
gaaagctgaagagggagcccaggacgacgagggtctgctggacaacttcgtcaccttcttcattgctggtcacga
gacctctgccaaccacttggcgttcacagtgatggagctgtctcgccagccagagatcgtggcaaggctgcaggc
cgaggtggatgaAgtGattggttctaagaggtacctggatttcgaggacctggggagactgcagtacctgtccca
ggtcctcaaagagtcgctgaggctgtacccaccagcatggggcacctttAGGctgctggaagaggagaccttgat
tgatggggtGagagtccccggcaacaccccgctcttgttcagcacctatgtGatggggcggatggacacatactt
tgaggacccgctgactttcaaccccgatcgcttcggccctggagcacccaagccacggttcacctacttcccctt
ctccctgggccaccgctcctgcatcgggcagcagtttgctcagatggaggtgaaggtggtcatggcaaagctgct
gcagaggctggagttccggctggtgcccgggcagcgcttcgggctgcaggagcaggccacactcaagccactgga
ccccgtgctgtgcaccctgcggccccgcggctggcagcccgcacccccaccacccccctgctgagtgatagcttg
gtaccgagctcgatccaattgcaatgatcatcatgacagatctgcgcgcgatcgatatcagcgctttaaatttgc
gcatgcagctatagttctagagggccctattctatagtgtcacctaaatgctagagctcgctgatcagcctcgac
tgtgccttctagttgccagccatctgttgtttgcccctcccccgtgccttccttgaccctggaaggtgccactcc
cactgtcctttcctaataaaatgaggaaattgcatcgcattgtctgagtaggtgtcattctattctggggggtgg
ggtggggcaggacagcaagggggaggattgggaagacaatagcaggcatgctggggatgcggtgggctctatggg
tttaaacGCGGCCGCGTAGATAAGTAGCATGGCGGGTTAATCATTAACTACAAGGAACCCCTAGTGATGGAGTTG
GCCACTCCCTCTCTGCGCGCTCGCTCGCTCACTGAGGCCGGGCGACCAAAGGTCGCCCGACGCCCGGGCTTTGCC
CGGGCGGCCTCAGTGAGCGAGCGAGCGCGCAGTTAATTAAGGCGCCctaggccgacccttagactctgtactcag
ttctataaacgagccattggatacgagatccgtagattgataagggacacggaatatccccggacgcaatagaca
ccggtggacagcttggtatcctgagcacagtcgcgcgtccgaatctagctctactttagaggccccggattctga
tggtcgtagaccgcagaaccgattggggggatgagatctactagttatcagcacacaattgcccattatacgc

1. A method for treating a neurological disease or disorder in a subject in need thereof, the method comprising administering to a subject having or at risk of developing the neurological disease or disorder a therapeutically effective amount of at least one of:
(a) an isolated nucleic acid encoding a transgene encoding one or more miRNAs; and
(b) an isolated nucleic acid encoding a CYP46A1 protein.

2. A method for treating a neurological disease or disorder in a subject in need thereof, the method comprising administering to a subject having or at risk of developing the neurological disease or disorder a therapeutically effective amount of at least one of:
(a) a recombinant viral vector comprising an isolated nucleic acid comprising (i) a first region comprising a first adeno-associated virus (AAV) inverted terminal repeat (ITR), or a variant thereof, and (ii) a second region comprising a transgene encoding one or more miRNAs; and
(b) a recombinant viral vector comprising an isolated nucleic acid encoding the CYP46A1 protein.

3. The method of any of claims 1-2, wherein the neurological disease or disorder is Alzheimer's disease, Parkinson's disease, Huntington's disease, Canavan disease, Leigh's disease, spinal cerebral ataxia, polyglutamine repeat spinocerebellar ataxia, Krabbe's disease, Batten's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, neuropathic pain, trauma due to spinal cord or head injury, ophthalmic diseases and disorders, Tay-Sachs disease, Lesch-Nyhan disease, epilepsy, cerebral infarcts, depression, bipolar affective disorder, persistent affective disorder, secondary mood disorder, schizophrenia, drug dependency, neuroses, psychosis, dementia, paranoia, attention deficit disorder, psychosexual disorders, sleeping disorders, pain disorders, eating or weight disorders.

4. The method of any of claims 1-3, wherein the neurological disease or disorder is a central nervous system (CNS) disease or disorder.

5. The method of any of claims 1-4, wherein the CNS disease or disorder is selected from Huntington's disease, Alzheimer's disease, Polyglutamine repeat spinocerebellar ataxias, Amyotrophic lateral sclerosis and Parkinson's disease.

6. The method of any of claims 1-5, wherein the CNS disease or disorder is Alzheimer's disease and the at least one miRNA comprises a seed sequence complementary to Amyloid Precursor Protein (APP), Presenilin 1, Presenilin 2, ABCA7, SORL1, and disease-associated alleles thereof.

7. The method of any of claims 1-5, wherein the CNS disease or disorder is Parkinson's disease and the at least one miRNA comprises a seed sequence complementary to SNCA, LRRK2/PARK8, PRKN, PINK1, DJ1/PARK7, VPS35, EIF4G1, DNAJC13, CHCHD2, UCHL1, GBA1, and disease-associated alleles thereof.

8. The method of any of claims 1-5, wherein the CNS disease is Huntington's disease and at least one miRNA comprises a seed sequence complementary to SEQ ID NO: 4, or wherein at least one miRNA comprises the sequence of any one of SEQ ID NOs: 6-17, 40-44, 50-66, 158-185, or 217-260 flanked by a miRNA backbone sequence.

9. The method of any of claims 1-8, wherein the CNS disease is Huntington's disease and at least one miRNA comprises the sequence of any one of SEQ ID NOs: 6-17, 40-44, 50-66, 158-185, or 217-260.

10. The method of any of claims 8-9, wherein at least one of the miRNAs hybridizes with and inhibits expression of human huntingtin.

11. The method of any of claims 8-10, wherein the subject comprises a huntingtin gene having more than 36 CAG repeats, more than 40 repeats, or more than 100 repeats.

12. The method of any of claims 8-11, wherein the subject is less than 20 years of age.

13. The method of any of claims 1-12, wherein the recombinant viral vector is selected from the group consisting of: an AAV vector, an adenovirus vector, a lentivirus vector, a retrovirus vector, a herpesvirus vector, an alphavirus vector, a poxvirus vector, a baculovirus vector, and a chimeric virus vector.

14. The method of any of claims 2-13, wherein the recombinant viral vector comprising (a) is the same as the recombinant viral vector comprising (b).

15. The method of any of claims 1-13, wherein the isolated nucleic acid of (a) and (b) are comprised in separate recombinant viral vectors.

16. The method of any of claims 1-14, wherein the isolated nucleic acid of (a) and (b) are comprised in the same recombinant viral vector.

17. The method of any one of claims 1-16, wherein (a) and (b) are administered at substantially the same time.

18. The method of any one of claims 1-13 and 15, wherein (a) and (b) are administered at different time points.

19. The method of claim 18, wherein the different time points are spaced by at least 1 min, at least 1 hour, at least 1 day, at least 1 week, at least 1 month, at least 1 year, or more.

20. The method of any of claims 18-19, wherein (a) is administered prior to the administration of (b).

21. The method of any of claims 18-19, wherein (b) is administered prior to the administration of (a).

22. The method of any of claims 1-21, wherein the administration of (a), (b), or (a) and (b) is repeated at least once.

23. The method of any of claims 1-22, wherein the transgene comprises two miRNAs in tandem that are flanked by introns.

24. The method of claim 23, wherein the flanking introns are identical.

25. The method of claim 23, wherein the flanking introns are from the same species.

26. The method of claim 23, wherein the flanking introns are hCG introns.

27. The method of any one of claims 1-26, wherein the transgene comprises a promoter.

28. The method of claim 27, wherein the promoter is a synapsin (Syn1) promoter, or a promoter of Tables 10-13.

29. The method of any one of claims 1-28, wherein the one or more miRNAs are located in an untranslated portion of the transgene.

30. The method of claim 29, wherein the untranslated portion is an intron.

31. The method of claim 30, wherein the untranslated portion is between the last codon of the nucleic acid sequence encoding a protein and a poly-A tail sequence, or between the last nucleotide base of a promoter sequence and a poly-A tail sequence.

32. The method of any one of claims 1-31, further comprising a third region comprising a second adeno-associated virus (AAV) inverted terminal repeat (ITR), or a variant thereof.

33. The method of any one of claims 1-32, wherein the ITR variant lacks a functional terminal resolution site (TRS), optionally wherein the ITR variant is a ATRS ITR.

34. The method of any of claims 1-33, wherein the administration results in delivery of the viral vector or isolated nucleic acid to the central nervous system (CNS) of the subject.

35. The method of any of claims 1-34, wherein the administration is via injection, optionally intravenous injection or intrastriatal injection.

36. The method of any of claims 2-35, wherein the viral vector is AAV1, AAV2, AAV3b, AAV4, AAV5, AAV6, AAV7, AAV8, AAV9, AAV10, AAVrh10, AAV11, or, AAV12, or a chimera thereof.

37. The method of any of claims 2-36, the viral vector comprises a capsid protein from AAV serotype AAV1, AAV2, AAV3b, AAV4, AAV5, AAV6, AAV7, AAV8, AAV9, AAV10, AAVrh10, AAV11, or, AAV12, or a chimera thereof.

38. The method of claim 37, wherein the capsid protein is an AAV9 capsid protein.

39. The method of any of claims 2-38, wherein the viral vector is a self-complementary AAV (scAAV).

40. The method of any of claims 2-39, wherein the viral vector is formulated for delivery to the central nervous system (CNS).

41. A composition or combination comprising at least one of:
(a) an isolated nucleic acid encoding a transgene encoding one or more miRNAs; and
(b) an isolated nucleic acid encoding a CYP46A1 protein.

42. A composition or combination comprising of at least one of:
(a) a recombinant viral vector comprising an isolated nucleic acid comprising (i) a first region comprising a first adeno-associated virus (AAV) inverted terminal repeat (ITR), or a variant thereof, and (ii) a second region comprising a transgene encoding one or more miRNAs; and
(b) a recombinant viral vector comprising an isolated nucleic acid encoding the CYP46A1 protein.

43. The composition or combination of any of claims 41-42, for use in a method for treating a neurological disease or disorder in a subject in need thereof, the method comprising administering to a subject having or at risk of developing the neurological disease or disorder a therapeutically effective amount of the composition or combination.

44. The composition or combination of claim 43, wherein the neurological disease or disorder is Alzheimer's disease, Parkinson's disease, Huntington's disease, Canavan disease, Leigh's disease, spinal cerebral ataxia, Krabbe's disease, polyglutamine repeat spinocerebellar ataxia, Batten's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, neuropathic pain, trauma due to spinal cord or head injury, ophthalmic diseases and disorders, Tay-Sachs disease, Lesch-Nyhan disease, epilepsy, cerebral infarcts, depression, bipolar affective disorder, persistent affective disorder, secondary mood disorder, schizophrenia, drug dependency, neuroses, psychosis, dementia, paranoia, attention deficit disorder, psychosexual disorders, sleeping disorders, pain disorders, eating or weight disorders.

45. The composition or combination of claim 44, wherein the neurological disease or disorder is a central nervous system (CNS) disease or disorder.

46. The composition or combination of claim 45, wherein the CNS disease or disorder is selected from Huntington's disease, Alzheimer's disease, Polyglutamine repeat spinocerebellar ataxias, Amyotrophic lateral sclerosis and Parkinson's disease.

47. The composition or combination of any of claims 41-46, wherein the at least one miRNA comprises a seed sequence complementary to Amyloid Precursor Protein (APP), Presenilin 1, Presenilin 2, ABCA7, SORL1, and disease-associated alleles thereof.

48. The composition or combination of any of claims 41-46, wherein the at least one miRNA comprises a seed sequence complementary to SNCA, LRRK2/PARK8, PRKN, PINK1, DJ1/PARK7, VPS35, EIF4G1, DNAJC13, CHCHD2, UCHL1, GBA1, and disease-associated alleles thereof.

49. The composition or combination of any of claims 41-46, wherein the at least one miRNA comprises a seed sequence complementary to SEQ ID NO: 4, or wherein the at least one miRNA comprises the sequence of any one of SEQ ID NOs: 6-17, 40-44, 50-66, 158-185, or 217-260 flanked by a miRNA backbone sequence.

50. The composition or combination of any of claims 41-46, wherein the at least one miRNA comprises the sequence of any one of SEQ ID NOs: 6-17, 40-44, 50-66, 158-185, or 217-260.

51. The composition or combination of any of claims 49-50, wherein at least one of the miRNAs hybridizes with and inhibits expression of human huntingtin.

52. The composition or combination of any of claims 49-51, wherein the subject comprises a huntingtin gene having more than 36 CAG repeats, more than 40 repeats, or more than 100 repeats.

53. The composition or combination of any of claims 49-52, wherein the subject is less than 20 years of age.

54. The composition or combination of any of claims 42-53, wherein the recombinant viral vector is selected from the group consisting of: an AAV vector, an adenovirus vector, a lentivirus vector, a retrovirus vector, a herpesvirus vector, an alphavirus vector, a poxvirus vector a baculovirus vector, and a chimeric virus vector.

55. The composition or combination of any of claims 42-54, wherein the recombinant viral vector comprising (a) is the same as the recombinant viral vector comprising (b).

56. The composition or combination of any of claims 41-54, wherein the isolated nucleic acid of (a) and (b) are comprised in separate recombinant viral vectors.

57. The composition or combination of any of claims 41-55, wherein the isolated nucleic acid of (a) and (b) are comprised in the same recombinant viral vector.

58. The composition or combination of any of claims 41-57, wherein (a) and (b) are administered at substantially the same time.

59. The composition or combination of any of claims 41-54 and 56, wherein (a) and (b) are administered at different time points.

60. The composition or combination of claim 59, wherein the different time points are spaced by at least 1 min, at least 1 hour, at least 1 day, at least 1 week, at least 1 month, at least 1 year, or more.

61. The composition or combination of any of claims 59-60, wherein (a) is administered prior to the administration of (b).

62. The composition or combination of any of claims 59-60, wherein (b) is administered prior to the administration of (a).

63. The composition or combination of any of claims 59-60, wherein the administration of (a), (b), or (a) and (b) is repeated at least once.

64. The composition or combination of any of claims 41-63, wherein the transgene comprises two miRNAs in tandem that are flanked by introns.

65. The composition or combination of claim 64, wherein the flanking introns are identical.

66. The composition or combination of claim 64, wherein the flanking introns are from the same species.

67. The composition or combination of claim 64, wherein the flanking introns are hCG introns.

68. The composition or combination of any of claims 41-67, wherein the transgene comprises a promoter.

69. The composition or combination of claim 68, wherein the promoter is a synapsin (Syn1) promoter or a promoter of Tables 10-13.

70. The composition or combination of any of claims 41-69, wherein the one or more miRNAs are located in an untranslated portion of the transgene.

71. The composition or combination of claim 70, wherein the untranslated portion is an intron.

72. The composition or combination of claim 70, wherein the untranslated portion is between the last codon of the nucleic acid sequence encoding a protein and a poly-A tail sequence, or between the last nucleotide base of a promoter sequence and a poly-A tail sequence.

73. The composition or combination of any of claims 41-72, further comprising a third region comprising a second adeno-associated virus (AAV) inverted terminal repeat (ITR), or a variant thereof.

74. The composition or combination of any of claims 41-73, wherein the ITR variant lacks a functional terminal resolution site (TRS), optionally wherein the ITR variant is a ATRS ITR.

75. The composition or combination of any of claims 41-74, wherein the administration results in delivery of the viral vector or isolated nucleic acid to the central nervous system (CNS) of the subject.

76. The composition or combination of any of claims 41-75, wherein the administration is via injection, optionally intravenous injection or intrastriatal injection.

77. The composition or combination of any of claims 42-76, wherein the viral vector is an AAV1, AAV2, AAV3b, AAV4, AAV5, AAV6, AAV7, AAV8, AAV9, AAV10, AAVrh10, AAV11, AAV12, or a chimera thereof.

78. The composition of any of claims 42-77, the viral vector comprises a capsid protein from AAV serotype AAV1, AAV2, AAV3b, AAV4, AAV5, AAV6, AAV7, AAV8, AAV9, AAV10, AAVrh10, AAV11, or, AAV12, or a chimera thereof.

79. The composition or combination of claim 78, wherein the capsid protein is an AAV9 capsid protein.

80. The composition or combination of any of claims 42-79, wherein the viral vector is a self-complementary AAV (scAAV).

81. The composition or combination of any of claims 42-80, wherein the viral vector is formulated for delivery to the central nervous system (CNS).

82. A composition comprising an isolated nucleic acid encoding a CYP46A1 protein, the nucleic acid comprising a sequence at least 80% identical to SEQ ID NO: 110.

83. A composition comprising a recombinant viral vector comprising an isolated nucleic acid encoding a CYP46A1 protein, the nucleic acid comprising a sequence at least 80% identical to SEQ ID NO: 110.

84. A method for treating a neurological disease or disorder in a subject in need thereof, the method comprising administering to a subject having or at risk of developing the neurological disease or disorder a therapeutically effective amount of a composition of claim 82 or 83.

85. The method of claim 84, wherein the neurological disease or disorder is Alzheimer's disease, Parkinson's disease, Huntington's disease, Canavan disease, Leigh's disease, spinal cerebral ataxia, polyglutamine repeat spinocerebellar ataxia, Krabbe's disease, Batten's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, neuropathic pain, trauma due to spinal cord or head injury, ophthalmic diseases and disorders, Tay-Sachs disease, Lesch-Nyhan disease, epilepsy, cerebral infarcts, depression, bipolar affective disorder, persistent affective disorder, secondary mood disorder, schizophrenia, drug dependency, neuroses, psychosis, dementia, paranoia, attention deficit disorder, psychosexual disorders, sleeping disorders, pain disorders, eating or weight disorders.

86. The method of any of claims 84-85, wherein the neurological disease or disorder is a central nervous system (CNS) disease or disorder.

87. The method of any of claims 84-86, wherein the CNS disease or disorder is selected from Huntington's disease, Alzheimer's disease, Polyglutamine repeat spinocerebellar ataxias, Amyotrophic lateral sclerosis and Parkinson's disease.

88. The composition or method of any of claims 83-87, wherein the recombinant viral vector is selected from the group consisting of: an AAV vector, an adenovirus vector, a lentivirus vector, a retrovirus vector, a herpesvirus vector, an alphavirus vector, a poxvirus vector a baculovirus vector, and a chimeric virus vector.

89. The method of any of claims 84-88, wherein the administration is repeated at least once.

90. The method of any of claims 84-89, wherein the administration results in delivery of the viral vector or isolated nucleic acid to the central nervous system (CNS) of the subject.

91. The method of any of claims 84-90, wherein the administration is via injection, optionally intravenous injection or intrastriatal injection.

92. The composition or method of any of claims 83-91, wherein the viral vector is AAV1, AAV2, AAV3b, AAV4, AAV5, AAV6, AAV7, AAV8, AAV9, AAV10, AAVrh10, AAV11, or, AAV12, or a chimera thereof.

93. The composition or method of any of claims 83-92, viral vector comprises a capsid protein from AAV serotype AAV1, AAV2, AAV3b, AAV4, AAV5, AAV6, AAV7, AAV8, AAV9, AAV10, AAVrh10, AAV11, or, AAV12, or a chimera thereof.

94. The composition or method of claim 93, wherein the capsid protein is an AAV9 capsid protein.

95. The composition or method of any of claims 83-94, wherein the viral vector is a self-complementary AAV (scAAV).

96. The composition or method of any of claims 83-95, wherein the viral vector is formulated for delivery to the central nervous system (CNS).

97. The composition or method of any of claims 82-96, wherein the nucleic acid comprises a sequence at least 90% identical to SEQ ID NO: 110.

98. The composition or method of any of claims 82-96, wherein the nucleic acid comprises a sequence at least 95% identical to SEQ ID NO: 110.

99. The composition or method of any of claims 82-96, wherein the nucleic acid comprises a sequence identical to SEQ ID NO: 110.

100. The composition or method of any of claims 2-40, 42-81, 83-99, where the viral vector comprises a modified viral capsid.

101. The composition or method of any of claims 2-40, 42-81, 83-99, where the viral vector comprises a modification to a viral capsid.

102. The composition or method of claim 100 or 101, wherein the modification is a chemical, non-chemical or amino acid modification of the viral capsid.

103. The composition or method of claim 100 or 101, wherein at least one of the capsid modifications preferentially targets cells in the CNS or PNS.

104. The composition or method of claim 100 or 101, wherein the chemical modification comprises a chemically-modified tyrosine residue modified to comprise a covalently-linked mono- or polysaccharide moiety.

105. The composition or method of claim 104, wherein the chemically-modified tyrosine residue comprises a mono-saccharide selected from galactose, mannose, N-acetylgalactosamine, bridge GalNac, and mannose-6-phosphate.

106. The composition or method of claim 100 or 101, wherein the chemical modification comprises a ligand covalently linked to a primary amino group of a capsid polypeptide via a —CSNH-bond.

107. The composition or method of claim 106, wherein the ligand comprises an arylene or heteroarylene radical covalently bound to the ligand.

108. The composition or method of any of claims 100-107, wherein the modified viral capsid is a chimeric capsid or a haploid capsid.

109. The composition or method of any of claims 100-107, wherein the modified viral capsid is a haploid capsid.

110. The composition or method of any of claims 100-107, wherein the modified viral capsid is a chimeric or haploid capsid further comprising a modification.

111. The composition or method of any of claims 100-110, wherein the modified viral capsid is an AAV serotype AAV1, AAV2, AAV3b, AAV4, AAV5, AAV6, AAV7, AAV8, AAV9, AAV10, AAVrh10, AAV11, AAV12, or a mutant modified from, a chimera, a mosaic, or a rational haploid thereof.

112. The composition or method of any of claims 100-111, wherein the modification changes the antigenic profile of the modified viral capsid as compared to the unmodified viral capsid.

113. The composition or method of any of claims 100-112, wherein the modified viral capsid can be used for repeat administration

114. A synthetic CNS-specific promoter comprising or consisting of a sequence according to any one of SEQ ID NOs: 187-189 or a functional variant thereof.

115. The synthetic CNS-specific promoter of claim 114, wherein the functional variant is at least 60%, 65%, 70%, 75%, 80%, 85%, 90%, 91%, 92%, 93%, 94%, 95%, 96%, 97%, 98% or 99% identical to any one of SEQ ID NOs: 187-189.

116. A CRE comprising or consisting of a sequence according to any one of SEQ ID NO: 191 or 192 or a functional variant thereof.

117. The CRE of claim 116, comprising a sequence which is at least 60%, 65%, 70%, 75%, 80%, 85%, 90%, 91%, 92%, 93%, 94%, 95%, 96%, 97%, 98% or 99% identical to any one of SEQ ID NOs: 191 or 192.

118. A CRM comprising a CRE of claims 116 or 117.

119. A minimal promoter comprising or consisting of a sequence according to SEQ ID NO: 211 or a functional variant thereof.

120. A CNS-specific promoter comprising a CRE of claims 116 or 117, a CRM of claim 118, or a minimal promoter of claim 119.

121. An expression cassette comprising a synthetic CNS-specific promoter of any one of claims 114, 115, or 120 operably linked to a sequence encoding an expression product.

122. A vector comprising a synthetic CNS-specific promoter of any one of claims 114, 115, or 120 or an expression cassette of claim 121.

123. The vector of claim 122, wherein the vector is a viral vector.

124. The vector of claim 123, wherein the viral vector is an AAV vector.

125. A virion comprising a vector of claims 123 or 124.

126. A pharmaceutical composition comprising a synthetic CNS-specific promoter of any one of claims 114, 115, or 120, an expression cassette of claim 121, a vector of any one of claims 122-124, or a virion of claim 125.

127. A synthetic CNS-specific promoter of any one of claims 114, 115, or 120, an expression cassette of claim 121, a vector of any one of claims 122-124, a virion of claim 125, or a pharmaceutical composition of claim 126 for use in therapy.

128. The synthetic CNS-specific promoter, the expression cassette, the vector, the virion, or the pharmaceutical composition of claim 127 for use in gene therapy, suitably wherein the gene therapy involves expression of a therapeutic expression product in the CNS.

129. The synthetic CNS-specific promoter, the expression cassette, the vector, the virion, or the pharmaceutical composition of claims 127 or 128 for use in gene therapy of a CNS-related disease.

130. A cell comprising a synthetic CNS-specific promoter of any one of claims 114, 115, or 120, an expression cassette of claim 121, a vector of any one of claims 122-124, or a virion of claim 125.

131. The cell of claim 130, wherein the cell is a CNS cell, optionally a human CNS cell, preferably a neuron, more preferably a dopaminergic neuron.

132. A synthetic CNS-specific promoter of any one of claims 114, 115, or 120, an expression cassette of claim 121, a vector of any one of claims 122-124, or a virion of claim 125 for use in the manufacture of a pharmaceutical composition for the treatment of a medical condition or disease, optionally wherein the disease is a CNS-related disease.

133. A method for producing an expression product, the method comprising: introducing a synthetic CNS-specific expression cassette of claim 121 into a cell, optionally a CNS cell; and expressing the gene present in the synthetic CNS-specific expression cassette.

134. A method of expressing a therapeutic transgene in a cell, optionally a CNS cell, wherein the method comprises introducing into the cell an expression cassette of claim 121, a vector of any one of claims 122-124, or a virion of claim 125.

135. A method of therapy of a subject in need thereof, wherein the method comprises: administering to the subject in need thereof, an expression cassette of claim 120, a vector of any one of claims 122-124, or a virion of claim 125, or a pharmaceutical composition of claims 127 or 128, which comprises a sequence encoding a therapeutic product operably linked to.


SEQUENCE LISTING (186 sequences)

SEQ ID NO: 1 - DNA, 2138 bp, synthetic construct
  source 1..2138: mol_type = other DNA
atgagccccgggctgctgctgctcggcagcgccgtcctgctcgccttcggcctctgctgcaccttcgtgcaccgcgctcgcagccgctacgagcacatccccgggccgccgcggcccagtttccttctaggacacctcccctgcttttggaaaaaggatgaggttggtggccgtgtgctccaagatgtgtttttggattgggctaagaagtatggacctgttgtgcgggtcaacgtcttccacaaaacctcagtcatcgtcacgagtcctgagtcggttaagaagttcctgatgtcaaccaagtacaacaaggactccaagatgtaccgtgcgctccagactgtgtttggtgagagactcttcggccaaggcttggtgtccgaatgcaactatgagcgctggcacaagcagcggagagtcatagacctggccttcagccggagctccttggttagcttaatggaaacattcaacgagaaggctgagcagctggtggagattctagaagccaaggcagatgggcagaccccagtgtccatgcaggacatgctgacctacaccgccatggacatcctggccaaggcagcttttgggatggagaccagtatgctgctgggtgcccagaagcctctgtcccaggcagtgaaacttatgttggagggaatcactgcgtcccgcaacactctggcaaagttcctgccagggaagaggaagcagctccgggaggtccgggagagcattcgcttcctgcgccaggtgggcagggactgggtccagcgccgccgggaagccctgaagaggggcgaggaggttcctgccgacatcctcacacagattctgaaagctgaagagggagcccaggacgacgagggtctgctggacaacttcgtcaccttcttcattgctggtcacgagacctctgccaaccacttggcgttcacagtgatggagctgtctcgccagccagagatcgtggcaaggctgcaggccgaggtggatgaggtcattggttctaagaggtacctggatttcgaggacctggggagactgcagtacctgtcccaggtcctcaaagagtcgctgaggctgtacccaccagcatggggcacctttcgcctgctggaagaggagaccttgattgatggggtcagagtccccggcaacaccccgctcttgttcagcacctatgtcatggggcggatggacacatactttgaggacccgctgactttcaaccccgatcgcttcggccctggagcacccaagccacggttcacctacttccccttctccctgggccaccgctcctgcatcgggcagcagtttgctcagatggaggtgaaggtggtcatggcaaagctgctgcagaggctggagttccggctggtgcccgggcagcgcttcgggctgcaggagcaggccacactcaagccactggaccccgtgctgtgcaccctgcggccccgcggctggcagcccgcacccccaccacccccctgctgagggggcctccaggcaggacgagactcctcgggcaagggccgtgcccgcccacctctgctgcccacggccacccacccttctcccctgccccgtcccctgggccacccttcacgctggcttccagcgggccctctgccgaccgcctgcttcacacccctcagcgctccctgtcgcctgcggactccatggcccttcctggactggcccttgcccaactcccagccaccaccactgtccctaccactgagcccttgcacaggccacttgctcagacgagacaccctaactcttgctcactccctaaagccctcttcaggggtcacctcctccaagaagccctccttgccaccccccgccggcaggggcccctcctctgtgctccctcggtcacctgtgctacctctaacaccacactgaccacactgtatcgtgagtgtccgttgacgtgaccaattgccctgccaggctgtcagcgcctcaagggtagggtctgcgtgtgatttgtctctgagccccctgtgcccacccagggcccggcacagagtcgatgctcaataaatgtgtgttgactgcaaaaaaaaaaaaaaaaaaaaaaaaaaaaaaaaaaaaaaaaaaaaaaaaaaaaaaaaaaaaaa

SEQ ID NO: 2 - protein, 500 aa, Homo sapiens
MSPGLLLLGSAVLLAFGLCCTFVHRARSRYEHIPGPPRPSFLLGHLPCFWKKDEVGGRVLQDVFLDWAKKYGPVVRVNVFHKTSVIVTSPESVKKFLMSTKYNKDSKMYRALQTVFGERLFGQGLVSECNYERWHKQRRVIDLAFSRSSLVSLMETFNEKAEQLVEILEAKADGQTPVSMQDMLTYTAMDILAKAAFGMETSMLLGAQKPLSQAVKLMLEGITASRNTLAKFLPGKRKQLREVRESIRFLRQVGRDWVQRRREALKRGEEVPADILTQILKAEEGAQDDEGLLDNFVTFFIAGHETSANHLAFTVMELSRQPEIVARLQAEVDEVIGSKRYLDFEDLGRLQYLSQVLKESLRLYPPAWGTFRLLEEETLIDGVRVPGNTPLLFSTYVMGRMDTYFEDPLTFNPDRFGPGAPKPRFTYFPFSLGHRSCIGQQFAQMEVKVVMAKLLQRLEFRLVPGQRFGLQEQATLKPLDPVLCTLRPRGWQPAPPPPPC

SEQ ID NO: 3 - RNA, 614 nt, Homo sapiens
  source 1..614: mol_type = genomic RNA
ttgctgtgtgaggcagaacctgcgggggcaggggcgggctggttccctggccagccattggcagagtccgcaggctagggctgtcaatcatgctggccggcgtggccccgcctccgccggcgcggccccgcctccgccggcgcacgtctgggacgcaaggcgccgtgggggctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccgtgagtttgggcccgctgcagctccctgtc

SEQ ID NO: 4 - DNA, 13498 bp, Homo sapiens
  source 1..13498: mol_type = genomic DNA
gctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccaaagaaagaactttcagctaccaagaaagaccgtgtgaatcattgtctgacaatatgtgaaaacatagtggcacagtctgtcagaaattctccagaatttcagaaacttctgggcatcgctatggaactttttctgctgtgcagtgatgacgcagagtcagatgtcaggatggtggctgacgaatgcctcaacaaagttatcaaagctttgatggattctaatcttccaaggttacagctcgagctctataaggaaattaaaaagaatggtgcccctcggagtttgcgtgctgccctgtggaggtttgctgagctggctcacctggttcggcctcagaaatgcaggccttacctggtgaaccttctgccgtgcctgactcgaacaagcaagagacccgaagaatcagtccaggagaccttggctgcagctgttcccaaaattatggcttcttttggcaattttgcaaatgacaatgaaattaaggttttgttaaaggccttcatagcgaacctgaagtcaagctcccccaccattcggcggacagcggctggatcagcagtgagcatctgccagcactcaagaaggacacaatatttctatagttggctactaaatgtgctcttaggcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccaggtttatgaactgacgttacatcatacacagcaccaagaccacaatgttgtgaccggagccctggagctgttgcagcagctcttcagaacgcctccacccgagcttctgcaaaccctgaccgcagtcgggggcattgggcagctcaccgctgctaaggaggagtctggtggccgaagccgtagtgggagtattgtggaacttatagctggagggggttcctcatgcagccctgtcctttcaagaaaacaaaaaggcaaagtgctcttaggagaagaagaagccttggaggatgactctgaatcgagatcggatgtcagcagctctgccttaacagcctcagtgaaggatgagatcagtggagagctggctgcttcttcaggggtttccactccagggtcagcaggtcatgacatcatcacagaacagccacggtcacagcacacactgcaggcggactcagtggatctggccagctgtgacttgacaagctctgccactgatggggatgaggaggatatcttgagccacagctccagccaggtcagcgccgtcccatctgaccctgccatggacctgaatgatgggacccaggcctcgtcgcccatcagcgacagctcccagaccaccaccgaagggcctgattcagctgttaccccttcagacagttctgaaattgtgttagacggtaccgacaaccagtatttgggcctgcagattggacagccccaggatgaagatgaggaagccacaggtattcttcctgatgaagcctcggaggccttcaggaactcttccatggcccttcaacaggcacatttattgaaaaacatgagtcactgcaggcagccttctgacagcagtgttgataaatttgtgttgagagatgaagctactgaaccgggtgatcaagaaaacaagccttgccgcatcaaaggtgacattggacagtccactgatgatgactctgcacctcttgtccattgtgtccgccttttatctgcttcgtttttgctaacagggggaaaaaatgtgctggttccggacagggatgtgagggtcagcgtgaaggccctggccctcagctgtgtgggagcagctgtggccctccacccggaatctttcttcagcaaactctataaagttcctcttgacaccacggaataccctgaggaacagtatgtctcagacatcttgaactacatcgatcatggagacccacaggttcgaggagccactgccattctctgtgggaccctcatctgctccatcctcagcaggtcccgcttccacgtgggagattggatgggcaccattagaaccctcacaggaaatacattttctttggcggattgcattcctttgctgcggaaaacactgaaggatgagtcttctgttacttgcaagttagcttgtacagctgtgaggaactgtgtcatgagtctctgcagcagcagctacagtgagttaggactgcagctgatcatcgatgtgctgactctgaggaacagttcctattggctggtgaggacagagcttctggaaacccttgcagagattgacttcaggctggtgagctttttggaggcaaaagcagaaaacttacacagaggggctcatcattatacagggcttttaaaactgcaagaacgagtgctcaataatgttgtcatccatttgcttggagatgaagaccccagggtgcgacatgttgccgcagcatcactaattaggcttgtcccaaagctgttttataaatgtgaccaaggacaagctgatccagtagtggccgtggcaagagatcaaagcagtgtttacctgaaacttctcatgcatgagacgcagcctccatctcatttctccgtcagcacaataaccagaatatatagaggctataacctactaccaagcataacagacgtcactatggaaaataacctttcaagagttattgcagcagtttctcatgaactaatcacatcaaccaccagagcactcacatttggatgctgtgaagctttgtgtcttctttccactgccttcccagtttgcatttggagtttaggttggcactgtggagtgcctccactgagtgcctcagatgagtctaggaagagctgtaccgttgggatggccacaatgattctgaccctgctctcgtcagcttggttcccattggatctctcagcccatcaagatgctttgattttggccggaaacttgcttgcagccagtgctcccaaatctctgagaagttcatgggcctctgaagaagaagccaacccagcagccaccaagcaagaggaggtctggccagccctgggggaccgggccctggtgcccatggtggagcagctcttctctcacctgctgaaggtgattaacatttgtgcccacgtcctggatgacgtggctcctggacccgcaataaaggcagccttgccttctctaacaaaccccccttctctaagtcccatccgacgaaaggggaaggagaaagaaccaggagaacaagcatctgtaccgttgagtcccaagaaaggcagtgaggccagtgcagcttctagacaatctgatacctcaggtcctgttacaacaagtaaatcctcatcactggggagtttctatcatcttccttcatacctcaaactgcatgatgtcctgaaagctacacacgctaactacaaggtcacgctggatcttcagaacagcacggaaaagtttggagggtttctccgctcagccttggatgttctttctcagatactagagctggccacactgcaggacattgggaagtgtgttgaagagatcctaggatacctgaaatcctgctttagtcgagaaccaatgatggcaactgtttgtgttcaacaattgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcgggatggtttgatgtcctccagaaagtgtctacccagttgaagacaaacctcacgagtgtcacaaagaaccgtgcagataagaatgctattcataatcacattcgtttgtttgaacctcttgttataaaagctttaaaacagtacacgactacaacatgtgtgcagttacagaagcaggttttagatttgctggcgcagctggttcagttacgggttaattactgtcttctggattcagatcaggtgtttattggctttgtattgaaacagtttgaatacattgaagtgggccagttcagggaatcagaggcaatcattccaaacatctttttcttcttggtattactatcttatgaacgctatcattcaaaacagatcattggaattcctaaaatcattcagctctgtgatggcatcatggccagtggaaggaaggctgtgacacatgccataccggctctgcagcccatagtccacgacctctttgtattaagaggaacaaataaagctgatgcaggaaaagagcttgaaacccaaaaagaggtggtggtgtcaatgttactgagactcatccagtaccatcaggtgttggagatgttcattcttgtcctgcagcagtgccacaaggagaatgaagacaagtggaagcgactgtctcgacagatagctgacatcatcctcccaatgttagccaaacagcagatgcacattgactctcatgaagcccttggagtgttaaatacattatttgagattttggccccttcctccctccgtccggtagacatgcttttacggagtatgttcgtcactccaaacacaatggcgtccgtgagcactgttcaactgtggatatcgggaattctggccattttgagggttctgatttcccagtcaactgaagatattgttctttctcgtattcaggagctctccttctctccgtatttaatctcctgtacagtaattaataggttaagagatggggacagtacttcaacgctagaagaacacagtgaagggaaacaaataaagaatttgccagaagaaacattttcaaggtttctattacaactggttggtattcttttagaagacattgttacaaaacagctgaaggtggaaatgagtgagcagcaacatactttctattgccaggaactaggcacactgctaatgtgtctgatccacatcttcaagtctggaatgttccggagaatcacagcagctgccactaggctgttccgcagtgatggctgtggcggcagtttctacaccctggacagcttgaacttgcgggctcgttccatgatcaccacccacccggccctggtgctgctctggtgtcagatactgctgcttgtcaaccacaccgactaccgctggtgggcagaagtgcagcagaccccgaaaagacacagtctgtccagcacaaagttacttagtccccagatgtctggagaagaggaggattctgacttggcagccaaacttggaatgtgcaatagagaaatagtacgaagaggggctctcattctcttctgtgattatgtctgtcagaacctccatgactccgagcacttaacgtggctcattgtaaatcacattcaagatctgatcagcctttcccacgagcctccagtacaggacttcatcagtgccgttcatcggaactctgctgccagcggcctgttcatccaggcaattcagtctcgttgtgaaaacctttcaactccaaccatgctgaagaaaactcttcagtgcttggaggggatccatctcagccagtcgggagctgtgctcacgctgtatgtggacaggcttctgtgcacccctttccgtgtgctggctcgcatggtcgacatccttgcttgtcgccgggtagaaatgcttctggctgcaaatttacagagcagcatggcccagttgccaatggaagaactcaacagaatccaggaataccttcagagcagcgggctcgctcagagacaccaaaggctctattccctgctggacaggtttcgtctctccaccatgcaagactcacttagtccctctcctccagtctcttcccacccgctggacggggatgggcacgtgtcactggaaacagtgagtccggacaaagactggtacgttcatcttgtcaaatcccagtgttggaccaggtcagattctgcactgctggaaggtgcagagctggtgaatcggattcctgctgaagatatgaatgccttcatgatgaactcggagttcaacctaagcctgctagctccatgcttaagcctagggatgagtgaaatttctggtggccagaagagtgccctttttgaagcagcccgtgaggtgactctggcccgtgtgagcggcaccgtgcagcagctccctgctgtccatcatgtcttccagcccgagctgcctgcagagccggcggcctactggagcaagttgaatgatctgtttggggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccgttgcagtgcagcctggagagcagcttcttagtccagaaagaaggacaaataccccaaaagccatcagcgaggaggaggaggaagtagatccaaacacacagaatcctaagtatatcactgcagcctgtgagatggtggcagaaatggtggagtctctgcagtcggtgttggccttgggtcataaaaggaatagcggcgtgccggcgtttctcacgccattgctaaggaacatcatcatcagcctggcccgcctgccccttgtcaacagctacacacgtgtgcccccactggtgtggaagcttggatggtcacccaaaccgggaggggattttggcacagcattccctgagatccccgtggagttcctccaggaaaaggaagtctttaaggagttcatctaccgcatcaacacactaggctggaccagtcgtactcagtttgaagaaacttgggccaccctccttggtgtcctggtgacgcagcccctcgtgatggagcaggaggagagcccaccagaagaagacacagagaggacccagatcaacgtcctggccgtgcaggccatcacctcactggtgctcagtgcaatgactgtgcctgtggccggcaacccagctgtaagctgcttggagcagcagccccggaacaagcctctgaaagctctcgacaccaggtttgggaggaagctgagcattatcagagggattgtggagcaagagattcaagcaatggtttcaaagagagagaatattgccacccatcatttatatcaggcatgggatcctgtcccttctctgtctccggctactacaggtgccctcatcagccacgagaagctgctgctacagatcaaccccgagcgggagctggggagcatgagctacaaactcggccaggtgtccatacactccgtgtggctggggaacagcatcacacccctgagggaggaggaatgggacgaggaagaggaggaggaggccgacgcccctgcaccttcgtcaccacccacgtctccagtcaactccaggaaacaccgggctggagttgacatccactcctgttcgcagtttttgcttgagttgtacagccgctggatcctgccgtccagctcagccaggaggaccccggccatcctgatcagtgaggtggtcagatcccttctagtggtctcagacttgttcaccgagcgcaaccagtttgagctgatgtatgtgacgctgacagaactgcgaagggtgcacccttcagaagacgagatcctcgctcagtacctggtgcctgccacctgcaaggcagctgccgtccttgggatggacaaggccgtggcggagcctgtcagccgcctgctggagagcacgctcaggagcagccacctgcccagcagggttggagccctgcacggcgtcctctatgtgctggagtgcgacctgctggacgacactgccaagcagctcatcccggtcatcagcgactatctcctctccaacctgaaagggatcgcccactgcgtgaacattcacagccagcagcacgtactggtcatgtgtgccactgcgttttacctcattgagaactatcctctggacgtagggccggaattttcagcatcaataatacagatgtgtggggtgatgctgtctggaagtgaggagtccaccccctccatcatttaccactgtgccctcagaggcctggagcgcctcctgctctctgagcagctctcccgcctggatgcagaatcgctggtcaagctgagtgtggacagagtgaacgtgcacagcccgcaccgggccatggcggctctgggcctgatgctcacctgcatgtacacaggaaaggagaaagtcagtccgggtagaacttcagaccctaatcctgcagcccccgacagcgagtcagtgattgttgctatggagcgggtatctgttctttttgataggatcaggaaaggctttccttgtgaagccagagtggtggccaggatcctgccccagtttctagacgacttcttcccaccccaggacatcatgaacaaagtcatcggagagtttctgtccaaccagcagccatacccccagttcatggccaccgtggtgtataaggtgtttcagactctgcacagcaccgggcagtcgtccatggtccgggactgggtcatgctgtccctctccaacttcacgcagagggccccggtcgccatggccacgtggagcctctcctgcttctttgtcagcgcgtccaccagcccgtgggtcgcggcgatcctcccacatgtcatcagcaggatgggcaagctggagcaggtggacgtgaaccttttctgcctggtcgccacagacttctacagacaccagatagaggaggagctcgaccgcagggccttccagtctgtgcttgaggtggttgcagccccaggaagcccatatcaccggctgctgacttgtttacgaaatgtccacaaggtcaccacctgctgagcgccatggtgggagagactgtgaggcggcagctggggccggagcctttggaagtctgcgcccttgtgccctgcctccaccgagccagcttggtccctatgggcttccgcacatgccgcgggcggccaggcaacgtgcgtgtctctgccatgtggcagaagtgctctttgtggcagtggccaggcagggagtgtctgcagtcctggtggggctgagcctgaggccttccagaaagcaggagcagctgtgctgcaccccatgtgggtgaccaggtcctttctcctgatagtcacctgctggttgttgccaggttgcagctgctcttgcatctgggccagaagtcctccctcctgcaggctggctgttggcccctctgctgtcctgcagtagaaggtgccgtgagcaggctttgggaacactggcctgggtctccctggtggggtgtgcatgccacgccccgtgtctggatgcacagatgccatggcctgtgctgggccagtggctgggggtgctagacacccggcaccattctcccttctctcttttcttctcaggatttaaaatttaattatatcagtaaagagattaattttaacgtaactctttctatgcccgtgtaaagtatgtgaatcgcaaggcctgtgctgcatgcgacagcgtccggggtggtggacagggcccccggccacgctccctctcctgtagccactggcatagccctcctgagcacccgctgacatttccgttgtacatgttcctgtttatgcattcacaaggtgactgggatgtagagaggcgttagtgggcaggtggccacagcaggactgaggacaggcccccattatcctaggggtgcgctcacctgcagcccctcctcctcgggcacagacgactgtcgttctccacccaccagtcagggacagcagcctccctgtcactcagctgagaaggccagccctccctggctgtgagcagcctccactgtgtccagagacatgggcctcccactcctgttccttgctagccctggggtggcgtctgcctaggagctggctggcaggtgttgggacctgctgctccatggatgcatgccctaagagtgtcactgagctgtgttttgtctgagcctctctcggtcaacagcaaagcttggtgtcttggcactgttagtgacagagcccagcatcccttctgcccccgttccagctgacatcttgcacggtgaccccttttagtcaggagagtgcagatctgtgctcatcggagactgccccacggccctgtcagagccgccactcctatccccaggccaggtccctggaccagcctcctgtttgcaggcccagaggagccaagtcattaaaatggaagtggattctggatggccgggctgctgctgatgtaggagctggatttgggagctctgcttgccgactggctgtgagacgaggcaggggctctgcttcctcagccctagaggcgagccaggcaaggttggcgactgtcatgtggcttggtttggtcatgcccgtcgatgttttgggtattgaatgtggtaagtggaggaaatgttggaactctgtgcaggtgctgccttgagacccccaagcttccacctgtccctctcctatgtggcagctggggagcagctgagatgtggacttgtatgctgcccacatacgtgagggggagctgaaagggagcccctcctctgagcagcctctgccaggcctgtatgaggcttttcccaccagctcccaacagaggcctcccccagccaggaccacctcgtcctcgtggcggggcagcaggagcggtagaaaggggtccgatgtttgaggaggcccttaagggaagctactgaattataacacgtaagaaaatcaccattccgtattggttgggggctcctgtttctcatcctagctttttcctggaaagcccgctagaaggtttgggaacgaggggaaagttctcagaactgttggctgctccccacccgcctcccgcctcccccgcaggttatgtcagcagctctgagacagcagtatcacaggccagatgttgttcctggctagatgtttacatttgtaagaaataacactgtgaatgtaaaacagagccattcccttggaatgcatatcgctgggctcaacatagagtttgtcttcctcttgtttacgacgtgatctaaaccagtccttagcaaggggctcagaacaccccgctctggcagtaggtgtcccccacccccaaagacctgcctgtgtgctccggagatgaatatgagctcattagtaaaaatgacttcacccacgcatatacataaagtatccatgcatgtgcatatagacacatctataattttacacacacacctctcaagacggagatgcatggcctctaagagtgcccgtgtcggttcttcctggaagttgactttccttagacccgccaggtcaagttagccgcgtgacggacatccaggcgtgggacgtggtcagggcagggctcattcattgcccactaggatcccactggcgaagatggtctccatatcagctctctgcagaagggaggaagactttatcatgttcctaaaaatctgtggcaagcacccatcgtattatccaaattttgttgcaaatgtgattaatttggttgtcaagttttgggggtgggctgtggggagattgcttttgttttcctgctggtaatatcgggaaagattttaatgaaaccagggtagaattgtttggcaatgcactgaagcgtgtttctttcccaaaatgtgcctcccttccgctgcgggcccagctgagtctatgtaggtgatgtttccagctgccaagtgctctttgttactgtccaccctcatttctgccagcgcatgtgtcctttcaaggggaaaatgtgaagctgaaccccctccagacacccagaatgtagcatctgagaaggccctgtgccctaaaggacacccctcgcccccatcttcatggagggggtcatttcagagccctcggagccaatgaacagctcctcctcttggagctgagatgagccccacgtggagctcgggacggatagtagacagcaataactcggtgtgtggccgcctggcaggtggaacttcctcccgttgcggggtggagtgaggttagttctgtgtgtctggtgggtggagtcaggcttctcttgctacctgtgagcatccttcccagcagacatcctcatcgggctttgtccctcccccgcttcctccctctgcggggaggacccgggaccacagctgctggccagggtagacttggagctgtcctccagaggggtcacgtgtaggagtgagaagaaggaagatcttgagagctgctgagggaccttggagagctcaggatggctcagacgaggacactcgcttgccgggcctgggcctcctgggaaggagggagctgctcagaatgccgcatgacaactgaaggcaacctggaaggttcaggggccgctcttcccccatgtgcctgtcacgctctggtgcagtcaaaggaacgccttcccctcagttgtttctaagagcagagtctcccgctgcaatctgggtggtaactgccagccttggaggatcgtggccaacgtggacctgcctacggagggtgggctctgacccaagtggggcctccttgtccaggtctcactgctttgcaccgtggtcagagggactgtcagctgagcttgagctcccctggagccagcagggctgtgatgggcgagtcccggagccccacccagacctgaatgcttctgagagcaaagggaaggactgacgagagatgtatatttaattttttaactgctgcaaacattgtacatccaaattaaaggaaaaaaatggaaaccatcaaaaaaaaaaaaaaaaaa

SEQ ID NO: 5 - protein, 3144 aa, Homo sapiens
MATLEKLMKAFESLKSFQQQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 6 - RNA, 19 nt, synthetic construct
  source 1..19: mol_type = other RNA
aaggacttgagggactcga

SEQ ID NO: 7 - RNA, 20 nt, synthetic construct
  source 1..20: mol_type = other RNA
aaggacttgagggactcgaa

SEQ ID NO: 8 - RNA, 21 nt, synthetic construct
  source 1..21: mol_type = other RNA
aaggacttgagggactcgaag

SEQ ID NO: 9 - RNA, 22 nt, synthetic construct
  source 1..22: mol_type = other RNA
aaggacttgagggactcgaagg

SEQ ID NO: 10 - RNA, 23 nt, synthetic construct
  source 1..23: mol_type = other RNA
aaggacttgagggactcgaaggc

SEQ ID NO: 11 - RNA, 19 nt, synthetic construct
  source 1..19: mol_type = other RNA
tcgagtccctcaagtcctt

SEQ ID NO: 12 - RNA, 20 nt, synthetic construct
  source 1..20: mol_type = other RNA
ttcgagtccctcaagtcctt

SEQ ID NO: 13 - RNA, 21 nt, synthetic construct
  source 1..21: mol_type = other RNA
cttcgagtccctcaagtcctt

SEQ ID NO: 14 - RNA, 22 nt, synthetic construct
  source 1..22: mol_type = other RNA
ccttcgagtccctcaagtcctt

SEQ ID NO: 15 - RNA, 23 nt, synthetic construct
  source 1..23: mol_type = other RNA
gccttcgagtccctcaagtcctt

SEQ ID NO: 16 - RNA, 19 nt, synthetic construct
  source 1..19: mol_type = other RNA
cttcgagtctcaagtcctt

SEQ ID NO: 17 - RNA, 19 nt, synthetic construct
  source 1..19: mol_type = other RNA
acgagtccctcaagtcctc

SEQ ID NO: 18 - RNA, 20 nt, Homo sapiens
  source 1..20: mol_type = genomic RNA
aaggacttgagggactcgaa

SEQ ID NO: 19 - DNA, 21 bp, Homo sapiens
  source 1..21: mol_type = genomic DNA
tccaagatggacggccgctca

SEQ ID NO: 20 - DNA, 21 bp, Homo sapiens
  source 1..21: mol_type = genomic DNA
ccaagatggacggccgctcag

SEQ ID NO: 21 - DNA, 21 bp, Homo sapiens
  source 1..21: mol_type = genomic DNA
agatggacggccgctcaggtt

SEQ ID NO: 22 - DNA, 21 bp, Homo sapiens
  source 1..21: mol_type = genomic DNA
atggacggccgctcaggttct

SEQ ID NO: 23 - DNA, 21 bp, Homo sapiens
  source 1..21: mol_type = genomic DNA
gacggccgctcaggttctgct

SEQ ID NO: 24 - DNA, 21 bp, Homo sapiens
  source 1..21: mol_type = genomic DNA
cggccgctcaggttctgcttt

SEQ ID NO: 25 - DNA, 21 bp, Homo sapiens
  source 1..21: mol_type = genomic DNA
gtgctgagcggcgccgcgagt

SEQ ID NO: 26 - DNA, 21 bp, Homo sapiens
  source 1..21: mol_type = genomic DNA
cgccgcgagtcggcccgaggc

SEQ ID NO: 27 - DNA, 21 bp, Homo sapiens
  source 1..21: mol_type = genomic DNA
accgccatggcgaccctggaa

SEQ ID NO: 28 - DNA, 21 bp, Homo sapiens
  source 1..21: mol_type = genomic DNA
ccgccatggcgaccctggaaa

SEQ ID NO: 29 - DNA, 21 bp, Homo sapiens
  source 1..21: mol_type = genomic DNA
gaaggccttcgagtccctcaa

SEQ ID NO: 30 - DNA, 21 bp, Homo sapiens
  source 1..21: mol_type = genomic DNA
cttcgagtccctcaagtcctt

SEQ ID NO: 31 - DNA, 21 bp, Homo sapiens
  source 1..21: mol_type = genomic DNA
ccgccgccgcctcctcagctt

SEQ ID NO: 32 - DNA, 21 bp, Homo sapiens
  source 1..21: mol_type = genomic DNA
gccgcctcctcagcttcctca

SEQ ID NO: 33 - DNA, 21 bp, Homo sapiens
  source 1..21: mol_type = genomic DNA
tcagccgccgccgcaggcaca

SEQ ID NO: 34 - DNA, 21 bp, Homo sapiens
  source 1..21: mol_type = genomic DNA
gccgcaggcacagccgctgct

SEQ ID NO: 35 - DNA, 21 bp, Homo sapiens
  source 1..21: mol_type = genomic DNA
ggcacagccgctgctgcctca

SEQ ID NO: 36 - DNA, 21 bp, Homo sapiens
  source 1..21: mol_type = genomic DNA
gccgctgctgcctcagccgca

SEQ ID NO: 37 - DNA, 21 bp, Homo sapiens
  source 1..21: mol_type = genomic DNA
cggcccggctgtggctgagga

SEQ ID NO: 38 - DNA, 21 bp, Homo sapiens
  source 1..21: mol_type = genomic DNA
ctgtggctgaggagccgctgc

SEQ ID NO: 39 - DNA, 21 bp, Homo sapiens
  source 1..21: mol_type = genomic DNA
tgtggctgaggagccgctgca

SEQ ID NO: 40 - RNA, 72 nt, synthetic construct
  source 1..72: mol_type = other RNA
cttgggaatggcaaggaaggacttgagggactcgaagacgagtccctcaagtcctctcttgctatacccaga

SEQ ID NO: 41 - RNA, 69 nt, synthetic construct
  source 1..69: mol_type = other RNA
tgctgaaggacttgagggactcgaaggttttggccactgactgaccttcgagtctcaagtccttcagga

SEQ ID NO: 42 - RNA, 272 nt, synthetic construct
  source 1..272: mol_type = other RNA
gctaagcacttcgtggccgtcgatcgtttaaagggaggtagtgagtcgaccagtggatcctggaggcttgctgaaggctgtatgctgaaggacttgagggactcgaaggttttggccactgactgaccttcgagtctcaagtccttcaggacacaaggcctgttactagcactcacatggaacaaatggcccagatctggccgcactcgagatatctagacccagctttcttgtacaaagtggttgatctagagggcccgcggttcgctgat

SEQ ID NO: 43 - RNA, 605 nt, synthetic construct
  source 1..605: mol_type = other RNA
gctcctgggcaacgtgctggttattgtgctgtctcatcattttggcaaagaattaagggcgaattcgagctcggtacctcgcgaatgcatctagatatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggaaggacttgagggactcgaagacgagtccctcaagtcctctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatccagatctcgagcggccgcccg

SEQ ID NO: 44 - RNA, 819 nt, synthetic construct
  source 1..819: mol_type = other RNA
agtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacccggatcccccgggctgcaggaattcgagctcggtacctcgcgaatgcatctagatatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggaaggacttgagggactcgaagacgagtccctcaagtcctctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatccagatctcgagcggccgcccg

SEQ ID NO: 45 - RNA, 40 nt, synthetic construct
  source 1..40: mol_type = other RNA
ccacgcctgctccctcatccactgtgtgcacttcatcctg

SEQ ID NO: 46 - RNA, 40 nt, synthetic construct
  source 1..40: mol_type = other RNA
ccacgcctgctccctcatctactgtgtgcacttcatcctg

SEQ ID NO: 47 - RNA, 37 nt, synthetic construct
  source 1..37: mol_type = other RNA
taagagatggggacagtaattcaacgctagaagaaca

SEQ ID NO: 48 - RNA, 37 nt, synthetic construct
  source 1..37: mol_type = other RNA
taagagatggggacagtacttcaacgctagaagaaca

SEQ ID NO: 49 - DNA, 27 bp, synthetic construct
  source 1..27: mol_type = other DNA
cagatgccatggcctgtgctgggccag

SEQ ID NO: 50 - RNA, 21 nt, synthetic construct
  source 1..21: mol_type = other RNA
tccctcatccactgtgtgaac

SEQ ID NO: 51 - RNA, 21 nt, synthetic construct
  source 1..21: mol_type = other RNA
gcacacagtggatgagggagc

SEQ ID NO: 52 - RNA, 21 nt, synthetic construct
  source 1..21: mol_type = other RNA
tccctcatctactgtgtgaac

SEQ ID NO: 53 - RNA, 21 nt, synthetic construct
  source 1..21: mol_type = other RNA
cgagggagtagatgacacacg

SEQ ID NO: 54 - RNA, 21 nt, synthetic construct
  source 1..21: mol_type = other RNA
gggacagtaattcaacgcgtc

SEQ ID NO: 55 - RNA, 21 nt, synthetic construct
  source 1..21: mol_type = other RNA
agcgttgaattactgtcccca

SEQ ID NO: 56 - RNA, 21 nt, synthetic construct
  source 1..21: mol_type = other RNA
gggacagtacttcaacgcgtc

SEQ ID NO: 57 - RNA, 21 nt, synthetic construct
  source 1..21: mol_type = other RNA
acccctgtcatgaagttgcga

SEQ ID NO: 58 - RNA, 21 nt, synthetic construct
  source 1..21: mol_type = other RNA
tgccatggcctgtgctggtcc

SEQ ID NO: 59 - RNA, 20 nt, synthetic construct
  source 1..20: mol_type = other RNA
cccagcacaggccatggcac

SEQ ID NO: 60 - RNA, 21 nt, synthetic construct
  source 1..21: mol_type = other RNA
tgccatggcatgtgctggtcc

SEQ ID NO: 61 - RNA, 21 nt, synthetic construct
  source 1..21: mol_type = other RNA
cccagcacatgcctaggcatc

SEQ ID NO: 62 - DNA, 20 bp, synthetic construct
  source 1..20: mol_type = other DNA
ctcagtaacattgacaccac

SEQ ID NO: 63 - DNA, 20 bp, synthetic construct
  source 1..20: mol_type = other DNA
ctcgactaaagcaggatttc

SEQ ID NO: 64 - DNA, 20 bp, synthetic construct
  source 1..20: mol_type = other DNA
ccttccctgaaggttcctcc

SEQ ID NO: 65 - DNA, 20 bp, synthetic construct
  source 1..20: mol_type = other DNA
gcagggttaccgccatcccc

SEQ ID NO: 66 - DNA, 20 bp, synthetic construct
  source 1..20: mol_type = other DNA
cgagacagtcgcttccactt

SEQ ID NO: 67 - DNA, 20 bp, synthetic construct
  source 1..20: mol_type = other DNA
cgaggccggggcggggcaca

SEQ ID NO: 68 - DNA, 20 bp, synthetic construct
  source 1..20: mol_type = other DNA
cggggcggggccgtggaggg

SEQ ID NO: 69 - DNA, 20 bp, synthetic construct
  source 1..20: mol_type = other DNA
actgtgccactatgttttca

SEQ ID NO: 70 - DNA, 20 bp, synthetic construct
  source 1..20: mol_type = other DNA
gccttcatcagcttttccag

SEQ ID NO: 71 - DNA, 20 bp, synthetic construct
  source 1..20: mol_type = other DNA
gctgctgctgctgctgctgc

SEQ ID NO: 72 - DNA, 20 bp, synthetic construct
  source 1..20: mol_type = other DNA
tgctggaaggacttgaggga

SEQ ID NO: 73 - DNA, 20 bp, synthetic construct
  source 1..20: mol_type = other DNA
tgttgctgctgctgctgctg

SEQ ID NO: 74 - DNA, 795 bp, synthetic construct
  source 1..795: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 75 - DNA, 810 bp, synthetic construct
  source 1..810: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 76 - DNA, 916 bp, synthetic construct
  source 1..916: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggaagcgcgcagagtctgcatgcgtgaggaagctcctgggcgcgtcacagccgcgctattctcagcgtctctccttttatggctccggaagtgagctggggttgctggcagcctggctggcactgggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 77 - DNA, 931 bp, synthetic construct
  source 1..931: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggaagcgcgcagagtctgcatgcgtgaggaagctcctgggcgcgtcacagccgcgctattctcagcgtctctccttttatggctccggaagtgagctggggttgctggcagcctggctggcacttgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 78 - DNA, 617 bp, synthetic construct
  source 1..617: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacgggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 79 - DNA, 632 bp, synthetic construct
  source 1..632: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcactgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 80 - DNA, 701 bp, synthetic construct
  source 1..701: mol_type = other DNA
ttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 81 - DNA, 716 bp, synthetic construct
  source 1..716: mol_type = other DNA
ttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 82 - DNA, 792 bp, synthetic construct
  source 1..792: mol_type = other DNA
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaagggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 83 - DNA, 686 bp, synthetic construct
  source 1..686: mol_type = other DNA
cttcctcttatatttcaccaagactcagttcctgagcaagaaaccacaggcacagcaagtgccatgaaaagcggctttgtgtggggtgggctcttcacactccaatctccacttccttctcaaggcctcaaaaaaagttgaaaaatgaaaacaaaagccctgctgtgttgagctgggctctggcgttgccatggacccagggcaaacagcggtgctcctgctctgcccccggctcagctcatgctgggcctgcacttctggaagggagcatggactttggaatgactggttagaacccaaatgaattaatggaatttgacatagttcaaaaataataaaatgtgatacccatgaaatgctgatattctgccttaatttgccagattgggggccgggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 84 - DNA, 807 bp, synthetic construct
  source 1..807: mol_type = other DNA
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 85 - DNA, 701 bp, synthetic construct
  source 1..701: mol_type = other DNA
cttcctcttatatttcaccaagactcagttcctgagcaagaaaccacaggcacagcaagtgccatgaaaagcggctttgtgtggggtgggctcttcacactccaatctccacttccttctcaaggcctcaaaaaaagttgaaaaatgaaaacaaaagccctgctgtgttgagctgggctctggcgttgccatggacccagggcaaacagcggtgctcctgctctgcccccggctcagctcatgctgggcctgcacttctggaagggagcatggactttggaatgactggttagaacccaaatgaattaatggaatttgacatagttcaaaaataataaaatgtgatacccatgaaatgctgatattctgccttaatttgccagattgggggcctgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 86 - DNA, 913 bp, synthetic construct
  source 1..913: mol_type = other DNA
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaaagcgcgcagagtctgcatgcgtgaggaagctcctgggcgcgtcacagccgcgctattctcagcgtctctccttttatggctccggaagtgagctggggttgctggcagcctggctggcactgggctaggcctgcgcacccacccaccgacccctcacccaccgacccgtcacccaccgaccaaggggcaccctggcctagaggggatgctgagcgggacccgcctcctgcctctggcagtcccagatgggacttggaccccgcagttgctctctcggaccctaagtttctacccctggatctaaggcggagctgggtttgcggatcccacggttcccggcggggcggggcccggtcgcccctccccctccccgccctcctgcgccgggagcagtgcattgtgggaaactcccga

SEQ ID NO: 87 - DNA, 928 bp, synthetic construct
  source 1..928: mol_type = other DNA
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaaagcgcgcagagtctgcatgcgtgaggaagctcctgggcgcgtcacagccgcgctattctcagcgtctctccttttatggctccggaagtgagctggggttgctggcagcctggctggcacttgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 88 - DNA, 529 bp, synthetic construct
  source 1..529: mol_type = other DNA
ctagcccacaggaaatgtctgtctatatccaggcaagtaccttgctcattggaccaacccgaaactgttcagggaagatcagggaaatcaactcagttacaaatgggataatcatgcccagtaaaaactacctgtggtgaataaagagttaacccctgttccatcttaggtcactatgcagagtaccaatgagtacaagagatggtgccaaagagggtggcccctccctagctgggaacagtcaacccttaggaactagactgtcaacacatcagccagccagagacaagggaaaccgtggcaaccaagtgttgctggcacattgtgaggtggtgatgggaactgcagaggccctgcacagcatgctaatgagcccaggcaaacatgagctctccccatagctgggctgcggcccaaccccaccccctcaggctatgccagggggtgttgccaggggcacccgggcatcgccagtctagcccactccttcataaagccctcgcatcccaggagcgagcagagccagagc

SEQ ID NO: 89 - DNA, 646 bp, synthetic construct
  source 1..646: mol_type = other DNA
tccaaagaaaagccagataagtagctgattattgcatagagctgacagtatcacaggaagatcagtagtagcagctcaagtacaaaaaggttaattagcaatacttaataagaaaaactacctctggcaggtgaagagttaatccctggtcaattttaagctactctgctgagagtactaataagtgtaggggttggagccaatgagggtgaccccttccttgatgggaacagtcatcccttaggaactgccctggaaagcatcagccagccagaaaacagggaaagaggctgagaaaccgtggtaaccaagttttgctggcactttgtaaaatggtaactgcaactgccgaggctgtgcagagaatgctaataagcctaggacaacctgtaaagagtggacctagaaaatgtccacccgctagagagagggagcgagcatgtgcgatgagcaatagctgtggaccttacagttgctgctaactgccctggtgtgtgtgagggagagagagggagggagggagagagagcgcgctagcgcgagagagcgagtgagcaagcgagcagaaaagaggtggagagggggggaataagaaagagagagaaggaaaggagagaaggcaggaagaaggcaagggacgagacaa

SEQ ID NO: 90 - DNA, 614 bp, synthetic construct
  source 1..614: mol_type = other DNA
ctagcccacaggaaatgtctgtctatatccaggcaagtaccttgctcattggaccaacccgaaactgttcagggaagatcagggaaatcaactcagttacaaatgggataatcatgcccagtaaaaactacctgtggtgaataaagagttaacccctgttccatcttaggtcactatgcagagtaccaatgagtacaagagatggtgccaaagagggtggcccctccctagctgggaacagtcaacccttaggaactagactgtcaacacatcagccagccagagacaagggaaaccgtggcaaccaagtgttgctggcacattgtgaggtggtgatgggaactgcagaggccctgcacagcatgctaatgagcccaggcaaacatcgctagagagagggagcgagcatgtgcgatgagcaatagctgtggaccttacagttgctgctaactgccctggtgtgtgtgagggagagagagggagggagggagagagagcgcgctagcgcgagagagcgagtgagcaagcgagcagaaaagaggtggagagggggggaataagaaagagagagaaggaaaggagagaaggcaggaagaaggcaagggacgagacaa

SEQ ID NO: 91 - DNA, 696 bp, synthetic construct
  source 1..696: mol_type = other DNA
ctgggcagagagggggcatcgggggcatggctaggggccagcactgtgcttcctgggcgcctcacctcctccctgactcctggagactcccagcccctgtctgggagatgagcatttaggaatctgcttgtgcaggggtggtgggaggggccggggtggagggcgcatccccacggggagattggatggaaatggcctgccagtgtgtgtgtgagtgtgcgcctgtggcagcagcagagtaaacagccgctgccctgtcctctctgcggccgtggccaggtacacaggcctgtttggacagctgccttgtctgtccgtctgtttgggagatgctggctgatagatggggatgggcggactgttaacccctcgttgcctgcactgctatgtgcttcctgcctcatccatggggtagaaggtagccagaaggtggtcctggctgtgcccccagctcctctctaggggggaaacctctagttctgagtcagggacagagtgaggagggctccagggcatcaagagcttgctcctccccgcaccagggagccaaggacagaggagaagggggtcttccccagtggtgactaggggcagaatatgtctctgagtgagtgtctggagccctcctcaccccaacaccatggggctgggcataaaagtcagggcagagccatctattgcttacatttgcttctg

SEQ ID NO: 92 - DNA, 709 bp, synthetic construct
  source 1..709: mol_type = other DNA
ggtgtgtggaagggtgagaggcacacacacagacactgaaagaatcctaggcctggtaggcacttaacaaatgtctgttacagaccagaattttattgctgttagagacccaagcccctcataggaacagtgagaaacaggtgcagaaaggcggagtaactttatctaaagtcataggctccctgaatagcagagctgacacctacaaggaagcgttggagaccagatctaccagctagcctccctgagaccacgaggtggcgccgcagcaccggctgtggccgatgccagccaggtagccggtttcccacgtcccccgcacgcacgcacctctttgctgcaggaatcccgggctgccccgacctggagtagggggggtggtgagtgggactgagtccctagaagcctggaccctcacttcgttcctgtacatccagctcgcctgtagacagtgggggaggatgaagggaagaggactcaagcgcaactttgaatcatcacgccttcgacagtccgcgcacgtttatttcatttatctttgaaaacgagggaggggaagcctggagaaggcgggatgggccaagggtgagttggcccccggggagctggtccctgttcctggctttagtcccaggggcgcggtctgtgtgtagggcgggctgggcataaaagtcagggcagagccatctattgcttacatttgcttctg

SEQ ID NO: 93 - DNA, 768 bp, synthetic construct
  source 1..768: mol_type = other DNA
tgctaccagagccgggagagctgctcggagacgcctccggggtgcgggctggacatgagcagcggctgccggtcctgggactaggccccgccattttggatccgctgacaggtcagcgaagtctcttcctagagttccggtgtcgtgaaggccgccctgacatcgcaatagggaattagtgggaagggcccttaaattgggcgagccaaggtggggggaggattggaacagagacaaaagggaggagagacggacagcgacaagtggagaaaatcggcgaaacttgagtggcagagaagtctgagcgctgagacccggcggccccgtgcgccttcccacctggcgccgatccactttcctcggggtagcggcccaacccacttcgctgccagccgatcccttttacccgtggctaccgggaccactctactctcgcccacttggctctgcctaagcgtcctagccggagcgcggtctctgccacgtggggaggggcgcggccgagttgctgaagagcgcttctgattggccagagggcggggttcttggcgtctcgccggccagacccctccctcaaaggcggggcctggagatccacagctggaaagggcggagccccagcagggcagctggaaaggggcggggcctgacgcgcgcggctcgccgcggcgggctgggggcgccctggtctgccataaagtgaatgggcgccggctgggggtggcagtacgcggtgaggctcactccctccgagagtccaggagcgcc

SEQ ID NO: 94 - DNA, 736 bp, synthetic construct
  source 1..736: mol_type = other DNA
aaggagaatggtaaacagcaggagcgaagcggctgaggagaaagaagaggaaagaaaggcgagacgtgggaggattggaacagagacaaaagggaggagagacggacagcgacaagtggagaaaatcggcgaaacttgagtggcagagaagtctgagcgctgagacccggcggccccgtgcgccttcccacctggcgccgatccactttcctcggggtagcggcccaacccacttcgctgccagccgatcccttttacccgtggctaccgggaccactctactctcgcccacttggctctgcctaagcgtcctagccggagcgcggtctctgccacgtggggaggggcgcggccgagttgctgaagagcgcttctgattggccagagggcggggttcttggcgtctcgccggccagacccctccctcaaaggcggggcctggagatccacagctggaaagggcggagccccagcagggcagctggaaaggggcggggcctgacgcgcgcggctcgccgcggcgggctgggggcgccctggtctgccataaagtgaatgggcgccggctgggggtggcagtacgcggtgaggctcactccctccgagagtccaggagcgcccgagcggagaggcggcccgggagcaggggggcggcccccactccggccgggtgcccggcccctggcccctgcctgccctctagatcgccgccgcagccgccgctactgggagtctg

SEQ ID NO: 95 - DNA, 1744 bp, synthetic construct
  source 1..1744: mol_type = other DNA
ttgtaatgggaataagggcaggactcctgggtataagtagctcagctgatcccaccctgcttctatgtgttaattcatttattcattcattcaacaagcatttgttgaaatgctctttgtgtcaggctcagcaggaagcagtggcaataaaatggtgaacaagaaagactcggggtttcttcatctatgttgatgtctgcagagaacagtatcagccttctaggaagtttgtaatcagatacattgttagagagatacttatctagtaaattcctactcatcctataaggctcaaaacaaatgcctctatgaaaccttccgtgattccctcaggcagagttaagagcttcctttcctgggcctctatctccttccattagtattataactgtttaccagtttcccctctagactaaatttctcaaaagagagaatgaggtctctttcagtcttctttgcatctttaaactagcctgggtcccagcctgtttgatgaaagaaacaagaacactgatacaagccacagccccttggcaaaaaagatacccaatagcaatggcaatgtaaaatcagttttagtaaatgaatcaagaattctgatgctttagggaaagtaatgtgaacctggcaccattaacaaattcagaactcttcttcttaggagctctctaactgaacagacagagggatgtcaacccctaattcagcttgatcgtatctcagcaactacatttaatgagacagtgggaaaaagagagctgtccacttttaaatcagcatatttctaactaaacaatggcaatggctaaatctttaaaatgcctatttctctcaagaacactgcaatggaacatttagactttgggaaagagattagtgatttacattgctatctcactgatttaatttaaatgctcttccaaaccaaacacacatgtgccgaagaggctactaagaaacccaacatgcagagttctctataagtgcagccgacagtgttgactgaaactaaacttggaaatccagggcactaatgcacaatatcaagcaataaaacggcatctctttggcaatatttaatttaaaaaagaagaaagagacaggcgaagatcaggcactgtctgttttggaggatcaaccattctgcatttcaaagcattggtccctgcaatatccaggttactgtgctagaatctcgactattatatcgcagttgtgagagggagggcaaagatgtgtttactcagtgattaggcccttagaataagcctctagctcctagagagacagctcaccacttattcatttgggccaattcacaaagcctaggaagattaaacatccatgctgagaagacaagcgaatgcagacggtgaaaaagaaataaaaattctttaaaaactctgagatgacttcattatttttccacaaggaaactttaggaaagtgtttagttagagaaaaacccacattgacctctctctaaacccttaatctttcctttgtggtggcactgctttgtggtaagcgactggctcgcctcgcccctcttttcactggaagctgagagaaaaaagactctggagaaacagttttcgttccagggacacaaacccctgacactgttaaacatgagatgccaggaaaacacacttaaaaaaaaaattcccactttaagctttagactgaatgtgagaaaggagatgataaaaagagtatcacaagagaatcttcaggctgtggg

SEQ ID NO: 96 - DNA, 1104 bp, synthetic construct
  source 1..1104: mol_type = other DNA
tttggcactgtgagcagtttacttgacaaattctgtcaaatatttgctttctgaaatctcgagaattggttgaatataattgtacttaatgtttgcaaaataaataaatatgggactaaggacgttctatcattaatttgtcagaaaagagagttgtcatttctgaaaatttaatgtcattgaagctctatttccaatagcaaaggagcactattgctaatagacttcagagcttgaaataaataaatctttggaatcctgttgcatctcttggggtgtgacatttgacagtcttttatagcacagaacgaaacaagtttgtgagctggaattcaattgtggcgtattgattccttgcatcagtcattattccctgctgattgacaggtgaaaattggttacgttaagtatttcatatgttatattggctgacatttgcttgcctgctcttgtgtcaatattgttgtaaagatctccagctttatgagatagcaatagacactgactgtggcttttgtgtgatgttccagtgtttttcctgacataatttaagacatattaaaaaccagcagcatcttccctcttgagaagcttaatgccaatattattgtcttccaggggaagatcatgtatgctcataatcgggtgctaatttccaccagtacgctcatgtttaggcattaggcactataactgtaaaattgagccttcttgattgattcatgtcaagcctcatctcggctcctgcaggggaagtcatccggctgaccctttttacactaaaagaagagatttgtgttcctttctttcacctggaaccatcaaattgactgaataatctgtaatacattagtgctgacatttgttagggagaattaaacaagacacagtaatcattccccagaataaaaattgtgtttgatttccagcagagttctattaaagggaggacagaatctgtctcttccaaggtggaaaatcgtgaatattccctgcattaatgaaccaagttaacactttaattgcttatagaaccgagttctccaatgacagcattaaaagatagggaggctctgatttatggtcaacacagatttgtaaccc

SEQ ID NO: 97 - DNA, 1910 bp, synthetic construct
  source 1..1910: mol_type = other DNA
tcaacatggataaccaaagttcttaaaactacgctttcaatgaacacatatcctttgagcaagactaataatgaggaatgggagccagctcctgtgatatttatgcaactactaaattctcactgaagtcaatgggagtttgcttacgtaagggctgcaaactttagcctccagagattaaaggggaaaaaaatccttaaactctttcaacattaatattgcctgtaaggaatccagccatgacctaagccatggagctttctgaacctagcaagtagaagggtaaacagtaaacaccagttattttaagcacaatctaatcagagttcaatgagaagcaatattatatttgatctctaaggtattaatacttgtatatcactattagacatctttatgtagtccattatccaaacaatggcttaagtctgtggtatttaataaatcaagtttccatggccgtgagactgagtgggagtggggatgaagccttttttcttcatttttttttcctcaggtgcaattctgtgttaatataagagaagtgtggccttccttctcatagcactaaaagtgagataatccctgtgtaagaaatcagtaagtacggtctgcttaatctagtcccagtgtgaaactgttgacatttgttcttttttctatcattatgtgactgggcctgttttgtgctggattaggcacaaatctcctatgcagcacatttggcatgttactagtagtttaacttcattaataatgtatgaagaaaatgtaatccatgacaaggaagcaaagaaaagtatttttttttttttttgcttctcccaaatcctttggaatgagtaattattcaacattttatgtttgatgttatattttacaattcaacttccatagtgatatttaaaaaagaaactttggcaaatgcttgcaaaaaacacaccttttacaattttaaatgtgatttactgatggccagaacttgttaaacatagtaggaaattaaatatttattcatcttatttcattttcagggccgtaaacgctccttctgagtcattcccaataacaagaatttctaccagtaaagctattaacaggcatcaaaataggggagtgctaaattaagatgagattgtaaaagcaaataagaacatacgcagactcgcataggagtgcaaatgatcgtttctgattgaaatgtttatagctaaatgagtttggctgaattaaacacaaatgttccaaaagataagccgtagctggtgcttcttttttctgttttttaagctgctttacagacgaaaatggaactatatttggaacaatgctttctgtttttccatactattgatatttgtggaaagtcacaaaatggcctaaggaagctaagctcgccccaagcagtggtcacttacaagtacttttgtactctgtactcctgtcacatttgggcgatcagagcaacagctggggagactttttcaacaaagatgagtgtcagataatcctgatgagattccacatccaacatcttttgtaattatgtcacattcagctgtaatggaataattcaagctgaaagaacaagctttgatcctttcttaaacctttccctgtggactggctatctaaaagatttaaagatatttctgttacaagatctagtgtttcctcagagaagtcatgcttctgaagcatcgtgatctacaagaacaatatcaagtttgccaaacacatttctgaaagcatcgtgttttggggggaggggttgtatttaatgaagatatcaataatatgctatgcttcaattttcatctaggtgatcaagattcattttcttgttctgtcatccaaataggcagacagaaaagtgattgaaatacattatggagatgtgtcattgcaca

SEQ ID NO: 98 - DNA, 540 bp, synthetic construct
  source 1..540: mol_type = other DNA
gctggtgcttcttttttctgttttttaagctgctttacagacgaaaatggaactatatttggaacaatgctttctgtttttccatactattgatatttgtggaaagtcacaaaatggcctaaggaagctaagctcgccccaagcagtggtcacttacaagtacttttgtactctgtactcctgtcacatttgggcgatcagagcaacagctggggagactttttcaacaaagatgagtgtcagataatcctgatgagattccacatccaacatcttttgtaattatgtcacattcagctgtaatggaataattcaagctgaaagaacaagctttgatcctttcttaaacctttccctgtggactggctatctaaaagatttaaagatatttctgttacaagatctagtgtttcctcagagaagtcatgcttctgaagcatcgtgatctacaagaacaatatcaagtttgccaaacacatttctgaaagcatcgtgttttggggggaggggttgtatttaatgaagatatcaataatatgc

SEQ ID NO: 99 - DNA, 359 bp, synthetic construct
  source 1..359: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgcca

SEQ ID NO: 100 - DNA, 463 bp, synthetic construct
  source 1..463: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcaccggcggtggagaacaaggctctattcagcctgtgcccaggaaaggggatcaggggatgcccaggcatggacagtgggtggcagggggggagaggagggctgtctgcttcccagaagtccaaggacacaaatgggtgagggga

SEQ ID NO: 101 - DNA, 386 bp, synthetic construct
  source 1..386: mol_type = other DNA
ctagcccacaggaaatgtctgtctatatccaggcaagtaccttgctcattggaccaacccgaaactgttcagggaagatcagggaaatcaactcagttacaaatgggataatcatgcccagtaaaaactacctgtggtgaataaagagttaacccctgttccatcttaggtcactatgcagagtaccaatgagtacaagagatggtgccaaagagggtggcccctccctagctgggaacagtcaacccttaggaactagactgtcaacacatcagccagccagagacaagggaaaccgtggcaaccaagtgttgctggcacattgtgaggtggtgatgggaactgcagaggccctgcacagcatgctaatgagcccaggcaaacat

SEQ ID NO: 102 - DNA, 418 bp, synthetic construct
  source 1..418: mol_type = other DNA
tccaaagaaaagccagataagtagctgattattgcatagagctgacagtatcacaggaagatcagtagtagcagctcaagtacaaaaaggttaattagcaatacttaataagaaaaactacctctggcaggtgaagagttaatccctggtcaattttaagctactctgctgagagtactaataagtgtaggggttggagccaatgagggtgaccccttccttgatgggaacagtcatcccttaggaactgccctggaaagcatcagccagccagaaaacagggaaagaggctgagaaaccgtggtaaccaagttttgctggcactttgtaaaatggtaactgcaactgccgaggctgtgcagagaatgctaataagcctaggacaacctgtaaagagtggacctagaaaatgtccacc

SEQ ID NO: 103 - DNA, 502 bp, synthetic construct
  source 1..502: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgagggga

SEQ ID NO: 104 - DNA, 324 bp, synthetic construct
  source 1..324: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcac

SEQ ID NO: 105 - DNA, 121 bp, synthetic construct
  source 1..121: mol_type = other DNA
agcgcgcagagtctgcatgcgtgaggaagctcctgggcgcgtcacagccgcgctattctcagcgtctctccttttatggctccggaagtgagctggggttgctggcagcctggctggcact

SEQ ID NO: 106 - DNA, 393 bp, synthetic construct
  source 1..393: mol_type = other DNA
cttcctcttatatttcaccaagactcagttcctgagcaagaaaccacaggcacagcaagtgccatgaaaagcggctttgtgtggggtgggctcttcacactccaatctccacttccttctcaaggcctcaaaaaaagttgaaaaatgaaaacaaaagccctgctgtgttgagctgggctctggcgttgccatggacccagggcaaacagcggtgctcctgctctgcccccggctcagctcatgctgggcctgcacttctggaagggagcatggactttggaatgactggttagaacccaaatgaattaatggaatttgacatagttcaaaaataataaaatgtgatacccatgaaatgctgatattctgccttaatttgccagattgggggcc

SEQ ID NO: 107 - DNA, 408 bp, synthetic construct
  source 1..408: mol_type = other DNA
ttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaa

SEQ ID NO: 108 - DNA, 499 bp, synthetic construct
  source 1..499: mol_type = other DNA
tcaggggtgcagctttttttctgtcttttactcagcctgagaaaggttgtcgtttgacaaggtttgttcagaggtcggatctgaatccagctccaaggccccagcacccaagccctgaccccatggctgcctgctggctgggagtggcattctttagaacaggaaaggcagtgagttctcattgcatcaatacttgcatttgctacaacagaagctttttgtctcttcctcacattcctttagaacacaagcctccttttctgcctgtggccatcggtcactggagtcagcctcgtgagtggcttggtggcggatggcaccgtcctttgtgggagaaaacaatgttgctgcccaggcctttctggaatgaccccttcccaccccctgaccagccccagcaaggcccggggctggctgcctagttgattggagccaagagtttgctgaatggatgaagggagaagggacatccaagtgactgaagaagatcttaacagaa

SEQ ID NO: 109 - protein, 500 aa, synthetic construct; identical to SEQ ID NO: 2

SEQ ID NO: 110 - DNA, 1500 bp, synthetic construct
  source 1..1500: mol_type = other DNA
atgagccccgggctgctgctgctcggtagcgccgtcctgctcgccttcggcctctgctgcaccttcgtgcaccgcgctcgcagccgctacgagcacatccccgggccgccgcggcccagtttccttctaggacacctcccctgcttttggaaaaaggatgaggttggtggccgtgtgctccaagatgtgtttctagattgggctaagaagtatggacctgtagtgcgggtcaacgtcttccacaaaacctcagtcatcgtcacgagtcctgagtcggttaagaagttcctgatgtcaaccaagtacaacaaggactccaagatgtaccgtgcgctccagactgtgtttggtgagagactcttcggccaaggcttggtgtccgaatgcaactatgagcgctggcacaagcagcggagagtgatagacctggccttcagccggagctccttggttagcttaatggaaacattcaacgaaaaggctgagcagctggtggagattctagaagccaaggcagatgggcagacccctgtgagcatgcaggacatgctgacctacaccgccatggacatcctggccaaggcagcttttgggatggagaccagtatgctgctgggtgcccagaagcctctgtcccaggcagtgaaacttatgttggagggaatcactgcgtcccgcaacactctggcaaagttcctgccagggaagaggaagcagctccgggaggtccgggagagcattcgcttcctgcgccaggtgggcagggactgggtccagcgccgccgggaagccctgaagaggggcgaggaggttcctgccgacatcctcacacagattctgaaagctgaagagggagcccaggacgacgagggtctgctggacaacttcgtcaccttcttcattgctggtcacgagacctctgccaaccacttggcgttcacagtgatggagctgtctcgccagccagagatcgtggcaaggctgcaggccgaggtggatgaagtgattggttctaagaggtacctggatttcgaggacctggggagactgcagtacctgtcccaggtcctcaaagagtcgctgaggctgtacccaccagcatggggcacctttaggctgctggaagaggagaccttgattgatggggtgagagtccccggcaacaccccgctcttgttcagcacctatgtgatggggcggatggacacatactttgaggacccgctgactttcaaccccgatcgcttcggccctggagcacccaagccacggttcacctacttccccttctccctgggccaccgctcctgcatcgggcagcagtttgctcagatggaggtgaaggtggtcatggcaaagctgctgcagaggctggagttccggctggtgcccgggcagcgcttcgggctgcaggagcaggccacactcaagccactggaccccgtgctgtgcaccctgcggccccgcggctggcagcccgcacccccaccacccccctgc

SEQ ID NO: 111 - DNA, 4036 bp, synthetic construct
  source 1..4036: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggcgcgccacgcgtgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattccttaagctatcataggaatgagccccgggctgctgctgctcggtagcgccgtcctgctcgccttcggcctctgctgcaccttcgtgcaccgcgctcgcagccgctacgagcacatccccgggccgccgcggcccagtttccttctaggacacctcccctgcttttggaaaaaggatgaggttggtggccgtgtgctccaagatgtgtttctagattgggctaagaagtatggacctgtagtgcgggtcaacgtcttccacaaaacctcagtcatcgtcacgagtcctgagtcggttaagaagttcctgatgtcaaccaagtacaacaaggactccaagatgtaccgtgcgctccagactgtgtttggtgagagactcttcggccaaggcttggtgtccgaatgcaactatgagcgctggcacaagcagcggagagtgatagacctggccttcagccggagctccttggttagcttaatggaaacattcaacgaaaaggctgagcagctggtggagattctagaagccaaggcagatgggcagacccctgtgagcatgcaggacatgctgacctacaccgccatggacatcctggccaaggcagcttttgggatggagaccagtatgctgctgggtgcccagaagcctctgtcccaggcagtgaaacttatgttggagggaatcactgcgtcccgcaacactctggcaaagttcctgccagggaagaggaagcagctccgggaggtccgggagagcattcgcttcctgcgccaggtgggcagggactgggtccagcgccgccgggaagccctgaagaggggcgaggaggttcctgccgacatcctcacacagattctgaaagctgaagagggagcccaggacgacgagggtctgctggacaacttcgtcaccttcttcattgctggtcacgagacctctgccaaccacttggcgttcacagtgatggagctgtctcgccagccagagatcgtggcaaggctgcaggccgaggtggatgaagtgattggttctaagaggtacctggatttcgaggacctggggagactgcagtacctgtcccaggtcctcaaagagtcgctgaggctgtacccaccagcatggggcacctttaggctgctggaagaggagaccttgattgatggggtgagagtccccggcaacaccccgctcttgttcagcacctatgtgatggggcggatggacacatactttgaggacccgctgactttcaaccccgatcgcttcggccctggagcacccaagccacggttcacctacttccccttctccctgggccaccgctcctgcatcgggcagcagtttgctcagatggaggtgaaggtggtcatggcaaagctgctgcagaggctggagttccggctggtgcccgggcagcgcttcgggctgcaggagcaggccacactcaagccactggaccccgtgctgtgcaccctgcggccccgcggctggcagcccgcacccccaccacccccctgctgagtgatagcttggtaccgagctcgatccaattgcaatgatcatcatgacagatctgcgcgcgatcgatatcagcgctttaaatttgcgcatgcagctatagttctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtttaaacgcggccgcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 112 - DNA, 696 bp, synthetic construct; identical to SEQ ID NO: 91
  source 1..696: mol_type = other DNA

SEQ ID NO: 113 - DNA, 709 bp, synthetic construct; identical to SEQ ID NO: 92
  source 1..709: mol_type = other DNA

SEQ ID NO: 114 - DNA, 768 bp, synthetic construct; identical to SEQ ID NO: 93
  source 1..768: mol_type = other DNA

SEQ ID NO: 115 - DNA, 736 bp, synthetic construct; identical to SEQ ID NO: 94
  source 1..736: mol_type = other DNA

SEQ ID NO: 116 - DNA, 1744 bp, synthetic construct; identical to SEQ ID NO: 95
  source 1..1744: mol_type = other DNA

SEQ ID NO: 117 - DNA, 1104 bp, synthetic construct; identical to SEQ ID NO: 96
  source 1..1104: mol_type = other DNA

SEQ ID NO: 118 - DNA, 1910 bp, synthetic construct; identical to SEQ ID NO: 97
  source 1..1910: mol_type = other DNA

SEQ ID NO: 119 - DNA, 540 bp, synthetic construct; identical to SEQ ID NO: 98
  source 1..540: mol_type = other DNA

SEQ ID NO: 120 - DNA, 982 bp, synthetic construct
  source 1..982: mol_type = other DNA
ctgggcagagagggggcatcgggggcatggctaggggccagcactgtgcttcctgggcgcctcacctcctccctgactcctggagactcccagcccctgtctgggagatgagcatttaggaatctgcttgtgcaggggtggtgggaggggccggggtggagggcgcatccccacggggagattggatggaaatggcctgccagtgtgtgtgtgagtgtgcgcctgtggcagcagcagagtaaacagccgctgccctgtcctctctgcggccgtggccaggtacacaggcctgtttggacagctgccttgtctgtccgtctgtttgggagatgctggctgatagatggggatgggcggactgttaacccctcgttgcctgcactgctatgtgcttcctgcctcatccatggggtagaaggtagccagaaggtggtcctggctgtgcccccagctcctctctaggggggaaacctctagttctgagtcagggacagagtgaggagggctccagggcatcaagagcttgctcctccccgcaccagggagccaaggacagaggagaagggggtcttccccagtggtgactaggggcagaatatgtctctgagtgagtgtctggagccctcctcaccccaacaccatggggctgggcataaaagtcagggcagagccatctattgcttacatttgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcagatgccacc

SEQ ID NO: 121 - DNA, 876 bp, synthetic construct
  source 1..876: mol_type = other DNA
aaggagaatggtaaacagcaggagcgaagcggctgaggagaaagaagaggaaagaaaggcgagacgtgggaggattggaacagagacaaaagggaggagagacggacagcgacaagtggagaaaatcggcgaaacttgagtggcagagaagtctgagcgctgagacccggcggccccgtgcgccttcccacctggcgccgatccactttcctcggggtagcggcccaacccacttcgctgccagccgatcccttttacccgtggctaccgggaccactctactctcgcccacttggctctgcctaagcgtcctagccggagcgcggtctctgccacgtggggaggggcgcggccgagttgctgaagagcgcttctgattggccagagggcggggttcttggcgtctcgccggccagacccctccctcaaaggcggggcctggagatccacagctggaaagggcggagccccagcagggcagctggaaaggggcggggcctgacgcgcgcggctcgccgcggcgggctgggggcgccctggtctgccataaagtgaatgggcgccggctgggggtggcagtacgctcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcagatgccacc

SEQ ID NO: 122 - DNA, 835 bp, synthetic construct
  source 1..835: mol_type = other DNA
ggaacatttagactttgggaaagagattagtgatttacattgctatctcactgatttaatttaaatgctcttccaaaccaaacacacatgtgccgaagaggctactaagaaacccaacatgcagagttctctataagtgcagccgacagtgttgactgaaactaaacttggaaatccagggcactaatgcacaatatcaagcaataaaacggcatctctttggcaatatttaatttaaaaaagaagaaagagacaggcgaagatcaggcactgtctgttttggaggatcaaccattctgcatttcaaagcattggtccctgcaatatccaggttactgtgctagaatctcgactattatatcgcagttgtgagagggagggcaaagatgtgtttactcagtgattaggcccttagaataagcctctagctcctagagagacagctcaccacttattcatttgggccaattcacaaagcctaggaagattaaacatccatgctgagaagacaagcgaatgcagacggtgaaaaagaaataaaaattctttaaaaactctgagatgacttcattatttttccacaaggaaactttaggaaagtgtttagttagagaaaaacccacattgacctctctctaaacccttaatctttcctttgtggtggcactgctttgtggtaagcgactggctcgcctcgcccctcttttcactggaagctgagagaaaaaagactctggagaaacagttttcgttccagggacacaaacccctgacactgttaagggctgggcataaaagtcagggcagagccatctattgcttacatttgcttctg

SEQ ID NO: 123 - DNA, 812 bp, synthetic construct
  source 1..812: mol_type = other DNA
gaaaatttaatgtcattgaagctctatttccaatagcaaaggagcactattgctaatagacttcagagcttgaaataaataaatctttggaatcctgttgcatctcttggggtgtgacatttgacagtcttttatagcacagaacgaaacaagtttgtgagctggaattcaattgtggcgtattgattccttgcatcagtcattattccctgctgattgacaggtgaaaattggttacgttaagtatttcatatgttatattggctgacatttgcttgcctgctcttgtgtcaatattgttgtaaagatctccagctttatgagatagcaatagacactgactgtggcttttgtgtgatgttccagtgtttttcctgacataatttaagacatattaaaaaccagcagcatcttccctcttgagaagcttaatgccaatattattgtcttccaggggaagatcatgtatgctcataatcgggtgctaatttccaccagtacgctcatgtttaggcattaggcactataactgtaaaattgagccttcttgattgattcatgtcaagcctcatctcggctcctgcaggggaagtcatccggctgaccctttttacactaaaagaagagatttgtgttcctttctttcacctggaaccatcaaattgactgaataatctgtaatacattagtgctgacatttgttagggagaattaaacaagacacagtaatcattccccagaataaaaattgtgtttgatgggctgggcataaaagtcagggcagagccatctattgcttacatttgcttctg

SEQ ID NO: 124 - DNA, 487 bp, synthetic construct
  source 1..487: mol_type = other DNA
tgagactgagtgggagtggggatgaagccttttttcttcatttttttttcctcaggtgcaattctgtgttaatataagagaagtgtggccttccttctcatagcactaaaagtgagataatccctgtgtaagaaatcagtaagtacggtctgcttaatctagtcccagtgtgaaactgttgacatttgttcttttttctatcattatgtgactgggcctgttttgtgctggattaggcacaaatctcctatgcagcacatttggcatgttactagtagtttaacttcattaataatgtatgaagaaaatgtaatccatgacaaggaagcaaagaaaagtatttttttttttttttgcttctcccaaatcctttggaatgagtaattattcaacattttatgtttgatgttatattttacaattcaacttccatagggctgggcataaaagtcagggcagagccatctattgcttacatttgcttctg

SEQ ID NO: 125 - DNA, 593 bp, synthetic construct
  source 1..593: mol_type = other DNA
gctggtgcttcttttttctgttttttaagctgctttacagacgaaaatggaactatatttggaacaatgctttctgtttttccatactattgatatttgtggaaagtcacaaaatggcctaaggaagctaagctcgccccaagcagtggtcacttacaagtacttttgtactctgtactcctgtcacatttgggcgatcagagcaacagctggggagactttttcaacaaagatgagtgtcagataatcctgatgagattccacatccaacatcttttgtaattatgtcacattcagctgtaatggaataattcaagctgaaagaacaagctttgatcctttcttaaacctttccctgtggactggctatctaaaagatttaaagatatttctgttacaagatctagtgtttcctcagagaagtcatgcttctgaagcatcgtgatctacaagaacaatatcaagtttgccaaacacatttctgaaagcatcgtgttttggggggaggggttgtatttaatgaagatatcaataatatgcgggctgggcataaaagtcagggcagagccatctattgcttacatttgcttctg

SEQ ID NO: 126 - DNA, 53 bp, synthetic construct
  source 1..53: mol_type = other DNA
gggctgggcataaaagtcagggcagagccatctattgcttacatttgcttctg

SEQ ID NO: 127 - DNA, 553 bp, synthetic construct
  source 1..553: mol_type = other DNA
gggaggattggaacagagacaaaagggaggagagacggacagcgacaagtggagaaaatcggcgaaacttgagtggcagagaagtctgagcgctgagacccggcggccccgtgcgccttcccacctggcgccgatccactttcctcggggtagcggcccaacccacttcgctgccagccgatcccttttacccgtggctaccgggaccactctactctcgcccacttggctctgcctaagcgtcctagccggagcgcggtctctgccacgtggggaggggcgcggccgagttgctgaagagcgcttctgattggccagagggcggggttcttggcgtctcgccggccagacccctccctcaaaggcggggcctggagatccacagctggaaagggcggagccccagcagggcagctggaaaggggcggggcctgacgcgcgcggctcgccgcggcgggctgggggcgccctggtctgccataaagtgaatgggcgccggctgggggtggcagtacgcggtgaggctcactccctccgagagtccaggagcgcc

SEQ ID NO: 128 - DNA, 643 bp, synthetic construct
  source 1..643: mol_type = other DNA
ctgggcagagagggggcatcgggggcatggctaggggccagcactgtgcttcctgggcgcctcacctcctccctgactcctggagactcccagcccctgtctgggagatgagcatttaggaatctgcttgtgcaggggtggtgggaggggccggggtggagggcgcatccccacggggagattggatggaaatggcctgccagtgtgtgtgtgagtgtgcgcctgtggcagcagcagagtaaacagccgctgccctgtcctctctgcggccgtggccaggtacacaggcctgtttggacagctgccttgtctgtccgtctgtttgggagatgctggctgatagatggggatgggcggactgttaacccctcgttgcctgcactgctatgtgcttcctgcctcatccatggggtagaaggtagccagaaggtggtcctggctgtgcccccagctcctctctaggggggaaacctctagttctgagtcagggacagagtgaggagggctccagggcatcaagagcttgctcctccccgcaccagggagccaaggacagaggagaagggggtcttccccagtggtgactaggggcagaatatgtctctgagtgagtgtctggagccctcctcaccccaacaccatg

SEQ ID NO: 129 - DNA, 656 bp, synthetic construct
  source 1..656: mol_type = other DNA
ggtgtgtggaagggtgagaggcacacacacagacactgaaagaatcctaggcctggtaggcacttaacaaatgtctgttacagaccagaattttattgctgttagagacccaagcccctcataggaacagtgagaaacaggtgcagaaaggcggagtaactttatctaaagtcataggctccctgaatagcagagctgacacctacaaggaagcgttggagaccagatctaccagctagcctccctgagaccacgaggtggcgccgcagcaccggctgtggccgatgccagccaggtagccggtttcccacgtcccccgcacgcacgcacctctttgctgcaggaatcccgggctgccccgacctggagtagggggggtggtgagtgggactgagtccctagaagcctggaccctcacttcgttcctgtacatccagctcgcctgtagacagtgggggaggatgaagggaagaggactcaagcgcaactttgaatcatcacgccttcgacagtccgcgcacgtttatttcatttatctttgaaaacgagggaggggaagcctggagaaggcgggatgggccaagggtgagttggcccccggggagctggtccctgttcctggctttagtcccaggggcgcggtctgtgtgtagggc

SEQ ID NO: 130 - DNA, 215 bp, synthetic construct
  source 1..215: mol_type = other DNA
tgctaccagagccgggagagctgctcggagacgcctccggggtgcgggctggacatgagcagcggctgccggtcctgggactaggccccgccattttggatccgctgacaggtcagcgaagtctcttcctagagttccggtgtcgtgaaggccgccctgacatcgcaatagggaattagtgggaagggcccttaaattgggcgagccaaggtggg

SEQ ID NO: 131 - DNA, 782 bp, synthetic construct
  source 1..782: mol_type = other DNA
ggaacatttagactttgggaaagagattagtgatttacattgctatctcactgatttaatttaaatgctcttccaaaccaaacacacatgtgccgaagaggctactaagaaacccaacatgcagagttctctataagtgcagccgacagtgttgactgaaactaaacttggaaatccagggcactaatgcacaatatcaagcaataaaacggcatctctttggcaatatttaatttaaaaaagaagaaagagacaggcgaagatcaggcactgtctgttttggaggatcaaccattctgcatttcaaagcattggtccctgcaatatccaggttactgtgctagaatctcgactattatatcgcagttgtgagagggagggcaaagatgtgtttactcagtgattaggcccttagaataagcctctagctcctagagagacagctcaccacttattcatttgggccaattcacaaagcctaggaagattaaacatccatgctgagaagacaagcgaatgcagacggtgaaaaagaaataaaaattctttaaaaactctgagatgacttcattatttttccacaaggaaactttaggaaagtgtttagttagagaaaaacccacattgacctctctctaaacccttaatctttcctttgtggtggcactgctttgtggtaagcgactggctcgcctcgcccctcttttcactggaagctgagagaaaaaagactctggagaaacagttttcgttccagggacacaaacccctgacactgttaa

SEQ ID NO: 132 - DNA, 759 bp, synthetic construct
  source 1..759: mol_type = other DNA
gaaaatttaatgtcattgaagctctatttccaatagcaaaggagcactattgctaatagacttcagagcttgaaataaataaatctttggaatcctgttgcatctcttggggtgtgacatttgacagtcttttatagcacagaacgaaacaagtttgtgagctggaattcaattgtggcgtattgattccttgcatcagtcattattccctgctgattgacaggtgaaaattggttacgttaagtatttcatatgttatattggctgacatttgcttgcctgctcttgtgtcaatattgttgtaaagatctccagctttatgagatagcaatagacactgactgtggcttttgtgtgatgttccagtgtttttcctgacataatttaagacatattaaaaaccagcagcatcttccctcttgagaagcttaatgccaatattattgtcttccaggggaagatcatgtatgctcataatcgggtgctaatttccaccagtacgctcatgtttaggcattaggcactataactgtaaaattgagccttcttgattgattcatgtcaagcctcatctcggctcctgcaggggaagtcatccggctgaccctttttacactaaaagaagagatttgtgttcctttctttcacctggaaccatcaaattgactgaataatctgtaatacattagtgctgacatttgttagggagaattaaacaagacacagtaatcattccccagaataaaaattgtgtttgat

SEQ ID NO: 133 - DNA, 434 bp, synthetic construct
  source 1..434: mol_type = other DNA
tgagactgagtgggagtggggatgaagccttttttcttcatttttttttcctcaggtgcaattctgtgttaatataagagaagtgtggccttccttctcatagcactaaaagtgagataatccctgtgtaagaaatcagtaagtacggtctgcttaatctagtcccagtgtgaaactgttgacatttgttcttttttctatcattatgtgactgggcctgttttgtgctggattaggcacaaatctcctatgcagcacatttggcatgttactagtagtttaacttcattaataatgtatgaagaaaatgtaatccatgacaaggaagcaaagaaaagtatttttttttttttttgcttctcccaaatcctttggaatgagtaattattcaacattttatgtttgatgttatattttacaattcaacttccata

SEQ ID NO: 134 - DNA, 540 bp, synthetic construct; identical to SEQ ID NO: 98
  source 1..540: mol_type = other DNA

SEQ ID NO: 135 - DNA, 20 bp, synthetic construct
  source 1..20: mol_type = other DNA
tgctgctgctgctgctgctg

SEQ ID NO: 136 - DNA, 20 bp, synthetic construct
  source 1..20: mol_type = other DNA
ggcggcggcggcggcggcgg

SEQ ID NO: 137 - DNA, 20 bp, synthetic construct
  source 1..20: mol_type = other DNA
gaggggtggggaggctgggg

SEQ ID NO: 138 - DNA, 20 bp, synthetic construct
  source 1..20: mol_type = other DNA
tccttgacctgctgctgcag

SEQ ID NO: 139 - DNA, 20 bp, synthetic construct
  source 1..20: mol_type = other DNA
ccttccactggccatgatgc

SEQ ID NO: 140 - DNA, 20 bp, synthetic construct
  source 1..20: mol_type = other DNA
actgtgccactatgttttca

SEQ ID NO: 141 - DNA, 20 bp, synthetic construct
  source 1..20: mol_type = other DNA
tgaggtatcagattgtctag

SEQ ID NO: 142 - DNA, 20 bp, synthetic construct
  source 1..20: mol_type = other DNA
aaattaatctcttacctgat

SEQ ID NO: 143 - DNA, 20 bp, synthetic construct
  source 1..20: mol_type = other DNA
cccagggctagcaaggaaca

SEQ ID NO: 144 - DNA, 20 bp, synthetic construct
  source 1..20: mol_type = other DNA
aattcagtagcttcccttaa

SEQ ID NO: 145 - DNA, 20 bp, synthetic construct
  source 1..20: mol_type = other DNA
ctgggcccgcagcggaaggg

SEQ ID NO: 146 - DNA, 20 bp, synthetic construct
  source 1..20: mol_type = other DNA
ttattgctgtctactatccg

SEQ ID NO: 147 - DNA, 20 bp, synthetic construct
  source 1..20: mol_type = other DNA
tcagtccttcccaaagctct

SEQ ID NO: 148 - DNA, 20 bp, synthetic construct
  source 1..20: mol_type = other DNA
taatctctttactgatataa

SEQ ID NO: 149 - DNA, 20 bp, synthetic construct
  source 1..20: mol_type = other DNA
tcagcagtgttatttcttac

SEQ ID NO: 150 - DNA, 22 bp, synthetic construct
  source 1..22: mol_type = other DNA
aaaccgttaccattactgagtt

SEQ ID NO: 151 - DNA, 21 bp, synthetic construct
  source 1..21: mol_type = other DNA
aaatcgctgatttgtgtagtc

SEQ ID NO: 152 - DNA, 467 bp, synthetic construct
  source 1..467: mol_type = other DNA
gagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcg

SEQ ID NO: 153 - protein, 13 aa, synthetic construct
SQAGASDIRDQSR

SEQ ID NO: 154 - protein, 13 aa, synthetic construct
  VARIANT 2: note = Any amino acid
  VARIANT 5: note = Any amino acid
  VARIANT 7: note = Any amino acid
  VARIANT 8: note = Any amino acid
  VARIANT 9: note = Any amino acid
  VARIANT 10: note = Any amino acid
  VARIANT 12: note = Any amino acid
SXAGXSXXXXQXR

SEQ ID NO: 155 - protein, 4 aa, synthetic construct
EYSW

SEQ ID NO: 156 - DNA, 120 bp, Homo sapiens
  source 1..120: mol_type = genomic DNA
cagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 157 - DNA, 117 bp, Homo sapiens
  source 1..117: mol_type = genomic DNA
  misc_difference 1..117: note = This sequence may encompass 36-39 CAG repeats
cagcagcagcagcagcagcagcagcagcagcagcagcagcagcagcagcagcagcagcagcagcagcagcagcagcagcagcagcagcagcagcagcagcagcagcagcagcagcag

SEQ ID NO: 158 - RNA, 19 nt, synthetic construct
  source 1..19: mol_type = other RNA
cgctcaggttctgctttta

SEQ ID NO: 159 - RNA, 19 nt, synthetic construct
  source 1..19: mol_type = other RNA
gcccagagccccattcatt

SEQ ID NO: 160 - RNA, 19 nt, synthetic construct
  source 1..19: mol_type = other RNA
gggacgggtccaagatgga

SEQ ID NO: 161 - RNA, 19 nt, synthetic construct
  source 1..19: mol_type = other RNA
gctgcaccgaccaaagaaa

SEQ ID NO: 162 - RNA, 19 nt, synthetic construct
  source 1..19: mol_type = other RNA
gagccgctgcaccgaccaa

SEQ ID NO: 163 - RNA, 19 nt, synthetic construct
  source 1..19: mol_type = other RNA
gcaccgaccaaagaaagaa

SEQ ID NO: 164 - RNA, 21 nt, synthetic construct
  source 1..21: mol_type = other RNA
taaaagcagaacctgagcggc

SEQ ID NO: 165 - RNA, 21 nt, synthetic construct
  source 1..21: mol_type = other RNA
aatgaatggggctctgggccg

SEQ ID NO: 166 - RNA, 21 nt, synthetic construct
  source 1..21: mol_type = other RNA
tccatcttggacccgtcccgg

SEQ ID NO: 167 - RNA, 21 nt, synthetic construct
  source 1..21: mol_type = other RNA
tttctttggtcggtgcagcgg

SEQ ID NO: 168 - RNA, 21 nt, synthetic construct
  source 1..21: mol_type = other RNA
ttggtcggtgcagcggctcct

SEQ ID NO: 169 - RNA, 21 nt, synthetic construct
  source 1..21: mol_type = other RNA
ttctttctttggtcggtgcag

SEQ ID NO: 170 - RNA, 19 nt, synthetic construct
  source 1..19: mol_type = other RNA
ggatggcggtaaccctgca

SEQ ID NO: 171 - RNA, 19 nt, synthetic construct
  source 1..19: mol_type = other RNA
ggtcagattgtaagtattt

SEQ ID NO: 172 - RNA, 19 nt, synthetic construct
  source 1..19: mol_type = other RNA
gcaaacccttgttctcatt

SEQ ID NO: 173 - RNA, 19 nt, synthetic construct
  source 1..19: mol_type = other RNA
ggacatagacagtaagcaa

SEQ ID NO: 174 - RNA, 19 nt, synthetic construct
  source 1..19: mol_type = other RNA
ggagagcagtgttagctta

SEQ ID NO: 175 - RNA, 19 nt, synthetic construct
  source 1..19: mol_type = other RNA
gctgcaccgaccgtgagtt

SEQ ID NO: 176 - RNA, 19 nt, synthetic construct
  source 1..19: mol_type = other RNA
gcaccgaccgtgagtttgg

SEQ ID NO: 177 - RNA, 19 nt, synthetic construct
  source 1..19: mol_type = other RNA
gccgctgcaccgaccgtga

SEQ ID NO: 178 - RNA, 21 nt, synthetic construct
  source 1..21: mol_type = other RNA
tgcagggttaccgccatcccc

SEQ ID NO: 179 - RNA, 21 nt, synthetic construct
  source 1..21: mol_type = other RNA
aaatacttacaatctgaccat

SEQ ID NO: 180 - RNA, 21 nt, synthetic construct
  source 1..21: mol_type = other RNA
aatgagaacaagggtttgcaa

SEQ ID NO: 181 - RNA, 21 nt, synthetic construct
  source 1..21: mol_type = other RNA
ttgcttactgtctatgtcctt

SEQ ID NO: 182 - RNA, 21 nt, synthetic construct
  source 1..21: mol_type = other RNA
taagctaacactgctctccga

SEQ ID NO: 183 - RNA, 21 nt, synthetic construct
  source 1..21: mol_type = other RNA
aactcacggtcggtgcagcgg

SEQ ID NO: 184 - RNA, 21 nt, synthetic construct
  source 1..21: mol_type = other RNA
ccaaactcacggtcggtgcag

SEQ ID NO: 185 - RNA, 21 nt, synthetic construct
  source 1..21: mol_type = other RNA
tcacggtcggtgcagcggctc

SEQ ID NO: 186 - DNA, 38 bp, synthetic construct
  source 1..38: mol_type = other DNA
ggccacgcgtcgactagtactttttttttttttttttt